(19) United States
(12) Patent Application Publication
(10) Pub. No.: US 2023/0339900 A1
(43) Pub. Date: Oct. 26, 2023

(54) CASEIN KINASE 1 DELTA MODULATORS

(71) Applicant: Janssen Pharmaceutica NV, Beerse (BE)

(72) Inventors: Terry Patrick Lebold, San Diego, CA (US); Stefan McCarver, San Diego, CA (US); Kelly J. McClure, Ramona, CA (US); Cathy Preville, San Diego, CA (US); Mark Seierstad, Escondido, CA (US); Brock T. Shireman, Poway, CA (US); Jocelyn Wang, San Diego, CA (US); Dongpei Wu, San Diego, CA (US); Wei Zhang, San Diego, CA (US); Jeannie M. Ziff, San Diego, CA (US)

(21) Appl. No.: 18/026,608

(22) PCT Filed: Sep. 16, 2021
(86) PCT No.: PCT/IB2021/058447

§ 371 Date: Mar. 16, 2023

Related U.S. Application Data
(60) Provisional application No. 63/079,812, filed on Sep. 17, 2020.

(51) Int. Cl. C07D 401/14; C07D 471/04; C07D 487/04; C07D 495/04

(52) U.S. Cl. CPC C07D 401/14; C07D 471/04; C07D 487/04; C07D 495/04

(57) ABSTRACT

A compound of Formula (I) or Formula (II), pharmaceutical compositions containing them, methods of making them, and methods of using them including methods for treating disease states, disorders, and conditions associated with casein kinase 1 delta (CSNK1D) modulation, such as those associated with mood/psychiatric disorders, neurodegenerative diseases, cancers, addiction and substance abuse disorders, pain, and metabolic diseases.

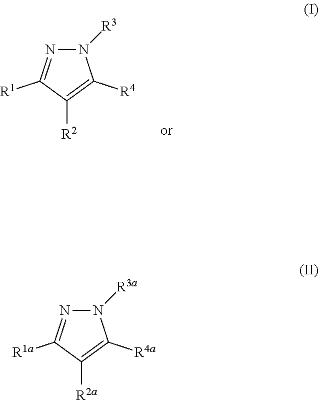

Wherein R¹, R², R³, R⁴, R^{1a}, R^{2a}, R³, and R^{4a} are defined herein.

### FIELD OF THE INVENTION

The present invention is related to certain chemical entities having Casein kinase 1 delta (CSNK1D) modulating properties, pharmaceutical compositions comprising these chemical entities, chemical processes for preparing these chemical entities and their use in the treatment of diseases, disorders, or conditions.

### BACKGROUND OF THE INVENTION

Disruption of the circadian rhythm is a major hallmark in mood disorders. Dampened and phase-shifted temperature, activity, and hormonal rhythms are frequently reported in major depressive disorder (MDD) and bipolar disorder (Hickie, I. B., et al., Manipulating the sleep-wake cycle and circadian rhythms to improve clinical management of major depression. BMC Med, 2013. 11: p. 79; Germain, A. and D. J. Kupfer, Circadian rhythm disturbances in depression. Hum Psychopharmacol, 2008. 23(7): p. 571-85). Depression symptoms are also diurnal with the most severe symptoms occurring typically in the morning (Rusting, C. L. and R. J. Larsen, Diurnal patterns of unpleasant mood: associations with neuroticism, depression, and anxiety. J Pers, 1998. 66(1): p. 85-103), and depression is more prevalent in areas of the world that receive little sunlight for extended periods of time (Booker, J. M., et al., Seasonal depression and sleep disturbances in Alaska and Siberia: a pilot study. Arctic Med Res, 1991. Suppl: p. 281-4). One of the most common mood disorders is seasonal affective disorder (SAD), a syndrome where depressive symptoms occur only in the winter months when there are shorter days and a later dawn (Lam, R. W. and R. D. Levitan, Pathophysiology of seasonal affective disorder: a review. J Psychiatry Neurosci, 2000. 25(5): p. 469-80; Magnusson, A. and D. Boivin, Seasonal affective disorder: an overview. Chronobiol Int, 2003. 20(2): p. 189-207). Therefore, identifying mechanisms that correct these circadian disruptions may have the added therapeutic benefit of attenuating mood disorders.

Many circadian genes have been associated with mood disorders (Benedetti, F., et al., Influence of CLOCK gene polymorphism on circadian mood fluctuation and illness recurrence in bipolar depression. Am J Med Genet B Neuropsychiatr Genet, 2003. 123B(1): p. 23-6; Soria, V., et al., Differential association of circadian genes with mood disorders: CRY1 and NPAS2 are associated with unipolar major depression and CLOCK and VIP with bipolar disorder. Neuropsychopharmacology, 2010. 35(6): p. 1279-89). Pre-clinically, disruption of the suprachiasmatic nucleus (SCN) molecular clock by knocking down Bmal1 expression in the SCN resulted in a dampening and lengthening of SCN PER2:LUC rhythms in mice and lengthened wheel-running rhythms (Landgraf, D., et al., Genetic Disruption of Circadian Rhythms in the Suprachiasmatic Nucleus Causes Helplessness, Behavioral Despair, and Anxiety-like Behavior in Mice. Biol Psychiatry, 2016. 80(11): p. 827-835). Most notably, disruption of SCN molecular rhythms increased depression-like behavior in the learned helplessness and tail suspension tests (Ko, C. H. and J. S. Takahashi, Molecular components of the mammalian circadian clock. Hum Mol Genet, 2006. 15 Spec No 2: p. R271-7; Reppert, S. M. and D. R. Weaver, Molecular analysis of mammalian circadian rhythms. Annu Rev Physiol, 2001. 63: p. 647-76). Additionally, SCN BMAL1 knockdown increased anxiety-like behavior in the light/dark box. Together, these findings suggest that reduced amplitude and increased period of SCN molecular rhythms can cause increased depression and anxiety-like behavior.

The primary molecular clock that controls circadian rhythm is in the SCN in the hypothalamus and consists of a transcriptional feedback loop which cycles over the course of approximately 24 hours (Ko, C. H. and J. S. Takahashi, Molecular components of the mammalian circadian clock. Hum Mol Genet, 2006. 15 Spec No 2: p. R271-7; Reppert, S. M. and D. R. Weaver, Molecular analysis of mammalian circadian rhythms. Annu Rev Physiol, 2001. 63: p. 647-76). The major transcriptional activator consists of a dimer between the Circadian Locomotor Output Cycles Kaput Protein (CLOCK) and Brain and Muscle ARNT-like Protein 1 (BMAL1). This complex binds to the promoters of many genes including the Period (Per) and Cryptochrome (Cry) genes. CRY and PER proteins form a heterodimer in the cytoplasm and translocate into the nucleus where they repress the actions of CLOCK/BMAL1, thus creating a negative feedback loop whose timing is regulated by numerous kinases. Casein kinase 1 delta (CSNK1D) is known to modulate the various feedback loops of the internal canonical circadian clock by phosphorylating PER2. A previous report has demonstrated that two distinct CSNK1D inhibitors PF-670462 and PF-5006739 significantly lengthened circadian rhythms in both cellular reporter assays and in vivo locomotor activity of a variety of species (Wager Travis T. et al., Casein Kinase 1 δ/ε Inhibitor PF-5006739 Attenuates Opioid Drug-Seeking Behavior, 2014 Dec. 17; 5(12): p. 1253-65). Period lengthening mediated by CSNK1D inhibition was accompanied by increased nuclear retention and localization of PER2 protein both in vitro and in vivo (Meng, Q. J., et al., Entrainment of disrupted circadian behavior through inhibition of casein kinase 1 (CK1) enzymes. Proc Natl Acad Sci USA, 2010. 107(34): p. 15240-5; Smyllie, N.J., et al., Visualizing and Quantifying Intracellular Behavior and Abundance of the Core Circadian Clock Protein PERIOD2. Curr Biol, 2016. 26(14): p. 1880-6). With the potential ability to normalize circadian disruption and the sleep/wake cycle in various mood disorders and sleep disturbances, small molecule inhibitors targeted towards the CSNK1D may possess therapeutic utility in a number of mood disorders including type 1 bipolar depression, type 2 bipolar depression, seasonal affective disorder, post-traumatic stress disorder, generalized anxiety disorder, dysthymia, obsessive compulsive disorder, schizophrenia, schizoaffective disorder, mixed episode bipolar disease, major depressive disorder, premenstrual dysphoric disorder, jet lag syndrome, familial advanced sleep phase syndrome, delayed sleep phase syndrome, non-24 hour sleep-wake phase disorder irregular sleep-wake rhythm disorder.

Casein kinases are a group of evolutionarily conserved serine/threonine kinases ubiquitously expressed in eukaryotes. This group includes two families: casein kinase 1 (CK1) and casein kinase 2 (CK2). Six different CK1 genes, CK1 α, γ1, γ2, γ3, δ, and ε have been identified in humans. Each isoform consists of a highly conserved kinase domain followed by a highly variable C-terminal non-catalytic domain. Members of the CK1 family are monomeric, constitutively active, co-factor independent kinases (Knippschild, U., et al., The casein kinase 1 family: participation in multiple cellular processes in eukaryotes. Cell Signal, 2005. 17(6): p. 675-89). By phosphorylating different substrates, such as cellular enzymes, transcriptional proteins, cytoskeletal and non-cytoskeletal proteins, viral oncogenes, and receptors, CK1 regulates diverse cellular processes, including cellular signaling, vesicular trafficking, cell division, and DNA repair pathways and circadian rhythms (Knippschild, U., et al., The casein kinase 1 family: participation in multiple cellular processes in eukaryotes. Cell Signal, 2005. 17(6): p. 675-89; Bischof, J., et al., CK1 delta kinase activity is modulated by Chk1-mediated phosphorylation. PLoS One, 2013. 8(7): p. e68803; Schittek, B. and T. Sinnberg, Biological functions of casein kinase 1 isoforms and putative roles in tumorigenesis. Mol Cancer, 2014. 13: p. 231). Due to its role in tumor progression, small molecule inhibitors targeting CSNK1D may exhibit therapeutic utility in several cancers including gastroenteric, breast, renal, skin, hematological, colorectal, pancreatic, prostate, ovarian, bladder, liver, head/neck.

Genetic studies have shown an important role for casein kinase action on PER proteins in regulating circadian period. A mutation in the Syrian hamster CK1ε gene, tau, shortens the circadian period of behavioral rhythms. Biochemically, the tau mutation (CK1ε^{tau}, a T178C substitution) differentially affects the activity of the kinase protein, reducing general kinase activity while increasing activity at specific residues of the PER proteins (Gallego, M., et al., An opposite role for tau in circadian rhythms revealed by mathematical modeling. Proc Natl Acad Sci USA, 2006. 103(28): p. 10618-23; Lowrey, P. L., et al., Positional syntenic cloning and functional characterization of the mammalian circadian mutation tau. Science, 2000. 288(5465): p. 483-92). The tau mutation is a gain-of-function mutation with respect to circadian substrates, resulting in decreased PER stability and a reduction in circadian period length in tau mutant hamsters and mice (Gallego, M., et al., An opposite role for tau in circadian rhythms revealed by mathematical modeling. Proc Natl Acad Sci USA, 2006. 103(28): p. 10618-23; Meng, Q. J., et al., Setting clock speed in mammals: the CK1 epsilon tau mutation in mice accelerates circadian pacemakers by selectively destabilizing PERIOD proteins. Neuron, 2008. 58(1): p. 78-88). In humans, familial advanced sleep phase syndrome (FASPS) is a circadian-based sleep disorder, in which affected individuals have a short circadian period and an advanced phase of the sleep-wake cycle. One study identified a FASPS pedigree with a mutation in human PER2 (hPER2; S662G mutation); this mutation prevents a priming phosphorylation, thus preventing CK1-mediated phosphorylation (Toh, K. L., et al., An hPer2 phosphorylation site mutation in familial advanced sleep phase syndrome. Science, 2001. 291(5506): p. 1040-3). A second study identified a dominant mutation within the kinase domain of CSNK1D in a family with FASPS (Xu, Y., et al., Functional consequences of a CK1 delta mutation causing familial advanced sleep phase syndrome. Nature, 2005. 434(7033): p. 640-4). Modeling this mutation in mice also revealed alterations in period length.

CSNK1D has been linked to neurodegenerative disorders, including Alzheimer's disease (AD) Parkinson's disease (PD) and frontotemporal dementia (FTD). In particular, brain tissue from AD patients have been shown to express CSNK1D mRNA levels 30-fold higher than normal cells (Flajolet, M., et al., Regulation of Alzheimer's disease amyloid-beta formation by casein kinase I. Proc Natl Acad Sci USA, 2007. 104(10): p. 4159-64). β-Amyloid protein, present in a misfolded insoluble form in AD cells, has been shown to stimulate CSNK1D activity. Altogether these conditions promote an abnormal phosphorylation of tau protein, which is an AD-related substrate of the CSNK1D isoform. Recent reports have also highlighted a potential role of CSNK1D in neurological pathologies including Parkinson's and amyotrophic lateral sclerosis (Nonaka, T., et al., Phosphorylation of TAR DNA-binding Protein of 43 kDa (TDP-43) by Truncated Casein Kinase 1 delta Triggers Mislocalization and Accumulation of TDP-43. J Biol Chem, 2016. 291(11): p. 5473-83; Morales-Garcia, J. A., et al., Biological and Pharmacological Characterization of Benzothiazole-Based CK-1 delta Inhibitors in Models of Parkinson's Disease. ACS Omega, 2017. 2(8): p. 5215-5220). Since CSNK1D has been linked to both circadian disruption in neurodegenerative disorders and direct hyperphosphorylation of tau, α-synuclein and TDP-43, both disease modifying and symptomatic approaches can be explored for therapeutic utility in neurodegenerative disorders including those listed previously and also Down Syndrome, Progressive supranuclear palsy, Parkinsonism dementia complex of Guam, and Pick's Disease.

Small molecule inhibitors targeting CSNK1D may also attenuate addiction/substance abuse. Previous reports have implicated CSNK1D in addiction/substance abuse due to its phosphorylation/regulation of the protein cAMP-regulated neuronal phosphoprotein 32 (DARPP-32) (Nairn, A. C., et al., The role of DARPP-32 in the actions of drugs of abuse. Neuropharmacology, 2004; 47 (Suppl. 1), p. 14-23; Falcon, E., McClung, C. A., A role for the circadian genes in drug addiction. Neuropharmacology, 2009. 56 (Suppl. 1): p. 91-96). Additional reports have demonstrated that commercially available small molecule inhibitors PF-670462 of CSNK1D have shown efficacy in a number of addiction/substance abuse models including conditioned place preference with cocaine and alcohol reinstatement (Abaca C., Albrecht U., Spangel, R. Cocaine sensitization and reward are under the influence of circadian genes and rhythm. Proc. Natl. Acad. Sci. 2002. 99 (13), p. 9026-9030; Spangel, R., et al., The clock gene Per2 influences the glutamatergic system and modulates alcohol consumption. Nat. Med. 2005. 11 (1), p. 35-4; Perreu-Lenz, Vengeliene, V., et al., Inhibition of the casein kinase 1 epsilon delta prevents relapse like alcohol drinking. Neuropsychopharmacology 2012, 37 (9) p. 2121-2131). Further published accounts have reported that PF-5006739 was also efficacious in attenuating fentanyl self-administration (Wager Travis T. et al., Casein Kinase 1 δ/ε Inhibitor PF-5006739 Attenuates Opioid Drug-Seeking Behavior, 2014 Dec. 17; 5(12): p. 1253-65). Therefore, compounds that are synthesized to inhibit the activity of CSNK1D may exhibit therapeutic utility in a number of addictive/substance abuse indications involving chemicals (cocaine, opiate, tobacco, alcohol, amphetamines, inhalants, phencyclidine), impulse control disorders (intermittent explosive disorder, kleptomania, pyromania, gambling) and behavioral disturbances (food, sex, shopping, cutting, exercising, pain seeking).

Recently, published reports have also indicated a potential role for CSNK1D in the pathogenesis of various metabolic disorders. In the ob/ob and diet-induced obese mouse (two models of metabolic dysfunction), daily administration of the CSNK1D inhibitor PF-5006739 improve glucose tolerance (Cunningham, P. S., et al. Targeting the circadian clock via CK1d/e to improve glucose homeostasis in obesity. Sci Rep. 2016, 6, p. 29983). In addition, when a human adipocyte cell line was treated with CSNK1D specific inhibitors, increased basal and insulin-stimulated glucose uptake was measured (Xu, P., et al., Gene expression levels of Casein kinase 1 (CK1) isoforms are correlated to adiponectin levels in adipose tissue of morbid obese patients and site-specific phosphorylation mediated by CK1 influences multimerization of adiponectin. Mol Cell Endocrinol; 2015, 406: p. 87-101). Therefore, small molecule inhibitors may exert beneficial effects on glucose utilization in a number of metabolic diseases including Type 1 diabetes mellitus, idiopathic, type 2 diabetes mellitus, genetic defect of B-cell function, genetic defects of insulin action (type A insulin resistance, leprechaunism, Rabson-Mendahall syndrome, lipoatrophic diabetes), disease of exocrine pancreas (pancreatitis, neoplasia, trauma, cystic fibrosis, hemochromatosis, fibrocalculous pancreatopathy), endocrinopathies (Acromegaly, Cushing's syndrome, Glucagonoma, Pheochromocytoma, Hyperthyroidism, Somatostatinoma, Aldosteronoma), drug/chemical induced (Vacor, Pentamidine, Nicotinic acid, Glucocorticoids, Thyroid hormone, Diazoxide, 3-adrenergic agonists, Thiazides, Dilantin, ∝-Interferon), infections (congenital rubella, cytomegalovirus) uncommon forms (“stiff-man” syndrome, anti-insulin receptor antibodies), genetic syndromes (Down's syndrome, Klinefelter's syndrome, Turner's syndrome, Wolfram's syndrome, Friedreich's ataxia, Huntington's chorea, Laurence-Moon-Biedl syndrome, Myotonic dystrophy, Porphyria, Prader-Willi syndrome), gestational diabetes mellitus.

Small molecule inhibitors of CSNK1D have also been shown to be efficacious in a variety of pre-clinical pain models including von Frey to assess mechanical allodynia and also a model of inflammatory pain (Young, E. E., et al., Systems genetic and pharmacological analysis identifies candidate genes underlying mechanosensation in the von Frey test. Genes Brain Behav; 2016, 15(6): p. 604-615 and Kurihara, T., et al., Alleviation of behavioral hypersensitivity in mouse models of inflammatory pain with two structurally different casein kinase 1 (CK1) inhibitors. Mol Pain. 2014; 10: p. 17). Therefore, translational elements for developed small molecule inhibitors of CSNK1D may also be therapeutically beneficial in a number of pain indications including nociceptive (arthritis, mechanical back pain, post-surgical pain), inflammatory (gout, rheumatoid arthritis), neuropathic (neuropathy, radicular pain, trigeminal neuralgia), and functional (fibromyalgia, irritable bowel syndrome).

### SUMMARY OF THE INVENTION

Embodiments of the present invention relate to chemical entities, pharmaceutical compositions containing them, methods of making and purifying them, and methods for using them for the treatment of diseases, disorders, and conditions associated with CSNK1D modulation. An additional embodiment of the invention is a method of treating a subject suffering from or diagnosed with a disease, disorder, or condition associated with CSNK1D modulation using at least one chemical entity of the invention.

Additional embodiments, features, and advantages of the invention will be apparent from the following detailed description and through practice of the invention.

Embodiments of this Invention are Compounds of Formula (I),

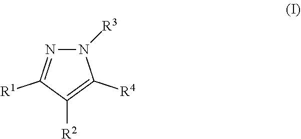

wherein
R¹ is selected from the group consisting of:
(a) 5-membered heteroaryl selected from the group consisting of:

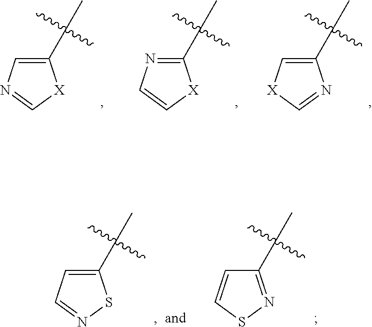

(b) pyridinyl substituted with one or two halo members;
(c) pyrimidinyl; pyrimidinyl substituted with halo; pyrazinyl; pyrazinyl substituted with C₁₋₆ alkyl; pyridazinyl; and pyridazinyl substituted with C₁₋₆ alkyl;
R² is selected from the group consisting of:
(d)

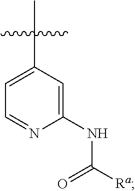

(e)

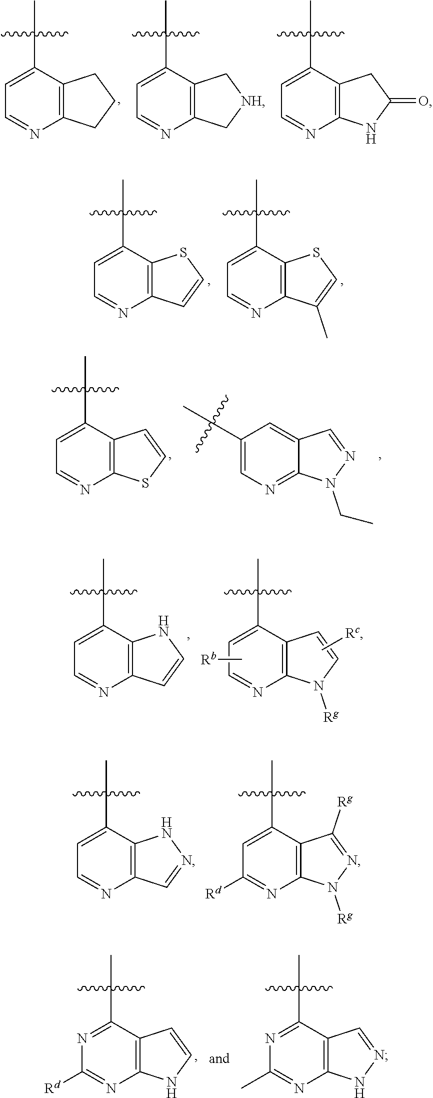

wherein
R^{a} is C₁₋₃ alkyl or C₃₋₆ cycloalkyl;
R^{b} is selected from the group consisting of: H, halo, C₁₋₃ alkyl, C₁₋₃ haloalkyl, CN, CH₂CN, NH₂, oxetanyl, and CH₂-oxetanyl;
R^{c} is selected from the group consisting of: H, halo, C₁₋₃ alkyl, C₁₋₃ haloalkyl, CN, CH₂CN, oxetanyl, oxetanyl substituted with OH, tetrahydrofuranyl substituted with OH, and CH₂-tetrahydrofuranyl;
R^{d} is selected from the group consisting of: H, C₁₋₃ alkyl, C₁₋₃ haloalkyl, and C₃₋₆ cycloalkyl;
R^{g} is H or C₁₋₃ alkyl;
X is O, S, or N—CH₃;
R³ is selected from the group consisting of: C₁₋₆ alkyl, CH₂CH₂OCH₃, C₁₋₆ haloalkyl, C₃₋₆ cycloalkyl, oxetanyl, CH₂-oxetanyl, and tetrahydrofuranyl; and
R⁴ is selected from the group consisting of: H, C₁₋₃ alkyl, and C₃₋₆ cycloalkyl; and pharmaceutically acceptable salts, isotopes, N-oxides, solvates, and stereoisomers of compounds of Formula (I).

### DETAILED DESCRIPTION OF THE INVENTION

As used herein, the terms “including”, “containing” and “comprising” are used in their open, non-limiting sense.

Unless qualified specifically in particular instances of use, the term “alkyl” refers to a straight- or branched-chain alkyl group having from 1 to 8 carbon atoms in the chain. Examples of alkyl groups include methyl (Me), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples. “C₁₋₆ alkyl” refers to straight- or branched-chain alkyl group having from 1 to 6 carbon atoms in the chain. “C₁₋₃ alkyl” refers to straight- or branched-chain alkyl group having from 1 to 3 carbon atoms in the chain.

The term “cycloalkyl” refers to a saturated or partially saturated, monocyclic, fused polycyclic, or spiro polycyclic carbocycle having from 3 to 12 ring atoms per carbocycle. Illustrative examples of cycloalkyl groups include the following entities, in the form of properly bonded moieties:

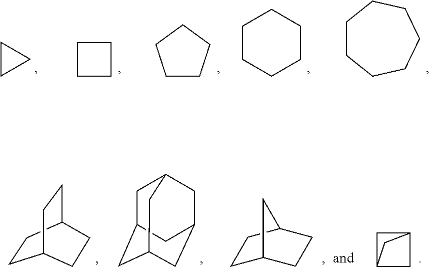

The term “halogen” or “halo” represents chlorine, fluorine, bromine, or iodine.

The term “haloalkyl” refers to a straight- or branched-chain alkyl group having from 1 to 6 carbon atoms in the chain optionally substituting hydrogens with halogens. The term “C₁₋₄ haloalkyl” as used here refers to a straight- or branched-chain alkyl group having from 1 to 4 carbon atoms in the chain, optionally substituting hydrogens with halogens. Examples of “haloalkyl” groups include trifluoromethyl (CF₃), difluoromethyl (CF₂H), monofluoromethyl (CH₂F), pentafluoroethyl (CF₂CF₃), tetrafluoroethyl (CHFCF₃), monofluoroethyl (CH₂CH₂F), trifluoroethyl (CH₂CF₃), tetrafluorotrifluoromethylethyl (CF(CF₃)₂), and groups that in light of the ordinary skill in the art and the teachings provided herein would be considered equivalent to any one of the foregoing examples.

The term “aryl” refers to a monocyclic, aromatic carbocycle (ring structure having ring atoms that are all carbon) having 6 atoms per ring (Carbon atoms in the aryl groups are sp2 hybridized.)

The term “phenyl” represents the following moiety:

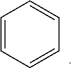

The term “heteroaryl”, refers to monocyclic, bicyclic and tricyclic ring systems having a total of five to fourteen ring members, wherein at least one ring in the system is aromatic, at least one ring in the system contains one or more heteroatoms, and wherein each ring in the system contains 3 to 7 ring members. Wherein from 1 to 4 of the ring atoms is independently O, N or S and the remaining ring atoms are carbon atoms. In one embodiment, a heteroaryl group has 5 to 10 ring atoms. In another embodiment, a heteroaryl group is monocyclic and has 5 or 6 ring atoms. In another embodiment, a heteroaryl group is monocyclic and has 5 or 6 ring atoms and at least one nitrogen ring atom. In another embodiment, a 6:5 or 5:6 ring-fused heteroaryl ring systems have zero, one or two heteroatoms in the five-membered ring, preferably one or two; and one or two heteroatoms in the fused six-membered ring. In another embodiment, a 6:6 ring-fused heteroaryl ring systems have zero, or one heteroatoms in one of the 6-membered rings; and one heteroatom in the fused six-membered ring.

A heteroaryl group is joined via a ring carbon atom and any nitrogen atom of a heteroaryl can be optionally oxidized to the corresponding N-oxide. The term “heteroaryl” also encompasses a heteroaryl group, as defined above, which has been fused to a benzene ring. The term “heteroaryl” may be used interchangeably with the term “heteroaryl ring” or the term “heteroaromatic”.

The term “5-membered heteroaryl” as used herein, refers to a heteroaryl group, as defined above, which has 5 ring atoms. Non-limiting examples of illustrative 5-membered heteroaryls include:

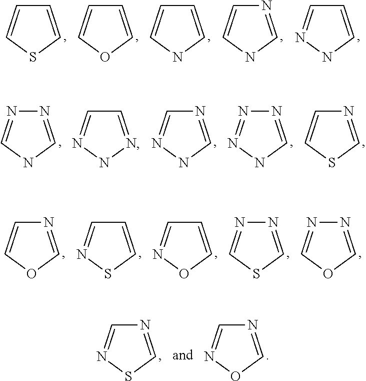

The term “6-membered heteroaryl” as used herein, refers to a heteroaryl group, as defined above, which has 6 ring atoms. Non-limiting examples of illustrative 6-membered heteroaryls include:

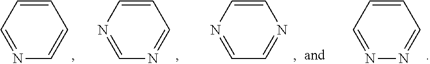

The term “5.6-fused bicyclic heteroaryl or 6.5-fused bicyclic heteroaryl” as used herein, refers to a heteroaryl group, as defined above, which has 9 ring atoms. Non-limiting examples of illustrative 5.6-fused bicyclic heteroaryl or 6.5-fused bicyclic heteroaryl include:

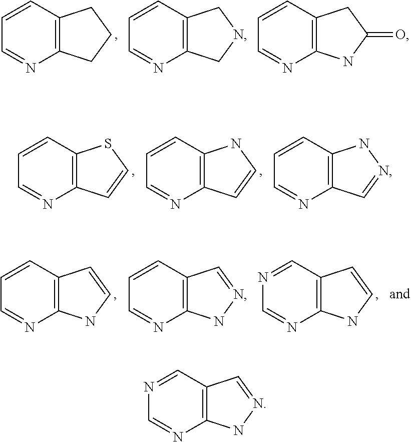

The term “6.6-fused bicyclic heteroaryl” as used herein, refers to a heteroaryl group, as defined above, which has 9 ring atoms. Non-limiting examples of illustrative 6.6-fused bicyclic heteroaryl include:

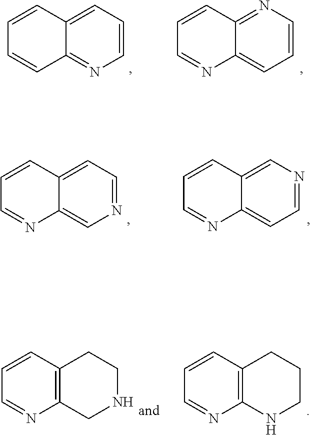 [SMILES: c1ccc2ncccc2c1 ; c1cnc2cccnc2c1 ; c1cnc2cnccc2c1 ; c1cnc2ccncc2c1 ; c1cnc2c(c1)CCNC2 ; c1cnc2c(c1)CCCN2]

The term “heterocycloalkyl” as used herein, refers to a ring system which is non-aromatic, 1 to 4 of the ring atoms is independently O, N or S and the remaining ring atoms are carbon atoms, which may optionally be fused to another ring (aromatic or heteroaromatic). Non-limiting examples of illustrative heterocycloalkyl include:

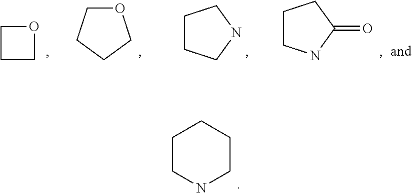

Those skilled in the art will recognize that the species of heteroaryl, heterocycloalkyl, cycloalkyl, and aryl groups listed or illustrated above are not exhaustive, and that additional species within the scope of these defined terms may also be selected.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system.

The term “variable point of attachment” means that a group is allowed to be attached at more than one alternative position in a structure. The attachment will always replace a hydrogen atom on one of the ring atoms. In other words, all permutations of bonding are represented by the single diagram, as shown in the illustrations below.

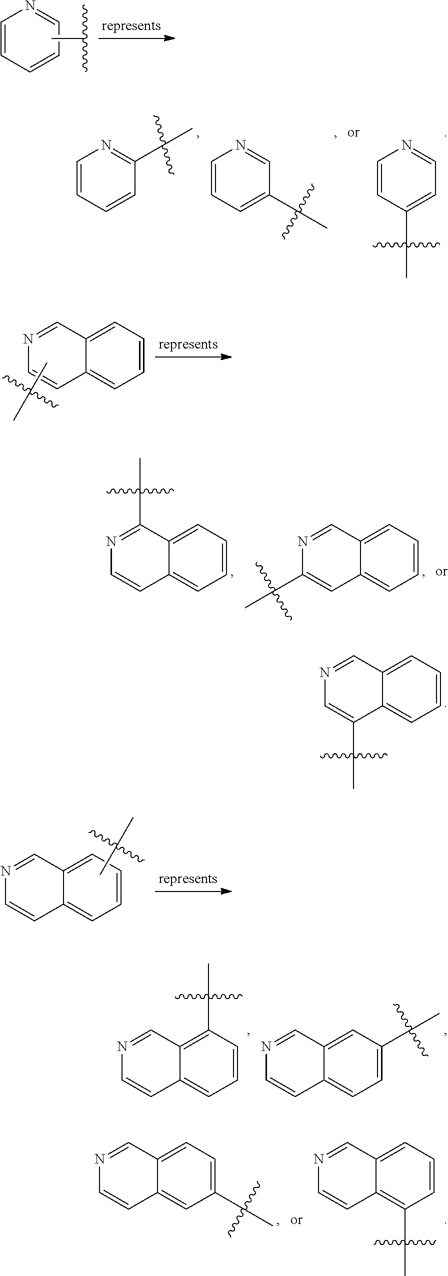

Those skilled in the art will recognize that that if more than one such substituent is present for a given ring; the bonding of each substituent is independent of all of the others. The groups listed or illustrated above are not exhaustive.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system.

Any formula given herein is intended to represent compounds having structures depicted by the structural formula as well as certain variations or forms. In particular, compounds of any formula given herein may have asymmetric centers and therefore exist in different enantiomeric forms. All optical isomers and stereoisomers of the compounds of the general formula, and mixtures thereof, are considered within the scope of such formula. The compounds of this invention may possess one or more asymmetric centers; such compounds can therefore be produced as individual (R)- or (S)-stereoisomers or as mixtures thereof. Thus, any formula given herein is intended to represent a racemate, one or more of its enantiomeric forms, one or more of its diastereomeric forms, and mixtures thereof. Additionally, any formula given herein is intended to refer also to any one of: hydrates, solvates, polymorphs and of such compounds, and mixtures thereof, even if such forms are not listed explicitly.

The term “R” at a stereocenter designates that the stereocenter is purely of the R-configuration as defined in the art; likewise, the term “S” means that the stereocenter is purely of the S-configuration. As used herein, the term “RS” refers to a stereocenter that exists as a mixture of the R- and S-configurations.

Compounds containing one stereocenter drawn without a stereo bond designation are a mixture of 2 enantiomers. Compounds containing 2 stereocenters both drawn without stereo bond designations are a mixture of 4 diastereomers. Compounds with 2 stereocenters both labeled “RS” and drawn with stereo bond designations are a 2-component mixture with relative stereochemistry as drawn. Unlabeled stereocenters drawn without stereo bond designations are a mixture of the R- and S-configurations. For unlabeled stereocenters drawn with stereo bond designations, the absolute stereochemistry is as depicted.

Reference to a compound herein stands for a reference to any one of: (a) the actually recited form of such compound, and (b) any of the forms of such compound in the medium in which the compound is being considered when named. For example, reference herein to a compound such as R—COOH, encompasses reference to any one of: for example, R—COOH(s), R—COOH(sol), and R—COO-(sol). In this example, R—COOH(s) refers to the solid compound, as it could be for example in a tablet or some other solid pharmaceutical composition or preparation; R—COOH(sol) refers to the undissociated form of the compound in a solvent; and R—COO-(sol) refers to the dissociated form of the compound in a solvent, such as the dissociated form of the compound in an aqueous environment, whether such dissociated form derives from R—COOH, from a salt thereof, or from any other entity that yields R—COO— upon dissociation in the medium being considered. In another example, an expression such as “exposing an entity to compound of formula R—COOH” refers to the exposure of such entity to the form, or forms, of the compound R—COOH that exists, or exist, in the medium in which such exposure takes place. In still another example, an expression such as “reacting an entity with a compound of formula R—COOH” refers to the reacting of (a) such entity in the chemically relevant form, or forms, of such entity that exists, or exist, in the medium in which such reacting takes place, with (b) the chemically relevant form, or forms, of the compound R—COOH that exists, or exist, in the medium in which such reacting takes place. In this regard, if such entity is for example in an aqueous environment, it is understood that the compound R—COOH is in such same medium, and therefore the entity is being exposed to species such as R—COOH(aq) and/or R—COO-(aq), where the subscript “(aq)” stands for “aqueous” according to its conventional meaning in chemistry and biochemistry. A carboxylic acid functional group has been chosen in these nomenclature examples; this choice is not intended, however, as a limitation but it is merely an illustration. It is understood that analogous examples can be provided in terms of other functional groups, including but not limited to hydroxyl, basic nitrogen members, such as those in amines, and any other group that interacts or transforms according to known manners in the medium that contains the compound. Such interactions and transformations include, but are not limited to, dissociation, association, tautomerism, solvolysis, including hydrolysis, solvation, including hydration, protonation, and deprotonation. No further examples in this regard are provided herein because these interactions and transformations in a given medium are known by any one of ordinary skill in the art.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number in an enriched form. Examples of isotopes that can be incorporated into compounds of the invention in a form that exceeds natural abundances include isotopes of hydrogen, carbon, nitrogen, oxygen, phosphorous, fluorine, chlorine, and iodine, such as ²H (or chemical symbol D), ³H (or chemical symbol T), ¹¹C, ¹³C, ¹⁴C, ¹⁵N, ¹⁸O, ¹⁷O, ³¹P, ³²P, ³⁵S, ¹⁸F, ³⁶Cl, and ¹²⁵I, respectively. Such isotopically labelled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. In particular, an ¹⁸F or ¹¹C labeled compound may be particularly preferred for PET or SPECT studies. Further, substitution with heavier isotopes such as deuterium (i.e., ²H, or D) may afford certain therapeutic advantages resulting from greater metabolic stability, for example increased in vivo half-life or reduced dosage requirements. Isotopically labeled compounds of this invention can generally be prepared by carrying out the procedures disclosed in the schemes or in the examples and preparations described below by substituting a readily available isotopically labeled reagent for a non-isotopically labeled reagent.

When referring to any formula given herein, the selection of a particular moiety from a list of possible species for a specified variable is not intended to define the same choice of the species for such variable appearing elsewhere. In other words, where a variable appears more than once, the choice of the species from a specified list is independent of the choice of the species for the same variable elsewhere in the formula, unless stated otherwise.

The term Cₙ₋ₘ alkyl refers to an aliphatic chain, whether straight or branched, with a total number N of carbon members in the chain that satisfies n≤N≤m, with m>n.

When the same plurality of substituents is assigned to various groups, the specific individual substituent assignment to each of such groups is meant to be independently made with respect to the specific individual substituent assignments to the remaining groups. By way of illustration, but not as a limitation, if each of groups Q and R can be H or F, the choice of H or F for Q is made independently of the choice of H or F for R, so the choice of assignment for Q does not determine or condition the choice of assignment for R, or vice-versa, unless it is expressly indicated otherwise. Illustrative claim recitation in this regard would read as “each of Q and R is independently H or F”, or “each of Q and R is independently selected from the group consisting of H and F”.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

In another example, a zwitterionic compound would be encompassed herein by referring to a compound that is known to form a zwitterion, even if it is not explicitly named in its zwitterionic form. Terms such as zwitterion, zwitterions, and their synonyms zwitterionic compound(s) are standard IUPAC-endorsed names that are well known and part of standard sets of defined scientific names. In this regard, the name zwitterion is assigned the name identification CHEBI:27369 by the Chemical Entities of Biological Interest (ChEBI) dictionary of molecular entities. As generally well known, a zwitterion or zwitterionic compound is a neutral compound that has formal unit charges of opposite sign. Sometimes these compounds are referred to by the term “inner salts”. Other sources refer to these compounds as “dipolar ions”, although the latter term is regarded by still other sources as a misnomer. As a specific example, aminoethanoic acid (the amino acid glycine) has the formula H₂NCH₂COOH, and it exists in some media (in this case in neutral media) in the form of the zwitterion ⁺H₃NCH₂COO⁻. Zwitterions, zwitterionic compounds, inner salts, and dipolar ions in the known and well-established meanings of these terms are within the scope of this invention, as would in any case be so appreciated by those of ordinary skill in the art. Because there is no need to name each and every embodiment that would be recognized by those of ordinary skill in the art, no structures of the zwitterionic compounds that are associated with the compounds of this invention are given explicitly herein. They are, however, part of the embodiments of this invention. No further examples in this regard are provided herein because the interactions and transformations in a given medium that lead to the various forms of a given compound are known by any one of ordinary skill in the art.

When referring to any formula given herein, the selection of a particular moiety from a list of possible species for a specified variable is not intended to define the same choice of the species for the variable appearing elsewhere. In other words, where a variable appears more than once, the choice of the species from a specified list is independent of the choice of the species for the same variable elsewhere in the formula, unless stated otherwise.

By way of a first example on substituent terminology, if substituent S¹ₑₓₐₘₚₗₑ is one of S₁ and S₂, and substituent S²ₑₓₐₘₚₗₑ is one of S₃ and S₄, then these assignments refer to embodiments of this invention given according to the choices S¹ₑₓₐₘₚₗₑ is S₁ and S²ₑₓₐₘₚₗₑ is S₃; S¹ₑₓₐₘₚₗₑ is S₁ and S²ₑₓₐₘₚₗₑ is S₄; S¹ₑₓₐₘₚₗₑ is S₂ and S²ₑₓₐₘₚₗₑ is S₃; S¹ₑₓₐₘₚₗₑ is S₂ and S²ₑₓₐₘₚₗₑ is S₄; and equivalents of each one of such choices. The shorter terminology “S¹ₑₓₐₘₚₗₑ is one of S₁ and S₂, and S²ₑₓₐₘₚₗₑ is one of S₃ and S₄” is accordingly used herein for the sake of brevity, but not by way of limitation. The foregoing first example on substituent terminology, which is stated in generic terms, is meant to illustrate the various substituent assignments described herein.

Furthermore, when more than one assignment is given for any member or substituent, embodiments of this invention comprise the various groupings that can be made from the listed assignments, taken independently, and equivalents thereof. By way of a second example on substituent terminology, if it is herein described that substituent Sₑₓₐₘₚₗₑ is one of S₁, S₂, and S₃, this listing refers to embodiments of this invention for which Sₑₓₐₘₚₗₑ is S₁; Sₑₓₐₘₚₗₑ is S₂; Sₑₓₐₘₚₗₑ is S₃; Sₑₓₐₘₚₗₑ is one of S₁ and S₂; Sₑₓₐₘₚₗₑ is one of S₁ and S₃; Sₑₓₐₘₚₗₑ is one of S₂ and S₃; Sₑₓₐₘₚₗₑ is one of S₁, S₂ and S₃; and Sₑₓₐₘₚₗₑ is any equivalent of each one of these choices. The shorter terminology “Sₑₓₐₘₚₗₑ is one of S₁, S₂, and S₃” is accordingly used herein for the sake of brevity, but not by way of limitation. The foregoing second example on substituent terminology, which is stated in generic terms, is meant to illustrate the various substituent assignments described herein.

The nomenclature “Cᵢ-Cⱼ” or “Cᵢ₋ⱼ” with j>i, when applied herein to a class of substituents, is meant to refer to embodiments of this invention for which each and every one of the number of carbon members, from i to j including i and j, is independently realized. By way of example, the term C₁-C₃ refers independently to embodiments that have one carbon member (C₁), embodiments that have two carbon members (C₂), and embodiments that have three carbon members (C₃).

A “pharmaceutically acceptable salt” is intended to mean a salt of an acid or base of a compound represented by Formula (I) that is non-toxic, biologically tolerable, or otherwise biologically suitable for administration to the subject. See, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002. Preferred pharmaceutically acceptable salts are those that are pharmacologically effective and suitable for contact with the tissues of patients without undue toxicity, irritation, or allergic response.

A compound of Formula (I) may possess a sufficiently acidic group, a sufficiently basic group, or both types of functional groups, and accordingly react with a number of inorganic or organic bases, and inorganic and organic acids, to form a pharmaceutically acceptable salt.

Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

Compounds of Formula (I) may contain at least one nitrogen of basic character, so desired pharmaceutically acceptable salts may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, ethanesulfonic acid, any compatible mixture of acids such as those given as examples herein, and any other acid and mixture thereof that are regarded as equivalents.

Compounds of Formula (I) may contain a carboxylic acid moiety, a desired pharmaceutically acceptable salt may be prepared by any suitable method, for example, treatment of the free acid with an inorganic or organic base, such as an amine (primary, secondary or tertiary), an alkali metal hydroxide, alkaline earth metal hydroxide, any compatible mixture of bases such as those given as examples herein, and any other base and mixture thereof that are regarded as equivalents or acceptable substitutes in light of the ordinary level of skill in this technology. Illustrative examples of suitable salts include organic salts derived from amino acids, such as glycine and arginine, ammonia, carbonates, bicarbonates, primary, secondary, and tertiary amines, and cyclic amines, such as benzylamines, pyrrolidines, piperidine, morpholine, piperazine, N-methyl-glucamine and tromethamine and inorganic salts derived from sodium, calcium, potassium, magnesium, manganese, iron, copper, zinc, aluminum, and lithium.

The compounds of the invention, including their pharmaceutically acceptable salts, whether alone or in combination, (collectively, “active agent” or “active agents”) of the present invention are useful as CSNK1D-modulators in the methods of the invention. Such methods for modulating CSNK1D comprise the use of a therapeutically effective amount of at least one chemical entity of the invention.

In some embodiments, the CSNK1D modulator is an inhibitor and is used in a subject diagnosed with or suffering from a disease, disorder, or condition associated with protein kinase CSNK1D activity, such as those described herein. Symptoms or disease states are intended to be included within the scope of “disease, disorders or conditions.”

Accordingly, the invention relates to methods of using the active agents described herein to treat subjects diagnosed with or suffering from a disease, disorder, or condition associated with the protein kinase CSNK1D activity. The term “treat” or “treating” as used herein is intended to refer to administration of an active agent or composition of the invention to a subject for the purpose of effecting a therapeutic or prophylactic benefit through modulation of protein kinase CSNK1D activity. Treating includes reversing, ameliorating, alleviating, inhibiting the progress of, lessening the severity of, or preventing a disease, disorder, or condition, or one or more symptoms of such disease, disorder or condition associated with the CSNK1D modulation. The term “subject” refers to a mammalian patient in need of such treatment, such as a human.

The term “composition” refers to a product that includes the specified ingredients in therapeutically effective amounts, as well as any product that results, directly, or indirectly, from combinations of the specified ingredients in the specified amounts.

The term “CSNK1D inhibitor” is intended to encompass a compound that interacts with protein kinase CSNK1D to substantially reduce or eliminate its catalytic activity, thereby increasing the concentrations of its substrate(s). The term “CSNK1D-modulated” is used to refer to the condition of being affected by the modulation of the activity of protein kinase CSNK1D including the condition of being affected by the inhibition of the CSNK1D activity. The disclosure is directed to methods for treating, ameliorating and/or preventing neurodegenerative diseases and/or disorders, psychiatric disorders, and cancers by the administration of therapeutically effective amounts of protein kinase CSNK1D modulators to subjects in need thereof.

The term “modulators” include both inhibitors and activators, where “inhibitors” refer to compounds that decrease, prevent, inactivate, desensitize, or down-regulate the CSNK1D expression or activity, and “activators” are compounds that increase, activate, facilitate, sensitize, or up-regulate CSNK1D expression or activity.

As used herein, unless otherwise noted, the term “affect” or “affected” (when referring to a disease, condition or disorder that is affected by inhibition of CSNK1D) includes a reduction in the frequency and/or severity of one or more symptoms or manifestations of said disease, condition or disorder; and/or include the prevention of the development of one or more symptoms or manifestations of said disease, condition or disorder or the development of the disease, condition or disorder.

In treatment methods according to the invention, a therapeutically effective amount of at least one active agent according to the invention is administered to a subject suffering from or diagnosed as having such a disease, disorder, or condition. A “therapeutically effective amount” means an amount or dose sufficient to generally bring about the desired therapeutic or prophylactic benefit in subjects in need of such treatment for the designated disease, disorder, or condition. Effective amounts or doses of the active agents of the present invention may be ascertained by routine methods such as modeling, dose escalation studies or clinical trials, and by taking into consideration routine factors, e.g., the mode or route of administration or drug delivery, the pharmacokinetics of the agent, the severity and course of the disease, disorder, or condition, the subject's previous or ongoing therapy, the subject's health status and response to drugs, and the judgment of the treating physician. For a 70-kg human, an illustrative range for a suitable dosage amount is from about 1 to 1000 mg/day in single or multiple dosage units (e.g., BID, TID, QID or as required by modality). For example, a suitable dosage amount is from about 100 to 300 mg/day in single or multiple dosage units.

Once improvement of the subject's disease, disorder, or condition has occurred, the dose may be adjusted for preventive or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Subjects may, however, require intermittent treatment on a long-term basis upon any recurrence of symptoms.

In addition, the compounds of the invention are envisaged for use alone, in combination with one or more of other compounds of this invention, or in combination with additional active ingredients in the treatment of the conditions discussed below. The additional active ingredients may be co-administered separately with at least one compound of the invention, with active agents of the invention or included with such an agent in a pharmaceutical composition according to the invention. In an illustrative embodiment, additional active ingredients are those that are known or discovered to be effective in the treatment of conditions, disorders, or diseases associated with protein kinase CSNK1D modulation, such as another protein kinase CSNK1D inhibitor or a compound active against another target associated with the particular condition, disorder, or disease. The combination may serve to increase efficacy (e.g., by including in the combination a compound potentiating the potency or effectiveness of an agent according to the invention), decrease one or more side effects, or decrease the required dose of the active agent according to the invention.

When referring to inhibiting the target, an “effective amount” means an amount sufficient to affect protein kinase CSNK1D modulation.

The active agents of the invention are envisaged for use, alone or in combination with one or more additional active ingredients, to formulate pharmaceutical compositions of the invention. A pharmaceutical composition of the invention comprises a therapeutically effective amount of at least one active agent in accordance with the invention.

Pharmaceutically acceptable excipients commonly used in pharmaceutical compositions are substances that are non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of such excipients include calcium carbonate, calcium phosphate, various sugars and types of starch, cellulose derivatives, gelatin, vegetable oils, and polyethylene glycols.

Delivery forms of the pharmaceutical compositions containing one or more dosage units of the active agents may be prepared using pharmaceutically acceptable excipients and compounding techniques known or that become available to those of ordinary skill in the art. The compositions may be administered in the inventive methods by a suitable route of delivery, e.g., oral, parenteral, rectal, topical, or ocular routes, or by inhalation.

The preparation may be in the form of tablets, capsules, sachets, dragees, powders, granules, lozenges, powders for reconstitution, liquid preparations, or suppositories. The compositions may be formulated for any one of a plurality of administration routes, such as intravenous infusion, topical administration, or oral administration. Preferably, the compositions may be formulated for oral administration.

For oral administration, the active agents of the invention can be provided in the form of tablets or capsules, or as a solution, emulsion, or suspension. To prepare the oral compositions, the active agents may be formulated to yield a dosage of, e.g., for a 70-kg human, an illustrative range for a suitable dosage amount is from about 1 to 1000 mg/day in single or multiple dosage units. Preferably, a suitable dosage amount is from about 100 to 300 mg/day in single or multiple dosage units.

Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid, or talc. If desired, the tablets may be coated with a material such as glyceryl monostearate or glyceryl distearate to delay absorption in the gastrointestinal tract or may be coated with an enteric coating.

Capsules for oral administration include hard and soft gelatin or (hydroxypropyl)methyl cellulose capsules. To prepare hard gelatin capsules, active ingredient(s) may be mixed with a solid, semi-solid, or liquid diluent. Liquids for oral administration may be in the form of suspensions, solutions, emulsions, or syrups or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The active agents of this invention may also be administered by non-oral routes. For example, compositions may be formulated for rectal administration as a suppository, enema or foam. For parenteral use, including intravenous, intramuscular, intraperitoneal, or subcutaneous routes, the agents of the invention may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 μg/kg/minute of agent admixed with a pharmaceutical carrier over a period ranging from several minutes to several days.

For topical administration, the agents may be mixed with a pharmaceutical carrier at a concentration of about 0.01% to about 20% of drug to vehicle, preferably 0.1% to 10%. Another mode of administering the agents of the invention may utilize a patch formulation to affect transdermal delivery.

Active agents may alternatively be administered in methods of this invention by inhalation, via the nasal or oral routes, e.g., in a spray formulation also containing a suitable carrier.

In a further embodiment, the invention is directed to a method of treating a subject suffering from or diagnosed with a disease, disorder, or condition associated with CSNK1D modulation, comprising administering to the subject in need of such treatment a therapeutically effective amount of the active agent.

The compounds of Formula (I) are useful in methods for treating, ameliorating and/or preventing a disease, a condition or a disorder that is affected by the inhibition of CSNK1D. Such methods comprise administering to a subject, including an animal, a mammal, and a human in need of such treatment, amelioration and/or prevention, a therapeutically effective amount of a compound of Formula (I), or an enantiomer, diastereomer, solvate or pharmaceutically acceptable salt thereof.

In particular, the compounds of Formula (I), or pharmaceutically acceptable salts, isotopes, N-oxides, solvates and stereoisomers thereof, are useful for treating, ameliorating and/or preventing neurodegenerative diseases and/or disorders, psychiatric disorders, and cancers. More particularly, the compounds of Formula (I), or pharmaceutically acceptable salts, isotopes, N-oxides, solvates and stereoisomers thereof, are useful for treating, ameliorating and/or preventing mood or psychiatric disorders, neurodegenerative diseases, oncology indications, addiction or substance abuse indications, metabolic indications and pain by administering to a subject in need thereof a therapeutically effective amount of a compound of Formula (I), or a pharmaceutically acceptable salt, isotope, N-oxide, solvate or stereoisomer thereof as herein defined.

Mood/psychiatric disorders include: type 1 bipolar depression, type 2 bipolar depression, seasonal affective disorder, post-traumatic stress disorder, generalized anxiety disorder, dysthymia, obsessive compulsive disorder, schizophrenia, schizoaffective disorder, mixed episode bipolar disease, major depressive disorder, premenstrual dysphoric disorder, jet lag syndrome, familial advanced sleep phase syndrome, delayed sleep phase syndrome, non-24 hour sleep-wake phase disorder and irregular sleep-wake rhythm disorder.

Neurodegenerative diseases include Alzheimer's disease, Parkinson's disease, Amyotrophic lateral sclerosis, Frontotemporal dementia, Down Syndrome, Progressive supranuclear palsy, parkinsonism dementia complex of Guam, and Pick's disease.

Oncology indications include: gastroenteric, breast, renal, skin, hematological, colorectal, pancreatic, prostate, ovarian, bladder, liver, and head/neck.

Addiction and substance abuse indications involving chemicals (such as cocaine, opiate, tobacco, alcohol, amphetamines, inhalants, and phencyclidine), impulse control disorders (such as intermittent explosive disorder, kleptomania, pyromania, and gambling), and behavioral disturbances (such as food, sex, shopping, cutting, exercising, and pain seeking).

Metabolic diseases include: type 1 diabetes mellitus, idiopathic, type 2 diabetes mellitus, genetic defect of B-cell function, genetic defects of insulin action (such as type A insulin resistance, leprechaunism, Rabson-Mendahall syndrome, and lipoatrophic diabetes), disease of exocrine pancreas (such as pancreatitis, neoplasia, trauma, cystic fibrosis, hemochromatosis, and fibrocalculous pancreatopathy), endocrinopathies (such as Acromegaly, Cushing's syndrome, Glucagonoma, Pheochromocytoma, Hyperthyroidism, Somatostatinoma, and Aldosteronoma), drug/chemical induced (such as Vacor, Pentamidine, Nicotinic acid, Glucocorticoids, Thyroid hormone, Diazoxide, 3-adrenergic agonists, Thiazides, Dilantin, and ∝-Interferon), infections (such as congenital rubella, and cytomegalovirus) uncommon forms (such as “stiff-man” syndrome and anti-insulin receptor antibodies), genetic syndromes (such as Down's syndrome, Klinefelter's syndrome, Turner's syndrome, Wolfram's syndrome, Friedreich's ataxia, Huntington's chorea, Laurence-Moon-Biedl syndrome, Myotonic dystrophy, Porphyria, and Prader-Willi syndrome), and gestational diabetes mellitus.

Pain includes nociceptive (such as arthritis, mechanical back pain, and post-surgical pain), inflammatory (such as gout and rheumatoid arthritis), neuropathic (such as neuropathy, radicular pain, and trigeminal neuralgia), and functional (such as fibromyalgia and irritable bowel syndrome).

Other embodiments of this invention provide for a method for modulating protein kinase CSNK1D activity, including when such receptor is in a subject, comprising exposing protein kinase CSNK1D to a therapeutically effective amount of at least one compound selected from compounds of the invention.

Embodiments of this invention are compounds of Formula (I),

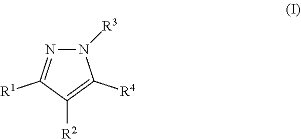

wherein
R¹ is selected from the group consisting of:
(a) 5-membered heteroaryl selected from the group consisting of:

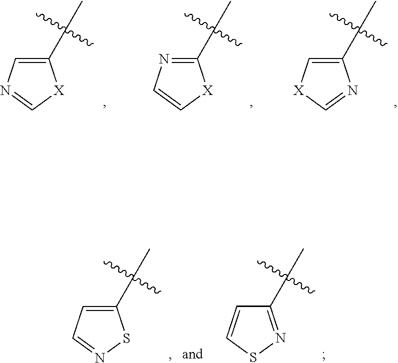

(b) pyridinyl substituted with one or two halo members;
(c) pyrimidinyl; pyrimidinyl substituted with halo; pyrazinyl; pyrazinyl substituted with C₁₋₆ alkyl; pyridazinyl; and pyridazinyl substituted with C₁₋₆ alkyl;
R² is selected from the group consisting of:
(d)

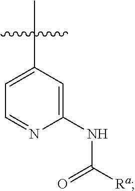

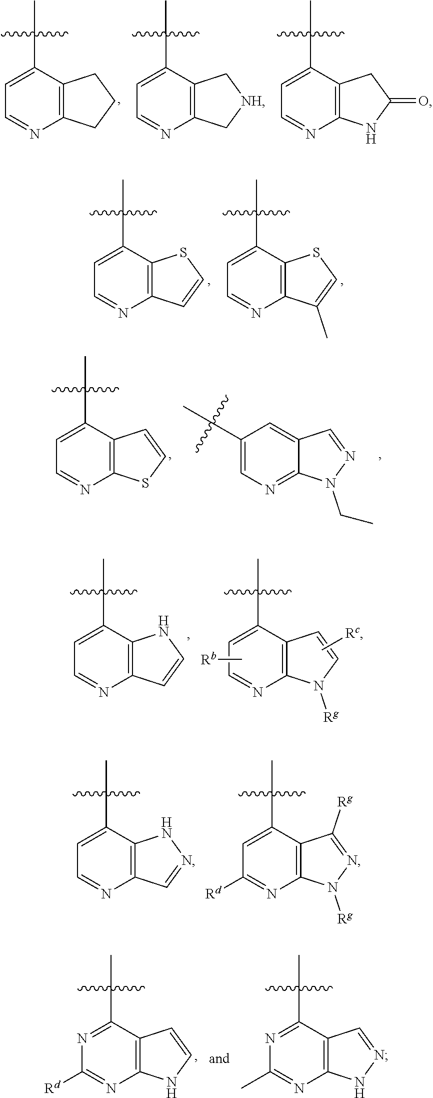

wherein
R^{a} is C₁₋₃ alkyl or C₃₋₆ cycloalkyl;
R^{b} is selected from the group consisting of: H, halo, C₁₋₃ alkyl, C₁₋₃ haloalkyl, CN, CH₂CN, NH₂, oxetanyl, and CH₂-oxetanyl;
R^{c} is selected from the group consisting of: H, halo, C₁₋₃ alkyl, C₁₋₃ haloalkyl, CN, CH₂CN, oxetanyl, oxetanyl substituted with OH, tetrahydrofuranyl substituted with OH, and CH₂-tetrahydrofuranyl;
R^{d} is selected from the group consisting of: H, C₁₋₃ alkyl, C₁₋₃ haloalkyl, and C₃₋₆ cycloalkyl;
R^{g} is H or C₁₋₃ alkyl;
X is O, S, or N—CH₃;
R³ is selected from the group consisting of: C₁₋₆ alkyl, CH₂CH₂OCH₃, C₁₋₆ haloalkyl, C₃₋₆ cycloalkyl, oxetanyl, CH₂-oxetanyl, and tetrahydrofuranyl; and
R⁴ is selected from the group consisting of: H, C₁₋₃ alkyl, and C₃₋₆ cycloalkyl; and pharmaceutically acceptable salts, isotopes, N-oxides, solvates, and stereoisomers thereof.

An additional embodiment of the invention is a compound of Formula (I) wherein R¹ is

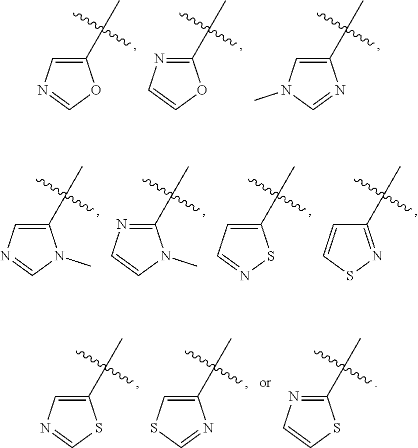

An additional embodiment of the invention is a compound of Formula (I) wherein R¹ is

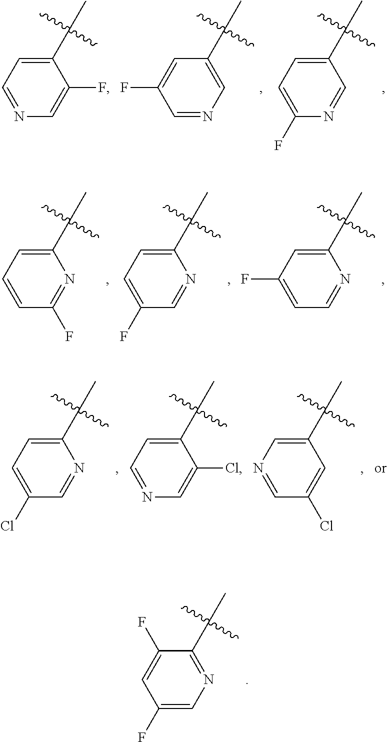

An additional embodiment of the invention is a compound of Formula (I) wherein R¹ is

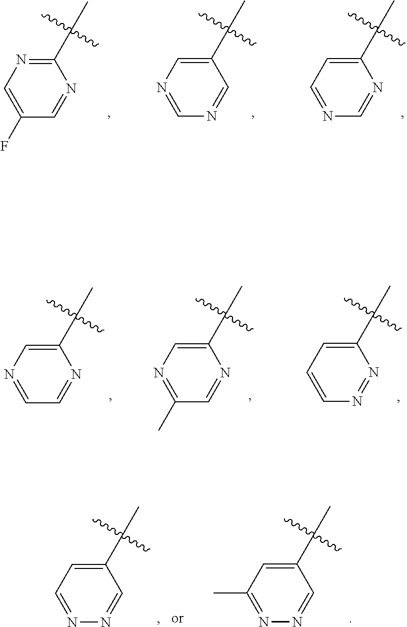

An additional embodiment of the invention is a compound of Formula (I) wherein R¹ is

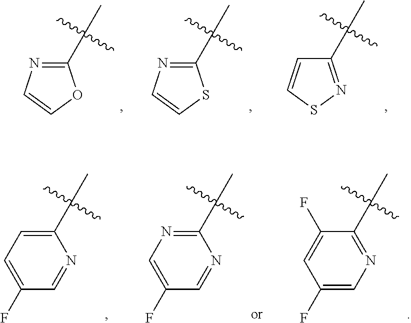 [SMILES: *c1ncco1 ; *c1nccs1 ; *c1ccsn1 ; *c1ccc(F)cn1 ; *c1ncc(F)cn1 ; *c1ncc(F)cc1F]

An additional embodiment of the invention is a compound of Formula (I) wherein R² is

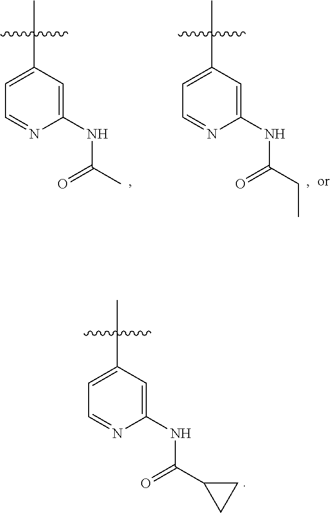 [SMILES: *c1ccnc(NC(C)=O)c1 ; *c1ccnc(NC(=O)CC)c1 ; *c1ccnc(NC(=O)C2CC2)c1]

An additional embodiment of the invention is a compound of Formula (I) wherein R² is

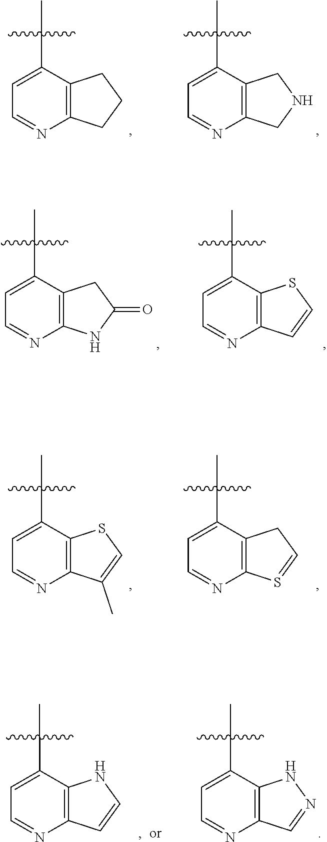

An additional embodiment of the invention is a compound of Formula (I) wherein R² is

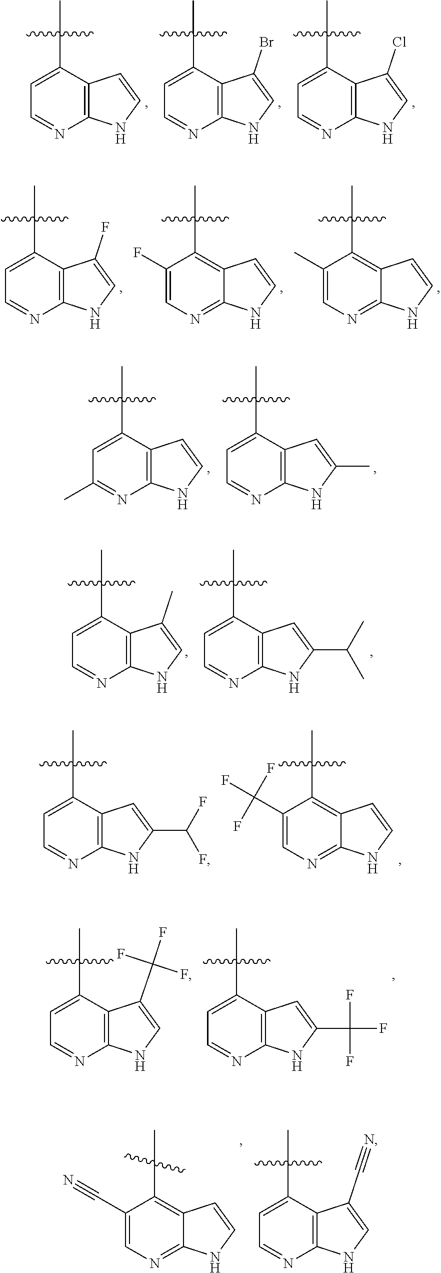

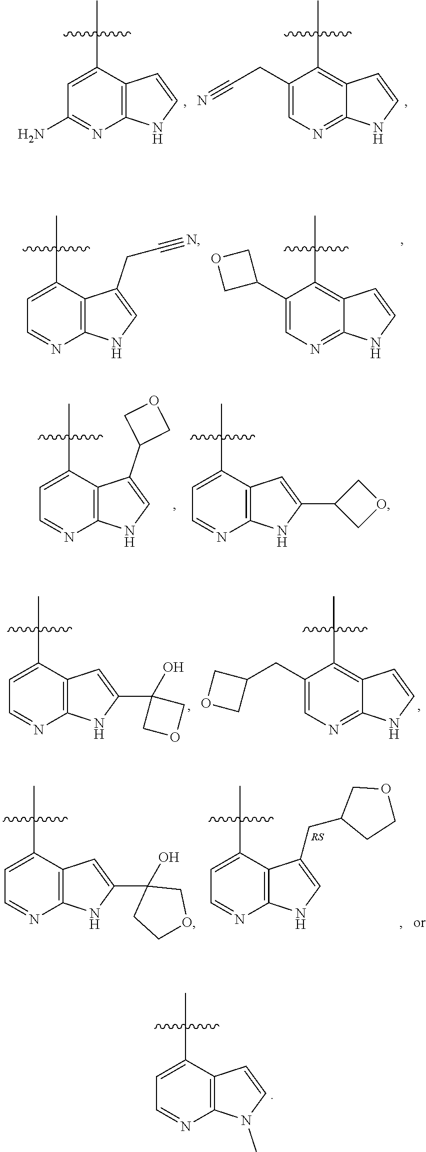

An additional embodiment of the invention is a compound of Formula (I) wherein R² is

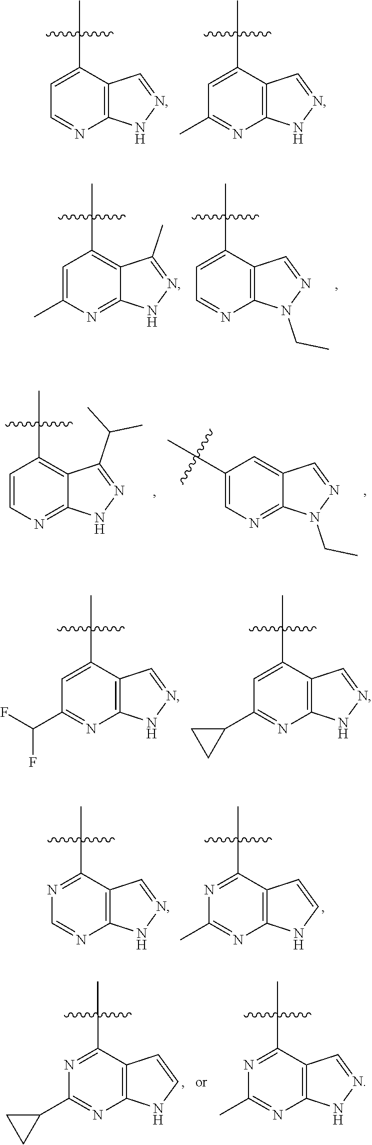

An additional embodiment of the invention is a compound of Formula (I) wherein R² is

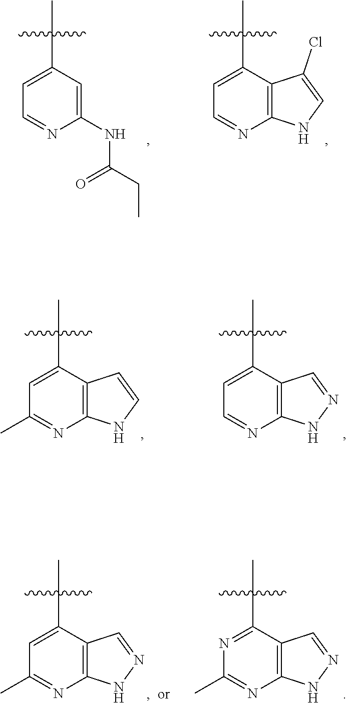 [SMILES: *c1ccnc(NC(=O)CC)c1 ; *c1ccnc2[nH]cc(Cl)c12 ; *c1cc(C)nc2[nH]ccc12 ; *c1ccnc2[nH]ncc12 ; *c1cc(C)nc2[nH]ncc12 ; *c1nc(C)nc2[nH]ncc12]

An additional embodiment of the invention is a compound of Formula (I) wherein R³ is CH₃, CH₂CH₃, CH(CH₃)₂, CH₂CH(CH₃)₂, CH₂CH₂OCH₃, CHF₂, CH₂CH₂F, CH₂CHF₂, CH₂CF₃, cyclopropyl, cyclobutyl,

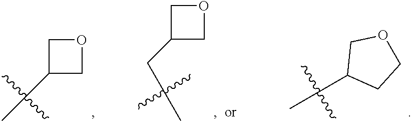 [SMILES: *C1COC1 ; *CC1COC1 ; *C1CCOC1]

An additional embodiment of the invention is a compound of Formula (I) wherein R³ is C₁₋₆ alkyl, or C₁₋₆ haloalkyl.

An additional embodiment of the invention is a compound of Formula (I) wherein R³ is C₁₋₆ alkyl.

An additional embodiment of the invention is a compound of Formula (I) wherein R³ is cyclopropyl, cyclobutyl,

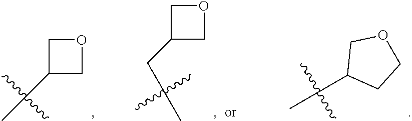 [SMILES: *C1COC1 ; *CC1COC1 ; *C1CCOC1]

An additional embodiment of the invention is a compound of Formula (I) wherein R³ is CH₃, CH₂CH₂F, or CH₂CHF₂.

An additional embodiment of the invention is a compound of Formula (I) wherein R⁴ is H.

An additional embodiment of the invention is a compound of Formula (I) wherein R⁴ is CH₃.

An additional embodiment of the invention is a compound of Formula (I) wherein R⁴ is cyclobutyl.

An additional embodiment of the invention is a compound of Formula (I) wherein X is O.

An additional embodiment of the invention is a compound of Formula (I) wherein X is S.

An additional embodiment of the invention is a compound of Formula (I) wherein X is N—CH₃.

A further embodiment of the current invention is a compound as shown below in Table 1.

TABLE 1

| Example | |
|---|---|
| # | Compound Name |
| 1 | N-(4-(3-(5-Fluoropyridin-2-yl)-1-methyl-1H-pyrazol-4-yl)pyridin-2-yl) |
| | propionamide; |
| 2 | 4-(3-(5-Fluoropyridin-2-yl)-1-methyl-1H-pyrazol-4-yl)-6,7-dihydro-5H- |
| | cyclopenta[b]pyridine; |
| 3 | 4-(3-(5-Fluoropyridin-2-yl)-1-methyl-1H-pyrazol-4-yl)-1,3-dihydro-2H- |
| | pyrrolo[2,3-b]pyridin-2-one; |
| 4 | 7-(3-(5-Fluoropyridin-2-yl)-1-methyl-1H-pyrazol-4-yl)-1H-pyrrolo[3,2-b] |
| | pyridine; |
| 5 | 7-(3-(5-Fluoropyridin-2-yl)-1-methyl-1H-pyrazol-4-yl)-1H-pyrazolo[4,3-b] |
| | pyridine; |
| 6 | 5-(1-Methyl-4-(1H-pyrrolo[2,3-b]pyridin-4-yl)-1H-pyrazol-3-yl)oxazole; |
| 7 | 2-(1-Methyl-4-(1H-pyrrolo[2,3-b]pyridin-4-yl)-1H-pyrazol-3-yl)oxazole; |
| 8 | 2-(1-Methyl-4-(6-methyl-1H-pyrazolo[3,4-b]pyridin-4-y])-1H-pyrazol-3-yl) |
| | oxazole; |
| 9 | 4-(1-Methyl-3-(1-methyl-1H-imidazol-4-yl)-1H-pyrazol-4-yl)-1H- |
| | pyrrolo[2,3-b]pyridine; |
| 10 | 4-(1-Methyl-3-(1-methyl-1H-imidazol-5-yl)-1H-pyrazol-4-yl)-1H- |
| | pyrrolo[2,3-b]pyridine; |
| 11 | 4-(1-Methyl-3-(1-methyl-1H-imidazol-2-yl)-1H-pyrazol-4-yl)-1H- |
| | pyrrolo[2,3-b]pyridine; |
| 12 | 5-(1-Methyl-4-(1H-pyrrolo[2,3-b]pyridin-4-yl)-1H-pyrazol-3-yl)isothiazole; |
| 13 | 4-(1-Methyl-4-(1H-pyrrolo[2,3-b]pyridin-4-yl)-1H-pyrazol-3-yl)thiazole; |
| 14 | 5-(1-Methyl-4-(1H-pyrrolo[2,3-b]pyridin-4-yl)-1H-pyrazol-3-yl)thiazole; |
| 15 | 3-(1-Methyl-4-(1H-pyrrolo[2,3-b]pyridin-4-yl)-1H-pyrazol-3-yl)isothiazole; |
| 16 | 2-(1-Methyl-4-(1H-pyrrolo[2,3-b]pyridin-4-yl)-1H-pyrazol-3-yl)thiazole; |
| 18 | 4-[3-(3-Fluoro-4-pyridyl)-1-methyl-pyrazol-4-yl]-1H-pyrrolo[2,3-b] |
| | pyridine; |
| 19 | 4-[3-(3-Fluoro-4-pyridyl)-1-methyl-pyrazol-4-yl]-1H-pyrazolo[3,4-b] |
| | pyridine; |
| 20 | 4-[3-(5-Fluoro-2-pyridyl)-1-methyl-pyrazol-4-yl]-1H-pyrrolo[2,3-b] |
| | pyridine; |
| 21 | 4-[3-(5-Fluoro-3-pyridyl)-1-methyl-pyrazol-4-yl]-1H-pyrrolo[2,3-b] |
| | pyridine; |
| 24 | 4-[3-(5-Chloro-2-pyridyl)-1-methyl-pyrazol-4-yl]-1H-pyrrolo[2,3-b] |
| | pyridine; |
| 25 | 4-(3-(5-Chloropyridin-2-yl)-1-methyl-1H-pyrazol-4-yl)-1H-pyrazolo[3,4-b] |
| | pyridine; |
| 26 | 4-(3-(5-Chloropyridin-2-yl)-1-methyl-1H-pyrazol-4-yl)-6-methyl-1H- |
| | pyrazolo[3,4-b]pyridine; |
| 27 | 4-[3-(4-Fluoro-2-pyridyl)-1-methyl-pyrazol-4-yl]-1H-pyrrolo[2,3-b] |
| | pyridine; |
| 28 | 4-[3-(6-Fluoro-3-pyridyl)-1-methyl-pyrazol-4-yl]-1H-pyrrolo[2,3-b] |
| | pyridine; |
| 29 | 4-[3-(6-Fluoro-2-pyridyl)-1-methyl-pyrazol-4-yl]-1H-pyrrolo[2,3-b] |
| | pyridine; |
| 37 | 4-[3-(3,5-Difluoro-2-pyridyl)-1-methyl-pyrazol-4-yl]-1H-pyrrolo[2,3-b] |
| | pyridine; |
| 38 | 4-(3-(3,5-Difluoropyridin-2-yl)-1-methyl-1H-pyrazol-4-yl)-1H- |
| | pyrazolo[3,4-b]pyridine; |
| 43 | 4-[3-(5-Fluoropyrimidin-2-yl)-1-methyl-pyrazol-4-yl]-1H-pyrrolo[2,3-b] |
| | pyridine; |
| 44 | 4-(1-Methyl-3-pyrimidin-4-yl-pyrazol-4-yl)-1H-pyrrolo[2,3-b]pyridine; |
| 45 | 4-(1-Methyl-3-pyrimidin-5-yl-pyrazol-4-yl)-1H-pyrrolo[2,3-b]pyridine; |
| 46 | 4-(1-Methyl-3-pyrazin-2-yl-pyrazol-4-yl)-1H-pyrrolo[2,3-b]pyridine; |
| 47 | 4-[1-Methyl-3-(5-methylpyrazin-2-yl)pyrazol-4-yl]-1H-pyrrolo[2,3-b] |
| | pyridine; |
| 48 | 4-(1-Methyl-3-pyridazin-3-yl-pyrazol-4-yl)-1H-pyrrolo[2,3-b]pyridine; |
| 49 | 4-(1-Methyl-3-pyridazin-4-yl-pyrazol-4-yl)-1H-pyrrolo[2,3-b]pyridine; |
| 50 | 4-[3-(5-Fluoro-2-pyridyl)-1-(oxetan-3-ylmethyl)pyrazol-4-yl]-6-methyl-1H- |
| | pyrazolo[3,4-b]pyridine; |
| 51 | 4-(3-(5-Fluoropyridin-2-yl)-1,5-dimethyl-1H-pyrazol-4-yl)-1H-pyrrolo[2,3-b] |
| | pyridine; |
| 52 | 4-(5-Cyclobutyl-3-(5-fluoropyridin-2-yl)-1-methyl-1H-pyrazol-4-yl)-1H- |
| | pyrrolo[2,3-b]pyridine; |
| 54 | 4-[3-(5-Fluoro-2-pyridyl)-1-(trideuteriomethyl)pyrazol-4-yl]-1H- |
| | pyrrolo[2,3-b]pyridine; |
| 55 | 4-[1-Ethyl-3-(5-fluoro-2-pyridyl)pyrazol-4-yl]-1H-pyrrolo[2,3-b]pyridine; |
| 56 | 4-(1-Ethyl-3-(5-fluoropyridin-2-yl)-1H-pyrazol-4-yl)-1H-pyrazolo[3,4-b] |
| | pyridine; |
| 57 | 4-(1-Ethyl-3-(5-fluoropyridin-2-yl)-1H-pyrazol-4-yl)-6-methyl-1H- |
| | pyrazolo[3,4-b]pyridine; |
| 58 | 4-[3-(5-Fluoro-2-pyridyl)-1-isopropyl-pyrazol-4-yl]-1H-pyrrolo[2,3-b] |
| | pyridine; |
| 59 | 4-(3-(5-Fluoropyridin-2-yl)-1-isopropyl-1H-pyrazol-4-yl)-1H-pyrazolo[3,4-b] |
| | pyridine; |
| 60 | 4-(3-(5-Fluoropyridin-2-yl)-1-isopropyl-1H-pyrazol-4-yl)-6-methyl-1H- |
| | pyrazolo[3,4-b]pyridine; |
| 61 | 4-[3-(5-Fluoro-2-pyridyl)-1-isobutyl-pyrazol-4-yl]-1H-pyrrolo[2,3-b] |
| | pyridine; |
| 62 | 4-(3-(5-Fluoropyridin-2-yl)-1-isobutyl-1H-pyrazol-4-yl)-1H-pyrazolo[3,4-b] |
| | pyridine; |
| 63 | 4-(3-(5-Fluoropyridin-2-yl)-1-isobutyl-1H-pyrazol-4-yl)-6-methyl-1H- |
| | pyrazolo[3,4-b]pyridine; |
| 64 | 4-(3-(5-Fluoropyridin-2-yl)-1-(2-methoxyethyl)-1H-pyrazol-4-yl)-6-methyl- |
| | 1H-pyrazolo[3,4-b]pyridine; |
| 65 | 4-[1-(Difluoromethyl)-3-(5-fluoro-2-pyridyl)pyrazol-4-yl]-1H-pyrrolo[2,3-b] |
| | pyridine; |
| 66 | 4-(1-(Difluoromethyl)-3-(5-fluoropyridin-2-yl)-1H-pyrazol-4-yl)-1H- |
| | pyrazolo[3,4-b]pyridine; |
| 67 | 4-(1-(Difluoromethyl)-3-(5-fluoropyridin-2-yl)-1H-pyrazol-4-yl)-6-methyl- |
| | 1H-pyrazolo[3,4-b]pyridine; |
| 68 | 4-[1-(2-Fluoroethyl)-3-(5-fluoro-2-pyridyl)pyrazol-4-yl]-1H-pyrrolo[2,3-b] |
| | pyridine; |
| 69 | 4-(1-(2,2-Difluoroethyl)-3-(5-fluoropyridin-2-yl)-1H-pyrazol-4-yl)-6- |
| | methyl-1H-pyrazolo[3,4-b]pyridine; |
| 70 | 4-(3-(5-Fluoropyridin-2-yl)-1-(2,2,2-trifluoroethyl)-1H-pyrazol-4-yl)-6- |
| | methyl-1H-pyrazolo[3,4-b]pyridine; |
| 71 | 4-(1-Cyclobutyl-3-(5-fluoropyridin-2-yl)-1H-pyrazol-4-yl)-6-methyl-1H- |
| | pyrazolo[3,4-b]pyridine; |
| 72 | 4-[3-(5-Fluoro-2-pyridyl)-1-(oxetan-3-yl)pyrazol-4-yl]-1H-pyrrolo[2,3-b] |
| | pyridine; |
| 73 | 4-(3-(5-Fluoropyridin-2-yl)-1-(oxetan-3-yl)-1H-pyrazol-4-yl)-6-methyl-1H- |
| | pyrazolo[3,4-b]pyridine; |
| 74 | (R/S)-4-[3-(5-Fluoro-2-pyridyl)-1-tetrahydrofuran-3-yl-pyrazol-4-yl]-1H- |
| | pyrrolo[2,3-b]pyridine; |
| 75 | 3-Bromo-4-[3-(5-fluoro-2-pyridyl)-1-methyl-pyrazol-4-yl]-1H-pyrrolo[2,3-b] |
| | pyridine; |
| 76 | 3-Chloro-4-[3-(5-fluoro-2-pyridyl)-1-methyl-pyrazol-4-yl]-1H-pyrrolo[2,3-b] |
| | pyridine; |
| 77 | 3-Fluoro-4-[3-(5-fluoro-2-pyridyl)-1-methyl-pyrazol-4-yl]-1H-pyrrolo[2,3-b] |
| | pyridine; |
| 78 | 5-Fluoro-4-[3-(5-fluoro-2-pyridyl)-1-methyl-pyrazol-4-yl]-1H-pyrrolo[2,3-b] |
| | pyridine; |
| 79 | 4-[3-(5-Fluoro-2-pyridyl)-1-methyl-pyrazol-4-yl]-5-methyl-1H-pyrrolo[2,3-b] |
| | pyridine; |
| 80 | 4-[3-(5-Fluoro-2-pyridyl)-1-methyl-pyrazol-4-yl]-6-methyl-1H-pyrrolo[2,3-b] |
| | pyridine; |
| 81 | 4-(3-(5-Fluoropyridin-2-yl)-1-methyl-1H-pyrazol-4-yl)-2-methyl-1H- |
| | pyrrolo[2,3-b]pyridine; |
| 82 | 4-[3-(5-Fluoro-2-pyridyl)-1-methyl-pyrazol-4-yl]-3-methyl-1H-pyrrolo[2,3-b] |
| | pyridine; |
| 83 | 4-(3-(5-Fluoropyridin-2-yl)-1-methyl-1H-pyrazol-4-yl)-2-isopropyl-1H- |
| | pyrrolo[2,3-b ]pyridine; |
| 84 | 2-(Difluoromethyl)-4-(3-(5-fluoropyridin-2-yl)-1-methyl-1H-pyrazol-4-yl)- |
| | 1H-pyrrolo[2,3-b]pyridine; |
| 85 | 4-(3-(5-Fluoropyridin-2-yl)-1-methyl-1H-pyrazol-4-yl)-2-(trifluoromethyl)- |
| | 1H-pyrrolo[2,3-b]pyridine; |
| 86 | 4-(3-(5-Fluoropyridin-2-yl)-1-methyl-1H-pyrazol-4-yl)-3-(trifluoromethyl)- |
| | 1H-pyrrolo[2,3-b]pyridine; |
| 87 | 4-[3-(5-Fluoro-2-pyridyl)-1-methyl-pyrazol-4-yl]-5-(trifluoromethyl)-1H- |
| | pyrrolo[2,3-b]pyridine; |
| 88 | 4-[3-(5-Fluoro-2-pyridyl)-1-methyl-pyrazol-4-yl]-1H-pyrrolo[2,3-b] |
| | pyridine-3-carbonitrile; |
| 89 | 4-[3-(5-Fluoro-2-pyridyl)-1-methyl-pyrazol-4-yl]-1H-pyrrolo[2,3-b] |
| | pyridine-5-carbonitrile; |
| 90 | 2-[4-[3-(5-Fluoro-2-pyridyl)-1-methyl-pyrazol-4-yl]-1H-pyrrolo[2,3-b] |
| | pyridin-5-yl]acetonitrile; |
| 91 | 4-(3-(5-Fluoropyridin-2-yl)-1-methyl-1H-pyrazol-4-yl)-5-(oxetan-3-yl)-1H- |
| | pyrrolo[2,3-b]pyridine; |
| 92 | 4-(3-(5-Fluoropyridin-2-yl)-1-methyl-1H-pyrazol-4-yl)-2-(oxetan-3-y])-1H- |
| | pyrrolo[2,3-b]pyridine; |
| 93 | 4-[3-(5-Fluoro-2-pyridyl)-1-methyl-pyrazol-4-yl]-3-(oxetan-3-yl)-1H- |
| | pyrrolo[2,3-b]pyridine; |
| 94 | 3-(4-(3-(5-Fluoropyridin-2-yl)-1-methyl-1H-pyrazol-4-yl)-1H-pyrrolo[2,3-b] |
| | pyridin-2-yl)oxetan-3-ol; |
| 95 | 4-(3-(5-Fluoropyridin-2-yl)-1-methyl-1H-pyrazol-4-yl)-5-(oxetan-3- |
| | ylmethyl)-1H-pyrrolo[2,3-b]pyridine; |
| 96 | 3-(4-(3-(5-Fluoropyridin-2-yl)-1-methyl-1H-pyrazol-4-yl)-1H-pyrrolo[2,3-b] |
| | pyridin-2-yl)tetrahydrofuran-3-o1; |
| 97 | (R/S)-4-[3-(5-Fluoro-2-pyridyl)-1-methyl-pyrazol-4-yl]-3-(tetrahydrofuran- |
| | 3-ylmethyl)-1H-pyrrolo[2,3-b]pyridine; |
| 98 | 4-(3-(5-Fluoropyridin-2-yl)-1-methyl-1H-pyrazol-4-yl)-1H-pyrazolo[3,4-b] |
| | pyridine; |
| 99 | 4-(3-(5-Fluoropyridin-2-yl)-1-methyl-1H-pyrazol-4-yl)-6-methyl-1H- |
| | pyrazolo[3,4-b]pyridine; |
| 100 | 4-[3-(5-Fluoro-2-pyridyl)-1-methyl-pyrazol-4-yl]-3,6-dimethyl-1H- |
| | pyrazolo[3,4-b]pyridine; |
| 101 | 6-Cyclopropyl-4-(3-(5-fluoropyridin-2-yl)-1-methyl-1H-pyrazol-4-yl)-1H- |
| | pyrazolo[3,4-b]pyridine; |
| 102 | 4-(3-(5-Fluoropyridin-2-yl)-1-methyl-1H-pyrazol-4-yl)-7H-pyrrolo[2,3-d] |
| | pyrimidine; |
| 115 | N-(4-(3-(5-Fluoropyridin-2-yl)-1-methyl-1H-pyrazol-4-yl)pyridin-2-yl) |
| | acetamide; |
| 116 | N-(4-(3-(5-Fluoropyridin-2-yl)-1-methyl-1H-pyrazol-4-yl)pyridin-2yl)- |
| | cyclopropanecarboxamide; |
| 117 | 4-(3-(5-Fluoropyridin-2-yl)-1-methyl-1H-pyrazol-4-yl)-6,7-dihydro-5H- |
| | pyrrolo[3,4-b]pyridine; |
| 118 | 7-(3-(5-Fluoropyridin-2-yl)-1-methyl-1H-pyrazol-4-yl)thieno[3,2-b] |
| | pyridine; |
| 119 | 4-[3-(5-Fluoro-2-pyridyl)-1-methyl-pyrazol-4-yl]-1H-pyrrolo[2,3-b]pyridin- |
| | 6-amine; |
| 120 | 2-[4-[3-(5-Fluoro-2-pyridyl)-1-methyl-pyrazol-4-yl]-1H-pyrrolo[2,3-b] |
| | pyridin-3-yl]acetonitrile; |
| 121 | 1-Ethyl-5-[3-(5-fluoro-2-pyridyl)-1-methyl-pyrazol-4-yl]pyrazolo[3,4-b] |
| | pyridine; |
| 122 | 4-[3-(5-Fluoro-2-pyridyl)-1-methyl-pyrazol-4-yl]-3-isopropyl-1H- |
| | pyrazolo[3,4-b]pyridine; |
| 123 | 4-[3-(5-Fluoro-2-pyridyl)-1-methyl-pyrazol-4-yl]-2-methyl-7H-pyrrolo[2,3-d] |
| | pyrimidine; |
| 124 | 4-[3-(5-Fluoro-2-pyridyl)-1-methyl-pyrazol-4-yl]-6-methyl-1H- |
| | pyrazolo[3,4-d]pyrimidine; |
| 125 | 2-Cyclopropyl-4-[3-(5-fluoro-2-pyridyl)-1-methyl-pyrazol-4-yl]-7H- |
| | pyrrolo[2,3-d]pyrimidine; |
| 132 | 7-[3-(5-Fluoro-2-pyridyl)-1-methyl-pyrazol-4-yl]-3-methyl-thieno[3,2-b] |
| | pyridine; |
| 134 | 4-[1-Cyclopropyl-3-(5-fluoro-2-pyridyl)pyrazol-4-yl]-6-methyl-1H- |
| | pyrazolo[3,4-b]pyridine; |
| 138 | 4-[3-(3-Chloro-4-pyridyl)-1-methyl-pyrazol-4-yl]-6-methyl-1H- |
| | pyrazolo[3,4-b]pyridine; |
| 139 | 4-[3-(5-Chloro-3-pyridyl)-1-methyl-pyrazol-4-yl]-6-methyl-1H- |
| | pyrazolo[3,4-b]pyridine; |
| 143 | 6-Methyl-4-[1-methyl-3-(6-methylpyridazin-4-yl)pyrazol-4-yl]-1H- |
| | pyrazolo[3,4-b]pyridine;b] |
| 144 | 4-[3-(5-Fluoro-2-pyridyl)-1-methyl-pyrazol-4-yl]-1-methyl-pyrrolo[2,3-b] |
| | pyridine; |
| 145 | 4-[3-(5-Fluoro-2-pyridyl)-1-methyl-pyrazol-4-yl]thieno[2,3-b]pyridine; and |
| 146 | 6-(Difluoromethyl)-4-[3-(5-fluoro-3-pyridyl)-1-methyl-pyrazol-4-yl]-1H- |
| | pyrazolo[3,4-b]pyridine; |

and pharmaceutically acceptable salts, isotopes, N-oxides, solvates, and stereoisomers thereof.

A further embodiment of the current invention is a compound selected from the group consisting of:

TABLE 2

| Example | |
|---|---|
| # | Compound Name |
| 17 | 4-[1-Methyl-3-(4-pyridyl)pyrazol-4-yl]-1H-pyrrolo[2,3-b]pyridine; |
| 22 | 4-[1-Methyl-3-(6-methyl-3-pyridyl)pyrazol-4-yl]-1H-pyrrolo[2,3-b] |
| | pyridine; |
| 23 | 4-[3-[6-(Difluoromethoxy)-3-pyridyl]-1-methyl-pyrazol-4-yl]-1H- |
| | pyrrolo[2,3-b]pyridine; |
| 30 | 6-Methyl-4-(1-methyl-3-(6-methylpyridin-2-yl)-1H-pyrazol-4-yl)-1H- |
| | pyrazolo[3,4-b]pyridine; |
| 31 | 4-[1-Methyl-3-(5-methyl-2-pyridyl)pyrazol-4-yl]-1H-pyrrolo[2,3-b] |
| | pyridine; |
| 32 | 4-(1-Methyl-3-(5-methylpyridin-2-yl)-1H-pyrazol-4-yl)-1H-pyrazolo[3,4-b] |
| | pyridine; |
| 33 | 4-(3-(5-(Difluoromethyl)pyridin-2-yl)-1-methyl-1H-pyrazol-4-yl)-1H- |
| | pyrazolo[3,4-b]pyridine; |
| 34 | 4-(3-(6-Methoxypyridin-2-yl)-1-methyl-1H-pyrazol-4-yl)-6-methyl-1H- |
| | pyrazolo[3,4-b]pyridine; |
| 35 | 4-[3-[5-(Difluoromethoxy)-2-pyridyl]-1-methyl-pyrazol-4-yl]-1H- |
| | pyrrolo[2,3-b]pyridine; |
| 36 | 4-[1-Methyl-3-[5-(trifluoromethoxy)-2-pyridyl]pyrazol-4-yl]-1H- |
| | pyrrolo[2,3-b]pyridine; |
| 39 | 4-(3-(5-Chloro-6-methylpyridin-2-yl)-1-methyl-1H-pyrazol-4-yl)-6- |
| | methyl-1H-pyrazolo[3,4-b]pyridine; |
| 40 | 4-(3-(5-Fluoro-6-methylpyridin-2-yl)-1-methyl-1H-pyrazol-4-yl)-6- |
| | methyl-1H-pyrazolo[3,4-b]pyridine; |
| 41 | 4-(3-(5-Fluoro-6-methoxypyridin-2-yl)-1-methyl-1H-pyrazol-4-yl)-6- |
| | methyl-1H-pyrazolo[3,4-b]pyridine; |
| 42 | 4-(3-(5-Chloro-6-methoxypyridin-2-yl)-1-methyl-1H-pyrazol-4-yl)-6- |
| | methyl-1H-pyrazolo[3,4-b]pyridine; |
| 53 | 4-[1-Methyl-3-(3-pyridyl)pyrazol-4-yl]-1H-pyrrolo[2,3-b]pyridine; |
| 103 | 8-[3-(5-Fluoro-2-pyridyl)-1-methyl-pyrazol-4-yl]-2-methoxy-1,5- |
| | naphthyridine; |
| 104 | 4-[3-(5-Fluoro-2-pyridyl)-1-methyl-pyrazol-4-yl]quinoline; |
| 105 | 7-Chloro-4-[3-(5-fluoro-2-pyridyl)-1-methyl-pyrazol-4-yl]quinoline; |
| 106 | 7-Fluoro-4-[3-(5-fluoro-2-pyridyl)-1-methyl-pyrazol-4-yl]quinoline; |
| 107 | 4-[3-(5-Fluoro-2-pyridyl)-1-methyl-pyrazol-4-yl]-7-(trifluoromethyl) |
| | quinoline; |
| 108 | 4-[3-(5-Fluoro-2-pyridyl)-1-methyl-pyrazol-4-yl]-7-methoxy-quinoline; |
| 109 | 4-[3-(5-Fluoro-2-pyridyl)-1-methyl-pyrazol-4-yl]-7-(trifluoromethoxy) |
| | quinoline; |
| 110 | 4-[3-(5-Fluoro-2-pyridyl)-1-methyl-pyrazol-4-yl]-7-methoxy-2-methyl- |
| | quinoline; |
| 111 | 7-Fluoro-4-[3-(5-fluoro-2-pyridyl)-1-methyl-pyrazol-4-yl]-6-methoxy- |
| | quinoline; |
| 112 | 4-[3-(5-Fluoro-2-pyridyl)-1-methyl-pyrazol-4-yl]-1,7-naphthyridine; |
| 113 | 4-[3-(5-Fluoro-2-pyridyl)-1-methyl-pyrazol-4-yl]-5,6,7,8-tetrahydro-1,7- |
| | naphthyridine; |
| 114 | 5-[3-(5-Fluoro-2-pyridyl)-1-methyl-pyrazol-4-yl]-1,2,3,4-tetrahydro-1,8- |
| | naphthyridine; |
| 126 | 4-[3-(5-Fluoro-2-pyridyl)-1-methyl-pyrazol-4-yl]-1,5-naphthyridine; |
| 127 | 2-Fluoro-8-[3-(5-fluoro-2-pyridyl)-1-methyl-pyrazol-4-yl]-1,5- |
| | naphthyridine; |
| 128 | 2-Ethoxy-8-[3-(5-fluoro-2-pyridyl)-1-methyl-pyrazol-4-yl]-1,5- |
| | naphthyridine; |
| 129 | 4-[3-(5-Fluoro-2-pyridyl)-1-methyl-pyrazol-4-yl]-6-methoxy-quinoline; |
| 130 | 4-[3-(5-Fluoro-2-pyridyl)-1-methyl-pyrazol-4-yl]-1,6-naphthyridine; |
| 131 | 8-[3-(5-Fluoro-2-pyridyl)-1-methyl-pyrazol-4-yl]-2-methyl-1,5- |
| | naphthyridine; |
| 133 | 4-[3-(5-Fluoro-2-pyridyl)-1-methyl-pyrazol-4-yl]-8-methyl-1,5- |
| | naphthyridine; |
| 135 | 6-Methyl-4-[1-methyl-3-(2-methyl-4-pyridyl)pyrazol-4-yl]-1H- |
| | pyrazolo[3,4-b]pyridine; |
| 136 | 6-Methyl-4-[1-methyl-3-(3-methyl-4-pyridyl)pyrazol-4-yl]-1H- |
| | pyrazolo[3,4-b]pyridine; |
| 137 | 6-Methyl-4-[1-methyl-3-(5-methyl-3-pyridyl)pyrazol-4-yl]-1H- |
| | pyrazolo[3,4-b]pyridine; |
| 140 | 4-[3-(3-Fluoro-5-methyl-4-pyridyl)-1-methyl-pyrazol-4-yl]-6-methyl-1H- |
| | pyrazolo[3,4-b]pyridine; |
| 141 | 4-[3-(6-Methoxy-5-methyl-3-pyridyl)-1-methyl-pyrazol-4-yl]-6-methyl- |
| | 1H-pyrazolo[3,4-b]pyridine; |
| 142 | 4-[3-(5-Chloro-6-methoxy-3-pyridyl)-1-methyl-pyrazol-4-yl]-6-methyl- |
| | 1H-pyrazolo[3,4-b]pyridine; |
| 147 | 8-Fluoro-4-[3-(5-fluoro-2-pyridyl)-1-methyl-pyrazol-4-yl]quinoline; |
| 148 | 4-[3-(5-Fluoro-2-pyridyl)-1-methyl-pyrazol-4-yl]-8-methoxy-quinoline; |
| 149 | 4-[3-(5-Fluoro-2-pyridyl)-1-methyl-pyrazol-4-yl]quinoline-7-carbonitrile; |
| 150 | 4-[3-(5-Fluoro-2-pyridyl)-1-methyl-pyrazol-4-yl]quinoline-6-carbonitrile; |
| 151 | 4-[3-(5-Fluoro-2-pyridyl)-1-methyl-pyrazol-4-yl]-5,7-dimethoxy- |
| | quinoline; |
| 152 | 3-Fluoro-8-[3-(5-fluoro-2-pyridyl)-1-methyl-pyrazol-4-yl]-1,5- |
| | naphthyridine; |
| 153 | 3-Bromo-8-[3-(5-fluoro-2-pyridyl)-1-methyl-pyrazol-4-yl]-1,5- |
| | naphthyridine; |
| 154 | 4-[3-(5-Fluoro-2-pyridyl)-1-methyl-pyrazol-4-yl]-7-methoxy-1,6- |
| | naphthyridine; |
| 155 | 8-[3-(5-Fluoro-2-pyridyl)-1-methyl-pyrazol-4-yl]-3-methoxy-1,5- |
| | naphthyridine; and |
| 156 | 7-Fluoro-8-[3-(5-fluoro-2-pyridyl)-1-methyl-pyrazol-4-yl]-2-methoxy- |
| | 1,5-naphthyridine; |

an pharmaceutically acceptable salts, isotopes, N-oxides, solvates, an stereoisomers thereof.

A further embodiment of the current invention is a compound selected from the group consisting of:
4-[3-(5-Fluoro-2-pyridyl)-1-methyl-pyrazol-4-yl]-2-methyl-7H-pyrrolo[2,3-d]pyrimidine;
3-Chloro-4-[3-(5-fluoro-2-pyridyl)-1-methyl-pyrazol-4-yl]-1H-pyrrolo[2,3-b]pyridine;
N-(4-(3-(5-Fluoropyridin-2-yl)-1-methyl-1H-pyrazol-4-yl)pyridin-2-yl)propionamide;
4-(3-(5-Fluoropyridin-2-yl)-1-methyl-1H-pyrazol-4-yl)-6-methyl-1H-pyrazolo[3,4-b]pyridine;
4-(1-(2,2-Difluoroethyl)-3-(5-fluoropyridin-2-yl)-1H-pyrazol-4-yl)-6-methyl-1H-pyrazolo[3,4-b]pyridine; and
4-[3-(5-Fluoro-2-pyridyl)-1-methyl-pyrazol-4-yl]-6-methyl-1H-pyrrolo[2,3-b]pyridine; and pharmaceutically acceptable salts, isotopes, N-oxides, solvates, and stereoisomers thereof.

An additional embodiment of the invention is a compound of Formula (I) having the Formula (IA):

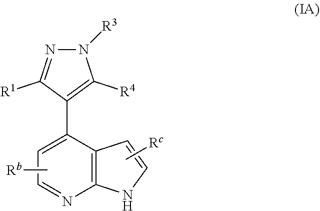

wherein
R¹ is selected from the group consisting of:
(a)

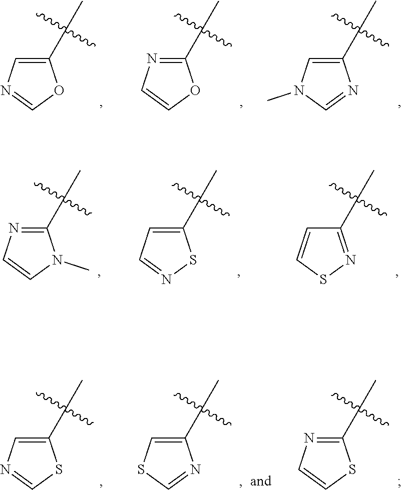

(b)

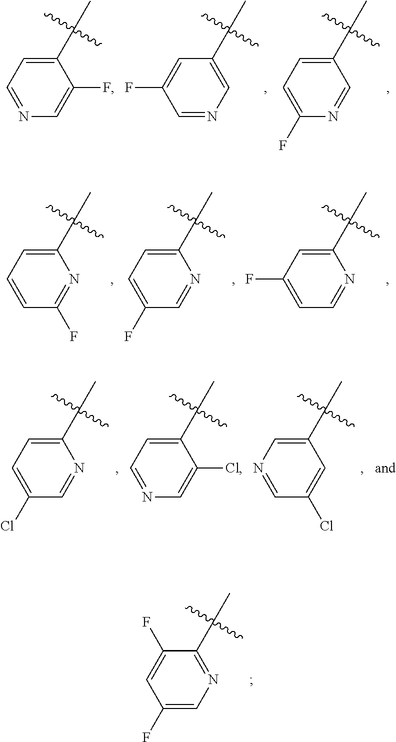

and
(c)

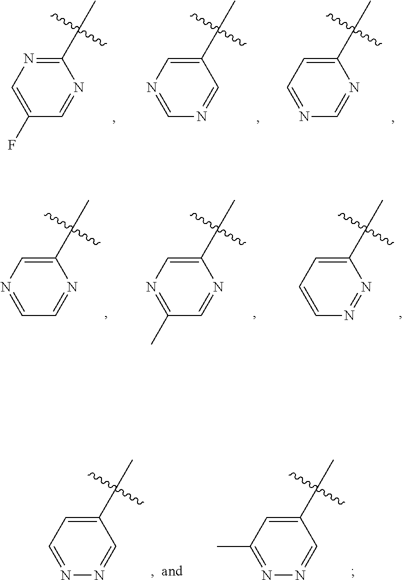

R^{b} is selected from the group consisting of: H, F, CH₃, CF₃, CN, NH₂,

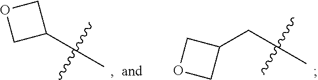 [SMILES: *C1COC1 ; *CC1COC1]

R^{c} is selected from the group consisting of: H, Br, Cl, F, CH₃, CH(CH₃)₂, CHF₂, CF₃,

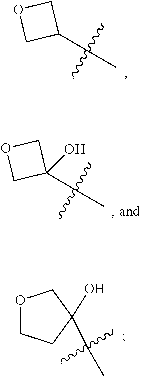 [SMILES: *C1COC1 ; *C1(O)COC1 ; *C1(O)CCOC1]

R³ is selected from the group consisting of: CH₃, CH₂CH₃, CH(CH₃)₂, CH₂CH(CH₃)₂, CHF₂, CH₂CH₂F,

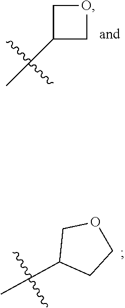 [SMILES: *C1COC1 ; *C1CCOC1]

and
R⁴ is selected from the group consisting of: H, CH₃, and cyclobutyl.

An additional embodiment of the invention is a compound of Formula (I) having the Formula (IB):

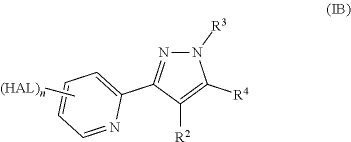

wherein
R² is selected from the group consisting of:
(a)

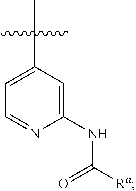

(b)

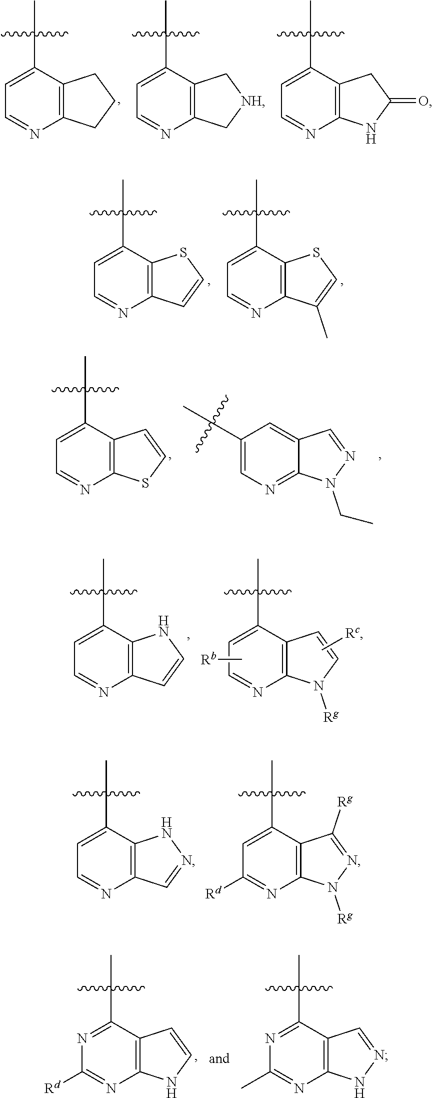

wherein
R^{a} is selected from the group consisting of: CH₃, CH₂CH₃, and cyclopropyl;
R^{b} is selected from the group consisting of: H, F, CH₃, CF₃, CN, NH₂,

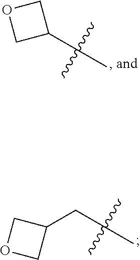 [SMILES: *C1COC1 ; *CC1COC1]

R^{c} is selected from the group consisting of: H, Br, Cl, F, CH₃, CH(CH₃)₂, CHF₂, CF₃, CN,

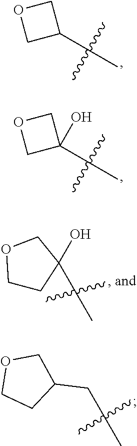 [SMILES: *C1COC1 ; *C1(O)COC1 ; *C1(O)CCOC1 ; *CC1CCOC1]

R^{d} is selected from the group consisting of: H, CH₃, CF₂H, and cyclopropyl;
R^{g} is selected from the group consisting of: H, CH₃, and CH₂CH₃;
HAL is independently selected from: Cl and F;
n is 1 or 2;
R³ is selected from the group consisting of: CH₃, CH₂CH₃, CH(CH₃)₂, CH₂CH(CH₃)₂, CHF₂, CH₂CH₂F, CH₂CHF₂, CH₂CH₂OCH₃, cyclopropyl, cyclobutyl,

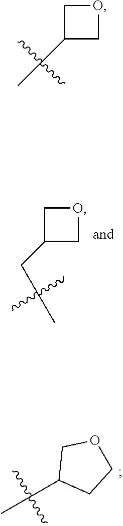 [SMILES: *C1COC1 ; *CC1COC1 ; *C1CCOC1]

and
R⁴ is selected from the group consisting of: H, CH₃, and cyclobutyl.

An additional embodiment of the invention is a compound selected from the group consisting of compounds of Formula (I), Formula (IA), and Formula (IB) or a combination thereof.
An additional embodiment of the invention is a pharmaceutical composition comprising:
(A) a therapeutically effective amount of at least one compound selected from compounds of Formula (I)

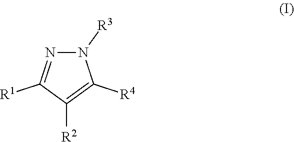

wherein
R¹ is selected from the group consisting of:
(a) 5-membered heteroaryl selected from the group consisting of:

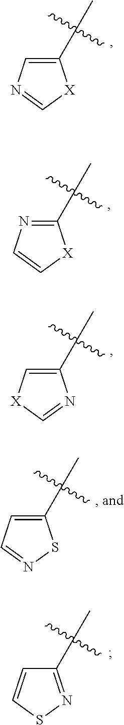

(b) pyridinyl substituted with one or two halo members;
(c) pyrimidinyl; pyrimidinyl substituted with halo; pyrazinyl; pyrazinyl substituted with C₁₋₆ alkyl; pyridazinyl; and pyridazinyl substituted with C₁₋₆ alkyl;
R² is selected from the group consisting of:
(d)

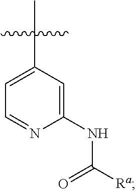

(e)

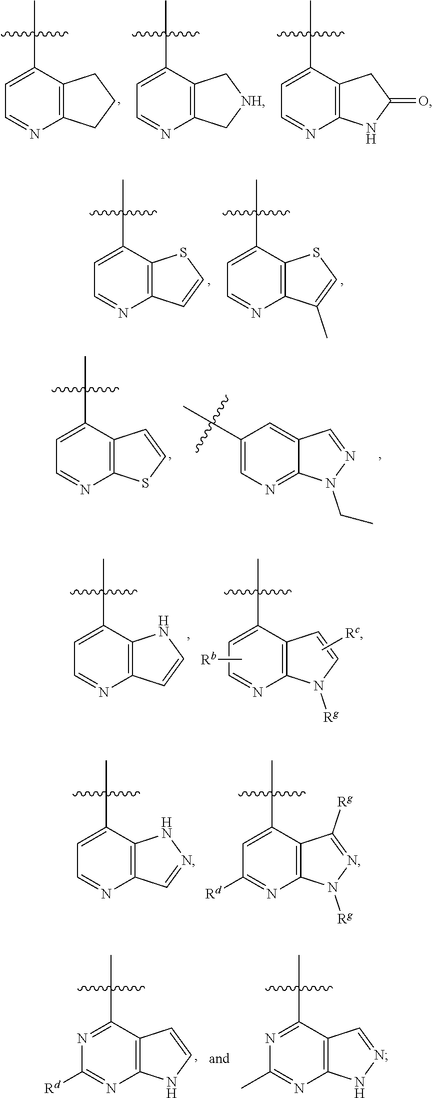

wherein
R^{a} is C₁₋₃ alkyl or C₃₋₆ cycloalkyl;
R^{b} is selected from the group consisting of: H, halo, C₁₋₃ alkyl, C₁₋₃ haloalkyl, CN, CH₂CN, NH₂, oxetanyl, and CH₂-oxetanyl;
R^{c} is selected from the group consisting of: H, halo, C₁₋₃ alkyl, C₁₋₃ haloalkyl, CN, CH₂CN, oxetanyl, oxetanyl substituted with OH, tetrahydrofuranyl substituted with OH, and CH₂-tetrahydrofuranyl;
R^{d} is selected from the group consisting of: H, C₁₋₃ alkyl, C₁₋₃ haloalkyl, and C₃₋₆ cycloalkyl;
R^{g} is H or C₁₋₃ alkyl;
X is O, S, or N—CH₃;
R³ is selected from the group consisting of: C₁₋₆ alkyl, CH₂CH₂OCH₃, C₁₋₆ haloalkyl, C₃₋₆ cycloalkyl, oxetanyl, CH₂-oxetanyl, and tetrahydrofuranyl; and
R⁴ is selected from the group consisting of: H, C₁₋₃ alkyl, and C₃₋₆ cycloalkyl; and pharmaceutically acceptable salts, isotopes, N-oxides, solvates, and stereoisomers of compounds of Formula (I);
and (B) at least one pharmaceutically acceptable excipient.

An additional embodiment of the invention is a pharmaceutical composition comprising a therapeutically effective amount of at least one compound in Table 1, as well as and pharmaceutically acceptable salts, isotopes, N-oxides, solvates, and stereoisomers of compounds of Table 1, pharmaceutically acceptable prodrugs of compounds of Table 1, and pharmaceutically active metabolites of Table 1; and at least one pharmaceutically acceptable excipient.

An additional embodiment of the invention is a pharmaceutical composition comprising a therapeutically effective amount of at least one compound in Table 2, as well as and pharmaceutically acceptable salts, isotopes, N-oxides, solvates, and stereoisomers of compounds of Table 2, pharmaceutically acceptable prodrugs of compounds of Table 2, and pharmaceutically active metabolites of Table 2; and at least one pharmaceutically acceptable excipient.

An additional embodiment of the invention is a pharmaceutical composition comprising a therapeutically effective amount of at least one compound of Formula (IA), as well as pharmaceutically acceptable salts, N-oxides or solvates of compounds of Formula (IA), pharmaceutically acceptable prodrugs of compounds of Formula (IA), and pharmaceutically active metabolites of Formula (IA); and at least one pharmaceutically acceptable excipient.

An additional embodiment of the invention is a pharmaceutical composition comprising a therapeutically effective amount of at least one compound of Formula (IB), as well as pharmaceutically acceptable salts, N-oxides or solvates of compounds of Formula (IB), pharmaceutically acceptable prodrugs of compounds of Formula (IB), and pharmaceutically active metabolites of Formula (IB); and at least one pharmaceutically acceptable excipient.

Also within the scope of the invention are enantiomers and diastereomers of the compounds of Formula (I) (as well as Formulas (IA) and (IB)) Also within the scope of the invention are the pharmaceutically acceptable salts, N-oxides or solvates of the compounds of Formula (I) (as well as Formulas (IA) and (IB)). Also within the scope of the invention are the pharmaceutically acceptable prodrugs of compounds of Formula (I) (as well as Formulas (IA) and (IB)), and pharmaceutically active metabolites of the compounds of Formula (I) (as well as Formulas (IA) and (IB)).

Also within the scope of the invention are isotopic variations of compounds of Formula (I) (as well as Formulas (IA) and (IB)), such as, e.g., deuterated compounds of Formula (I). Also within the scope of the invention are the pharmaceutically acceptable salts, N-oxides or solvates of the isotopic variations of the compounds of Formula (I) (as well as Formulas (IA) and (IB)). Also within the scope of the invention are the pharmaceutically acceptable prodrugs of the isotopic variations of the compounds of Formula (I) (Formula (I) (as well as Formulas (IA) and (IB)), and pharmaceutically active metabolites of the isotopic variations of the compounds of Formula (I) (as well as Formulas (IA) and (IB)).

An additional embodiment of the invention is a method of treating a subject suffering from or diagnosed with a disease, disorder, or condition mediated by protein kinase CSNK1D activity, comprising administering to a subject in need of such treatment a therapeutically effective amount of at least one compound selected from compounds of Formula (II):

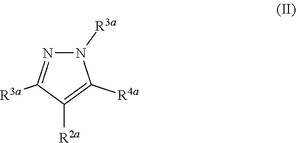

wherein
R^{1a} is selected from the group consisting of:
(a) 5-membered heteroaryl selected from the group consisting of:

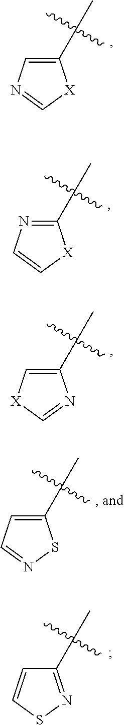

(b) pyridinyl, pyridinyl substituted with one or two members each independently selected from the group consisting of: halo, C₁₋₆ alkyl, C₁₋₆ haloalkyl, OC₁₋₆ alkyl, and OC₁₋₆ haloalkyl;
(c) pyrimidinyl; pyrimidinyl substituted with halo; pyrazinyl; pyrazinyl substituted with C₁₋₆ alkyl; pyridazinyl; and pyridazinyl substituted with C₁₋₆ alkyl;
R^{2a} is selected from the group consisting of:
(d)

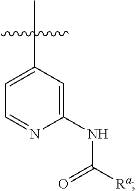

(e)

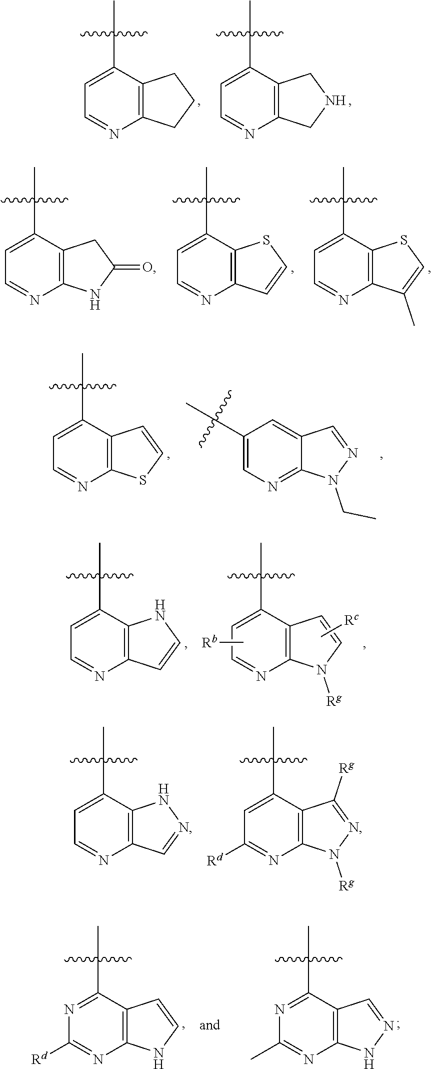

and
(f)

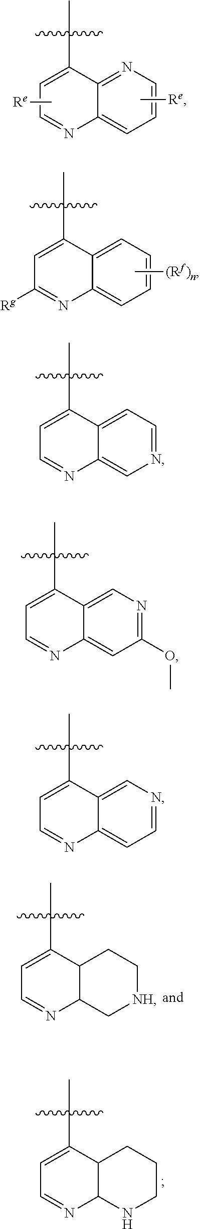

wherein
R^{a} is C₁₋₃ alkyl or C₃₋₆ cycloalkyl;
R^{b} is selected from the group consisting of: H, halo, C₁₋₃ alkyl, C₁₋₃ haloalkyl, CN, CH₂CN, NH₂, oxetanyl, and CH₂-oxetanyl;
R^{c} is selected from the group consisting of: H, halo, C₁₋₃ alkyl, C₁₋₃ haloalkyl, CN, CH₂CN, oxetanyl, oxetanyl substituted with OH, tetrahydrofuranyl substituted with OH, and CH₂-tetrahydrofuranyl;
R^{d} is selected from the group consisting of: H, C₁₋₃ alkyl, C₁₋₃ haloalkyl, and C₃₋₆ cycloalkyl;
R^{e} is selected from the group consisting of: H, halo, C₁₋₃ alkyl, and OC₁₋₃ alkyl;
R^{f} is independently selected from the group consisting of: H, halo, C₁₋₃ alkyl, C₁₋₃ haloalkyl, OC₁₋₃ alkyl, OC₁₋₃ haloalkyl, and CN;
R^{g} is H or C₁₋₃ alkyl;
X is O, S, or N—CH₃;
n is 1 or 2;
R^{3a} is selected from the group consisting of: H, C₁₋₆ alkyl, CH₂CH₂OCH₃, C₁₋₆ haloalkyl, C₃₋₆ cycloalkyl, oxetanyl, CH₂-oxetanyl, and tetrahydrofuranyl; and
R^{4a} is selected from the group consisting of: H, C₁₋₃ alkyl, and C₃₋₆ cycloalkyl; and pharmaceutically acceptable salts, isotopes, N-oxides, solvates, and stereoisomers thereof, to a subject in need thereof.

An additional embodiment of the invention is a method of treating a subject suffering from or diagnosed with a disease, disorder, or condition mediated by protein kinase CSNK1D receptor activity, comprising administering to a subject in need of such treatment a therapeutically effective amount of at least one compound selected from compounds of Formula (II) (as well as Formulas (I), (IA) and (IB)), enantiomers and diastereomers of the compounds of Formula (II) (as well as Formulas (I), (IA) and (IB)), isotopic variations of the compounds of Formula (II) (as well as Formulas (I), (IA) and (IB)), and pharmaceutically acceptable salts of all of the foregoing.

Exemplary compounds useful in methods of the invention will now be described by reference to the illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Unless otherwise specified, the variables are as defined above in reference to Formula (I) or Formula (II). Reactions may be performed between the melting point and the reflux temperature of the solvent, and preferably between 0° C. and the reflux temperature of the solvent. Reactions may be heated employing conventional heating or microwave heating. Reactions may also be conducted in sealed pressure vessels above the normal reflux temperature of the solvent.

Abbreviations and Acronyms Used Herein Include the Following:

TABLE 3

| Acronym | Term |
|---|---|
| ° C. | Degree Celsius |
| BOC | tert-butyloxycarbonyl |
| BOC₂O | di-tert-butyl dicarbonate |
| μL | Microliter |
| μmol | micromoles |
| ACN, MeCN | Acetonitrile |
| AcOH | Acetic acid |
| Aq, or Aq. | Aqueous |
| atm | Atmosphere |
| (binaphthyl)P(t-Bu)₂, | rac-2-(Di-tert-butylphosphino)-1,1′-binaphthyl |
| TrixiePhos | |
| BOP | benzotriazol-1-yloxy-tris(dimethylamino)phosphonium |
| | hexafluorophosphate |
| br | Broad |
| Bs | benzenesulfonyl |
| calcd. | calculated |
| Celite ® | Diatomaceous Earth |
| CK1 delta or casein kinase | Casein kinase 1 delta |
| 1δ or CSNK1D | |
| DCC | N,N′-dicyclohexylcarbodiimide |
| DCE | dichloroethane |
| DCM | dichloromethane |
| DIEA, DIPEA | N-ethyldiisopropylamine |
| DMA | dimethylacetamide |
| DMAP | 4-dimethylaminopyridine |
| DME | dimethoxyethane |
| DMF | dimethylformamide |
| DMF-DMA | N,N-Dimethylformamide dimethyl acetal |
| DMSO | Dimethylsulfoxide |
| DPPF | 1,1′-bis(diphenylphosphino)ferrocene |
| EDC, EDAC or EDCI | 1-ethyl-3-(3-dimethylaminopropyl)carbodiimide |
| ESI | Electrospray ionization |
| Ether, Et₂O | Diethyl ether |
| EtOAc, or EA | Ethyl Acetate |
| EtOH | Ethanol |
| FCC | Normal-phase silica gel chromatography |
| g | Grams |
| h, hr, hrs | Hours |
| HAL | Halogen |
| HATU | 1-[bis(dimethylamino)methylene]-1H-1,2,3-triazolo[4,5-b] |
| | pyridinium 3-oxid hexafluorophosphate |
| HBTU | N,N,N′,N′-tetramethyl-O-(1H-benzotriazol-1-yl)uronium |
| | hexafluorophosphate |
| Hex | hexanes |
| HOBt | hydroxybenzotriazole |
| HPLC | High-pressure liquid chromatography |
| Hz | Hertz |
| iPrOH, IPA | Isopropyl alcohol |
| Ir(ppy)₂(dtbbpy)PF₆ | [4,4′-Bis(1,1-dimethylethyl)-2,2′-bipyridine-N1,N1′]bis[2-(2- |
| | pyridinyl-N)phenyl-C]iridium(III) hexafluorophosphate |
| (Ir[dF(CF₃)ppy]₂(dtbpy))PF₆ | [4,4′-Bis(1,1-dimethylethyl)-2,2′-bipyridine-N1,N1′]bis[3,5- |
| | difluoro-2-[5-(trifluoromethyl)-2-pyridinyl-N]phenyl-C]Iridium(III) |
| | hexafluorophosphate |
| Josiphos SL-J009-1 PD G3 | { ®-1-[(Sp)-2-(Dicyclohexylphosphino)ferrocenyl]ethyldi-tert- |
| | butylphosphine}[2-(2′-amino-1,1′-biphenyl)]palladium(II) |
| | methanesulfonate |
| KOtBu or t-BuOK | Potassium tert-Butoxide |
| KOAc | Potassium acetate |
| LCMS or LC-MS | Liquid chromatography and mass spectrometry |
| LED | Light emitting diode |
| LDA | lithium diisopropylamide |
| LiHMDS or LHMDS | Lithium bis(trimethylsilyl)amide |
| M | Molar |
| m/z | Mass to charge ratio |
| mCPBA | 3-chloroperoxybenzoic acid |
| Me | methyl |
| MeOH | Methanol |
| mg | Milligram |
| min | Minute |
| mL | Milliliter |
| mmol | Millimole |
| MS | Mass spectrometry |
| ESI | Electrospray ionization |
| MTBE, or TBME | tert-butyl methyl ether |
| NMP | 1-methyl-2-pyrrolidinone |
| N | Normal |
| NMR | Nuclear magnetic resonance |
| NaOAc tri-hydrate | Sodium acetate trihydrate |
| OTf | CF₃SO₃— or triflate |
| PhenoFluor ™ | N,N′-,3-Bis(2,6-diisopropylphenyl)-2,2-difluoroimidazolidene |
| Pd(OAc)₂ | Palladium(II) acetate |
| cataCXium ® A Pd G3 | [(Di(1-adamantyl)-butylphosphine)-2-(2′-amino-1,1′- |
| | biphenyl)]palladium(II) methanesulfonate |
| PdCl₂ | Palladium(II) chloride |
| Pd(PPh₃)₂Cl₂ | palladium(II)bis(triphenylphosphine) dichloride |
| Pd(PPh₃)₄ | tetrakis(triphenylphosphine)palladium(0) |
| Pd(dppf)Cl₂ | [1,1′-bis(diphenylphosphino)ferrocene]dichloropalladium(II) |
| Pd(dtbpf)Cl₂ | [1,1′-bis(di-tert-butylphosphino)ferrocene]dichloropalladium(II) |
| Pd(TFA)₂ | trifluoroacetic acid palladium(II) salt |
| Pd2(dba)₃ | tris(dibenzylidene)dipalladium(0) |
| Pd(t-Bu3P)₂ | bis(tri-tert-butylphosphine)palladium(0) |
| Pd(dppf)Cl₂•DCM | [1,1′-Bis(diphenylphosphino)ferrocene]dichloropalladium(II), |
| | complex with dichloromethane |
| Pd(amphos)Cl₂ | Bis(di-tert-butyl(4-dimethylaminophenyl)phosphine) |
| | dichloropalladium(II) |
| PE | Petroleum ether |
| PG | Protecting group |
| ppm | Parts per million |
| ppt | Precipitate |
| PTFE | Polytetrafluoroethylene |
| PyBroP ® | bromotripyrrolidinophosphonium hexafluorophosphate |
| p-TsOH or TsOH | p-toluenesulfonic acid |
| RuPhos Pd G3 | (2-Dicyclohexylphosphino-2′,6′-diisopropoxy-1,1′-biphenyl)[2-(2′- |
| | amino-1,1′-biphenyl)]palladium(II) methanesulfonate |
| RP | Reverse Phase |
| Rₜ | Retention time |
| rt | Room temperature |
| sat | Saturated |
| SFC | Supercritical Fluid Chromatography |
| T | Temperature |
| TBAF | Tetrabutylammonium fluoride |
| T3P ® | 2,4,6-tripropyl-1,3,5,2,4,6-trioxatriphosphorinane-2,4,6-trioxide |
| TEA | triethylamine |
| Tf₂NPh | N-phenylbis(trifluoromethanesufonimide) |
| Tf₂O | trifluoromethanesulfonic anhydride |
| TFA | trifluoroacetic acid |
| THF | tetrahydrofuran |
| TLC | Thin layer chromatography |
| Triflate, or Tf | trifluoromethanesulfonyl |
| Ts | toluenesulfonyl |
| UV | ultraviolet |
| V, or volumes | Volume in milliliters of solvent per gram of substrate |
| Xphos-Pd-G3 | (2-Dicyclohexylphosphino-2′,4′,6′-triisopropyl-1,1′-biphenyl)[2-(2′- |
| | amino-1,1′-biphenyl)]palladium(II) methanesulfonate |
| ¹H NMR | Proton nuclear magnetic resonance |
| d | doublet |
| dt | Doublet of triplets |
| hept | heptet |
| S | singlet |
| J | Coupling constant |
| dd | Doublet of doublet |
| m | Multiplet |
| q | quadruplet |
| t | triplet |
| td | Triplet of doublets |
| ddd | Doublet of doublets of doublets |
| mHz | megahertz |
| L | liter |
| NBS | N-bromosuccinimide |
| EtOH | ethanol |
| Dowtherm ® A | eutectic mixture of 26.5% diphenyl + 73.5% diphenyl oxide |
| XtalFluor-E ® | (Diethylamino)difluorosulfonium tetrafluoroborate |
| SEMCl | 2-(Trimethylsilyl)ethoxymethyl chloride |
| SEM | 2-(Trimethylsilyl)ethoxymethyl |
| BF₃-OEt₂ | Boron trifluoride diethyl etherate |
| [M] | metal |
| HET | Heterocycle |

### PREPARATIVE EXAMPLES

Exemplary compounds useful in methods of the invention will now be described by reference to the illustrative synthetic schemes for their general preparation below and the specific examples to follow.

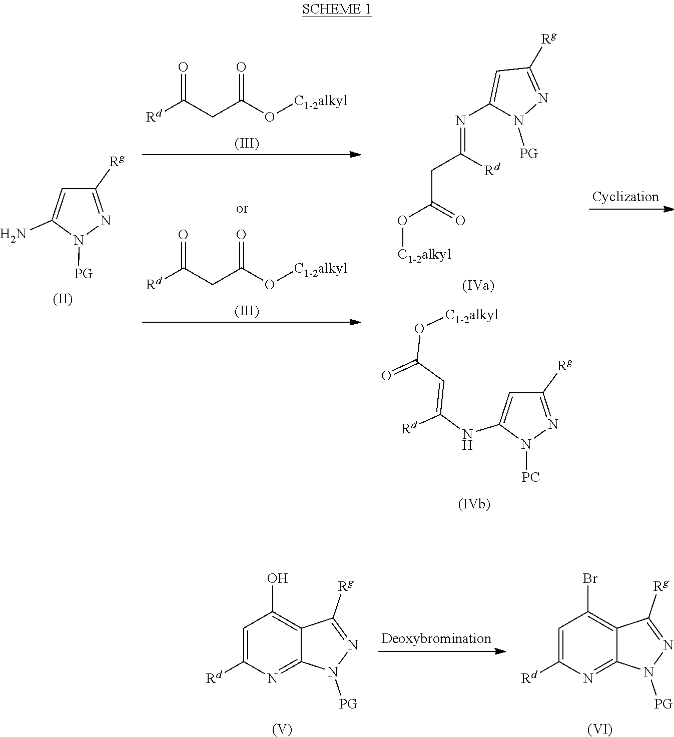

According to SCHEME 1, a compound of formula (IVa) where R^{g} is H or C₁₋₃ alkyl, R^{d} is C₁₋₃ alkyl or C₃₋₆ cycloalkyl, and PG is benzyl; is prepared from a compound of formula (II), where R^{g} is H or C₁₋₃ alkyl, and PG is benzyl; by condensation with a compound of formula (III) where R^{d} is C₁₋₃ alkyl or C₃₋₆ cycloalkyl; using a catalyst such asp-toluenesulfonic acid (TsOH) or acetic acid and the like; in a suitable solvent such as toluene and the like; at a temperature ranging from 70° C. to the reflux temperature of the solvent; for a period of about 14-24 h. In an alternate method a compound of formula (II), where R^{g} is H, and PG is p-methoxybenzyl; is condensed with a compound of formula (III) where R^{d} is C₁₋₃ alkyl; in a suitable solvent such as toluene and the like; a suitable acid such as TsOH and the like; at a temperature of 70° C. for a period of about 4 h to provide a compound of formula (IVb).

Thermal cyclization of a compound of formula (IVa) or a compound of formula (IVb) is achieved in a high-boiling solvent mixture such as Dowtherm® A and the like, at a temperature of around 275° C., for a period of about 1-6 h. Deoxybromination of compound of formula (V), where Y¹ is H or CH₃, is achieved using a brominating agent such as phosphorus oxybromide (POBr₃) and the like, in a mixture of solvents such as toluene and DMF, and the like, at a temperature ranging from 60 to 115° C., for a period of 1-2 h, to afford a compound of formula (VI), where R^{g} is H or C₁₋₃ alkyl, R^{d} is C₁₋₃ alkyl or C₃₋₆ cycloalkyl, and PG is benzyl.

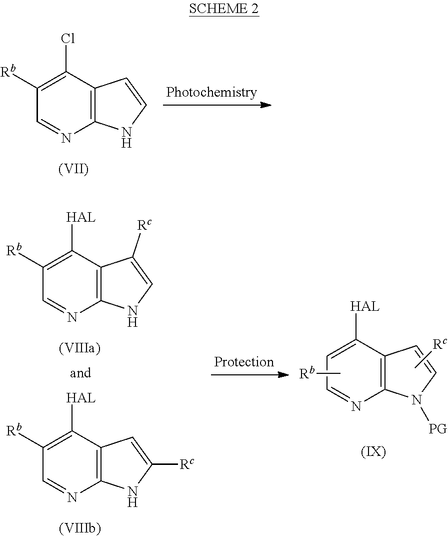

According to SCHEME 2, a commercially available or synthetically accessible compound of formula (VII), where R^{b} is H, is reacted under photochemical conditions known to one skilled in the art, to provide a mixture of compounds of formula (VIIIa) and (VIIIb), where HAL is Cl, R^{b} is H, and R^{c} is C₁₋₃ haloalkyl. For example, 4-chloro-7-azaindole is treated with (Ir[dF(CF₃)ppy]₂(dtbpy))PF₆, sodium trifluoromethanesulfonate, and an oxidant such as ammonium persulfate, and the like; in a suitable solvent such as DMSO, and the like; irradiated with blue light for a period of 2.5 h at rt to provide a mixture of compounds of formulas (VIIIa) and (VIIIb).

In a similar fashion, a compound of formula (VII), where R^{b} is Br, is reacted under photochemical conditions known to one skilled in the art, to provide a compound of formula (VIIIa), where R^{c} is H. For example, a compound of formula (VII), where R^{b} is Br, is treated with (Ir[dF(CF₃)ppy]₂(dtbpy))PF₆, anhydrous lithium hydroxide, nickel(II) chloride ethylene glycol dimethyl ether complex, 4,4′-di-tert-butyl-2,2′-bipyridine, tris(trimethylsilyl)silane, and 3-bromooxetane or 3-(bromomethyl)oxetane, in a suitable solvent such as dimethoxyethane, irradiated with blue light for a period of 3 h, at ambient temperature, to provide a compound of formula (VIIIa), where HAL is Cl, R^{b} is oxetanyl or CH₂-oxetanyl, and R^{c} is H.

Protection of compounds of formula (VIIIa) or (VIIIb) with a suitable nitrogen protecting group such as SEM, is achieved according to methods known to one skilled in the art or as previously described to afford a compound of formula (IX), where PG is SEM.

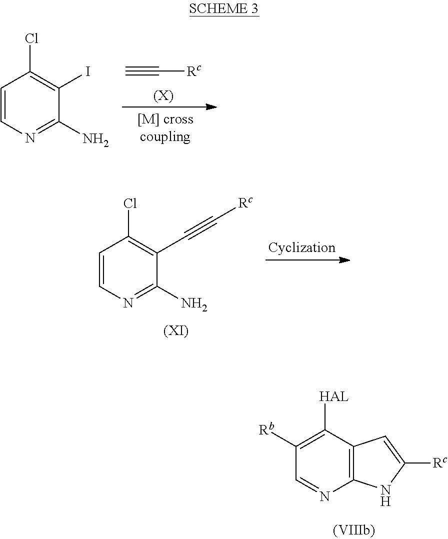

According to SCHEME 3, 4-chloro-3-iodopyridin-2-amine is reacted in a metal mediated cross coupling, such as a Sonogashira conditions, with an alkyne of formula (X), where R^{c} is C₁₋₃ alkyl or oxetanyl substituted with OH; a palladium catalyst such as PdCl₂(PPh₃)₂, and the like; a base such as triethylamine; CuI; in a suitable solvent such as ACN, and the like; to provide a compound of formula (XI), where R^{c} is C₁₋₃ alkyl or oxetanyl substituted with OH. A compound of formula (XI), is cyclized in the presence of a suitable base such as potassium tert-butoxide, and the like; in a suitable solvent such as NMP, and the like; to provide a compound of formula (VIIIb), where HAL is Cl, R^{b} is H, and R^{c} is H, C₁₋₃ alkyl or oxetanyl substituted with OH.

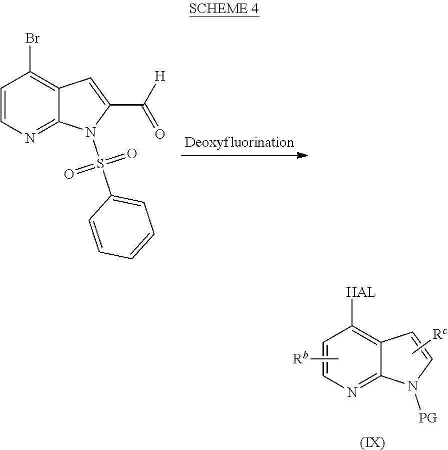

According to SCHEME 4, commercially available or synthetically accessible 4-bromo-1-(phenylsulfonyl)-1H-pyrrolo[2,3-b]pyridine-2-carbaldehyde is deoxyfluorinated by reaction with a reagent such as XtalFluor-E®, in the presence of a promoter such as triethylamine trihydrofluoride, in a suitable solvent such as CH₂Cl₂, at temperatures ranging from 0° C. to rt; to afford a compound of formula (IX), where R^{b} is H, HAL is Br, R^{c} is CHF₂, and PG is benzenesulfonyl (Bs).

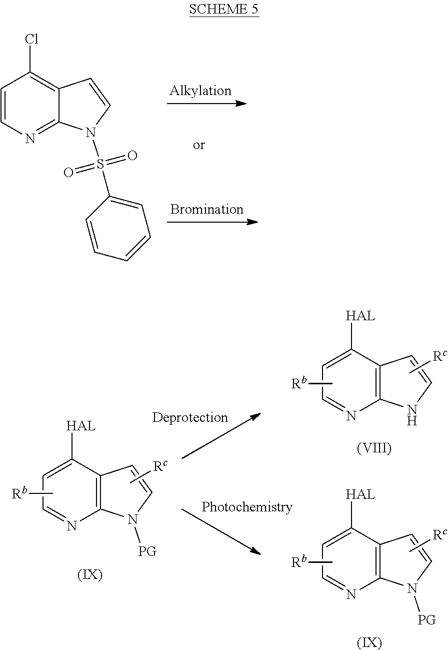

According to SCHEME 5, commercially available or synthetically accessible 4-chloro-1-(phenylsulfonyl)-1H-pyrrolo[2,3-b]pyridine is alkylated with dihydrofuran-3(2H)-one, in the presence of a suitable base such as n-butyllithium, and the like; in a suitable inert solvent such as tetrahydrofuran, and the like; at temperatures ranging from −78° C. to room temperature; to provide a compound of formula (IX), where HAL is Cl, R^{b} is H, R^{c} is 3-hydroxytetrahydrofuran-3-yl, and PG is benzenesulfonyl (Bs). Deprotection of a compound of formula (IX) where HAL is Cl, R^{b} is H, R^{c} is tetrahydrofuran substituted with one OH, and PG is benzenesulfonyl, is achieved is treated with a suitable base or reductant such as KOH, NaOH, K₂CO₃, LiOH, Triton B, magnesium, and the like; in a suitable solvent such as methanol, tetrahydrofuran, dioxane, and the like; at temperatures ranging from room temperature to 70° C.; for a period of 6-8 h; to provide a compound of formula (VIII).

Alternately, commercially available or synthetically accessible 4-chloro-1-(phenylsulfonyl)-1H-pyrrolo[2,3-b]pyridine is brominated employing conditions known to one skilled in the art, for example, 4-chloro-1-(phenylsulfonyl)-1H-pyrrolo[2,3-b]pyridine is treated with a base such as lithium diisopropylamide (LDA), and the like; at a temperature ranging from −78° C. to room temperature; an electrophilic brominating reagent such as bromohaloalkanes for example 1,2-dibromotetrachloroethane and perfluoroalkylbromides, and the like; in a suitable solvent such as THF, and the like; to provide a compound of formula (IX), where R^{b} is H, HAL is Cl, R^{c} is Br, and PG is benzenesulfonyl. A compound of formula (IX), where HAL is Cl, R^{b} is H, R^{c} is Br, and PG is benzenesulfonyl, is reacted under photochemical conditions as previously described to provide a compound of formula (IX), where HAL is Cl, R^{b} is H, R^{c} is oxetanyl and PG is benzenesulfonyl.

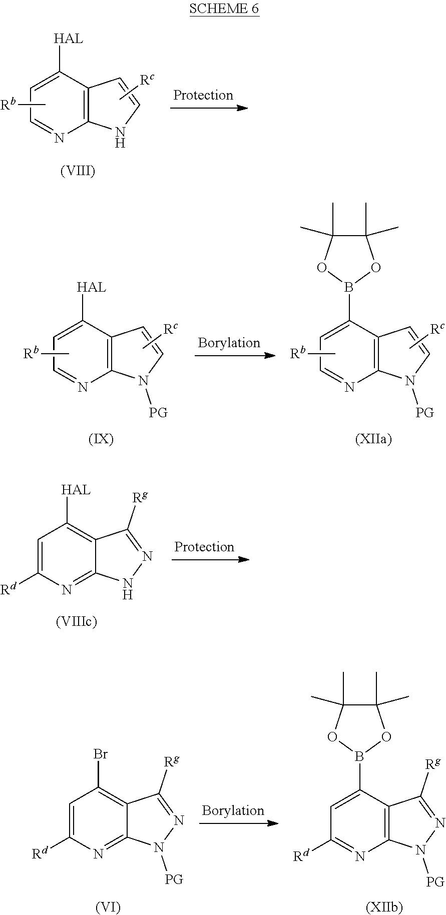

According to SCHEME 6, a compound of formula (VIII) (which also encompasses compound of formula (VIIIa) and (VIIIb)), where HAL is Br or Cl, R^{b} is H or C₁₋₃ alkyl, and R^{c} is H or C₁₋₃ haloalkyl; is protected with a suitable nitrogen protecting group (PG) such as SEM (2-(trimethylsilyl)ethoxymethyl), tert-butyloxycarbonyl (BOC), Ts (toluenesulfonyl) or benzenesulfonyl, and the like, under conditions known to one skilled in the art, to provide a compound of formula (IX). A compound of formula (VIII) is protected with a SEM protecting group, employing conditions known to one skilled in the art, for example, by reaction of a compound of formula (VIII) with 2-chloromethoxymethyl)trimethylsilane, in the presence of a base such as NaH, and the like, in a suitable solvent such as DMF, and the like; at temperatures ranging from 0° C. to rt, to provide a compound of formula (IX), where PG is SEM. A compound of formula (VIII) is protected with a BOC protecting group, employing conditions known to one skilled in the art, for example, by reacting a compound of formula (VIII) with BOC-anhydride, at room temperature, for a period of about 4-7 h, to provide a compound of formula (IX), where PG is BOC. A compound of formula (VIII) is protected with a sulfonyl protecting group such as methanesulfonyl (Ms), benzenesulfonyl (Bs), toluenesulfonyl (Ts), nitrobenzenesulfonyl (Ns), and trifluoromethanesulfonyl (Tf); employing conditions known to one skilled in the art. For example, by reacting a compound of formula (VIII) is treated with a base such as cesium carbonate, and the like; 4-methylbenzenesulfonyl chloride; in a suitable solvent such as acetonitrile, and the like; to provide a compound of formula (IX), where PG is Ts. In a similar fashion, N-sulfonylation of a compound of formula (VIII), is achieved with benzenesulfonyl chloride, a base such as NaH, in a suitable solvent such as DMF, and the like; affords a compound of formula (IX), where PG is benzenesulfonyl (Bs).

An heteroaryl boron compound of formula (XIIa) is prepared from a compound of formula (IX) where HAL is Br or Cl, R^{b} is H or C₁₋₃ alkyl, and R^{c} is H or C₁₋₃ haloalkyl, and PG is SEM, Ts, benzenesulfonyl, or BOC. For example, a compound of formula (IX) where HAL is Br or Cl, R^{b} is H or C₁₋₃ alkyl, and R^{c} is H or C₁₋₃ haloalkyl, and PG is SEM, Ts, benzenesulfonyl, or BOC is treated with a transition metal catalyst such as Pd(dppf)Cl₂, and the like; in a suitable solvent such as DMSO or 1,4-dioxane, and the like; and a base such as KOAc, and the like; and a boron source such as bis(pinacolato)diboron, pinacol borane, and the like; at a temperature ranging from 80° C. to 100° C.; for a period of 2-8 h; to provide a compound of formula (XIIa), where R^{b} is H or C₁₋₃ alkyl, and R^{c} is H or C₁₋₃ haloalkyl.

A compound of formula (VIIIc), where HAL is Br, R^{d} is CHF₂, and R^{g} is H, is prepared from 4-bromo-1H-pyrazolo[3,4-b]pyridine employing oxidative fluorination conditions using a fluorine source such as sodium difluoromethanesulfinate, an oxidant such as t-BuOOH, the like, in a solvent such as DCM or the like.

A compound of formula (VIIIc), where HAL is Br, R^{g} is H, and R^{d} is H, C₁₋₃ alkyl or C₁₋₃ haloalkyl; is protected with a SEM protecting group, employing conditions known to one skilled in the art or as previously described to provide a compound of formula (VI), where PG is SEM. A compound of formula (VI) is borylated employing conditions known to one skilled in the art or as previously described to provide a compound of formula (XIIb).

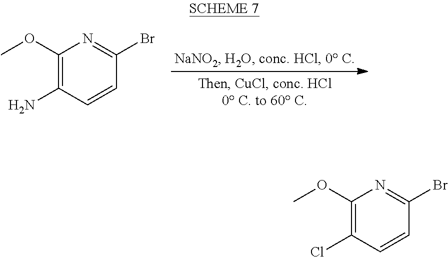

According to SCHEME 7, 6-bromo-2-methoxypyridin-3-amine is treated with sodium nitrite in presence of water and concentrated HCl, at 0° C., for a period of 10 min, then a suspension of CuCl and concentrated HCl is added dropwise at 0° C., then heated to 60° C., for a period of 1.5 h, to provide 6-bromo-3-chloro-2-methoxypyridine.

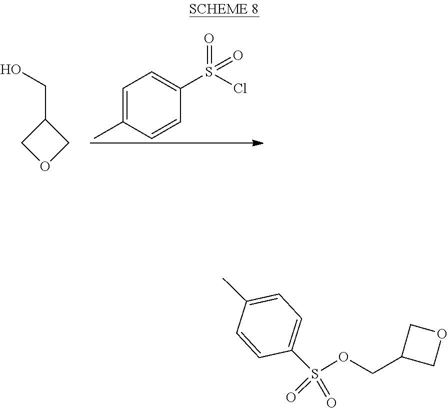

According to SCHEME 8, oxetan-3-ylmethanol is reacted with 4-methylbenzenesulfonyl chloride using a suitable base like triethylamine and the like; in a solvent such as DCM and the like with the presence of DMAP at a temperature ranging from 0° C. to rt for 12 h to provide oxetan-3-ylmethyl 4-methylbenzenesulfonate.

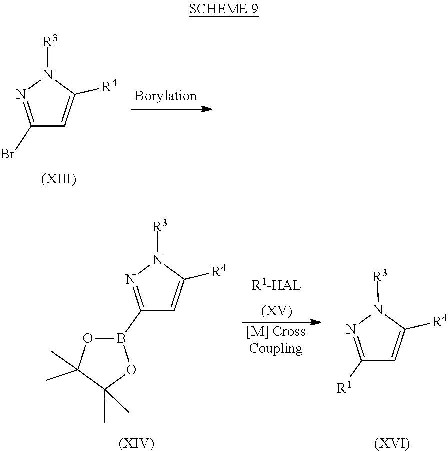

According to SCHEME 9, a commercially available or synthetically accessible compound of formula (XIII) where R³ is C₁₋₆ alkyl and R⁴ is H is borylated employing methods known to those skilled in the art. For example, compound of formula (XIII) is treated with a transition metal catalyst such as Pd(dppf)Cl₂, and the like; in a suitable solvent such as 1,4-dioxane; and a base such as KOAc, and the like; and a borylating agent such as bis(pinacolato)diboron, and the like; at a temperature ranging from 80° C. to 100° C.; for a period of 2-8 h; to provide a compound of formula (XIV), where R³ is C₁₋₆ alkyl and R⁴ is H.

Alternatively, compound of formula (XIII), is borylated via a metal halogen exchange of the bromide with organolithium or magnesium reagents, with or without the presence of lithium chloride, at a temperature of about −78° C., in a suitable solvent such as diethyl ether or tetrahydrofuran (THF), and the like, followed by treatment with 2-isopropoxy-4,4,5,5-tetramethyl-1,3,2-dioxaborolane to provide compound of formula (XIV).

A compound of formula (XIV), where R³ is H, C₁₋₆ alkyl or C₁₋₆ haloalkyl; and R⁴ is H is reacted in a metal-mediated cross coupling reaction with a aryl or heteroaryl halide compound of formula (XV), where R¹—HAL is a suitably substituted aryl, or heteroaryl halide, where HAL is Br or Cl, and R¹ is as described in claim 1; to provide a compound of formula (XVI). For example, a compound of formula (XIV) is reacted employing Suzuki reaction conditions, with a compound of formula (XV) where R¹—HAL is a suitably substituted aryl, or heteroaryl halide, where HAL is Br or Cl, and R¹ is as described in claim 1; in the presence of a palladium catalyst such as Pd(dppf)Cl₂, Pd(PPh₃)₄, Pd(dppf)Cl₂·CH₂Cl₂, Pd(PPh₃)₂Cl₂, Pd(OAc)₂, and the like; with or without the addition of a ligand such as DPPF; a base such as K₃PO₄, K₂CO₃, aq. Na₂CO₃, Na₂CO₃, Cs₂CO₃, and the like; in a suitable solvent such as 1,2-dimethoxyethane, toluene, ethanol, 1,4-dioxane, DMF, water, or a mixture thereof, at a temperature ranging from 80 to 100° C., employing microwave or conventional heating; for a period of about 1 h to 16 h, to provide a compound of formula (XVI).

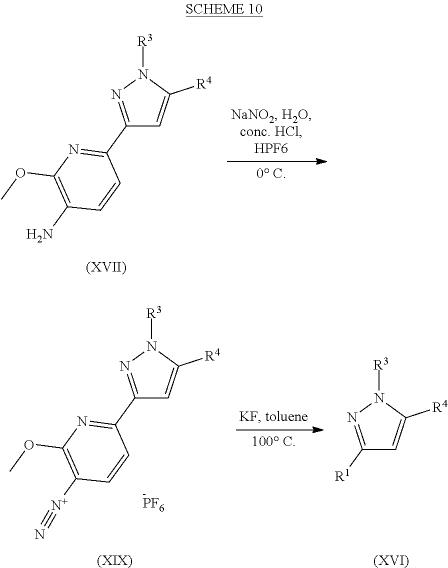

According to SCHEME 10, a diazonium hexafluorophosphate salt compound of formula (XVIII) is prepared from a compound of formula (XVII), where R³ is CH₃ and R⁴ is H; by first reacting with sodium nitrite, in presence of water and concentrated HCl, at a temperature of 0° C., for a period of 10 min, then hexafluorophosphoric acid is added at 0° C., for a period of 30 minutes. A diazonium hexafluorophosphate salt compound of formula (XVIII), where R³ is CH₃ and R⁴ is H; is treated with potassium fluoride, in a suitable solvent such as toluene, and the like; at a temperature of about 100° C.; for a period of about 16 h; to provide a compound of formula (XVI) where R¹ is 3-fluoro-2-methoxypyridine; R³ is CH₃, and R⁴ is H.

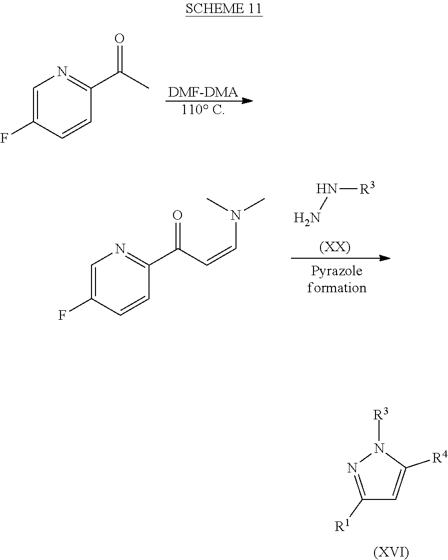

According to SCHEME 11, 1-(5-fluoropyridin-2-yl)ethan-1-one is treated with N,N-dimethylformamide dimethyl acetal (DMF-DMA), at a temperature of 110° C., for about 16 h, to provide (Z)-3-(dimethylamino)-1-(5-fluoropyridin-2-yl)prop-2-en-1-one. Pyrazole formation is achieved under conditions known to one skilled in the art, to provide a compound of formula (XVI). For example, reaction of (Z)-3-(dimethylamino)-1-(5-fluoropyridin-2-yl)prop-2-en-1-one with a suitably substituted hydrazine compound of formula (XX), where R³ is C₃₋₆ cycloalkyl; in a solvent such as EtOH, THF, and the like; with or without the presence of an acid such as acetic acid, and the like; at a temperature ranging from 65° C. to 80° C.; for a period of 16-24 h, affords a compound of formula (XVI), where R³ is C₃₋₆ cycloalkyl, R⁴ is H, and R¹ is a suitably substituted aryl or heteroaryl as defined in claim 1.

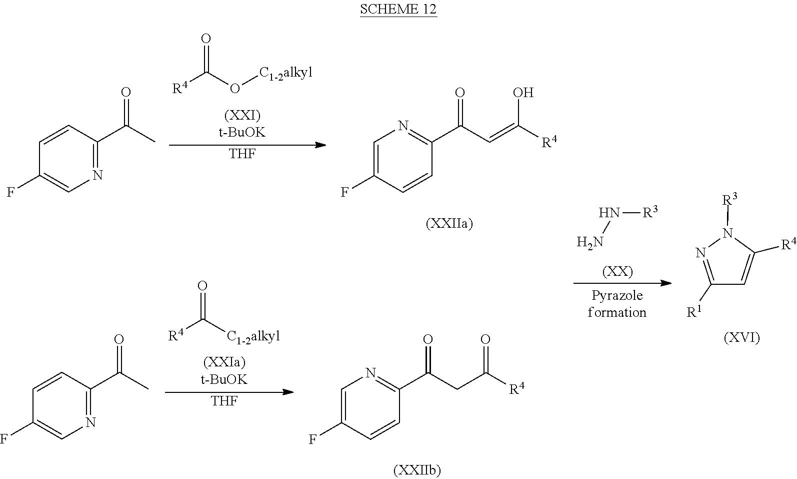

According to SCHEME 12, 1-(5-fluoropyridin-2-yl)ethan-1-one is reacted with potassium tert-butoxide and a compound of formula (XXI), where R⁴ is CH₃, in a suitable solvent such as THF, and the like; at temperatures ranging from 0° C. to rt; for a period of about 16 to 24 h; to provide a compound of formula (XXIIa), where R⁴ is CH₃. A compound of formula (XXIIa) is reacted with a hydrazine hydrate compound of formula (XX), wherein R³ is H, employing pyrazole formation conditions as previously described, to provide a compound of formula (XVI) where R¹ is 5-fluoropyridin-2-yl, R³ is H and R⁴ is CH₃. A compound of formula (X) is alkylated, employing conditions as previously described, employing an alkyl halide such as methyl iodide, to provide a compound of formula (XVI), where R³ is C₁₋₆ alkyl and R⁴ is CH₃.

Methyl 5-fluoropicolinate is reacted with potassium tert-butoxide and a compound of formula (XXIa), where R⁴ is C₃₋₆ cycloalkyl; employing conditions previously described to provide a compound of formula (XXIIb). A compound of formula (XXIIb) is reacted with a hydrazine compound of formula (XX) where R³ is C₁₋₃ alkyl, employing pyrazole formation conditions as previously described, to provide a compound of formula (XVI) where R¹ is 5-fluoropyridin-2-yl.

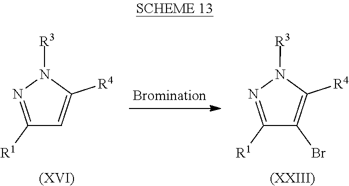

Bromination of compound of formula (XVI), where R¹ is as described in claim 1, R³ is H, C₁₋₄ alkyl or C₁₋₄ haloalkyl; and R⁴ is H, is achieved under conditions known to one skilled in the art to provide a compound of formula (XXIII). For example, reaction of a compound of formula (XVI) with a suitable brominating agent such as NBS, and the like; in a suitable solvent such as DCM, DMF, ACN and the like; at room temperature; for a period of about 30 minutes to 48 h; provides a compound of formula (XXIII).

A compound of formula (XXIII) where R³ is H and R⁴ is H, is further alkylated with an alkylating agent such as 2,2,2-trifluoroethyl trifluoromethanesulfonate, iodomethane-d₃, 3-bromotetrahydrofuran, 3-bromooxetane, 1-fluoro-2-iodoethane or oxetan-3-ylmethyl 4-methylbenzenesulfonate; a suitable base such as NaH, and the like; at a temperature ranging from rt to 70° C.; for a period of about 16 h, provides a compound of formula (XXIII), where R³ is CD3, C₁₋₄ haloalkyl, oxetanyl, tetrahydrofuranyl, CH₂-oxetanyl and R⁴ is H.

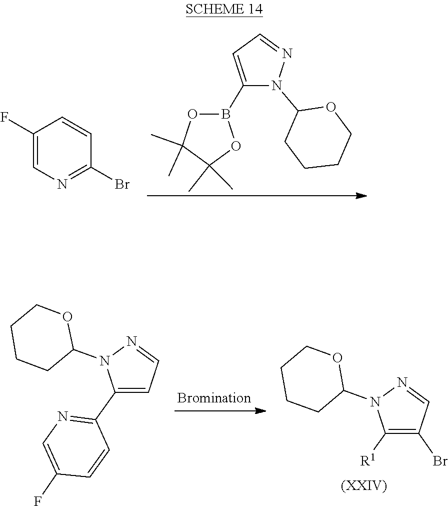

According to SCHEME 14, 2-bromo-5-fluoropyridine is reacted in a metal-mediated cross coupling reaction as previously described with 1-(tetrahydro-2H-pyran-2-yl)-5-(4,4,5,5-tetramethyl-1,3,2-dioxaborolan-2-yl)-1H-pyrazole to provide 5-fluoro-2-(1-(tetrahydro-2H-pyran-2-yl)-1H-pyrazol-5-yl)pyridine. 5-Fluoro-2-(1-(tetrahydro-2H-pyran-2-yl)-1H-pyrazol-5-yl)pyridine is brominated, employing conditions known to one skilled in the art or as previously described to provide a compound of formula (XXIV), where R¹ is 5-fluoropyridin-2-yl.

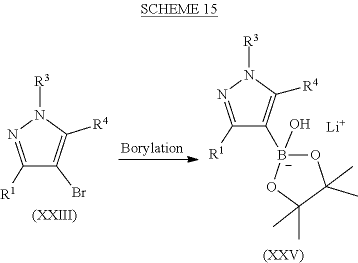

According to SCHEME 15, a compound of formula (XXIII), where R is CH₃, R⁴ is H, and R¹ is 5-fluoropyridin-2-yl; is borylated by metal-halogen exchange with a suitable organolithium reagent such as n-butyllithium, and the like; in the presence of a suitable borylating agent such as triisopropyl borate, 2-isopropoxy-4,4,5,5-tetramethyl-1,3,2-dioxaborolane, and the like; in a suitable solvent such as THF, toluene, and the like; at a temperature ranging from −78° C. to room temperature; for a period of 2 h; to afford the lithium salt of a compound of formula (XXV).

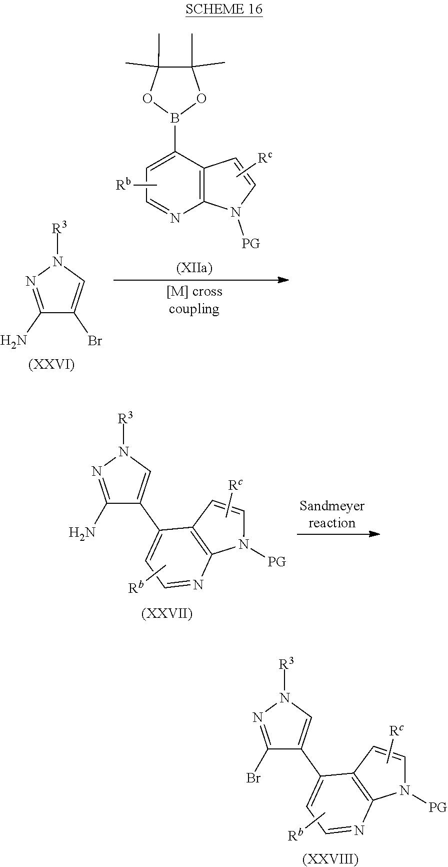

According to SCHEME 16, a compound of formula (XXVI), where R³ is C₁₋₆ alkyl, is reacted in a metal-mediated cross coupling reaction employing methods known to one skilled in the art or as previously described, with a compound of formula (XIIa), where R^{b} is H, R^{c} is H, and PG is benzenesulfonyl (Bs), to provide a compound of formula (XXVII). A compound of formula (XXVII), where R³ is C₁₋₆ alkyl, R^{b} is H, R^{c} is H and PG is benzenesulfonyl, is reacted to form a diazonium intermediate followed by a Sandmeyer reaction under conditions known to one skilled in the art, to provide a compound of formula (XXVIII). For example, a compound of formula (XXVII), where R³ is C₁₋₆ alkyl, R^{b} is H, R^{c} is H and PG is benzenesulfonyl is reacted with tert-butyl nitrite, isoamyl nitrite or sodium nitrite, and the like; in a suitable solvent such as acetonitrile, or 1,4-dioxane, and the like; CuBr; at temperatures ranging from room temperature to 50° C.; for period of 16 to 24 h; to provide a compound of formula (XXVIII).

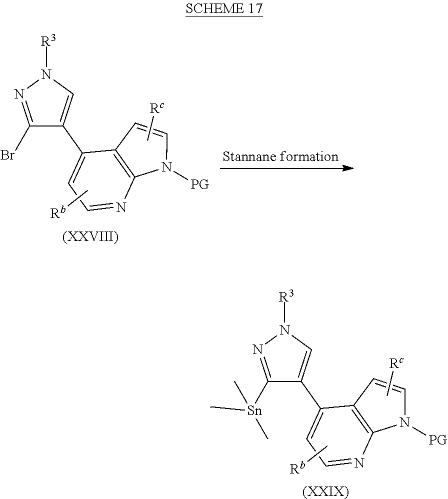

According to SCHEME 17, a compound of formula (XXVIII), where R³ is C₁₋₆ alkyl, R is H, R^{c} is H and PG is benzenesulfonyl, is reacted with a tin reagent such as hexamethylditin; a suitable source of Pd such as Pd(PPh₃)₂Cl₂, and the like; in a solvent suitable solvent such as 1,4-dioxane, and the like; at a temperature of 80° C.; for a period of 64 h; to provide a compound of formula (XXIX).

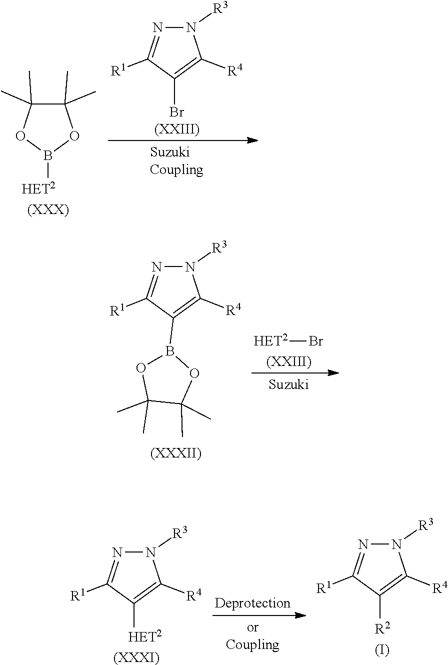

According to Scheme 41, a commercially available or synthetically accessible compound of formula (XXIII), where R¹ is a suitably substituted 5 or 6-membered heteroaryl ring as described in claim 1; R³ is H, C₁₋₆ alkyl, C₁₋₆ haloalkyl, C₃₋₆ cycloalkyl, oxetanyl, CH₂-oxetanyl, and tetrahydrofuranyl; and R⁴ is H, C₁₋₃ alkyl, or C₃₋₆ cycloalkyl; is reacted under metal-mediated cross coupling conditions known to one skilled in the art or as previously described, with a commercially available or synthetically accessible compound of formula (XXX) (which encompasses compounds of formulas (XIIIa) and (XIIIb)), where HET² is a 6-membered heteroaryl, fused 5.6- or fused 6,5-heteroaryl, fused 6,6-heteroaryl, or fused 5.6 heterocycloalkyl ring optionally substituted with a suitable nitrogen protecting group; to provide a compound of formula (XXXI) (or Formula (I), where there is no deprotection or coupling step necessary). A compound of formula (XXXI) is deprotected according to methods previously described, to provide a compound of Formula (I) or Formula (II).

A compound of formula (XXXI), where HET² is a pyridyl group substituted with NH₂, is reacted under conventional amide bond forming techniques such as coupling reactions which are well known to those skilled in the art (such as HATU (1-[bis(dimethylamino)methylene]-1H-1,2,3-triazolo[4,5-b]pyridinium 3-oxid hexafluorophosphate), BOP (benzotriazol-1-yloxy-tris(dimethylamino)phosphonium hexafluorophosphate), or conversion of the acid to an acid chloride). For example, reaction of a compound formula (XXXI), where HET² is a pyridyl group substituted with NH₂, is reacted with a suitable acid such as 3-((tert-butoxycarbonyl)amino)propanoic acid, cyclopropane carboxylic acid, and the like; where the acid is activated with an appropriate activating reagent, for example a carbodiimide, such as N,N′-dicyclohexylcarbodiimide (DCC) or 1-ethyl-3-(3-dimethylaminopropyl)carbodiimide (EDC, EDAC or EDCI) optionally in the presence of hydroxybenzotriazole (HOBt) and/or a catalyst such as 4-dimethylaminopyridine (DMAP); a halotrisaminophosphonium salt such as (benzotriazol-1-yloxy)tris(dimethylamino)phosphonium hexafluorophosphate (BOP), or bromotripyrrolidinophosphonium hexafluorophosphate (PyBroP®); a suitable pyridinium salt such as 2-chloro-1-methyl pyridinium chloride; or another suitable coupling agent such as N,N,N′,N′-tetramethyl-O-(1H-benzotriazol-1-yl)uronium hexafluorophosphate (HBTU), 1-[bis(dimethylamino)methylene]-1H-1,2,3-triazolo[4,5-b]pyridinium 3-oxid hexafluorophosphate (HATU), 2,4,6-tripropyl-1,3,5,2,4,6-trioxatriphosphorinane-2,4,6-trioxide (T3P®) and the like. Coupling reactions are conducted in a suitable solvent such as DCM, THF, DMF and the like, optionally in the presence of a tertiary amine such as N-methylmorpholine, DIPEA, or TEA, at a temperature ranging from about 0° C. to rt, to provide compound a of Formula (I) or Formula (II).

A compound of formula (XXXII), where R¹ is a commercially available or synthetically accessible suitably substituted 5 or 6-membered heteroaryl ring as described in claim 1; R³ is C₁₋₆ alkyl; and R⁴ is H, C₁₋₃ alkyl, or C₃₋₆ cycloalkyl; is reacted under metal-mediated cross coupling conditions as previously described, with a heteroaryl bromide of compound formula (XXXIII), where HET² is a 6-membered heteroaryl, fused 5.6- or fused 6,5-heteroaryl, fused 6,6-heteroaryl, or fused 5.6 heterocycloalkyl ring optionally substituted with a suitable nitrogen protecting group; to provide a compound of formula (XXXI) (or Formula (I), where there is no deprotection or coupling step necessary). A compound of formula (XXXI) is deprotected according to methods previously described, to provide a compound of Formula (I) or Formula (II).

A compound of Formula (I), where R¹ is 5-fluoropyridin-2-yl, R² is 1H-pyrrolo[2,3-b]pyridine, R³ is CH₃, and R⁴ is H, is brominated under conditions previously described, to provide a compound of Formula (I), where R² is 3-bromo-1H-pyrrolo[2,3-b]pyridine.

A compound of Formula (I), where R¹ is 5-fluoropyridin-2-yl, R² is 3-bromo-1H-pyrrolo[2,3-b]pyridine, R³ is CH₃, and R⁴ is H, is reacted with 3-bromooxetane or 3-(bromomethyl)tetrahydrofuran; in a reductive photochemical Ni-catalyzed cross coupling reaction, employing conditions known to one skilled in the art or as previously described, to provide a compound of Formula (I), where R² is 3-(oxetan-3-yl)-1H-pyrrolo[2,3-b]pyridine or 3-(tetrahydrofuran-3-ylmethyl)-1H-pyrrolo[2,3-b]pyridine.

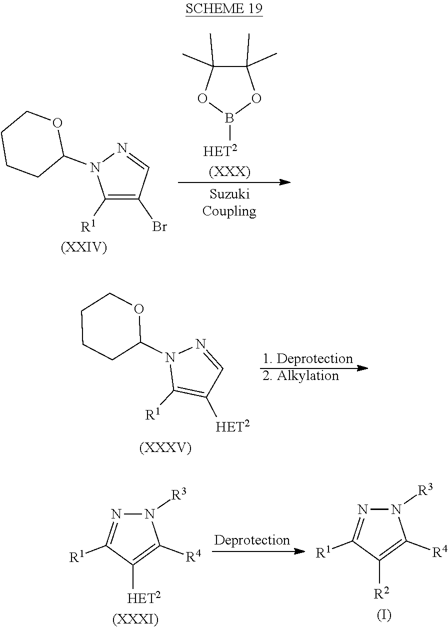

According to SCHEME 19, a compound of formula (XXIV) where R¹ is 5-fluoropyridin-2-yl; is reacted under metal-mediated cross coupling conditions known to one skilled in the art or as previously described, with a commercially available or synthetically accessible compound of formula (XXX) (which encompasses compounds of formulas (XIIIa) and (XIIIb)), where HET² is a 6-membered heteroaryl, fused 5.6- or fused 6,5-heteroaryl, fused 6,6-heteroaryl, or fused 5.6 heterocycloalkyl ring optionally substituted with a suitable nitrogen protecting group; to provide a compound of formula (XXXV). A compound of formula (XXXI) is prepared in two steps from a compound of formula (XXXV). In a first step, deprotection of the tetrahydro-2H-pyran-2-yl moiety is achieved employing acetic acid and water, at temperatures ranging from room temperature to 70° C.; for a period of 3 h. In a second step, alkylation is achieved employing a suitable alkylating agent such as 1-bromo-2-methoxyethane, 2-bromo-1,1-difluoroethane, and the like; a suitable base such as Cs₂CO₃, and the like; in a suitable solvent such as ACN, and the like; to afford a compound of formula (XXXI), where R³ is CH₂CH₂OCH₃ or CH₂CHF₂. A compound of formula (XXXI), where the protecting group is a suitable nitrogen protecting group on HET², is deprotected according to methods known to one skilled in the art or as previously described, to provide a compound of Formula (I).

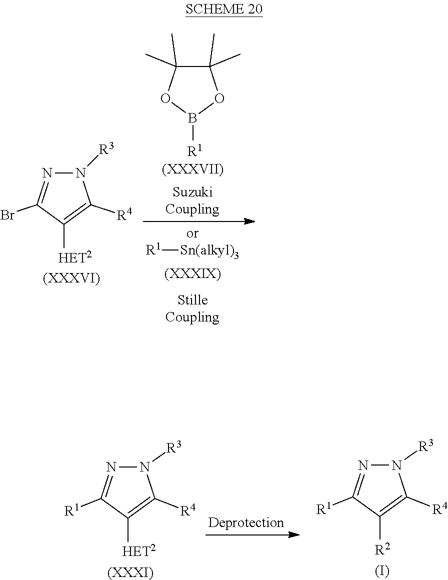

According to Scheme 20, a compound of formula (XXXVI)(which includes compounds of formula (XXVIII)), where HET² is a 6-membered heteroaryl, fused 5.6- or fused 6,5-heteroaryl, fused 6,6-heteroaryl, or fused 5.6 heterocycloalkyl ring optionally substituted with a suitable nitrogen protecting group, R³ is H, C₁₋₆ alkyl, or C₁₋₆ haloalkyl; and R⁴ is H, C₁₋₃ alkyl, or C₃₋₆ cycloalkyl; is reacted under metal-mediated cross coupling conditions such as Suzuki reaction conditions or Stille reaction known to one skilled in the art, with a suitably substituted commercially available boronic acid, boronic ester of formula (XXXVII) or an organostannane compound of formula (XXXIX). For example, employing Suzuki reaction conditions, a compound of formula (XXXVI), is reacted with a commercially available or synthetically accessible suitably heteroaryl boronic acid or boronic ester of formula (XXXVII), where R¹ is a suitably substituted 5- or 6-membered heteroaryl ring as described in claim 1; in the presence of a palladium catalyst such as XPhos Pd G3, cataCXium® A Pd G3, RuPhos Pd G3, Pd(PPh₃)₄, Pd(dppf)Cl₂, PdCl₂(dppf)-CH₂Cl₂, PdCl₂(dtbpf), Pd(amphos)Cl₂, and the like; a suitable base such as KF, Na₂CO₃, aq. Na₂CO₃, potassium phosphate, Cs₂CO₃, K₂CO₃, and the like; in a solvent such as 1,4-dioxane, water, ethanol, toluene, 2-methyl-2-butanol, DMF, or a mixture thereof; employing conventional or microwave heating; at temperatures ranging from room temperature to 130° C.; for a period of 1 h to 18 h; to provide a compound of formula (XXXI).

In a similar fashion, employing Stille coupling conditions, a compound of formula (XXXVI), is reacted with a commercially available or synthetically accessible suitably substituted organostannane compound of formula (XXXIX), where R1 is a suitably substituted 5 or 6-membered heteroaryl ring as described in claim 1; in the presence of a palladium catalyst such as Pd(PPh₃)₄, Pd(dppf)Cl₂, XPhos Pd G3, PdCl₂(PPh₃)₂, and the like; with or without a copper salt such as CuI; in a suitable solvent such as DMF, DCE, toluene, and the like; employing microwave or conventional heating; at temperatures ranging from such as 90 to 120° C.; for a period of 16 to 22 hours; to afford a compound formula (XXXI) or Formula (I) or Formula (II).

A compound of formula (XXXI), where the HET² moiety has a suitable nitrogen protecting group such as BOC, SEM, phenylsulfonyl, para-methoxybenzyl, benzyl, and the like; is deprotected employing conditions known to one skilled in the art to provide a compound of Formula (I) or Formula (II). For example, when the protecting group is BOC or para-methoxybenzyl, deprotection is achieved by reaction with an acid such as TFA, HCl, and the like, in a suitable solvent such as DCM, DCE, THF, and the like; at temperatures ranging from rt to 50° C. Deprotection of the SEM group, is achieved under conditions known to one skilled in the art, such as reaction with TBAF, in a suitable solvent such as THF, at a temperature of about 60° C.; or by reaction with TFA/DCM or HCl/MeOH, or BF₃-OEt₂/DCM; at temperatures ranging from rt to 60° C. Deprotection of the phenylsulfonyl group is achieved under conditions known to one skilled in the art, such as reaction with a base, for example sodium hydroxide, sodium tert-butoxide, and the like; in a suitable solvent such as MeOH, THF, water, or a mixture thereof, at temperatures ranging from 50 to 100° C.; for a period of 3 to 22 hours; employing microwave or conventional heating. Deprotection of the benzyl group is achieved under conditions known to one skilled in the art, for example, under hydrogenation conditions known to one skilled in the art. For example, deprotection is achieved employing a palladium catalyst such Pd/C, and the like; under H₂; in a suitable solvent such as EtOH, MeOH, EtOAc, or a mixture thereof, preferably EtOH; with or without the presence HCl; for a period of 4 to 72 hrs. Deprotection of the benzyl group can also be achieved using a palladium catalyst such as PdCl₂, and the like, in the presence of an acid such as HCl, and the like, in a suitable solvent such as MeOH, and the like, under H₂ at a temperature of 50° C. for a period of 18 h to provide the free NH compound.

Compounds of Formula (I) or Formula (II) may be converted to their corresponding salts using methods known to one of ordinary skill in the art. For example, an amine of Formula (I) or Formula (II) is treated with trifluoroacetic acid, HCl, or citric acid in a solvent such as Et₂O, CH₂Cl₂, THF, MeOH, chloroform, or isopropanol to provide the corresponding salt form. Alternately, trifluoroacetic acid or formic acid salts are obtained as a result of reverse phase HPLC purification conditions. Crystalline forms of pharmaceutically acceptable salts of compounds of Formula (I) or Formula (II) may be obtained in crystalline form by recrystallization from polar solvents (including mixtures of polar solvents and aqueous mixtures of polar solvents) or from non-polar solvents (including mixtures of non-polar solvents).

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention.

Compounds prepared according to the schemes described above may be obtained as single forms, such as single enantiomers, by form-specific synthesis, or by resolution. Compounds prepared according to the schemes above may alternately be obtained as mixtures of various forms, such as racemic (1:1) or non-racemic (not 1:1) mixtures. Where racemic and non-racemic mixtures of enantiomers are obtained, single enantiomers may be isolated using conventional separation methods known to one of ordinary skill in the art, such as chiral chromatography, recrystallization, diastereomeric salt formation, derivatization into diastereomeric adducts, biotransformation, or enzymatic transformation. Where regioisomeric or diastereomeric mixtures are obtained, as applicable, single isomers may be separated using conventional methods such as chromatography or crystallization.

The following specific examples are provided to further illustrate the invention and various preferred embodiments.

### EXAMPLES

In obtaining the compounds described in the examples below and the corresponding analytical data, the following experimental and analytical protocols were followed unless otherwise indicated.

Unless otherwise stated, reaction mixtures were magnetically stirred at room temperature (rt) under a nitrogen atmosphere. Where solutions were “dried,” they were generally dried over a drying agent such as Na₂SO₄ or MgSO₄. Where mixtures, solutions, and extracts were “concentrated”, they were typically concentrated on a rotary evaporator under reduced pressure. Reactions under microwave irradiation conditions were carried out in a Biotage Initiator or CEM (Microwave Reactor) Discover instrument.

Photochemical reactions were conducted in a PennOC (Penn Optical Coatings) Photoreactor ml. Blue light LED is a wavelength of 450 nm.

Normal-phase silica gel chromatography (FCC) was performed on silica gel (SiO₂) using prepacked cartridges.

Preparative reverse-phase high performance liquid chromatography (RP HPLC) was performed on either:
Reverse Phase Preparative HPLC Method A:
Boston Prime C18 column (5 μm, 150 mm×40 mm): eluent: 40% to 60% (v/v) CH₃CN and H₂O with (0.05% NH₃H₂O+10 mM NH₄HCO₃).
Reverse Phase Preparative HPLC Method B:
An Agilent HPLC; Waters XBridge C18 column (5 μm, 50×100 mm) eluent: 5-90% MeCN/20 mM NH₄OH over 15 min, flow rate 80 mL/min.
Reverse Phase Preparative HPLC Method C:
An ACCQ Prep HPLC, XBridge C18 OBD column (5 μM, 50×100): eluent: 0-100% MeCN/water, 20 mM NH₄OH modifier.
Reverse Phase Preparative HPLC Method D:
Phenomenex Gemini NX-C18 column (3 μm, 75 mm×30 mm) or (5 μm, 75 mm×30 mm): 40 mL/min; Gradient: A 0.1% NH₄OH, 10 mM aqueous ammonium carbonate; B 0.1% NH₄OH, 10 mM aqueous ammonium carbonate in 10% water 90% acetonitrile; 90% A to 0% A over 16 min; or 30 mL/min; Gradient: A 0.1% NH₄OH, 10 mM aqueous ammonium carbonate; B 0.1% NH₄OH, 10 mM aqueous ammonium carbonate in 10% water 90% acetonitrile; 98% A to 0% A over 10 min; or Gradient: A 0.1% NH₄OH, 10 mM aqueous ammonium carbonate; B 0.1% NH₄OH, 10 mM aqueous ammonium carbonate in 10% water 90% acetonitrile; 90% A to 0% A over 18 min; or Gradient: A 0.1% TFA in Water; B 0.1% TFA in acetonitrile; 98% A to 0% A over 13 min; or 30 mL/min; Gradient: A 0.1% TFA in Water; B 0.1% TFA in acetonitrile; 90% A to 0% A over 16 min.; or eluent: 30% to 60% (v/v) CH₃CN and H₂O with 0.04% NH₃+10 mM NH₄HCO₃; or Condition: A: water (0.05% NH₃H₂O+10 mM NH₄HCO₃), B: CH₃CN at the beginning: A (68%) and B (32%) and at the end: A: (38%) and B (62%); Gradient Time 6 min; 100% B Hold 1.8 min; Flow Rate 25 mL/min;
Reverse Phase Preparative HPLC Method E:
Boston Prime C18 column (5 m, 150 mm×30 mm): eluent: 15% to 45% (v/v) CH₃CN and H₂O with 0.05% NH₃+10 mM NH₄HCO₃; or eluent: 30% to 60% (v/v) water (0.04% NH₃H₂O+10 mM NH₄HCO₃)−ACN.
Reverse Phase Preparative HPLC Method F:
An ACCQ Prep HPLC; with an XBridge C18 OBD column (5 μM, 50×100), eluent 20-80% MeCN:H₂O w/0.05% TFA.

Preparative supercritical fluid high performance liquid chromatography (SFC) was performed either on a Jasco preparative SFC system or a Waters Prep SFC 150 AP system. The separations were conducted at 100 to 150 bar with a flow rate ranging from 40 to 60 mL/min.

The column was heated to 35 to 40° C.
SFC Method A:
Whelk O1 SS column (5 μm, 250×21 mm) Mobile phase: 35% methanol with 0.2% triethylamine, 65% CO₂). Flow rate 42 mL/min.
SFC Method B:
DAICEL CHIRALPAK® AD column: (10 μm, 250 mm×30 mm): isocratic elution: containing 0.1% of 25% NH_{3(aq)}): supercritical CO₂, 35%: 65% to 35%: 65% (v/v).
SFC Method C:
DAICEL CHIRALCEL® OJ-H column (5 μm, 250 mm×30 mm): isocratic elution: EtOH (containing 0.1% of 25% aq. NH₃): supercritical CO₂, 20%:20% to 20%: 20% (v/v).

Mass spectra (MS) were obtained on an Agilent series 1100 MSD using electrospray ionization (ESI) in positive mode unless otherwise indicated. Calculated (calcd.) mass corresponds to the exact mass.

Analytical LCMS was obtained on an Agilent 1260 Series using an ACE Excel 3 C18 column (3 m, 2.1×35 mm, T=50° C.). Mobile phase A: 0.05% TFA in H₂O and mobile phase B: 100% acetonitrile. Method gradient starts at 5% B to 100% B in 2.2 minutes at flow rate 1.0 mL/min. MS detector is an Agilent G6125B MSD set in positive mode.

Nuclear magnetic resonance (NMR) spectra were obtained on Bruker Avance Neo spectrometers. Definitions for multiplicity are as follows: s=singlet, d=doublet, t=triplet, q=quartet, m=multiplet, br=broad, dd=doublet of doublets, ddd=doublet of doublets of doublets, dt=doublet of triplets, td=triplet of doublets. It will be understood that for compounds comprising an exchangeable proton, said proton may or may not be visible on an NMR spectrum depending on the choice of solvent used for running the NMR spectrum and the concentration of the compound in the solution.

Chemical names were generated using ChemDraw Ultra 17.1 (CambridgeSoft Corp., Cambridge, MA) or OEMetaChem V1.4.0.4 (Open Eye).

### Intermediate 1: 4-(4,4,5,5-Tetramethyl-1,3,2-dioxaborolan-2-yl)-1-((2-(trimethylsilyl)ethoxy)methyl)-1H-pyrazolo[3,4-b]pyridine

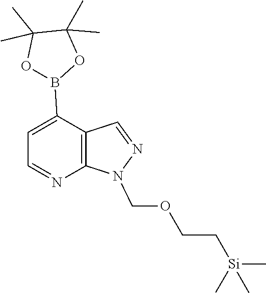

Step A. 4-Bromo-1-((2-(trimethylsilyl)ethoxy)methyl)-1H-pyrazolo[3,4-b]pyridine. To a stirred solution of 4-bromo-1H-pyrazolo[3,4-b]pyridine (650 mg, 3.28 mmol) in DMF (6.5 mL) was added sodium hydride (197 mg of a 60% dispersion in mineral oil, 4.92 mmol). The reaction mixture was stirred at ambient temperature under a stream of N₂ gas for 20 minutes, cooled to 0° C., and then 2-(trimethylsilyl)ethoxymethyl chloride (639 μL, 3.61 mmol) was added slowly. The reaction was warmed to ambient temperature and stirring was continued for 3 h. The reaction was quenched with H₂O and the resulting mixture was extracted with ethyl acetate (2×25 mL). The combined extracts were washed with brine (50 mL), dried (MgSO₄), and filtered. The resulting organics were concentrated under reduced pressure, and the residue was purified by chromatography (silica gel, 0-100% ethyl acetate/hexanes) to afford the title compound as a colorless oil (780 mg, 72%). MS (ESI): mass calcd. for C₁₂H₁₈BrN₃OSi, 327.0; m/z found, 328.0 [M+H]⁺.

Step B. 4-(4,4,5,5-Tetramethyl-1,3,2-dioxaborolan-2-yl)-1-((2-(trimethylsilyl)ethoxy)methyl)-1H-pyrazolo[3,4-b]pyridine. In a round bottom flask was dissolved 4-bromo-1-((2-(trimethylsilyl)ethoxy)methyl)-1H-pyrazolo[3,4-b]pyridine (780 mg, 2.38 mmol), bis(pinacolato)diboron (724 mg, 2.85 mmol), potassium acetate (466 mg, 4.75 mmol), and, 1,1′-bis(diphenylphosphino)ferrocene-palladium(II)dichloride dichloromethane complex (194 mg, 0.238 mmol) in 1,4-dioxane (24 mL). The resulting mixture was degassed with N₂ and heated for 2 h at 100° C. The reaction was cooled to room temperature, passed through a Celite® cartridge and concentrated under reduced pressure. The crude residue was purified by chromatography (silica gel, 0-100% ethyl acetate/hexanes) to afford the title compound as a tan solid (884 mg, 99%). MS (ESI): mass calcd. for C₁₈H₃₀BN₃O₃Si, 375.2; m/z found, 294.1 [M−C₆H₁₀+H]⁺. ¹H NMR (500 MHz, DMSO-d₆) δ ppm 8.63 (d, J=4.4 Hz, 1H), 8.26 (s, 1H), 7.50 (d, J=4.4 Hz, 1H), 5.79 (s, 2H), 3.53-3.62 (m, 2H), 1.37 (s, 12H), 0.77-0.85 (m, 2H), −0.11 (s, 9H).

### Intermediate 2: 4-Bromo-6-methyl-1H-pyrazolo[3,4-b]pyridine

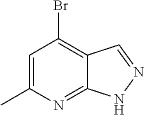

Step A. Ethyl (E)-3-((1-(4-methoxybenzyl)-1H-pyrazol-5-yl)amino)but-2-enoate. p-Toluenesulfonic acid (2.80 g, 16.3 mmol), 1-(4-methoxybenzyl)-1H-pyrazol-5-amine (33.0 g, 162 mmol), ethyl 3-oxobutanoate (38.0 mL, 293 mmol), and toluene (350 mL) were added to a 1000 mL three-necked round-bottomed flask under N₂. The resultant mixture was stirred for 4 hours at 70° C. then cooled to room temperature. The mixture was filtered through a pad of Celite® and the pad washed with toluene (100 mL). The filtrate was concentrated to dryness under reduced pressure to give the crude product, which was purified by FCC (eluent:petroleum ether:ethyl acetate=1:0 to 92:8) to afford the title compound (40 g, 74%) as a yellow solid. MS (ESI): mass calcd. for C₁₇H₂₁N₃O₃, 315.2; m/z found, 316.2 [M+H]⁺.

Step B. 1-(4-Methoxybenzyl)-6-methyl-1H-pyrazolo[3,4-b]pyridin-4-ol. Dowtherm® A (220 mL) was added to a 500 mL three-necked round-bottomed flask and then heated to 210° C. under N₂. The mixture was then treated with (E)-ethyl 3-((1-(4-methoxybenzyl)-1H-pyrazol-5-yl)amino)but-2-enoate (50.0 g, 159 mmol) and stirred at 210° C. for 6 hours then cooled to room temperature. The mixture was stirred at room temperature for another 5 hours before diluting with petroleum ether (260 mL) and the resultant suspension isolating via filtration. The filter cake was further triturated with petroleum ether:ethyl acetate (220 mL, 10:1) and the suspension isolated via filtration. The filter cake was dried under reduced pressure to afford the title compound (27 g, 60%) as a yellow solid. ¹H NMR (400 MHz, DMSO-d₆) δ 11.37 (br s, 1H), 8.01 (br. s, 1H), 7.16 (d, J=8.5 Hz, 2H), 6.85 (br. d, J=8.3 Hz, 2H), 6.39 (br s, 1H), 5.46 (s, 2H), 3.69 (s, 3H), 2.46 (br. s, 3H).

Step C. 4-Bromo-1-(4-methoxybenzyl)-6-methyl-1H-pyrazolo[3,4-b]pyridine. 1-(4-Methoxybenzyl)-6-methyl-1H-pyrazolo[3,4-b]pyridin-4-ol (27.0 g, 100 mmol), phosphorus oxybromide (44.0 g, 153 mmol), DMF (100 mL), and toluene (270 mL) were added to a 1 L round-bottomed flask. The resultant mixture was stirred at 60° C. for 2 hours under N₂. The reaction mixture was cooled to room temperature, quenched with water (260 mL), and extracted with toluene (300 mL). The organic extract were washed with brine (200 mL×3), dried over anhydrous Na₂SO₄, filtered, and concentrated to dryness under reduced pressure to afford the title compound (31 g, 91%) as a yellow solid. MS (ESI): mass calcd. for C₁₅H₁₄BrN₃O, 331.0; m/z found, 332.0 [M+H]⁺. ¹H NMR (400 MHz, DMSO-d₆) δ 8.08 (s, 1H), 7.49-7.41 (m, 1H), 7.18 (s, 2H), 6.86 (d, J=8.7 Hz, 2H), 5.55 (s, 2H), 3.69 (s, 3H), 2.61 (s, 3H).

Step D. 4-Bromo-6-methyl-1H-pyrazolo[3,4-b]pyridine. 4-Bromo-1-(4-methoxybenzyl)-6-methyl-1H-pyrazolo[3,4-b]pyridine (31 g, 93 mmol), TFA (200 mL) were added to a 500 mL three-necked round-bottomed flask. The resultant mixture was stirred at 60° C. for 12 hours before being concentrated to dryness under reduced pressure. The residue was diluted with water (100 mL), the resultant mixture adjusted to pH=6 with sat. NaHCO₃ slowly and extracted with ethyl acetate (500 mL×3). The combined organic extracts were washed with brine (500 mL×3), dried over anhydrous Na₂SO₄, filtered, and concentrated to dryness under reduced pressure to afford the title compound (30 g, crude) as a yellow solid, which was used in the next step without further purification. MS (ESI): mass calcd. for C₇H₆BrN₃, 211.0; m/z found, 212.1 [M+H]⁺. ¹H NMR (400 MHz, DMSO-d₆) δ 13.82 (br. s, 1H), 8.05 (s, 1H), 7.39 (s, 1H), 2.56 (s, 3H).

### Intermediate 3: 6-Methyl-4-(4,4,5,5-tetramethyl-1,3,2-dioxaborolan-2-yl)-1-((2-(trimethylsilyl)ethoxy)methyl)-1H-pyrazolo[3,4-b]pyridine

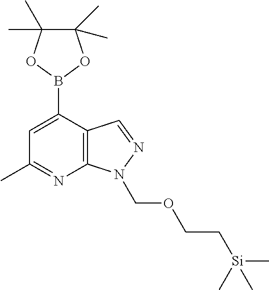

The title compound was prepared in a manner analogous to Intermediate 1, Steps A-B except using 4-bromo-6-methyl-1H-pyrazolo[3,4-b]pyridine (Intermediate 2) instead of 4-bromo-1H-pyrazolo[3,4-b]pyridine in Step A. MS (ESI): mass calcd. for C₁₃H₂₂BN₃O₃Si, 389.2; m/z found, 308.1 [M−C₆H₁₀+H]⁺.

### Intermediate 4: tert-Butyl 4-(4,4,5,5-tetramethyl-1,3,2-dioxaborolan-2-yl)-1H-pyrrolo[2,3-b]pyridine-1-carboxylate

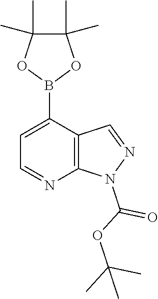 [SMILES: CC(C)(C)OC(=O)n1ncc2c(B3OC(C)(C)C(C)(C)O3)ccnc21]

The title compound was prepared in a manner analogous to Intermediate 1, Step B, using tert-butyl 4-bromo-1H-pyrrolo[2,3-b]pyridine-1-carboxylate instead of 4-bromo-1-((2-(trimethylsilyl)ethoxy)methyl)-1H-pyrazolo[3,4-b]pyridine. ¹H NMR (500 MHz, DMSO-d₆) δ 8.38 (d, J=4.6 Hz, 1H), 7.81 (d, J=4.0 Hz, 1H), 7.44 (d, J=4.6 Hz, 1H), 6.83 (d, J=4.0 Hz, 1H), 1.59 (s, 9H), 1.33 (s, 12H).

### Intermediate 5: 2-(3-(5-Fluoropyridin-2-yl)-1-methyl-1H-pyrazol-4-yl)-2-hydroxy-4,4,5,5-tetramethyl-1,3,2-dioxaborolan-2-uide Lithium Salt

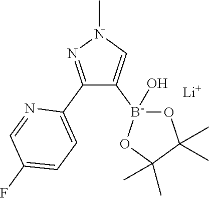 [SMILES: Cn1cc([B-]2(O)OC(C)(C)C(C)(C)O2)c(-c2ccc(F)cn2)n1 ; [Li+]]

A solution of 2-(4-bromo-1-methyl-1H-pyrazol-3-yl)-5-fluoropyridine (Intermediate 18, 500 mg, 1.95 mmol) and triisopropyl borate (0.59 mL, 2.54 mmol) in THE (4 mL) and toluene (4 mL) was cooled to −78° C. then n-butyllithium (1.8 mL of 1.6 M in hexane, 2.93 mmol) was added dropwise. After 2 h, pinacol (346 mg, 2.9 mmol) was added and the reaction allowed to warm to room temperature. Water (0.035 mL, 1.95 mmol) was added and the mixture was stirred for 3 h. The resulting precipitate was collected by filtration, rinsed with Et₂O, and air dried to afford the title compound (473 mg, 74%). MS (ESI): mass calcd. for C₁₅H₁₉BFN₃O₂, 303.2; m/z found, 222.1 [(M−C₆H₁₀)+H]⁺. ¹H NMR (400 MHz, Methanol-d₄) δ 8.59-8.48 (m, 1H), 8.35-8.30 (m, 1H), 7.53-7.45 (m, 1H), 7.39 (s, 1H), 3.92 (s, 3H), 1.23 (s, 9H), 1.14 (s, 3H).

Method B:

Under N₂, n-BuLi (1.2 equiv) was added dropwise to a pre-cooled solution (−78° C.) of 2-(4-bromo-1-methyl-1H-pyrazol-3-yl)-5-fluoropyridine (Intermediate 18, 60 g, 0.23 mol) and 2-isopropoxy-4,4,5,5-tetramethyl-1,3,2-dioxaborolane (52.3 g, 0.28 mol, 1.2 equiv) in THE (1.2 L, 20V). After addition was complete, the reaction was warmed to −65° C. LiOH (1.12 g, 0.047 mol) was then added, and the reaction was warmed to −10 to −20° C. Water (21.1 ml, 1.17 mol) was then added dropwise at −10 to −20° C. and the reaction was stirred for 30 minutes before the solids were filtered off and washed with THF. The cake was slurried with MTBE (0.27 L, 5V) at 60° C. and then filtered washing the cake with MTBE. The cake was then dried at 45° C. to afford the title compound as a white solid (63.1 g, 83%). MS (ESI): mass calcd. for C₁₅H₁₉BFN₃O₂, 303.2; m/z found, 222.1 [(M−C₆H₁₀)+H]⁺. ¹H NMR (400 MHz, Methanol-d₄) δ 8.51 (dd, J=8.8, 4.9 Hz, 1H), 8.32 (d, J=2.9 Hz, 1H), 7.56-7.42 (m, 1H), 7.36 (s, 1H), 3.89 (s, 3H), 1.20 (s, 6H), 1.13 (s, 3H), 1.11 (s, 3H).

### Intermediate 6: 4-(4,4,5,5-Tetramethyl-1,3,2-dioxaborolan-2-yl)-1-((2-(trimethylsilyl)ethoxy)methyl)-1H-pyrrolo[2,3-b]pyridine

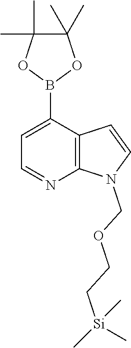 [SMILES: CC1(C)OB(c2ccnc3c2ccn3COCC[Si](C)(C)C)OC1(C)C]

The title compound was prepared in a manner analogous to Intermediate 1, Steps A-B except using 4-bromo-1H-pyrrolo[2,3-b]pyridine instead of 4-bromo-1H-pyrazolo[3,4-b]pyridine in Step A. MS (ESI): mass calcd. for C₁₉H₃₁BN₂O₃Si, 374.2; m/z found, 293.1 [(M−C₆H₁₀)+H]⁺. ¹H NMR (500 MHz, CDCl₃) δ 8.34 (d, J=4.6 Hz, 1H), 7.46 (d, J=4.6 Hz, 1H), 7.39 (d, J=3.5 Hz, 1H), 6.92 (d, J=3.6 Hz, 1H), 5.69 (s, 2H), 3.58-3.45 (m, 2H), 1.40 (s, 12H), 0.97-0.84 (m, 2H), −0.08 (s, 9H).

### Intermediate 7: 1-Methyl-3-(4,4,5,5-tetramethyl-1,3,2-dioxaborolan-2-yl)-1H-pyrazole

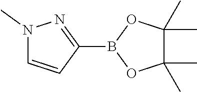

To a mixture of 3-bromo-1-methyl-1H-pyrazole (1.15 g, 7.14 mmol), bis(pinacolato)diboron (2.18 g, 8.58 mmol), [1,1′-bis(diphenylphosphino)ferrocene]dichloropalladium(II) (520 mg, 0.711 mmol) and potassium acetate (2.1 g, 21.4 mmol) was added 1,4-dioxane (28 mL). The reaction was split evenly 2 portions and stirred at 95° C. for 5 h under argon. The combined reaction mixtures were filtered through a pad of Celite®. The Celite® was washed with chloroform (3×30 mL) and the combined filtrate layers were evaporated. The residue was taken up in chloroform (100 mL) and extracted with 0.1N sodium hydroxide (2×100 mL). The combined aqueous layers were acidified to pH 5 with 1N hydrochloric acid. The aqueous layer was extracted with chloroform (3×100 mL). The combined organic layers were dried over magnesium sulfate, filtered, and evaporated to give the title compound (543 mg, crude) as a brown oil that was used without further purification. MS (ESI): mass calcd. for C₁₀H₁₇BN₂O₂, 208.1; m/z found, 127.2 [M−C₆H₁₀+H].

### Intermediate 8: 2-(4-Bromo-1-methyl-1H-pyrazol-3-yl)-5-chloropyridine

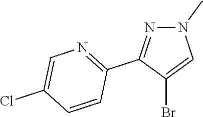

Step A. 5-Chloro-2-(1-methyl-1H-pyrazol-3-yl)pyridine. In a pressure vessel was dissolved 1-methyl-3-(4,4,5,5-tetramethyl-1,3,2-dioxaborolan-2-yl)-1H-pyrazole (Intermediate 7, 100 mg, 0.481 mmol), 2-bromo-5-chloropyridine (92 mg, 0.481 mmol) and 1,1′-bis(diphenylphosphino)ferrocene-palladium(II)dichloride dichloromethane complex (Pd(dppf)Cl₂·CH₂Cl₂) (39 mg, 0.0481 mmol) in 1,4-dioxane (2 mL) and 2 M Na₂CO₃ (aq) (0.961 mL, 1.92 mmol). The resulting mixture was degassed with N₂ and heated overnight at 100° C. The reaction was cooled to room temperature and partitioned between ethyl acetate and H₂O. The layers were separated and the aqueous was extracted with ethyl acetate (2×25 mL). The organic layers were combined and washed with brine (50 mL), dried (MgSO₄) and filtered. The resulting organics were concentrated under reduced pressure, and the residue was purified by chromatography (silica gel, 0-100% ethyl acetate/hexanes) to afford the title compound as a colorless oil (65 mg, 70%). MS (ESI): mass calcd. for C₉H₈ClN₃, 193.0; m/z found, 194.0 [M+H]⁺.

Step B. 2-(4-Bromo-1-methyl-1H-pyrazol-3-yl)-5-chloropyridine. To a stirred solution of 5-chloro-2-(1-methyl-1H-pyrazol-3-yl)pyridine (67 mg, 0.346 mmol) in acetonitrile (3.5 mL) was added N-bromosuccinimide (NBS) (67 mg, 0.381 mmol). The reaction mixture was stirred at ambient temperature for 48 h. The resulting mixture was concentrated under reduced pressure and the crude residue was dissolved in DCM for purification (silica gel, 0-100% ethyl acetate/hexanes) to afford the title compound as a white solid (91 mg, 96%). MS (ESI): mass calcd. for C₉H₇BrClN₃, 271.0; m/z found, 271.9 [M+H]⁺. ¹H NMR (400 MHz, CDCl₃) δ 8.69 (dd, J=2.6, 0.8 Hz, 1H), 7.98 (dd, J=8.5, 0.7 Hz, 1H), 7.75 (dd, J=8.5, 2.5 Hz, 1H), 7.52 (s, 1H), 3.99 (s, 3H).

### Intermediate 9: 2-(4-Bromo-1-methyl-1H-pyrazol-3-yl)-6-methylpyridine

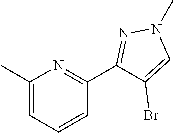

Step A. 2-Methyl-6-(1-methyl-1H-pyrazol-3-yl)pyridine. 2-Bromo-6-methylpyridine (1.0 g, 5.8 mmol), 1-methyl-3-(4,4,5,5-tetramethyl-1,3,2-dioxaborolan-2-yl)-1H-pyrazole (1.2 g, 5.8 mmol), and K₂CO₃ (2.4 g, 17 mmol) were added to a 20 mL microwave tube and the resulting mixture dissolved in 1,4-dioxane (12 mL) and H₂O (3 mL). The mixture was sparged with N₂ for 5 minutes and then treated with Pd(dppf)Cl₂ (425 mg, 0.58 mmol). The mixture was sparged with N₂ for another 5 minutes and then stirred while heating at 80° C. via microwave irradiation for 1 hour then cooled to room-temperature. The mixture was concentrated to dryness under reduced pressure to give the crude product, which was purified by FCC (eluent:petroleum ether:ethyl acetate=1:0 to 3:1) to afford the title compound as a yellow oil (954 mg, 95%). MS (ESI): mass calcd. for C₁₀H₁₁N₃, 173.1; m/z found, 174.1 [M+H]⁺.

Step B. 2-(4-Bromo-1-methyl-1H-pyrazol-3-yl)-6-methylpyridine. The title compound was prepared in a manner analogous to Intermediate 8, Step B, except using dichloromethane instead of acetonitrile and stirring for 2 hours instead of 48 h. MS (ESI): mass calcd. for C₁₀H₁₀BrN₃, 251.0; m/z found, 252.1 [M+H]⁺.

### Intermediate 10: 2-(4-Bromo-1-(methyl-d₃)-1H-pyrazol-3-yl)-5-fluoropyridine

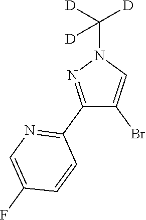 [SMILES: [2H]C([2H])([2H])n1cc(Br)c(-c2ccc(F)cn2)n1]

Step A: 5-Fluoro-2-(1H-pyrazol-3-yl)pyridine. In a pressure vessel was placed 2-bromo-5-fluoropyridine (360 mg, 2.1 mmol), 3-(4,4,5,5-tetramethyl-1,3,2-dioxaborolan-2-yl)-1H-pyrazole (476 mg, 2.5 mmol), Pd(PPh₃)₄ (236 mg, 0.21 mmol), K₂CO₃ (424 mg, 3.1 mmol), water (2 mL) and 1,4-dioxane (14 mL). The vial was capped and the reaction mixture was degassed with N₂ for 2 minutes then heated to 80° C. for 16 hours. It was then filtered through Celite® and the solvent was evaporated. Purification by chromatography (silica gel, 0% to 100% EtOAc/Hexanes) gave the title compound (157 mg, 47% yield). MS (ESI): mass calcd. for C₈H₆FN₃, 163.1; m/z found, 164.1 [M+H]⁺.

Step B: 2-(4-Bromo-1H-pyrazol-3-yl)-5-fluoropyridine. To a solution of 5-fluoro-2-(1H-pyrazol-3-yl)pyridine (157 mg, 0.96 mmol) in DMF (3.8 mL) was added 1-bromopyrrolidine-2,5-dione (171 mg, 0.96 mmol) and stirred at room temperature. After 30 minutes the reaction mixture was diluted with EtOAc and water. The aqueous phase was extracted twice with EtOAc and the combined organic layers were washed with a saturated aqueous solution of NaCl, dried over MgSO₄, filtered and evaporated. Purification by chromatography (silica gel, 0% to 100% EtOAc/Hexanes) gave the title compound (165 mg, 71% yield). MS (ESI): mass calcd. for C₈H₅BrFN₃, 241.0; m/z found, 242.0 [M+H]⁺.

Step C: 2-(4-Bromo-1-(methyl-d₃)-1H-pyrazol-3-yl)-5-fluoropyridine. To a solution of 2-(4-bromo-1H-pyrazol-3-yl)-5-fluoropyridine (50 mg, 0.21 mmol) and iodomethane-d₃ (30 mg, 0.21 mmol) in DMF (2.1 mL) was added NaH (60% dispersion in mineral oil, 17 mg, 0.41 mmol). The reaction mixture was stirred at room temperature for 16 hours then water and EtOAc were added. The aqueous phase was extracted twice with EtOAc and the combined organic layers were dried over MgSO₄, filtered and evaporated. Purification by chromatography (silica gel, 0% to 100% EtOAc/Hexanes) gave the title compound (48 mg, 90% yield). MS (ESI): mass calcd. for C₉H₄D3BrFN₃, 258.0; m/z found, 259.0 [M+H]⁺. ¹H NMR (500 MHz, CDCl₃) δ 8.55 (d, J=3.0 Hz, 1H), 7.98 (ddd, J=8.8, 4.4, 0.6 Hz, 1H), 7.47 (s, 1H), 7.45 (ddd, J=8.8, 8.1, 3.0 Hz, 1H).

### Intermediate 11: 2-(4-Bromo-1-(oxetan-3-yl)-1H-pyrazol-3-yl)-5-fluoropyridine

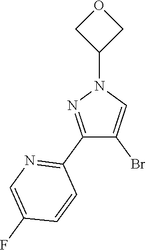 [SMILES: Fc1ccc(-c2nn(C3COC3)cc2Br)nc1]

To a solution of 2-(4-bromo-1H-pyrazol-3-yl)-5-fluoropyridine (Intermediate 10, product from Step B, 50 mg, 0.21 mmol) and 3-bromooxetane (28 mg, 0.21 mmol) in DMF (2.1 mL) was added NaH (60% dispersion in mineral oil, 17 mg, 0.41 mmol). The reaction mixture was stirred at room temperature for 2 hours then at 50° C. for 16 hours. Another portion of 3-bromooxetane (28 mg, 0.21 mmol) and NaH (60% dispersion in mineral oil, 17 mg, 0.41 mmol) were added and the reaction mixture was stirred at 50° C. for 4 days. Water and EtOAc were then added. The aqueous phase was extracted twice with EtOAc and the combined organic layers were dried over MgSO₄, filtered and evaporated. Purification by chromatography (silica gel, 0% to 100% EtOAc/Hexanes) gave the title compound (47 mg, 76% yield). MS (ESI): mass calcd. for C₁₁H₉BrFN₃O, 297.0; m/z found, 298.0 [M+H]⁺. ¹H NMR (500 MHz, CDCl₃) δ 8.58 (d, J=2.9 Hz, 1H), 8.01 (ddd, J=8.7, 4.4, 0.6 Hz, 1H), 7.78 (s, 1H), 7.48 (ddd, J=8.7, 8.0, 2.9 Hz, 1H), 5.58-5.44 (m, 1H), 5.12-5.00 (m, 4H).

### Intermediate 12: (R/S)-2-(4-Bromo-1-(tetrahydrofuran-3-yl)-1H-pyrazol-3-yl)-5-fluoropyridine

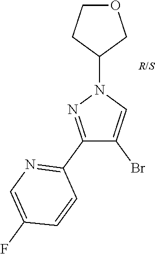 [SMILES: Fc1ccc(-c2nn(C3CCOC3)cc2Br)nc1]

The title compound was prepared in a manner analogous to Intermediate 10, Step C, using 3-bromotetrahydrofuran instead of iodomethane-d₃, and heating to 50° C. instead of room temperature. MS (ESI): mass calcd. for C₁₂H₁₁BrFN₃O, 310.0; m/z found, 311.0 [M+H]⁺. ¹H NMR (400 MHz, CDCl₃) δ 8.56 (d, J=3.0 Hz, 1H), 7.98 (ddd, J=8.8, 4.5, 0.6 Hz, 1H), 7.62 (s, 1H), 7.46 (ddd, J=8.8, 8.1, 2.9 Hz, 1H), 5.13-4.99 (m, 1H), 4.20-4.09 (m, 2H), 4.06-3.98 (m, 1H), 3.96-3.89 (m, 1H), 2.60-2.41 (m, 1H), 2.39-2.24 (m, 1H).

### Intermediate 13: 2-(4-Bromo-1-(2,2,2-trifluoroethyl)-1H-pyrazol-3-yl)-5-fluoropyridine

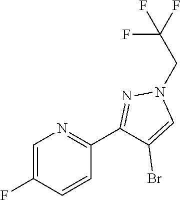 [SMILES: Fc1ccc(-c2nn(CC(F)(F)F)cc2Br)nc1]

2-(4-Bromo-1H-pyrazol-3-yl)-5-fluoropyridine (Intermediate 10, product from Step B, 1.0 g, 70% purity, 2.9 mmol), 2,2,2-trifluoroethyl trifluoromethanesulfonate (0.81 g, 3.5 mmol), Cs₂CO₃ (2.76 g, 8.48 mmol), and MeCN (15 mL) were added to a 50 mL round-bottomed flask. The resultant mixture was stirred at 70° C. for 16 hours. The reaction mixture was cooled to room-temperature, poured into water (60 mL), and extracted with ethyl acetate (3×50 mL). The combined organic extracts were dried over anhydrous Na₂SO₄, filtered, and concentrated to dryness under reduced pressure to give the crude product, which was purified by FCC (eluent:petroleum ether:ethyl acetate=1:0 to 2:1) to afford the title compound (104 mg, 11%) as a light yellow solid. MS (ESI): mass calcd. for C₁₀H₆BrF₄N₃, 323.0; m/z found, 324.1 [M+H]⁺. ¹H NMR (400 MHz, CDCl₃) δ 8.60 (d, J=3.0 Hz, 1H), 8.05-7.96 (m, 1H), 7.67 (s, 1H), 7.55-7.45 (m, 1H), 4.77 (q, J=8.3 Hz, 2H).

### Intermediate 14: 6-(4-Bromo-1-methyl-1H-pyrazol-3-yl)-3-fluoro-2-methylpyridine

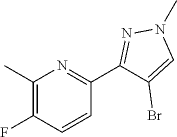

The title compound was prepared in a manner analogous to Intermediate 9, Steps A-B, except using 2-bromo-5-fluoro-6-methylpyridine instead of 2-bromo-6-methylpyridine in Step A. MS (ESI): mass calcd. for C₁₀H₉BrFN₃, 269.0; m/z found, 272.0 [M+H]⁺. ¹H NMR (400 MHz, DMSO-d₆) δ 8.03 (s, 1H), 7.73-7.64 (m, 2H), 3.92-3.85 (m, 3H), 2.48 (s, 3H).

### Intermediate 15: 6-(4-Bromo-1-methyl-1H-pyrazol-3-yl)-3-chloro-2-methoxypyridine

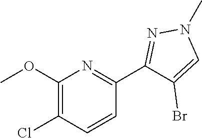

Step A. 6-Bromo-3-chloro-2-methoxypyridine. A solution of sodium nitrite (2.03 g, 29.4 mmol) and water (3 mL) was added dropwise to a 0° C. (ice/water) mixture consisting of 6-bromo-2-methoxypyridin-3-amine (3.00 g, 14.8 mmol) and conc. HCl (10.5 mL). The resultant mixture was stirred at 0° C. for 10 minutes and then added dropwise to a suspension of CuCl (3.66 g, 37.0 mmol) and conc. HCl (4.5 mL) at 0° C. (ice/water). The mixture was stirred at 60° C. for 1.5 hours. The reaction mixture was cooled to room-temperature and diluted with water (50 mL) and extracted with ethyl acetate (100 mL×3). The combined organic extracts were washed with brine (100 mL×2), dried over anhydrous Na₂SO₄, filtered, and concentrated to dryness under reduced pressure to give the crude product, which was purified by FCC (eluent:petroleum ether:ethyl acetate=1:0 to 10:1) to afford the title compound (2.00 g, 52% yield) as a white solid. ¹H NMR (400 MHz, CDCl₃) δ 7.50-7.45 (m, 1H), 7.07-6.98 (m, 1H), 4.03 (s, 3H).

Step B. 6-(4-Bromo-1-methyl-1H-pyrazol-3-yl)-3-chloro-2-methoxypyridine. The title compound was prepared in a manner analogous to Intermediate 9, Steps A-B, except using 6-bromo-3-chloro-2-methoxypyridine instead of 2-bromo-6-methylpyridine in Step A. ¹H NMR (400 MHz, CDCl₃) δ 7.65 (d, J=7.9 Hz, 1H), 7.49 (d, J=8.0 Hz, 1H), 7.47 (s, 1H), 4.14 (s, 3H), 3.93 (s, 3H).

### Intermediate 16: 2-(4-Bromo-1-(difluoromethyl)-1H-pyrazol-3-yl)-5-fluoropyridine

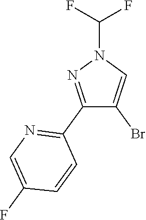 [SMILES: Fc1ccc(-c2nn(C(F)F)cc2Br)nc1]

The title compound was prepared in a manner analogous to Intermediate 8, Step A, using 2-bromo-5-fluoropyridine instead of 2-bromo-5-chloropyridine and using 1-(difluoromethyl)-3-(4,4,5,5-tetramethyl-1,3,2-dioxaborolan-2-yl)-1H-pyrazole instead of 1-methyl-3-(4,4,5,5-tetramethyl-1,3,2-dioxaborolan-2-yl)-1H-pyrazole, and heating at 80° C. instead of 100° C. in Step A. MS (ESI): mass calcd. for C₉H₅BrF₃N₃, 291.0; m/z found, 292.0 [M+H]⁺. ¹H NMR (400 MHz, CDCl₃) δ 8.61 (d, J=2.9 Hz, 1H), 8.06-8.00 (m, 1H), 7.97 (s, 1H), 7.58-7.05 (m, 2H).

### Intermediate 17: 6-(4-Bromo-1-methyl-1H-pyrazol-3-yl)-3-fluoro-2-methoxypyridine

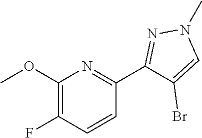

Step A. 2-Methoxy-6-(1-methyl-1H-pyrazol-3-yl)pyridin-3-amine. The title compound was prepared in a manner analogous to Intermediate 9, Step A, except using 6-bromo-2-methoxypyridin-3-amine instead of 2-bromo-6-methylpyridine, and heating at 100° C. instead of 80° C. MS (ESI): mass calcd. for C₁₀H₁₂N₄O, 204.1; m/z found, 205.1 [M+H]⁺. ¹H NMR (400 MHz, CDCl₃) δ 7.40-7.33 (m, 2H), 6.93 (d, J=7.5 Hz, 1H), 6.73 (d, J=2.3 Hz, 1H), 4.07 (s, 3H), 3.94 (s, 3H), 3.91-3.68 (m, 2H).

Step B. 2-Methoxy-6-(1-methyl-1H-pyrazol-3-yl)pyridine-3-diazonium hexafluorophosphate salt. 2-Methoxy-6-(1-methyl-1H-pyrazol-3-yl)pyridin-3-amine (720 mg, 3.53 mmol) was added to a 0° C. (ice/water) solution consisting of water (10 mL) and conc. HCl (764 uL, 9.17 mmol). The mixture was treated with sodium nitrite (486 mg, 7.04 mmol) at 0° C. The resultant mixture was stirred at 0° C. for 10 minutes before treating with hexafluorophosphoric acid (1.1 mL, 65% in water, 8.1 mmol) at 0° C. The mixture was stirred at 0° C. for another 30 minutes and the resulting suspension isolated via filtration. The filter cake was washed with water (5 mL×3) before drying under reduced pressure to afford the title compound (1.0 g, crude) a yellow solid, which was used in the next step without further purification.

Step C. 3-Fluoro-2-methoxy-6-(1-methyl-1H-pyrazol-3-yl)pyridine. 2-Methoxy-6-(1-methyl-1H-pyrazol-3-yl)pyridine-3-diazonium hexafluorophosphate salt (300 mg, 0.831 mmol), potassium fluoride (72 mg, 1.2 mmol) and toluene (2 mL) were added to a 8 mL round-bottomed flask. The resultant mixture was stirred for 16 hours at 100° C. The mixture was combined with two additional batches and concentrated to dryness under reduced pressure. Purification (FCC, eluent:petroleum ether:ethyl acetate=1:0 to 4:1) afforded the title compound (190 mg). MS (ESI): mass calcd. for C₁₀H₁₀FN₃O, 207.1; m/z found, 208.0 [M+H]⁺. ¹H NMR (400 MHz, CDCl₃) δ 7.51-7.44 (m, 1H), 7.41-7.37 (m, 1H), 7.33-7.16 (m, 1H), 6.79 (d, J=2.4 Hz, 1H), 4.10 (d, J=4.9 Hz, 3H), 3.96 (d, J=4.6 Hz, 3H).

Step D. 6-(4-Bromo-1-methyl-1H-pyrazol-3-yl)-3-fluoro-2-methoxypyridine. The title compound was prepared in a manner analogous to Intermediate 9, Step B. MS (ESI): mass calcd. for C₁₀H₉BrFN₃O, 285.0; m/z found, 286.0 [M+H]⁺. ¹H NMR (400 MHz, CDCl₃) δ 7.54-7.44 (m, 2H), 7.41-7.34 (m, 1H), 4.15 (s, 3H), 3.95 (s, 3H).

### Intermediate 18: 2-(4-Bromo-1-methyl-1H-pyrazol-3-yl)-5-fluoropyridine

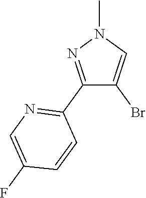

Method A:

The title compound was prepared in a manner analogous to Intermediate 8, Steps A-B except using 2-bromo-5-fluoropyridine instead of 2-bromo-5-chloropyridine in Step A. MS (ESI): mass calcd. for C₉H₇BrFN₃, 255.0; m/z found, 256.0 [M+H]⁺. ¹H NMR (500 MHz, CDCl₃) δ 8.60 (d, J=3.0 Hz, 1H), 8.02 (ddd, J=8.7, 4.4, 0.6 Hz, 1H), 7.52 (s, 1H), 7.49 (ddd, J=8.8, 8.1, 2.9 Hz, 1H), 3.99 (s, 3H).

Method B:

Step A. 5-Fluoro-2-(1-methyl-1H-pyrazol-3-yl)pyridine. To a reaction vessel containing THE (2 L, 10V) and H₂O (1 L, 5 V) was added 2-bromo-5-fluoropyridine (200 g, 1.14 mol), 1-methyl-3-(4,4,5,5-tetramethyl-1,3,2-dioxaborolan-2-yl)-1H-pyrazole (284 g, 1.36 mol), K₃PO₄ (482 g, 2.27 mol), and XPhos-Pd G3 (24 g, 28.4 mmol). The reaction vessel was degassed under vacuum, placed under a N₂ atmosphere, and heated to 60° C. for 18 hours. H₂O (2 L, 10 V) and EtOAc (2 L, 10 V) were then added and the layers were separated. The aqueous layer was extracted with additional EtOAc (1 L, 5V) and the organics combined. The combined organics were washed with brine (4 L, 20 V), dried with Na₂SO₄, filtered, and concentrated to dryness. Recrystallization from heptane (1 L, 5V), followed by filtration, yielded the title compound (150 g, 62%).

Step B. 2-(4-Bromo-1-methyl-1H-pyrazol-3-yl)-5-fluoropyridine. To 5-fluoro-2-(1-methyl-1H-pyrazol-3-yl)pyridine (5 g, 28.2 mmol) in DMF (25 mL, 5V) was added NBS (6.03 g, 33.9 mmol) portion-wise. After 30 minutes the reaction was dropped into H₂O (250 mL) and stirred for 1 hour. The solids were then filtered, the cake washed with additional H₂O (50 mL), and then dried under vacuum at 50° C. to provide the title compound. To obtain additional material, the filtrate was extracted with EtOAc (100 mL). The organics were washed with brine (50 mL), dried with Na₂SO₄, filtered, and concentrated to produce the title compound (combined mass of 5.7 g, 79% yield).

### Intermediate 19: 4-(4-Bromo-1-methyl-1H-pyrazol-3-yl)-3-fluoropyridine

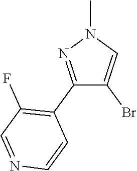

Step A. 3-Fluoro-4-(1-methyl-1H-pyrazol-3-yl)pyridine. To a mixture of 4-bromo-3-fluoropyridine (254 mg, 1.44 mmol), 1-methyl-3-(4,4,5,5-tetramethyl-1,3,2-dioxaborolan-2-yl)-1 h-pyrazole (250 mg, 1.2 mmol) in 1,4-dioxane (3.6 mL) and sat. Na₂CO₃ (aq) (1.2 mL, 3.84 mmol) in a microwave vial was added Pd(dppf)Cl₂·DCM (50 mg, 0.06 mmol). The reaction mixture was flushed with N₂ for 2 minutes, sealed then heated to 80° C. for 18 hours. The reaction was cooled, diluted with water (5 mL), and extracted with EtOAc (3×5 mL). The combined organic layers were dried (Na₂SO₄) and filtered. Purification by chromatography (silica gel, 0-100% EtOAc/Hex) afforded 137 mg (64%) of the title compound. MS (ESI): mass calcd. for C₉H₈FN₃, 177.1; m/z found, 178.1 [M+H]⁺.

Step B. 4-(4-Bromo-1-methyl-1H-pyrazol-3-yl)-3-fluoropyridine. To a solution of 3-fluoro-4-(1-methyl-1H-pyrazol-3-yl)pyridine (137 mg, 0.77 mmol) in DCM (5 mL) at room temperature was added N-bromosuccinimide (209 mg, 1.16 mmol). After 7 hours stirring at room temperature, added another portion of N-bromosuccinimide (104 mg, 0.58 mmol) and the resulting solution was stirred at room temperature for another 18 hours. Then to the resulting solution the third portion of N-bromo succinimide (35 mg, 0.2 mmol) was added and the resulting solution was stirred at room temperature for 24 hours. Then it was quenched with sat. sodium bicarbonate solution (5 mL) and extracted with DCM (3×5 mL). The combined organic layers were dried (Na₂SO₄) and filtered. Purification by chromatography (silica gel, 0-100% EtOAc/DCM) afforded 334 mg (93% yield, 55% pure) of the title compound. MS (ESI): mass calcd. for C₉H₇BrFN₃, 255.0; m/z found, 256.0 [M+H]⁺.

### Intermediate 20: 2-(4-Bromo-1-methyl-1H-pyrazol-3-yl)-5-(difluoromethyl)pyridine

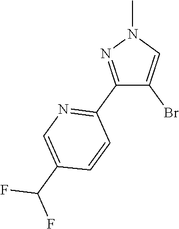 [SMILES: Cn1cc(Br)c(-c2ccc(C(F)F)cn2)n1]

The title compound was prepared in a manner analogous to Intermediate 8, Steps A-B except using 2-chloro-5-(difluoromethyl)pyridine instead of 2-bromo-5-chloropyridine in Step A. MS (ESI): mass calcd. for C₁₀H₈BrF₂N₃, 287.0; m/z found, 288.0.

### Intermediate 21: 2-(4-Bromo-1-methyl-1H-pyrazol-3-yl)-5-methylpyridine

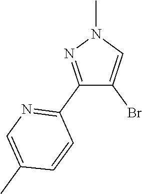

The title compound was prepared in a manner analogous to Intermediate 8, Steps A-B except using 2-bromo-5-methylpyridine instead of 2-bromo-5-chloropyridine in Step A. MS (ESI): mass calcd. for C₁₀H₁₀BrN₃, 251.0; m/z found, 252.0.

### Intermediate 22: 2-(4-Bromo-1-methyl-1H-pyrazol-3-yl)-3,5-difluoropyridine

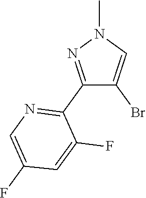

The title compound was prepared in a manner analogous to Intermediate 8, Steps A-B except using 2-bromo-3,5-difluoropyridine instead of 2-bromo-5-chloropyridine in Step A. MS (ESI): mass calcd. for C₉H₆BrF₂N₃, 273.0; m/z found, 274.0.

### Intermediate 23: 2-(4-Bromo-1-ethyl-1H-pyrazol-3-yl)-5-fluoropyridine

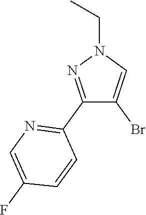

The title compound was prepared in a manner analogous to Intermediate 8, Steps A-B except using 2-bromo-5-fluoropyridine instead of 2-bromo-5-chloropyridine and 1-ethyl-3-(4,4,5,5-tetramethyl-1,3,2-dioxaborolan-2-yl)-1H-pyrazole instead of 1-methyl-3-(4,4,5,5-tetramethyl-1,3,2-dioxaborolan-2-yl)-1 h-pyrazole in Step A. MS (ESI): mass calcd. for C₁₀H₉BrFN₃, 269.0; m/z found, 270.0.

### Intermediate 24: 2-(4-Bromo-1-isopropyl-1H-pyrazol-3-yl)-5-fluoropyridine

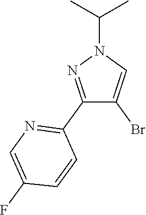 [SMILES: CC(C)n1cc(Br)c(-c2ccc(F)cn2)n1]

The title compound was prepared in a manner analogous to Intermediate 8, Steps A-B except using 2-bromo-5-fluoropyridine instead of 2-bromo-5-chloropyridine and 1-isopropyl-3-(4,4,5,5-tetramethyl-1,3,2-dioxaborolan-2-yl)-1H-pyrazole instead of 1-methyl-3-(4,4,5,5-tetramethyl-1,3,2-dioxaborolan-2-yl)-1H-pyrazole in Step A. MS (ESI): mass calcd. for C₁₁H₁₁BrFN₃, 283.0; m/z found, 284.0.

### Intermediate 25: 2-(4-Bromo-1-isobutyl-1H-pyrazol-3-yl)-5-fluoropyridine

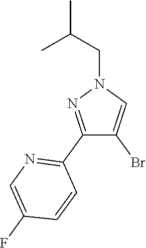 [SMILES: CC(C)Cn1cc(Br)c(-c2ccc(F)cn2)n1]

The title compound was prepared in a manner analogous to Intermediate 8, Steps A-B, except using 2-bromo-5-fluoropyridine instead of 2-bromo-5-chloropyridine and 1-isobutyl-3-(4,4,5,5-tetramethyl-1,3,2-dioxaborolan-2-yl)-1H-pyrazole instead of 1-methyl-3-(4,4,5,5-tetramethyl-1,3,2-dioxaborolan-2-yl)-1H-pyrazole in Step A. MS (ESI): mass calcd. for C₁₂H₁₃BrFN₃, 297.0; m/z found, 298.0 [M+H]⁺. ¹H NMR (500 MHz, CDCl₃) δ 8.58 (d, J=2.9 Hz, 1H), 7.99 (ddd, J=8.8, 4.4, 0.6 Hz, 1H), 7.53-7.39 (m, 2H), 3.95 (d, J=7.3 Hz, 2H), 2.35-2.19 (m, 1H), 0.94 (d, J=6.7 Hz, 6H).

### Intermediate 26: 2-(4-Bromo-1-methyl-1H-pyrazol-3-yl)-6-methoxypyridine

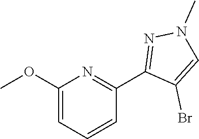

The title compound was prepared in a manner analogous to Intermediate 9, Steps A-B except using 2-bromo-6-methoxypyridine instead of 2-bromo-6-methylpyridine in Step A. MS (ESI): mass calcd. for C₁₀H₁₀BrN₃O, 267.0; m/z found, 268.0 [M+H]⁺. ¹H NMR (400 MHz, DMSO-d₆) δ 8.03 (s, 1H), 7.76-7.71 (m, 1H), 7.47-7.43 (m, 1H), 6.77 (dd, J=0.7, 8.4 Hz, 1H), 3.95 (s, 3H), 3.90 (s, 3H).

### Intermediate 27: 6-(4-Bromo-1-methyl-1H-pyrazol-3-yl)-3-chloro-2-methylpyridine

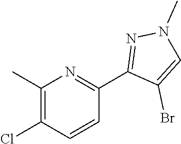

The title compound was prepared in a manner analogous to Intermediate 9, Steps A-B except using 6-bromo-3-chloro-2-methylpyridine instead of 2-bromo-6-methylpyridine in Step A. MS (ESI): mass calcd. for C₁₀H₉BrClN₃, 285.0; m/z found, 286.0 [M+H]⁺.

### Intermediate 28: 2-(4-Bromo-1-(oxetan-3-ylmethyl)-1H-pyrazol-3-yl)-5-fluoropyridine

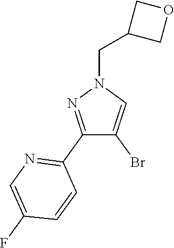 [SMILES: Fc1ccc(-c2nn(CC3COC3)cc2Br)nc1]

The title compound was prepared in a manner analogous to Intermediate 13, except using oxetan-3-ylmethyl 4-methylbenzenesulfonate (Intermediate 69) instead of 2,2,2-trifluoroethyl trifluoromethanesulfonate. MS (ESI): mass calcd. for C₁₂H₁₁BrFN₃O, 311.0 m/z found, 312.0 [M+H]⁺.

### Intermediate 29: 2-(4-Bromo-1-methyl-1H-pyrazol-3-yl)-4-fluoropyridine

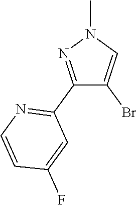

The title compound was prepared in a manner analogous to Intermediate 9, Steps A-B except using 2-bromo-4-fluoropyridine instead of 2-bromo-6-methylpyridine in Step A. MS (ESI): mass calcd. for C₉H₇BrFN₃, 255.0; m/z found, 256.0 [M+H]⁺.

### Intermediate 30: 5-(4-Bromo-1-methyl-1H-pyrazol-3-yl)-2-fluoropyridine

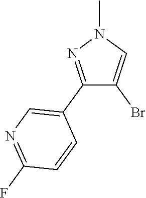

The title compound was prepared in a manner analogous to Intermediate 9, Steps A-B except using 5-bromo-2-fluoropyridine instead of 2-bromo-6-methylpyridine in Step A. MS (ESI): mass calcd. for C₉H₇BrFN₃, 255.0; m/z found, 256.0 [M+H]⁺.

### Intermediate 31: 5-(4-Bromo-1-methyl-1H-pyrazol-3-yl)-2-methylpyridine

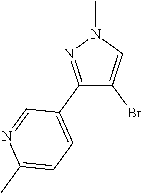

The title compound was prepared in a manner analogous to Intermediate 9, Steps A-B except using 5-bromo-2-methylpyridine instead of 2-bromo-6-methylpyridine in Step A. MS (ESI): mass calcd. for C₁₀H₁₀BrN₃, 251.0; m/z found, 256.0 [M+H]⁺.

### Intermediate 32: 5-(4-Bromo-1-methyl-1H-pyrazol-3-yl)-2-(difluoromethoxy)pyridine

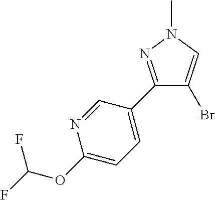 [SMILES: Cn1cc(Br)c(-c2ccc(OC(F)F)nc2)n1]

The title compound was prepared in a manner analogous to Intermediate 9, Steps A-B except using 5-bromo-2-(difluoromethoxy)pyridine instead of 2-bromo-6-methylpyridine in Step A. MS (ESI): mass calcd. for C₁₀H₈BrF₂N₃O, 303.0; m/z found, 304.0 [M+H]⁺.

### Intermediate 33: 2-(4-Bromo-1-methyl-1H-pyrazol-3-yl)-5-(difluoromethoxy)pyridine

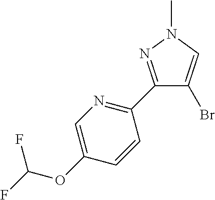 [SMILES: Cn1cc(Br)c(-c2ccc(OC(F)F)cn2)n1]

The title compound was prepared in a manner analogous to Intermediate 9, Steps A-B except using 2-bromo-5-(difluoromethoxy)pyridine instead of 2-bromo-6-methylpyridine in Step A. MS (ESI): mass calcd. for C₁₀H₈BrF₂N₃O, 303.0; m/z found, 304.0 [M+H]⁺.

### Intermediate 34: 3-(4-Bromo-1-methyl-1H-pyrazol-3-yl)pyridine

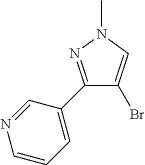

The title compound was prepared in a manner analogous to Intermediate 9, Steps A-B except using 3-bromopyridine instead of 2-bromo-6-methylpyridine in Step A. MS (ESI): mass calcd. for C₉H₈BrN₃, 237.0; m/z found, 238.1 [M+H]⁺.

### Intermediate 35: 2-(4-Bromo-1-methyl-1H-pyrazol-3-yl)-6-fluoropyridine

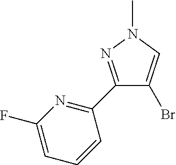

The title compound was prepared in a manner analogous to Intermediate 9, Steps A-B except using 2-bromo-6-fluoropyridine instead of 2-bromo-6-methylpyridine in Step A. MS (ESI): mass calcd. for C₉H₇BrFN₃, 255.0; m/z found, 256.0 [M+H]⁺.

### Intermediate 36: 3-(4-Bromo-1-methyl-1H-pyrazol-3-yl)-5-fluoropyridine

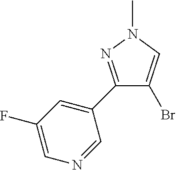

The title compound was prepared in a manner analogous to Intermediate 9, Steps A-B except using 3-bromo-5-fluoropyridine instead of 2-bromo-6-methylpyridine in Step A. MS (ESI): mass calcd. for C₉H₇BrFN₃, 255.0; m/z found, 256.1 [M+H]⁺.

### Intermediate 37: 2-(4-Bromo-1-methyl-1H-pyrazol-3-yl)oxazole

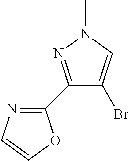

The title compound was prepared in a manner analogous to Intermediate 9, Steps A-B except using 2-bromooxazole instead of 2-bromo-6-methylpyridine in Step A. MS (ESI): mass calcd. for C₇H₆BrN₃O, 227.0; m/z found, 228.1 [M+H]⁺.

### Intermediate 38: 2-(4-Bromo-1,5-dimethyl-1H-pyrazol-3-yl)-5-fluoropyridine

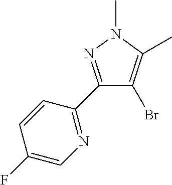

Step A. 1-(5-Fluoropyridin-2-yl)-3-hydroxybut-2-en-1-one. Potassium tert-butoxide (5.3 mL of 1 M in tetrahydrofuran, 5.32 mmol) was added to a mixture of 1-(5-fluoropyridin-2-yl)ethenone (370 mg, 2.66 mmol) and ethyl acetate (2.62 mL, 26.6 mmol) and the reaction stirred at ambient temperature for 24 h. The reaction was quenched with 2 M HCl (2.6 mL) and diluted further with EtOAc. The organic layer was washed with H₂O and brine then dried (Na₂SO₄), filtered, and condensed. Purification by column chromatography (silica gel, 0-60% EtOAc/hexanes) afforded 113 mg (23%) of the title compound. MS (ESI): mass calcd. for C₉H₈FNO₂, 181.1; m/z found, 182.1 [M+H]⁺. ¹H NMR (400 MHz, CDCl₃) δ 15.77-15.62 (m, 1H), 8.51 (d, J=2.8 Hz, 1H), 8.14 (dd, J=8.8, 4.6 Hz, 1H), 7.58-7.49 (m, 1H), 6.78 (s, 1H), 2.24 (s, 3H).

Step B. 5-Fluoro-2-(5-methyl-1H-pyrazol-3-yl)pyridine. To a solution of 1-(5-fluoropyridin-2-yl)-3-hydroxybut-2-en-1-one (55 mg, 0.304 mmol) in tetrahydrofuran (1.2 mL) was added hydrazine monohydrate (0.025 mL of a 60% aqueous solution, 0.304 mmol) and the reaction heated to 65° C. for 24 hours. The mixture was condensed and used without further purification. MS (ESI): mass calcd. for C₉H₈FN₃, 177.1; m/z found, 178.1 [M+H]⁺.

Step C. 2-(1,5-Dimethyl-1H-pyrazol-3-yl)-5-fluoropyridine. Sodium hydride (13 mg of 60% in mineral oil, 0.335 mmol) was added to a stirred solution of 5-fluoro-2-(5-methyl-1H-pyrazol-3-yl)pyridine (54 mg, 0.305 mmol) in dimethylformamide (0.6 mL). The reaction was stirred at ambient temperature for 10 minutes then iodomethane (0.038 mL, 0.61 mmol) was added and the resulting mixture stirred at ambient temperature for 16 h. The reaction was quenched with H₂O then extracted with EtOAc 3×. The combined organics were dried (Na₂SO₄), filtered, and condensed. Purification by column chromatography (silica gel, 0-100% EtOAc/hexanes) afforded 23 mg (39%) of the title compound. MS (ESI): mass calcd. for C₁₀H₁₀FN₃, 191.1; m/z found, 192.1 [M+H]⁺. ¹H NMR (400 MHz, CDCl₃) δ 8.46 (d, J=2.9 Hz, 1H), 7.87 (dd, J=8.8, 4.5 Hz, 1H), 7.41 (td, J=8.50, 2.88 Hz, 1H), 6.58 (d, J=0.6 Hz, 1H), 3.85 (s, 3H), 2.33 (d, J=0.6 Hz, 3H).

Step D. 2-(4-Bromo-1,5-dimethyl-1H-pyrazol-3-yl)-5-fluoropyridine. To a stirred solution of 2-(1,5-dimethyl-1H-pyrazol-3-yl)-5-fluoropyridine (23 mg, 0.12 mmol) in dimethylformamide (0.6 mL) was added N-bromosuccinimide (NBS) (24 mg, 0.132 mmol). The reaction mixture was stirred at ambient temperature for 2 h. The resulting mixture was diluted with H₂O and extracted with EtOAc then the combined organics were washed with brine, dried (Na₂SO₄), filtered, and condensed. The material was used without further purification. MS (ESI): mass calcd. for C₁₀H₉BrFN₃, 269.0; m/z found, 270.0 [M+H]⁺.

### Intermediate 39: 2-(4-Bromo-5-cyclobutyl-1-methyl-1H-pyrazol-3-yl)-5-fluoropyridine

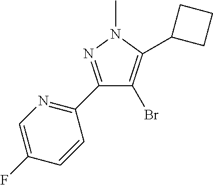 [SMILES: Cn1nc(-c2ccc(F)cn2)c(Br)c1C1CCC1]

Step A. 1-Cyclobutyl-3-(5-fluoropyridin-2-yl)propane-1,3-dione. Potassium tert-butoxide (1.55 mL of 1 M in tetrahydrofuran, 1.55 mmol) was added to a 0° C. mixture of cyclobutyl methyl ketone (0.17 mL, 1.55 mmol) and methyl 5-fluoropicolinate (200 mg, 1.29 mmol) then the reaction was allowed to slowly warm to room temperature at the rate of ice bath expiration and stirred for 16 h. The reaction was quenched with 2 M HCl (1.6 mL) and condensed. The residue was taken up in EtOH, filtered, and condensed. The material was used without further purification. MS (ESI): mass calcd. for C₁₂H₁₂FNO₂, 221.1; m/z found, 222.1 [M+H]⁺.

Step B. 2-(5-Cyclobutyl-1-methyl-1H-pyrazol-3-yl)-5-fluoropyridine. Methylhydrazine (0.14 mL, 2.58 mmol) was added to a solution of 1-cyclobutyl-3-(5-fluoropyridin-2-yl)propane-1,3-dione (285 mg, 1.29 mmol) in acetic acid (2.6 mL) and ethanol (2.6 mL) in a microwave vial. The reaction was capped and heated to 70° C. for 24 hours then condensed. The residue was taken up in EtOAc and washed with saturated aqueous NaHCO₃, water, and brine. The organics were dried (Na₂SO₄), filtered, and condensed. The resulting material was utilized without further purification. MS (ESI): mass calcd. for C₁₃H₁₄FN₃, 231.1; m/z found, 232.1 [M+H]⁺.

Step C. 2-(4-Bromo-5-cyclobutyl-1-methyl-1H-pyrazol-3-yl)-5-fluoropyridine. To a stirred solution of 2-(5-cyclobutyl-1-methyl-1H-pyrazol-3-yl)-5-fluoropyridine (298 mg, 1.29 mmol) in dimethylformamide (2.6 mL) was added N-bromosuccinimide (NBS) (252 mg, 1.42 mmol). The reaction mixture was stirred at ambient temperature for 2 h. The resulting mixture was diluted with H₂O and extracted with EtOAc then the combined organics were washed with brine, dried (Na₂SO₄), filtered, and condensed. Purification by column chromatography (silica gel, 0-100% EtOAc/hexanes) afforded 225 mg (56% yield) of the title compound. MS (ESI): mass calcd. for C₁₃H₁₃BrFN₃, 309.0; m/z found, 310.0 [M+H]⁺. ¹H NMR (400 MHz, CDCl₃) δ 8.57 (d, J=2.9 Hz, 1H), 7.94 (dd, J=8.8, 4.4 Hz, 1H), 7.46 (td, J=8.4, 3.0 Hz, 1H), 3.91 (s, 3H), 3.77-3.65 (m, 1H), 2.78-2.63 (m, 2H), 2.49-2.36 (m, 2H), 2.18-1.92 (m, 2H).

### Intermediate 40: 1-Benzyl-4-bromo-6-cyclopropyl-1H-pyrazolo[3,4-b]pyridine

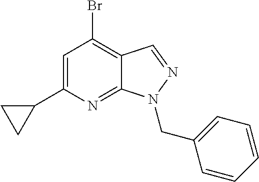 [SMILES: Brc1cc(C2CC2)nc2c1cnn2Cc1ccccc1]

Step A. (Z)-3-((1-Benzyl-1H-pyrazol-5-yl)imino)-3-cyclopropylpropanoic acid. A mixture of 1-benzyl-1H-pyrazol-5-amine (2.00 g, 11.0 mmol), ethyl 3-cyclopropyl-3-oxopropanoate (1.71 g, 11.0 mmol), and acetic acid (0.063 mL, 1.10 mmol) in toluene (15 mL) in a round bottomed flask equipped with a Dean-Stark trap was heated to reflux for 24 hours. The reaction was condensed and purified by column chromatography (0-100% EtOAc/hexanes). MS (ESI): mass calcd. for C₁₆H₁₇N₃O₂, 283.1; m/z found, 284.2 [M+H]⁺. ¹H NMR (500 MHz, CDCl₃) δ 9.71 (br s, 1H) 7.48 (d, J=1.88 Hz, 1H) 7.27-7.35 (m, 3H) 7.18-7.22 (m, 2H) 6.45 (d, J=1.88 Hz, 1H) 5.29 (s, 2H) 3.66 (s, 2H) 1.97 (tt, J=7.71, 4.49 Hz, 1H) 1.13-1.19 (m, 2H) 1.04-1.10 (m, 2H).

Step B. 1-Benzyl-6-cyclopropyl-1H-pyrazolo[3,4-b]pyridin-4-ol. Dowtherm® A (3 mL) was heated to 275° C. then 1-(5-amino-1-benzyl-1H-pyrazol-4-yl)-3-cyclopropylpropane-1,3-dione was dissolved in 3 mL Dowtherm® A and added dropwise to the refluxing mixture. After 1 h, the reaction was cooled to room temperature then purified by column chromatography (silica gel, 0-100% EtOAc/hexanes) to afford 820 mg (28% yield) of the title compound. MS (ESI): mass calcd. for C₁₆H₁₅N₃O, 265.1; m/z found, 266.2 [M+H]⁺. ¹H NMR (500 MHz, CDCl₃) δ 7.97 (s, 1H), 7.39-7.21 (m, 5H), 7.18-7.12 (m, 1H), 5.53 (s, 2H), 1.96-1.84 (m, 1H), 1.01-0.89 (m, 4H).

Step C. 1-Benzyl-4-bromo-6-cyclopropyl-1H-pyrazolo[3,4-b]pyridine. A suspension of 1-benzyl-6-cyclopropyl-1H-pyrazolo[3,4-b]pyridine-4-ol (810 mg, 3.05 mmol) and phosphorus oxybromide (1.31 g, 4.57 mmol) in toluene (6 mL) was heated to 115° C. then dimethylformamide (2.4 mL, 30.5 mmol) was added over 1 h. At this point the reaction was cooled to room temperature then quenched with saturated aqueous NaHCO₃. The aqueous layer was extracted with EtOAc (2×) then the combined organics were washed with water and brine, dried (Na₂SO₄), filtered, and condensed. Purification by column chromatography (silica gel, 0-100% EtOAc/hexanes) afforded 234 mg (23% yield) of the title compound. MS (ESI): mass calcd. for C₁₆H₁₄BrN₃, 327.0; m/z found, 328.1 [M+H]⁺. ¹H NMR (400 MHz, CDCl₃) δ 7.91 (s, 1H), 7.37-7.24 (m, 5H), 7.21 (s, 1H), 5.59 (s, 2H), 2.17-2.07 (m, 1H), 1.21-1.15 (m, 2H), 1.11-1.05 (m, 2H).

### Intermediate 41: 1-Benzyl-4-bromo-3,6-dimethyl-1H-pyrazolo[3,4-b]pyridine

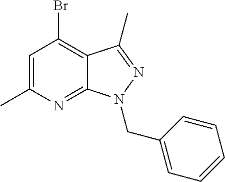 [SMILES: Cc1cc(Br)c2c(C)nn(Cc3ccccc3)c2n1]

Step A. Methyl (E)-3-((1-benzyl-3-methyl-1H-pyrazol-5-yl)imino)butanoate. A mixture of 1-benzyl-3-methyl-1H-pyrazol-5-amine (5.00 g, 26.7 mmol), methyl 3-oxobutanoate (5.59 g, 48.1 mmol) and TsOH (0.10 g, 0.53 mmol) in toluene was heated at 70° C. under N₂ for 14 hrs. The mixture was cooled to room temperature and filtered. The filter cake was washed with toluene (2V). The combined filtrate was concentrated to give a residue which was purified by chromatography on silica gel (PE-EA=20:1, 15:1, 10:1, 8:1) to give the title compound (6.3 g, 83% yield). MS (ESI): mass calcd. for C₁₆H₁₉N₃O₂, 285.2; m/z found, 286.2 [M+H]⁺. ¹H NMR (400 MHz, CDCl₃) δ 10.00 (s, 1H), 7.34-7.23 (m, 5H), 5.81 (s, 1H), 5.18 (s, 2H), 4.57 (s, 1H), 3.69 (s, 3H), 2.27 (s, 3H), 1.72 (s, 3H).

Step B. 1-Benzyl-3,6-dimethyl-1H-pyrazolo[3,4-b]pyridin-4-ol. Dowtherm® A (48 mL, 8V) was heated up to 240° C. in a round bottom flask under N₂ and methyl (E)-3-((1-benzyl-3-methyl-1H-pyrazol-5-yl)imino)butanoate (6.0 g, 21.0 mmol) was added. After stirring for 2 hours the reaction mixture was cooled to room temperature and petroleum ether (48 mL, 8V) was added. The solid was collected by filtration and washed with petroleum ether twice. Further purification by slurry with ethyl acetate and petroleum ether (V/V=1:5) give the title compound (4.5 g, 85% yield). MS (ESI): mass calcd. for C₁₅H₁₅N₃O, 253.1; m/z found, 254.2 [M+H]⁺.

Step C. 1-Benzyl-4-bromo-3,6-dimethyl-1H-pyrazolo[3,4-b]pyridine. To a mixture of 1-benzyl-3,6-dimethyl-1H-pyrazolo[3,4-b]pyridin-4-ol (4.5 g, 17.8 mmol) in toluene (45.0 ml, 10V) and DMF (13.5 ml, 3V) was added POBr₃ (6.1 g, 21.3 mmol) under N₂. The mixture was heated at 110° C. for 1 h. The reaction mixture was cooled to room temperature and cold water (225 mL, 50V) was added. Extracted with DCM (225 mL*2, 50V*2) and concentrated to give a crude oil. Further purification by chromatography on silica gel (PE-EA=100:1 to 60:1 to 40:1 to 20:1) give the title compound (4.6 g 82% yield). MS (ESI): mass calcd. for C₁₅H₁₄BrN₃, 315.0; m/z found, 316.0 [M+H]⁺. ¹H NMR (400 MHz, CDCl₃) δ 7.33-7.24 (m, 5H), 7.15 (s, 1H), 5.32 (s, 2H), 2.72 (s, 3H), 2.65 (s, 3H).

### Intermediate 42: 1-(Phenylsulfonyl)-4-(4,4,5,5-tetramethyl-1,3,2-dioxaborolan-2-yl)-1H-pyrrolo[2,3-b]pyridine

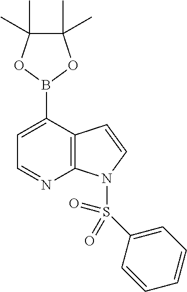 [SMILES: CC1(C)OB(c2ccnc3c2ccn3S(=O)(=O)c2ccccc2)OC1(C)C]

Step A: 4-Bromo-1-(phenylsulfonyl)-1H-pyrrolo[2,3-b]pyridine. To a solution of 4-bromo-1H-pyrrolo[2,3-b]pyridine (8 g, 40.6 mmol) in N,N-dimethylformamide (160 mL) was added sodium hydride (60% in mineral oil, 1.79 g, 44.8 mmol) at 0° C. under argon. The reaction mixture was stirred at 0° C. for 30 min under argon. To the reaction mixture was added benzenesulfonyl chloride (5.7 mL, 44.7 mmol, 1.384 g/mL) dropwise at 0° C. over 10 min under argon. The reaction mixture was allowed to warm to room temperature. The reaction mixture was stirred at room temperature for 16 h under argon. The reaction mixture was diluted with water (150 mL) and extracted with ethyl acetate (2×200 mL). The combined organic layers were washed with water (1×100 mL) and brine (1×100 mL), dried over magnesium sulfate, filtered and evaporated. The residue was purified by gradient silica gel column chromatography eluting with n-heptane:ethyl acetate (9:0→9:1) to afford the title compound (12.1 g, 88%) as a white crystalline solid. MS (ESI): mass calcd. for C₁₃H₉BrN₂O₂S, 336.0; m/z found, 337.0 [M+H]⁺.

Step B: 1-(Phenylsulfonyl)-4-(4,4,5,5-tetramethyl-1,3,2-dioxaborolan-2-yl)-1H-pyrrolo[2,3-b]pyridine. To a mixture of 4-bromo-1-(phenylsulfonyl)-1H-pyrrolo[2,3-b]pyridine (12 g, 35.6 mmol), bis(pinacolato)diboron (45.2 g, 178 mmol), [1,1′-bis(diphenylphosphino)ferrocene]dichloropalladium(II) dichloride (2.6 g, 3.55 mmol) and potassium acetate (10.5 g, 107 mmol) was added 1,4-dioxane (300 mL). The reaction was split evenly 2 portions and stirred at 80° C. for 8 h under argon. The combined reaction mixtures were filtered through a pad of Celite. The Celite was washed with 1,4-dioxane (6×30 mL) and the combined filtrate layers were evaporated. The residue was purified by gradient silica gel flash chromatography eluting with n-heptane:ethyl acetate (100:0→1:1). The residue was triturated with n-hexane (3×20 mL) to afford the title compound (11.1 g, 81%) as a white powder. MS (ESI): mass calcd. for C₁₉H₂₁BN₂O₄S, 384.1; m/z found, 303.1 [(M−C₆H₁₀)+H]⁺.

### Intermediate 43: 4-Bromo-2-(difluoromethyl)-1-(phenylsulfonyl)-1H-pyrrolo[2,3-b]pyridine

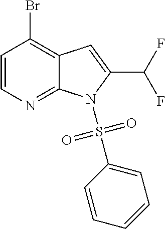 [SMILES: O=S(=O)(c1ccccc1)n1c(C(F)F)cc2c(Br)ccnc21]

To a 0° C. solution of triethylamine trihydrofluoride (3.7 mL, 22.2 mmol) in dichloromethane (41 mL) was successively added XtalFluor-E® (3.76 g, 16.4 mmol) and 4-bromo-1-phenylsulfonyl-7-azaindole-2-carboxaldehyde (3.0 g, 8.22 mmol). After 30 minutes the reaction was warmed to room temperature and allowed to proceed overnight at ambient temperature then quenched with saturated aqueous NaHCO₃. The aqueous layer was extracted with CH₂Cl₂ (2×) then the combined organics were washed with water and brine, dried (Na₂SO₄), filtered, and condensed. Purification by column chromatography (silica gel, 0-100% EtOAc/hexanes) afforded 1.98 g (62% yield) of the title compound. MS (ESI): mass calcd. for C₁₄H₉BrF₂N₂O₂S, 386.0; m/z found, 387.0 [M+H]⁺. ¹H NMR (500 MHz, CDCl₃) δ 8.32-8.26 (m, 3H) 7.64-7.60 (m, 1H) 7.55-7.42 (m, 3H) 7.44 (br t, J=54.5 Hz, 1H) 7.06 (d, J=0.8 Hz, 1H).

### Intermediate 44: 4-chloro-2-isopropyl-1H-pyrrolo[2,3-b]pyridine

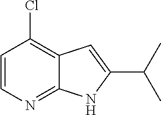

Step A. 4-Chloro-3-(3-methylbut-1-yn-1-yl)pyridin-2-amine. 4-Chloro-3-iodopyridin-2-amine (300 mg, 1.18 mmol), copper iodide (11 mg, 0.059 mmol), triethylamine (1.15 mL, 8.25 mmol), 3-methyl-1-butyne (0.12 mL, 1.18 mmol), and acetonitrile (3.4 mL) were combined in a sealed vessel. The reaction mixture was degassed with nitrogen, bis(triphenylphosphine)palladium(II) dichloride (33 mg, 0.047 mmol) was added, then the vial was capped and heated to 75° C. for 16 h. The mixture was condensed then purified by column chromatography (silica gel, 0-10% MeOH/CH₂Cl₂) to afford 203 mg (88% yield) of the title compound. MS (ESI): mass calcd. for C₁₀H₁₁ClN₂, 194.1; m/z found, 195.1 [M+H]⁺. ¹H NMR (500 MHz, CDCl₃) δ 7.86 (d, J=5.5 Hz, 1H), 6.71 (d, J=5.5 Hz, 1H), 5.12 (br s, 2H), 2.84-2.98 (m, 1H), 1.34 (d, J=6.9 Hz, 6H).

Step B. 4-Chloro-2-isopropyl-1H-pyrrolo[2,3-b]pyridine. To a solution of 4-chloro-3-(3-methylbut-1-yn-1-yl)pyridine-2-amine (205 mg, 1.05 mmol) in NMP (2 mL) was added potassium tert-butoxide (2.2 mL of a 1 M THF solution, 2.2 mmol) and the reaction was heated to 75° C. for 2 h. The mixture was diluted with EtOAc then washed with water and brine, dried (Na₂SO₄), filtered, and condensed. Purification by column chromatography (0-80% EtOAc/hexanes) afforded 114 mg (56% yield) of the title compound. MS (ESI): mass calcd. for C₁₀H₁₁ClN₂, 194.1; m/z found, 195.1 [M+H]⁺. ¹H NMR (500 MHz, CDCl₃) δ 10.14 (br s, 1H) 8.10 (d, J=5.3 Hz, 1H) 7.07 (d, J=5.3 Hz, 1H) 6.31 (dd, J=2.3, 0.8 Hz, 1H) 3.15 (dt, J=13.9, 6.9 Hz, 1H) 1.42 (d, J=6.9 Hz, 6H).

### Intermediate 45: 2-(4-Bromo-1-(tetrahydro-2H-pyran-2-yl)-1H-pyrazol-5-yl)-5-fluoropyridine

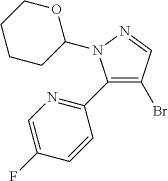 [SMILES: Fc1ccc(-c2c(Br)cnn2C2CCCCO2)nc1]

Step A. 5-Fluoro-2-(1-(tetrahydro-2H-pyran-2-yl)-1H-pyrazol-5-yl)pyridine. 2-Bromo-5-fluoropyridine (11.0 g, 62.5 mmol), 1-(tetrahydro-2H-pyran-2-yl)-5-(4,4,5,5-tetramethyl-1,3,2-dioxaborolan-2-yl)-1H-pyrazole (20.9 g, 75.1 mmol)), aq.Na₂CO₃ (2 M, 44 mL), toluene (110 mL), and EtOH (110 mL) were added to a 500 mL round-bottomed flask. The mixture was sparged with N₂ for 5 minutes and then treated with Pd(PPh₃)₄ (7.26 g, 6.28 mmol). The resultant mixture was sparged with N₂ for another 5 minutes and then stirred while heating at 90° C. for 2 hours. The reaction mixture was cooled to room-temperature, poured into H₂O (20 mL), and extracted with ethyl acetate (50 mL×3). The combined organic extracts were dried over anhydrous Na₂SO₄, filtered, and concentrated to dryness under reduced pressure to give the crude product, which was purified by FCC (eluent:petroleum ether:ethyl acetate=1:0 to 10:1) to afford the title compound (14 g, 77%) as a brown oil. ¹H NMR (400 MHz, CDCl₃) δ 8.57-8.43 (m, 1H), 7.61-7.53 (m, 2H), 7.48-7.39 (m, 1H), 6.55-6.46 (m, 1H), 6.08 (d, J=10.0 Hz, 1H), 4.13-3.94 (m, 2H), 3.61-3.48 (m, 1H), 2.59-2.34 (m, 1H), 2.10-2.02 (m, 1H), 1.77-1.57 (m, 2H), 1.57-1.40 (m, 1H).

Step B. 2-(4-Bromo-1-(tetrahydro-2H-pyran-2-yl)-1H-pyrazol-5-yl)-5-fluoropyridine. 5-Fluoro-2-(1-(tetrahydro-2H-pyran-2-yl)-1H-pyrazol-5-yl)pyridine (7.00 g, 28.3 mmol), NBS (6.10 g, 34.3 mmol), and dichloromethane (130 mL) were added to a 250 mL round-bottomed flask. The resultant mixture was stirred at room-temperature for 16 hours. The reaction mixture was poured into water (60 mL) and extracted with dichloromethane (50 mL×3). The combined organic extracts were dried over anhydrous Na₂SO₄, filtered, and concentrated to dryness under reduced pressure to give the crude product, which was purified by FCC (eluent:petroleum ether:ethyl acetate=1:0 to 3:1) to afford the title compound (4.7 g, 43% yield) a yellow oil. MS (ESI): mass calcd. for C₁₃H₁₃BrFN₃O, 325.0; m/z found, 326.0 [M+H]⁺. ¹H NMR (400 MHz, CDCl₃) δ 8.50 (d, J=2.8 Hz, 1H), 7.92 (dd, J=4.5, 8.8 Hz, 1H), 7.68 (s, 1H), 7.38 (dt, J=3.0, 8.4 Hz, 1H), 5.34 (dd, J=3.3, 9.0 Hz, 1H), 4.02-3.94 (m, 1H), 3.67-3.56 (m, 1H), 2.07-1.87 (m, 3H), 1.64-1.47 (m, 3H).

### Intermediate 46: 4-Chloro-2-(trifluoromethyl)-1-((2-(trimethylsilyl)ethoxy)methyl)-1H-pyrrolo[2,3-b]pyridine

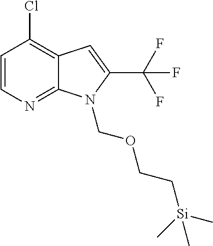 [SMILES: C[Si](C)(C)CCOCn1c(C(F)(F)F)cc2c(Cl)ccnc21]

Step A. 4-Chloro-2-(trifluoromethyl)-1H-pyrrolo[2,3-b]pyridine and 4-chloro-3-(trifluoromethyl)-1H-pyrrolo[2,3-b]pyridine. An oven dried 40 mL vial was charged with 4-chloro-7-azaindole (250 mg, 1.64 mmol), (Ir[dF(CF₃)ppy]₂(dtbpy))PF₆ (18 mg, 0.016 mmol), sodium trifluoromethanesulfonate (767 mg, 4.92 mmol), and ammonium persulfate (374 mg, 1.64 mmol). The vial was capped and the headspace was purged with N₂ before adding DMSO (8 mL). The reaction was degassed by 3 cycles of vacuum and backfilling with N₂, irradiated with blue light for 2.5 h, then diluted with water and extracted with EtOAc (2×). The combined organics were washed with H₂O (2×) and brine, dried (MgSO₄), filtered, and condensed. Purification by column chromatography (silica gel, 0-100% EtOAc/hexanes) afforded 53 mg (15% yield) of 4-chloro-2-(trifluoromethyl)-1H-pyrrolo[2,3-b]pyridine MS (ESI): mass calcd. for C₈H₄ClF₃N₂, 220.0; m/z found, 221.0 [M+H]⁺. ¹H NMR (400 MHz, DMSO-d₆) δ 8.40 (d, J=5.1 Hz, 1H), 7.39 (d, J=5.1 Hz, 1H), 7.13 (s, 1H) and 71 mg (20%) of 4-chloro-3-(trifluoromethyl)-1H-pyrrolo[2,3-b]pyridine MS (ESI): mass calcd. for C₈H₄ClF₃N₂, 220.0; m/z found, 221.0 [M+H]⁺. ¹H NMR (400 MHz, DMSO-d₆) δ 12.87 (br s, 1H), 8.34 (d, J=5.1 Hz, 1H), 8.27 (s, 1H), 7.40 (d, J=5.1 Hz, 1H).

Step B. 4-Chloro-2-(trifluoromethyl)-1-((2-(trimethylsilyl)ethoxy)methyl)-1H-pyrrolo[2,3-b]pyridine. Sodium hydride (14 mg of 60% in mineral oil, 0.362 mmol) was added to a solution of 4-chloro-2-(trifluoromethyl)-1H-pyrrolo[2,3-b]pyridine (53 mg, 0.242 mmol) in dimethylformamide (1 mL). The mixture was stirred until gas evolution ceased then 2-(trimethylsilyl)ethoxymethyl chloride (0.1 mL, 0.483 mmol) was added. The reaction stirred at ambient temperature for 2 h then quenched with saturated aqueous NaHCO₃. The aqueous layer was extracted with EtOAc then the organics were condensed. Purification by column chromatography (silica gel, 0-40% EtOAc/hexanes) afforded 68 mg (56%) of the title compound. MS (ESI): mass calcd. for C₁₄H₁₈ClF₃N₂OSi, 350.1; m/z found, 351.1 [M+H]⁺. ¹H NMR (400 MHz, CDCl₃) δ 8.37 (d, J=5.1 Hz, 1H), 7.23 (d, J=5.1 Hz, 1H), 7.09 (d, J=1.0 Hz, 1H), 5.82 (s, 2H), 3.73-3.54 (m, 2H), 1.11-0.81 (m, 2H), −0.03-−0.08 (m, 9H).

### Intermediate 47: 4-Chloro-3-(trifluoromethyl)-1-((2-(trimethylsilyl)ethoxy)methyl)-1H-pyrrolo[2,3-b]pyridine

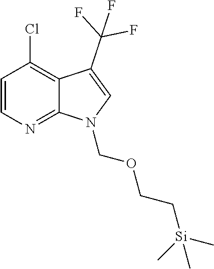 [SMILES: C[Si](C)(C)CCOCn1cc(C(F)(F)F)c2c(Cl)ccnc21]

The title compound was prepared in a manner analogous to Intermediate 46, except using 4-chloro-3-(trifluoromethyl)-1H-pyrrolo[2,3-b]pyridine (Step B) instead of 4-chloro-2-(trifluoromethyl)-1H-pyrrolo[2,3-b]pyridine (Step B). MS (ESI): mass calcd. for C₁₄H₁₈ClF₃N₂OSi, 350.1; m/z found, 351.1 [M+H]⁺. ¹H NMR (400 MHz, CDCl₃) δ 8.30 (d, J=5.2 Hz, 1H), 7.79 (q, J=1.2 Hz, 1H), 7.27-7.24 (m, 1H), 5.69 (s, 2H), 3.59-3.53 (m, 2H), 0.96-0.89 (m, 2H), −0.05 (s, 9H).

### Intermediate 48: 4-Chloro-5-(oxetan-3-yl)-1H-pyrrolo[2,3-b]pyridine

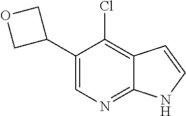 [SMILES: Clc1c(C2COC2)cnc2[nH]ccc12]

A 20 mL vial was charged with (Ir[dF(CF₃)ppy]₂(dtbpy))PF₆ (7 mg, 0.006 mmol), 5-bromo-4-chloro-1H-pyrrolo[2,3-b]pyridine (150 mg, 0.648 mmol), and anhydrous lithium hydroxide (39 mg, 1.62 mmol) and put under N₂ atmosphere. In a separate vial, nickel(II) chloride ethylene glycol dimethyl ether complex (7.1 mg, 0.032 mmol) and 4,4′-di-tert-butyl-2,2′-bipyridine (10.4 mg, 0.039 mmol) were mixed in dimethoxyethane (1 mL) until a uniform slurry formed. This catalyst mixture was added to the reaction vial along with dimethoxyethane (4 mL), tris(trimethylsilyl)silane (0.24 mL, 0.778 mmol), and 3-bromooxetane (0.11 mL, 1.30 mmol). The solution was degassed by N₂ sparging then irradiated with blue light for 3 h. After this time the reaction mixture was condensed. Purification by column chromatography (silica gel, 0-100% EtOAc/hexanes) afforded 36 mg (27% yield) of the title compound. MS (ESI): mass calcd. for C₁₀H₉ClN₂O, 208.0; m/z found, 208.9 [M+H]⁺. ¹H NMR (400 MHz, DMSO-d₆) δ 8.75 (s, 1H), 8.00 (d, J=3.4 Hz, 1H), 6.88 (d, J=3.4 Hz, 1H), 5.41 (dd, J=8.4, 6.0 Hz, 2H), 5.25 (dd, J=7.4, 6.0 Hz, 2H), 5.15-5.04 (m, 1H).

### Intermediate 49: 3-(4-Chloro-1H-pyrrolo[2,3-b]pyridin-2-yl)oxetan-3-ol

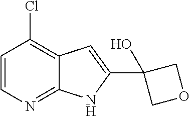 [SMILES: OC1(c2cc3c(Cl)ccnc3[nH]2)COC1]

Step A. 3-((2-Amino-4-chloropyridin-3-yl)ethynyl)oxetan-3-ol. To a vial containing 4-chloro-3-iodopyridin-2-amine (500 mg, 1.97 mmol), 3-ethynyloxetan-3-ol (193 mg, 1.97 mmol), Cu(I) iodide (19 mg, 0.098 mmol), and [1,1′-bis(diphenylphosphino)ferrocene]dichloropalladium(II) (55 mg, 0.079 mmol) was added acetonitrile (6 mL) and triethylamine (1.9 mL, 13.8 mmol). The vial was capped and the reaction mixture was degassed under vacuum then refilled with N₂. The mixture was heated to 80° C. for 8 h. The reaction mixture was cooled to room temperature then condensed onto silica gel. Purification by chromatography (silica gel, 0-20% MeOH/CH₂Cl₂) afforded 425 mg (96% yield) of the title compound. MS (ESI): mass calcd. for C₁₀H₉ClN₂O₂, 224.0; m/z found, 225.0 [M+H]⁺. ¹H NMR (400 MHz, CDCl₃) δ 7.93 (d, J=5.5 Hz, 1H), 6.73 (d, J=5.5 Hz, 1H), 5.11 (br s, 2H), 4.99-4.93 (m, 2H), 4.87-4.80 (m, 2H).

Step B. 3-(4-Chloro-1H-pyrrolo[2,3-b]pyridin-2-yl)oxetan-3-ol. To a solution of 3-((2-amino-4-chloropyridin-3-yl)ethynyl)oxetan-3-ol (425 mg, 1.89 mmol) in 1-methyl-2-pyrrolidinone (4 mL) was added potassium tert-butoxide (4 mL of 1 M in THF, 4 mmol) and then the reaction was heated to 75° C. for 2 h. The mixture was diluted with H₂O then the aqueous layer extracted with EtOAc (3×). The combined organics were washed with H₂O and brine, dried (Na₂SO₄), filtered, and condensed. Purification by column chromatography (0-100% EtOAc/hexanes) afforded 183 mg (43%) of the title compound. MS (ESI): mass calcd. for C₁₀H₉ClN₂O₂, 224.0; m/z found, 225.0 [M+H]⁺. ¹H NMR (400 MHz, DMSO-d₆) δ 12.13 (br s, 1H), 8.15 (d, J=5.1 Hz, 1H), 7.18 (d, J=5.1 Hz, 1H), 6.59 (s, 2H), 4.88 (d, J=7.1 Hz, 2H), 4.72 (d, J=7.1 Hz, 2H).

### Intermediate 50: 4-Chloro-5-(oxetan-3-ylmethyl)-1H-pyrrolo[2,3-b]pyridine

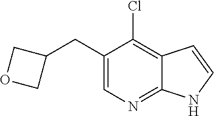 [SMILES: Clc1c(CC2COC2)cnc2[nH]ccc12]

The title compound was prepared in a manner analogous to Intermediate 48, except using 3-(bromomethyl)oxetane instead of 3-bromooxetane. MS (ESI): mass calcd. for C₁₁H₁₁ClN₂O, 222.1; m/z found, 223.0 [M+H]⁺. ¹H NMR (400 MHz, DMSO-d₆) δ 11.98-11.87 (m, 1H), 8.14 (s, 1H), 7.54 (d, J=3.4 Hz, 1H), 6.45 (d, J=3.4 Hz, 1H), 4.62 (dd, J=7.6, 5.9 Hz, 2H), 4.40 (t, J=6.1 Hz, 2H), 3.39-3.32 (m, 1H), 3.15 (d, J=7.8 Hz, 2H).

### Intermediate 51: (R/S)-3-(4-Chloro-1H-pyrrolo[2,3-b]pyridin-2-yl)tetrahydrofuran-3-ol

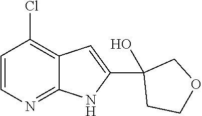 [SMILES: OC1(c2cc3c(Cl)ccnc3[nH]2)CCOC1]

Step A. 3-(4-Chloro-1-(phenylsulfonyl)-1H-pyrrolo[2,3-b]pyridin-2-yl)tetrahydrofuran-3-ol. To a −78° C. solution of 4-chloro-1-(phenylsulfonyl)-1H-pyrrolo[2,3-b]pyridine (1.5 g, 5.12 mmol) in THE (15 mL) was added n-butyllithium (2.6 mL of 1.6 M in hexane, 4.10 mmol). After 30 minutes, dihydrofuran-3(2H)-one (0.61 g, 6.83 mmol) was added and the reaction was allowed to slowly warm to room temperature overnight. The reaction was quenched with saturated aqueous ammonium chloride then the aqueous layer was extracted with EtOAc (3×) then the combined organics dried (Na₂SO₄), filtered, and condensed. Used without further purification. MS (ESI): mass calcd. for C₁₇H₁₅ClN₂O₄S, 378.0; m/z found, 379.0 [M+H]⁺.

Step B. 3-(4-Chloro-1H-pyrrolo[2,3-b]pyridin-2-yl)tetrahydrofuran-3-ol. To a solution of 3-(4-chloro-1-(phenylsulfonyl)-1H-pyrrolo[2,3-b]pyridin-2-yl)tetrahydrofuran-3-ol (1.30 g, 3.42 mmol) in MeOH (7 mL) was added powdered potassium hydroxide (0.77 g, 13.7 mmol) and the reaction was heated to 70° C. for 6 h. After this time the mixture was cooled to room temperature and condensed onto silica gel. Purification by column chromatography (silica gel, 0-6% MeOH saturated with NH₃/CH₂Cl₂) afforded 240 mg (29% yield) of the title compound. MS (ESI): mass calcd. for C₁₁H₁₁ClN₂O₂, 238.1; m/z found, 239.1 [M+H]⁺. ¹H NMR (400 MHz, CDCl₃) δ 8.19 (d, J=5.3 Hz, 1H), 7.12 (d, J=5.3 Hz, 1H), 6.46 (d, J=2.0 Hz, 1H), 4.29-4.21 (m, 1H), 4.20-4.13 (m, 1H), 4.09-4.04 (m, 1H), 4.02-3.97 (m, 1H), 2.54 (dt, J=13.3, 8.7 Hz, 1H), 2.45-2.36 (m, 1H).

### Intermediate 52: 4-Chloro-6-methyl-1-tosyl-1H-pyrrolo[2,3-b]pyridine

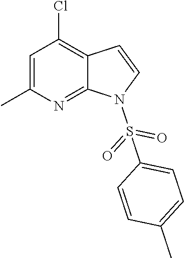 [SMILES: Cc1ccc(S(=O)(=O)n2ccc3c(Cl)cc(C)nc32)cc1]

In a pressure vessel was placed 4-chloro-6-methyl-1H-pyrrolo[2,3-b]pyridine (190 mg, 1.14 mmol), cesium carbonate (750 mg, 2.3 mmol), acetonitrile (4 mL) followed by 4-methylbenzenesulfonyl chloride (434 mg, 2.3 mmol). The vial was capped and purged with N₂ and allowed to stir at 70° C. for 18 hours. The reaction mixture was partitioned between EtOAc and a saturated aqueous solution of NaCl. The organic layer was separated, dried over MgSO₄, filtered and concentrated. Purification via silica gel chromatography (20% to 100% EtOAc/Hexanes) gave the title compound (340 mg, 93% yield). MS (ESI): mass calcd. for C₁₅H₁₃ClN₂O₂S, 320.0; m/z found, 321.0 [M+H]⁺. ¹H NMR (400 MHz, DMSO-d₆) δ 8.08-7.99 (m, 2H), 7.89 (d, J=4.0 Hz, 1H), 7.48-7.40 (m, 2H), 7.36 (d, J=0.5 Hz, 1H), 6.79 (d, J=4.0 Hz, 1H), 2.55 (s, 3H), 2.35 (s, 3H).

### Intermediate 53: 4-Chloro-5-methyl-1-tosyl-1H-pyrrolo[2,3-b]pyridine

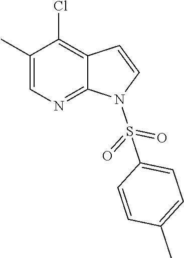 [SMILES: Cc1ccc(S(=O)(=O)n2ccc3c(Cl)c(C)cnc32)cc1]

The title compound was prepared in a manner analogous to Intermediate 52, Step A, using 4-chloro-5-methyl-1H-pyrrolo[2,3-b]pyridine instead of 4-chloro-6-methyl-1H-pyrrolo[2,3-b]pyridine. MS (ESI): mass calcd. for C₁₅H₁₃ClN₂O₂S, 320.0; m/z found, 321.0 [M+H]⁺. ¹H NMR (400 MHz, DMSO-d₆) δ 8.34-8.29 (m, 1H), 8.02-7.92 (m, 3H), 7.45-7.38 (m, 2H), 6.81 (d, J=4.0 Hz, 1H), 2.36 (d, J=0.6 Hz, 3H), 2.34 (s, 3H).

### Intermediate 54: 4-Chloro-2-(oxetan-3-yl)-1-(phenylsulfonyl)-1H-pyrrolo[2,3-b]pyridine

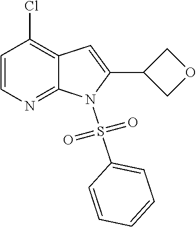 [SMILES: O=S(=O)(c1ccccc1)n1c(C2COC2)cc2c(Cl)ccnc21]

Step A. 2-Bromo-4-chloro-1-(phenylsulfonyl)-1H-pyrrolo[2,3-b]pyridine. To a −78° C. solution of 4-chloro-1-(phenylsulfonyl)-1H-pyrrolo[2,3-b]pyridine (1.5 g, 5.12 mmol) in THF (15 mL) was added lithium diisopropylamide (3 mL of 2 M in THF/heptane, 6.15 mmol) and the reaction stirred at this temperature for 1 h. After this time 1,2-dibromotetrachloroethane (2.0 g, 6.15 mmol) was added and the reaction stirred at −78° C. for 2 h. The mixture was allowed to warm to room temperature then quenched with saturated aqueous ammonium chloride. The aqueous layer was extracted with EtOAc (2×) then the combined organics dried (Na₂SO₄), filtered, and condensed. Purification by column chromatography (silica gel, 0-25% EtOAc/hexanes) afforded 1.85 g (97%) of the title compound. MS (ESI): mass calcd. for C₁₃H₈BrClN₂O₂S, 370.0; m/z found, 371.0 [M+H]⁺. ¹H NMR (400 MHz, CDCl₃) δ 8.33 (d, J=5.3 Hz, 1H), 8.20 (d, J=7.7 Hz, 2H), 7.64-7.57 (m, 1H), 7.51 (t, J=7.3 Hz, 2H), 7.19 (d, J=5.3 Hz, 1H), 6.84 (s, 1H).

Step B. 4-Chloro-2-(oxetan-3-yl)-1-(phenylsulfonyl)-1H-pyrrolo[2,3-b]pyridine. The title compound was prepared in a manner analogous to Intermediate 48, except using 2-bromo-4-chloro-1-(phenylsulfonyl)-1H-pyrrolo[2,3-b]pyridine instead of 5-bromo-4-chloro-1H-pyrrolo[2,3-b]pyridine. MS (ESI): mass calcd. for C₁₆H₁₃ClN₂O₃S, 348.0; m/z found, 349.0 [M+H]⁺. ¹H NMR (400 MHz, CDCl₃) δ 8.29 (d, J=5.3 Hz, 1H), 8.16-8.12 (m, 2H), 7.64-7.58 (m, 1H), 7.54-7.47 (m, 2H), 7.23-7.19 (m, 1H), 6.71-6.70 (m, 1H), 5.23-5.11 (m, 2H), 4.98-4.84 (m, 3H)

### Intermediate 55: 4-(3-Bromo-1-methyl-1H-pyrazol-4-yl)-1-(phenylsulfonyl)-1H-pyrrolo[2,3-b]pyridine

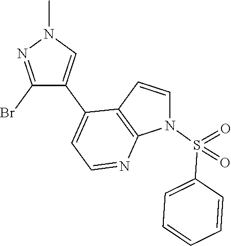 [SMILES: Cn1cc(-c2ccnc3c2ccn3S(=O)(=O)c2ccccc2)c(Br)n1]

Step A. 1-Methyl-4-(1-(phenylsulfonyl)-1H-pyrrolo[2,3-b]pyridin-4-yl)-1H-pyrazol-3-amine. A mixture of 4-bromo-1-methyl-1H-pyrazol-3-amine (4.58 g, 26 mmol), 1-(phenylsulfonyl)-4-(4,4,5,5-tetramethyl-1,3,2-dioxaborolan-2-yl)-1H-pyrrolo[2,3-b]pyridine (Intermediate 42, 11 g, 28.6 mmol), [1,1′-bis(diphenylphosphino)ferrocene]dichloropalladium(II) (1.9 g, 2.6 mmol) and sodium carbonate (2 M in water, 36.4 mL, 72.8 mmol) in 1,4-dioxane (70 mL) was stirred at 90° C. for 4 h under argon. To the reaction mixture was added water (300 mL) and ethyl acetate (300 mL). The mixture was filtered through a pad of Celite®. The Celite® was washed with ethyl acetate (2×50 mL) and the combined filtrate layers were separated. The aqueous layer was extracted with ethyl acetate (2×200 mL). The combined organic layers were washed with brine (1×200 mL). The organic layer was dried over magnesium sulfate, filtered, and evaporated. The residue was purified by gradient silica gel flash chromatography eluting with dichloromethane:ethyl acetate (4:1→0:1). The residue was triturated with diethyl ether (3×10 mL) to afford the title compound (4.04 g, 11.4 mmol, 44%) as a tan crystalline solid. MS (ESI): mass calcd. for C₁₇H₁₅N₅O₂S, 353.1; m/z found, 354.1 [M+H]⁺.

Step B. 4-(3-Bromo-1-methyl-1H-pyrazol-4-yl)-1-(phenylsulfonyl)-1H-pyrrolo[2,3-b]pyridine. To a solution of 1-methyl-4-(1-(phenylsulfonyl)-1H-pyrrolo[2,3-b]pyridin-4-yl)-1H-pyrazol-3-amine (4 g, 11.3 mmol) in acetonitrile (120 mL) was added tert-butyl nitrite (1.79 mL, 13.6 mmol, 0.867 g/mL) dropwise at 0° C. under argon. To the reaction mixture was added copper(I) bromide (1.95 g, 13.6 mmol) at 0° C. under argon. The reaction mixture was allowed to warm to room temperature. The reaction mixture was stirred at room temperature for 16 h under argon, then at 50° C. for 2 h under argon. To the reaction mixture was added water (200 mL) and ethyl acetate (100 mL). The mixture was filtered through a pad of Celite®. The Celite® was washed with ethyl acetate (2×50 mL). The combined filtrate layers were separated. The aqueous layer was extracted with ethyl acetate (1×150 mL). The combined organic layers were washed with brine (1×150 mL). The organic layer was dried over magnesium sulfate, filtered, and evaporated. The residue was purified by gradient silica gel column chromatography eluting with n-heptane:ethyl acetate (2:1→1:2) to give the title compound (1.63 g, 3.906 mmol, 35%) as an off-white crystalline solid. MS (ESI): mass calcd. for C₁₇H₁₃BrN₄O₂S, 416.0; m/z found, 417.0 [M+H]⁺.

### Intermediate 56: 4-(1-Methyl-3-(trimethylstannyl)-1H-pyrazol-4-yl)-1-(phenylsulfonyl)-1H-pyrrolo[2,3-b]pyridine

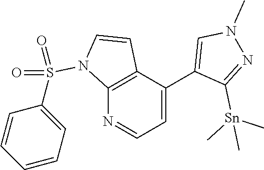 [SMILES: Cn1cc(-c2ccnc3c2ccn3S(=O)(=O)c2ccccc2)[c]([Sn]([CH3])([CH3])[CH3])n1]

To a mixture of 4-(3-bromo-1-methyl-1H-pyrazol-4-yl)-1-(phenylsulfonyl)-1H-pyrrolo[2,3-b]pyridine (Intermediate 55, 450 mg, 1.08 mmol), hexamethylditin (224 μL, 1.08 mmol, 1.58 g/mL) and dichlorobis(triphenylphosphine)palladium(II) (76 mg, 0.108 mmol) was added 1,4-dioxane (10.5 mL). The reaction mixture was stirred at 80° C. for 64 h under argon. The reaction mixture was filtered through a pad of Celite®. The Celite® was washed with chloroform (5×25 mL) and the combined filtrates were evaporated. The residue was purified by gradient column chromatography on neutral alumina eluting with heptane:ethyl acetate (4:1→2:1) to afford the title compound (152 mg, 0.303 mmol, 28%) as a colorless gum. ¹H NMR (500 MHz, DMSO-d₆) δ 8.40 (d, J=5.1 Hz, 1H), 8.29 (s, 1H), 8.17-8.12 (m, 2H), 8.00 (d, J=4.1 Hz, 1H), 7.76-7.71 (m, 1H), 7.66-7.61 (m, 2H), 7.46 (d, J=5.1 Hz, 1H), 6.92 (d, J=4.2 Hz, 1H), 3.91 (s, 3H), 3.32 (s, 9H).

### Intermediate 57: 2-(4-Bromo-1-methyl-1H-pyrazol-3-yl)-5-(trifluoromethoxy)pyridine

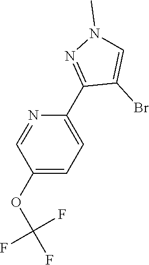 [SMILES: Cn1cc(Br)c(-c2ccc(OC(F)(F)F)cn2)n1]

The title compound was prepared in a manner analogous to Intermediate 8, Steps A-B except using 2-bromo-5-(trifluoromethoxy)pyridine instead of 2-bromo-5-chloropyridine in Step A. MS (ESI): mass calcd. for C₁₀H₇BrF₃N₃O, 321.0; m/z found, 322.0 [M+H]⁺.

### Intermediate 58: 4-(4-Bromo-1-methyl-1H-pyrazol-3-yl)pyrimidine

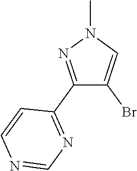

Step A: 4-(1-Methyl-1H-pyrazol-3-yl)pyrimidine. To a mixture of 4-chloropyrimidine (165 mg, 1.44 mmol), 1-methyl-3-(4,4,5,5-tetramethyl-1,3,2-dioxaborolan-2-yl)-1 h-pyrazole (250 mg, 1.2 mmol) in 1,4-dioxane (3.6 mL) and sat. Na₂CO₃ (aq) (1.2 mL, 3.84 mmol) in a microwave vial was added Pd(dppf)Cl₂·DCM (50 mg, 0.06 mmol). The reaction mixture was flushed with N₂ for 2 minutes, sealed then heated to 80° C. for 18 hours. The reaction was cooled, diluted with water (5 mL), and extracted with EtOAc (3×5 mL). The combined organic layers were dried (Na₂SO₄) and filtered. Purification by chromatography (silica gel, 0-100% EtOAc/DCM) afforded 125 mg (65%) of the title compound. MS (ESI): mass calcd. for C₈H₈N₄, 160.1; m/z found, 161.1 [M+H]⁺.

Step B: 4-(4-Bromo-1-methyl-1H-pyrazol-3-yl)pyrimidine. To a solution of 4-(1-methyl-1H-pyrazol-3-yl)pyrimidine (125 mg, 0.78 mmol) in DCM (5 mL) at room temperature was added 1-bromopyrrolidine-2,5-dione (210 mg, 1.2 mmol). After 18 hours stirring at room temperature, added another portion of N-bromosuccinimide (105 mg, 0.59 mmol) and the resulting solution was stirred at room temperature for another 18 hours. Then it was quenched with sat. sodium bicarbonate solution (5 mL) and extracted with DCM (2×5 mL). The combined organic layers were dried (Na₂SO₄) and filtered. Purification by chromatography (silica gel, 0-100% EtOAc/DCM) afforded 160 mg (86%) of the title compound. MS (ESI): mass calcd. for C₈H₇BrN₄, 238.0; m/z found, 239.0 [M+H]⁺.

### Intermediate 59: 4-(4-Bromo-1-methyl-1H-pyrazol-3-yl)pyridine

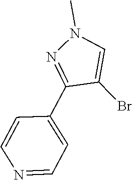

The title compound was prepared in a manner analogous to Intermediate 58, except using 4-bromopyridine instead of 4-chloropyrimidine in Step A. MS (ESI): mass calcd. for C₉H₈BrN₃, 237.0; m/z found, 238.0 [M+H]⁺.

### Intermediate 60: 4-(4-Bromo-1-methyl-1H-pyrazol-3-yl)-3-fluoropyridine

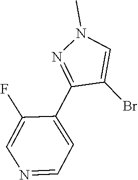

The title compound was prepared in a manner analogous to Intermediate 58, except using 4-bromo-3-fluoropyridine instead of 4-chloropyrimidine in Step A. MS (ESI): mass calcd. for C₉H₇BrFN₃, 255.0; m/z found, 256.0 [M+H]^{+.}

### Intermediate 61: 5-(4-bromo-1-methyl-1H-pyrazol-3-yl)pyrimidine

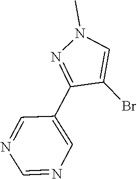

The title compound was prepared in a manner analogous to Intermediate 58, except using 5-bromopyrimidine instead of 4-chloropyrimidine in Step A. MS (ESI): mass calcd. for C₈H₇BrN₄, 238.0; m/z found, 239.0 [M+H]^{+.}

### Intermediate 62: 2-(4-Bromo-1-methyl-1H-pyrazol-3-yl)pyrazine

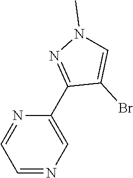

The title compound was prepared in a manner analogous to Intermediate 58, except using 2-bromopyrazine instead of 4-chloropyrimidine in Step A. MS (ESI): mass calcd. for C₈H₇BrN₄, 238.0; m/z found, 239.0 [M+H]^{+.}

### Intermediate 63: 3-(4-Bromo-1-methyl-1H-pyrazol-3-yl)pyridazine

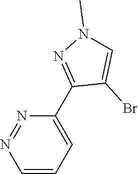

The title compound was prepared in a manner analogous to Intermediate 58, except using 3-bromo-pyridazine hydrobromide instead of 4-chloropyrimidine in Step A. MS (ESI): mass calcd. for C₈H₇BrN₄, 238.0; m/z found, 239.0 [M+H]^{+.}

### Intermediate 64: 4-(4-Bromo-1-methyl-1H-pyrazol-3-yl)pyridazine

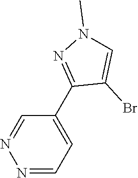

The title compound was prepared in a manner analogous to Intermediate 58, except using 4-bromopyridazine hydrobromide instead of 4-chloropyrimidine in Step A. MS (ESI): mass calcd. for C₈H₇BrN₄, 238.0; m/z found, 239.0 [M+H]^{+.}

### Intermediate 65: 2-(4-Bromo-1-methyl-1H-pyrazol-3-yl)-5-fluoropyrimidine

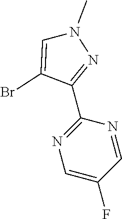

The title compound was prepared in a manner analogous to Intermediate 8, Steps A-B except using 2-bromo-5-fluoropyrimidine instead of 2-bromo-5-chloropyridine in Step A. MS (ESI): mass calcd. for C₈H₆BrFN₄, 256.0; m/z found, 257.0 [M+H]⁺.

### Intermediate 66: 2-(4-Bromo-1-methyl-1H-pyrazol-3-yl)-5-methylpyrazine

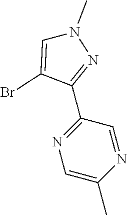

The title compound was prepared in a manner analogous to Intermediate 8, Steps A-B except using 2-bromo-5-methylpyrazine instead of 2-bromo-5-chloropyridine in Step A. MS (ESI): mass calcd. for C₉H₉BrN₄, 252.0; m/z found, 253.0 [M+H]⁺.

### Intermediate 67: 2-(4-Bromo-1-(2-fluoroethyl)-1H-pyrazol-3-yl)-5-fluoropyridine

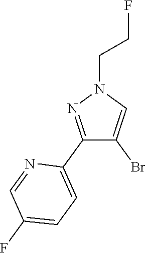 [SMILES: FCCn1cc(Br)c(-c2ccc(F)cn2)n1]

The title compound was prepared in a manner analogous to Intermediate 11, except using 1-fluoro-2-iodoethane instead of 3-bromooxetane and stirred at room temperature instead of 50° C. MS (ESI): mass calcd. for C₁₀H₈BrF₂N₃, 287.0; m/z found, 288.0 [M+H]⁺.

### Intermediate 68: 2-(4-Bromo-1-cyclopropyl-1H-pyrazol-3-yl)-5-fluoropyridine

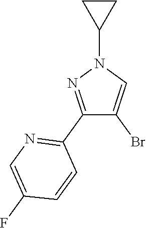 [SMILES: Fc1ccc(-c2nn(C3CC3)cc2Br)nc1]

Step A. (Z)-3-(Dimethylamino)-1-(5-fluoropyridin-2-yl)prop-2-en-1-one. In a sealed vessel was placed 1-(5-fluoropyridin-2-yl)ethan-1-one (1.0 g, 7.20 mmol) and DMF-DMA (10 mL). The reaction mixture was stirred at 110° C. for 16 hours. The reaction mixture was poured into water (20 mL) and extracted with ethyl acetate (20 mL×3). The combined organic layers were washed with brine, and dried over with anhydrous Na₂SO₄, filtrated, concentrated to dryness under reduced pressure to afford crude product. Purification via silica gel chromatography (petroleum ether:ethyl acetate=1:0 to 1:1) gave the title compound (1.4 g, 100%). ¹H NMR (400 MHz, DMSO-d₆) δ 8.65-8.55 (m, 1H), 8.11-8.00 (m, 1H), 7.86-7.74 (m, 2H), 6.30 (br. d, J=11.9 Hz, 1H), 3.17 (s, 3H), 2.99-2.83 (m, 3H).

Step B. 2-(1-Cyclopropyl-1H-pyrazol-3-yl)-5-fluoropyridine. To a solution of (Z)-3-(dimethylamino)-1-(5-fluoropyridin-2-yl)prop-2-en-1-one (1.1 g, 5.7 mmol) in ethanol (15 mL) was added cyclopropylhydrazine hydrochloride (1.230 g, 11.33 mmol). The reaction mixture was stirred at 80° C. for 16 hours then the solvent was evaporated. Water was added and the aqueous phase was extracted with ethyl acetate (20 mL×3). The combined organic layers were washed with a saturated solution of sodium chloride, dried over with anhydrous Na₂SO₄, filtrated and evaporated. The crude was purified by preparative HPLC Method A to afford the title compound. A further purification via SFC Method C; gave the pure title compound (26 mg, 2.3%). MS (ESI): mass calcd. for C₁₁H₁₀FN₃, 203.1; m/z found, 204.0 [M+H]⁺. ¹H NMR (400 MHz, DMSO-d₆): 8.54 (d, J=3.1 Hz, 1H), 7.95 (dd, J=4.6, 8.8 Hz, 1H), 7.85 (d, J=2.4 Hz, 1H), 7.78-7.67 (m, 1H), 6.74 (d, J=2.4 Hz, 1H), 2.07 (s, 1H), 1.13-1.06 (m, 2H), 1.05-0.95 (m, 2H).

Step C. 2-(4-Bromo-1-cyclopropyl-1H-pyrazol-3-yl)-5-fluoropyridine. The title compound was prepared in a manner analogous to Intermediate 8, Step B, except using dichloromethane instead of acetonitrile and stirring for 2 hours instead of 48 h. MS (ESI): mass calcd. for C₁₁H₉BrFN₃, 281.0; m/z found, 282.0 [M+H]⁺.

### Intermediate 69: Oxetan-3-ylmethyl 4-methylbenzenesulfonate

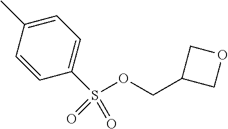 [SMILES: Cc1ccc(S(=O)(=O)OCC2COC2)cc1]

To a solution of oxetan-3-ylmethanol (5.0 g, 57 mmol), triethylamine (24 mL, 172 mmol), DMAP (1.4 g, 11 mmol) in dichloromethane cooled to 0° C. was added in portions 4-methylbenzenesulfonyl chloride. The ice/water bath was removed, and the reaction mixture was stirred at room temperature for 12 hours. It was then poured into water (100 mL) and extracted with dichloromethane (150 mL×3). The combined organic extracts were washed with a saturated solution of sodium chloride (50 mL), dried over anhydrous Na₂SO₄, filtered, and concentrated to dryness under reduced pressure to give the crude product. Purification via silica gel chromatography (eluent:petroleum ether:ethyl acetate=10:1 to 3:1) gave the title compound (10 g, 70%) as a yellow oil. MS (ESI): mass calcd. for C₁₁H₁₄O₄S, 242.1 m/z found, 243.0 [M+H]⁺. ¹H NMR (400 MHz, CDCl₃) δ 7.84-7.78 (m, 2H), 7.41-7.34 (m, 2H), 4.74 (dd, J=6.5, 7.8 Hz, 2H), 4.33 (t, J=6.1 Hz, 2H), 4.25 (d, J=7.0 Hz, 2H), 3.35-3.23 (m, 1H), 2.46 (s, 3H).

### Intermediate 70: 4-Bromo-6-methyl-1-((2-(trimethylsilyl)ethoxy)methyl)-1H-pyrazolo[3,4-b]pyridine

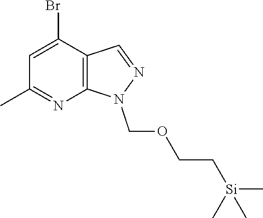

The title compound was prepared in a manner analogous to Intermediate 1, Step A except using 4-bromo-6-methyl-1H-pyrazolo[3,4-b]pyridine instead of 4-bromo-1H-pyrazolo[3,4-b]pyridine. MS (ESI): mass calcd. for C₁₃H₂₀BrN₃OSi, 341.1; m/z found, 342.0 [M+H].

### Intermediate 71: 4-(3-Bromo-1-methyl-1H-pyrazol-4-yl)-1-(4-methoxybenzyl)-6-methyl-1H-pyrazolo[3,4-b]pyridine

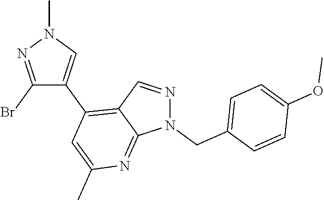 [SMILES: COc1ccc(Cn2ncc3c(-c4cn(C)nc4Br)cc(C)nc32)cc1]

Step A: 1-(4-Methoxybenzyl)-6-methyl-4-(4,4,5,5-tetramethyl-1,3,2-dioxaborolan-2-yl)-1H-pyrazolo[3,4-b]pyridine. To a solution of compound 4-Bromo-1-(4-methoxybenzyl)-6-methyl-1H-pyrazolo[3,4-b]pyridine (Intermediate 2, Step C, 220 g, 662.26 mmol) in DME (3.0 L) was added bis(pinacolato) diboron (250 g, 984 mmol) and AcOK (200 g, 2.04 mol, 3.08 eq), then added Pd(dppf)Cl₂ (25.0 g, 34.1 mmol). The mixture was stirred at 90° C. for 2 hrs after which LCMS showed consumption of the starting material. The reaction was filtered and the filtrate was poured into water (1000 mL), then extracted with EtOAc (1000 mL), the organic layer was washed with water (300 mL) and brine (300 mL), dried over Na₂SO₄, filtered and concentrated to give a residue. The residue was triturated with Petroleum ether (300 mL) to yield the title compound (100 g, 40% yield) as a yellow solid. MS (ESI): mass calcd. for C₂₁H₂₆BN₃O₃ 379.2; m/z found 380.2 [M+H]⁺. ¹H NMR: (400 MHz, CDCl₃) δ 8.14 (s, 1H), 7.31 (s, 1H), 7.22 (d, J=8.4 Hz, 2H), 6.73 (d, J=8.4 Hz, 2H), 5.56 (s, 2H), 3.68 (s, 3H), 2.62 (s, 3H), 1.32 (s, 12H)

Step B: 4-(1-(4-Methoxybenzyl)-6-methyl-1H-pyrazolo[3,4-b]pyridin-4-yl)-1-methyl-1H-pyrazol-3-amine. To a mixture of 1-(4-methoxybenzyl)-6-methyl-4-(4,4,5,5-tetramethyl-1,3,2-dioxaborolan-2-yl)-1H-pyrazolo[3,4-b]pyridine (90.0 g, 237 mmol) and 4-bromo-1-methyl-1H-pyrazol-3-amine (45.0 g, 255 mmol) in dioxane (1 L) and H₂O (300 mL) was added Na₂CO₃ (75.4 g, 711 mmol) and Pd(dppf)Cl₂·CH₂Cl₂ (9.69 g, 11.8 mmol) in one portion at 25° C. under N₂. The mixture was stirred at 90° C. for 16 hrs under N₂. The mixture was poured into water (200 mL), filtered, and the filtrate was extracted with EtOAc (1000 mL). The organic layer was washed with brine (500 mL), dried over Na₂SO₄, filtered, and concentrated to give a residue. The residue was triturated with Petroleum ether/EtOAc=5/1 (500 mL) to afford the title compound (70.0 g, 80% yield, 94% purity) as a yellow solid. MS (ESI): mass calcd. for C₁₉H₂₀N₆O 348.2; m/z found 349.2 [M+H]⁺. ¹H NMR (400 MHz, DMSO-d₆) δ 8.26 (s, 1H), 8.19 (s, 1H), 7.24 (s, 1H), 7.19 (d, J=8.4 Hz, 2H), 6.86 (d, J=8.4 Hz, 2H), 5.55 (s, 2H), 3.73-3.70 (m, 6H), 2.60 (s, 3H).

Step C: 4-(3-Bromo-1-methyl-1H-pyrazol-4-yl)-1-(4-methoxybenzyl)-6-methyl-1H-pyrazolo[3,4-b]pyridine. To a solution of 4-(1-(4-methoxybenzyl)-6-methyl-1H-pyrazolo[3,4-b]pyridin-4-yl)-1-methyl-1H-pyrazol-3-amine (65.0 g, 186 mmol) in MeCN (2 L) was added CuBr (27.3 g, 190 mmol, 5.80 mL) and tert-butyl nitrite (57.2 g, 554 mmol, 65.9 mL). The mixture was stirred at 50° C. for 16 hrs. The reaction was concentrated to give a residue. The residue was purified by silica gel chromatography (100-200 mesh silica gel, Petroleum ether/Ethyl acetate=5/1˜2/1, 35 L) to afford the title compound (20.0 g, 25% yield, 97% purity) as a yellow solid. MS (ESI): mass calcd. for C₁₉H₁₈BrN₅O 411.1; m/z found 412.1 [M+H]⁺. ¹H NMR (400 MHz, DMSO-d₆) δ 8.47 (s, 1H), 8.22 (s, 1H), 7.38 (s, 1H), 7.21 (d, J=8.4 Hz, 2H), 6.87 (d, J=8.4 Hz, 2H), 5.58 (s, 2H), 3.94 (s, 3H), 3.70 (s, 3H), 2.65 (s, 3H).

### Intermediate 72: 3-(4-Bromo-1-methyl-1H-pyrazol-3-yl)-5-fluoropyridine

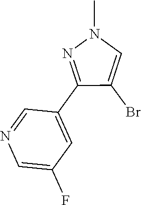

The title compound was prepared in a manner analogous to 2-(4-bromo-1-methyl-1H-pyrazol-3-yl)-5-chloropyridine (Intermediate 8), Steps A-B; using 3-bromo-5-fluoropyridine instead of 2-bromo-5-chloropyridine and using Cs₂CO₃ instead of 2 M Na₂CO₃ in Step A; and in Step B, stirring for 1 hr instead of 48 hrs. MS (ESI): mass calcd. for C₉H₇BrFN₃, 255.0; m/z found, 255.9 [M+H]⁺. ¹H NMR (400 MHz, DMSO-d₆): δ 8.89 (t, J=1.7 Hz, 1H), 8.61 (d, J=2.7 Hz, 1H), 8.14 (s, 1H), 8.05-7.96 (m, 1H), 3.92 (s, 3H).

### Intermediate 73: 6-(Difluoromethyl)-4-(4,4,5,5-tetramethyl-1,3,2-dioxaborolan-2-yl)-1-((2-(trimethylsilyl)ethoxy)methyl)-1H-pyrazolo[3,4-b]pyridine

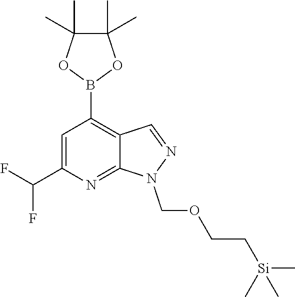 [SMILES: CC1(C)OB(c2cc(C(F)F)nc3c2cnn3COCC[Si](C)(C)C)OC1(C)C]

Step A. 4-Bromo-6-(difluoromethyl)-1H-pyrazolo[3,4-b]pyridine. To a mixture of sodium difluoromethanesulfinate (10.5 g, 76.0 mmol), 4-bromo-1H-pyrazolo[3,4-b]pyridine (5.00 g, 25.2 mmol) and H₂O (30 mL) was added TFA (1.5 mL, 40 mmol), followed by the addition of dichloromethane (30 mL) and TFA (1.5 mL, 20 mmol). The reaction mixture was stirred at room-temperature for 0.5 hour before t-BuOOH (21 mL 25 mmol) was added. The reaction mixture was stirred at room-temperature for 16 hours. The reaction mixture was poured it into sat. Na₂SO₃ (150 mL) and extracted with dichloromethane (150 mL×3). The combined organic extracts were washed with brine (150 mL), dried over anhydrous Na₂SO₄, filtered, and concentrated under reduced pressure. Purification (FCC, SiO₂, petroleum ether:ethyl acetate=1:0 to 5:1) to afford the title compound (3.0 g, 48%) as a yellow solid. MS (ESI): mass calcd. for C₇H₄BrF₂N₃ 248.0; m/z found 248.9 [M+H]⁺.

Step B. 4-Bromo-6-(difluoromethyl)-1-((2-(trimethylsilyl)ethoxy)methyl)-1H-pyrazolo[3,4-b]pyridine. To a cooled solution, 0° C., of 4-bromo-6-(difluoromethyl)-1H-pyrazolo[3.4-b]pyridine (2.50 g, 10.1 mmol) in THE (40 mL) was added sodium hydride in mineral oil (505 mg, 60% purity, 12.6 mmol) in portions. The reaction mixture was stirred at 0° C., for 30 mins, then SEM-Cl (2.7 mL, 15 mmol) was added. The reaction mixture was stirred for 2 hours with gradual warming to room-temperature. The reaction mixture was poured into sat·NH₄Cl (100 mL) and extracted with ethyl acetate (100 mL×3). The combined organics were washed with brine (100 mL), dried over anhydrous Na₂SO₄, filtered, and concentrated under reduced pressure. Purification (FCC, SiO₂, petroleum ether:ethyl acetate=1:0 to 10:1) to afford the title compound (2.6 g, 68%) as a yellow oil. ¹H NMR (400 MHz, CDCl₃): δ 8.15 (s, 1H), 7.71 (s, 1H), 6.87-6.51 (m, 1H), 5.87 (s, 2H), 3.71-3.64 (m, 2H), 0.97-0.92 (m, 2H), −0.02-−0.05 (m, 9H).

Step C. 6-(Difluoromethyl)-4-(4,4,5,5-tetramethyl-1,3,2-dioxaborolan-2-yl)-1-((2-(trimethylsilyl)ethoxy)methyl)-1H-pyrazolo[3,4-b]pyridine. To a mixture of 4-bromo-6-(difluoromethyl)-1-((2-(trimethylsilyl)ethoxy)methyl)-1H-pyrazolo[3,4-b]pyridine (2.60 g, 6.87 mmol), bis(pinacolato) diboron (2.70 g, 10.6 mmol), KOAc (1.5 g, 15 mmol) in 1,2-dimethoxyethane (80 mL) under N₂, was added Pd(dppf)Cl₂ (550 mg, 0.752 mmol). The reaction mixture was stirred at 90° C. for 2.5 hours. The reaction mixture was cooled, filtered, and the filter cake was washed with ethyl acetate (30 mL×3). The combined filtrate was concentrated to dryness under reduced pressure. Purification (FCC, SiO₂, petroleum ether:ethyl acetate=1:0 to 85:15) afforded the title compound as a light yellow solid. ¹H NMR (400 MHz, CDCl₃): δ 8.42 (s, 1H), 7.90 (s, 1H), 6.85-6.54 (m, 1H), 5.89 (s, 2H), 3.68-3.59 (m, 2H), 1.42 (s, 12H), 0.96-0.88 (m, 2H), −0.03-−0.09 (m, 9H).

### Example 1: N-(4-(3-(5-Fluoropyridin-2-yl)-1-methyl-1H-pyrazol-4-yl)pyridin-2-yl)propionamide

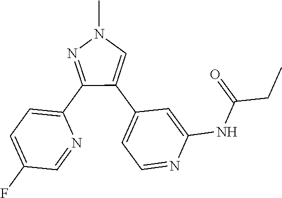 [SMILES: CCC(=O)Nc1cc(-c2cn(C)nc2-c2ccc(F)cn2)ccn1]

Step A. 4-(3-(5-Fluoropyridin-2-yl)-1-methyl-1H-pyrazol-4-yl)pyridin-2-amine. A mixture of 2-(3-(5-Fluoropyridin-2-yl)-1-methyl-1H-pyrazol-4-yl)-2-hydroxy-4,4,5,5-tetramethyl-1,3,2-dioxaborolan-2-uide lithium salt (Intermediate 5, 500 mg, 1.53 mmol), 4-bromopyridin-2-amine (318 mg, 1.83 mmol), XPhos Pd G3 (65 mg, 0.07 mmol) in 1,4-dioxane (10.7 mL) and 2 M Na₂CO₃ (aq) (3.57 mL, 11.4 mmol) was sparged with N₂ for 2 min. The reaction vial was sealed, and the mixture was stirred at 90° C. for 2 h. The reaction was cooled, diluted with water (25 mL), and extracted with EtOAc (3×25 mL). The combined organic layers were dried (Na₂SO₄) and filtered. Purification by chromatography (silica gel, 10% 2 M NH₃MeOH in DCM)/DCM 0-50% 2-12 min till 15 min) afforded (412 mg, 44%) the title compound. MS (ESI): mass calcd. for C₁₄H₁₂FN₅, 269.1; m/z found, [M+H]=270.1 [M+H]⁺.

Step B. N-(4-(3-(5-Fluoropyridin-2-yl)-1-methyl-1H-pyrazol-4-yl)pyridin-2-yl)propionamide. To a solution of 4-(3-(5-fluoropyridin-2-yl)-1-methyl-1H-pyrazol-4-yl)pyridin-2-amine (80 mg, 0.297 mmol) and propionic acid (33 μL, d=0.992 g/ml, 0.446 mmol) in DCM (2 mL) were added DIPEA (0.205 mL, 1.2 mmol) and HATU (141 mg, 0.37 mmol) and the resulting mixture was stirred at room temperature for 18 hours. The solvent was evaporated. Purification via silica gel chromatography (0-100% EtOAc in DCM) gave 91 mg white solid which was further purified by reversed phase HPLC Method C; to afford (62.5 mg, 65%) the title compound. MS (ESI): mass calcd. for C₁₇H₁₆FN₅O, 325.1; m/z found, 326.1 [M+H]⁺. ¹H NMR (500 MHz, Methanol-d₄) δ 8.42 (d, J=2.9 Hz, 1H), 8.14 (dd, J=5.3, 0.8 Hz, 1H), 8.05 (d, J=1.4 Hz, 1H), 8.02 (s, 1H), 7.75-7.69 (m, 1H), 7.69-7.62 (m, 1H), 7.02 (dd, J=5.3, 1.6 Hz, 1H), 4.00 (s, 3H), 2.41 (q, J=7.6 Hz, 2H), 1.17 (t, J=7.6 Hz, 3H).

### Example 2: 4-(3-(5-Fluoropyridin-2-yl)-1-methyl-1H-pyrazol-4-yl)-6,7-dihydro-5H-cyclopenta[b]pyridine

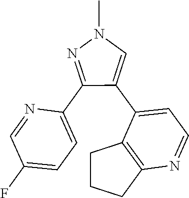 [SMILES: Cn1cc(-c2ccnc3c2CCC3)c(-c2ccc(F)cn2)n1]

The title compound was prepared in a manner analogous to Example 1, Step A, except using 4-chloro-6,7-dihydro-5 h-cyclopenta[b]pyridine instead of 4-bromopyridin-2-amine. MS (ESI): mass calcd. for C₁₇H₁₅FN₄, 294.1; m/z found, 295.1 [M+H]⁺. ¹H NMR (400 MHz, CDCl₃) δ 8.34 (d, J=2.9 Hz, 1H), 8.20 (dd, J=5.2, 0.9 Hz, 1H), 7.45-7.32 (m, 2H), 7.26 (ddd, J=8.7, 8.1, 2.9 Hz, 1H), 6.89-6.75 (m, 1H), 3.95 (s, 3H), 2.95 (t, J=7.7 Hz, 2H), 2.55 (t, J=7.4 Hz, 2H), 1.93 (p, J=7.6 Hz, 2H).

### Example 3: 4-(3-(5-Fluoropyridin-2-yl)-1-methyl-1H-pyrazol-4-yl)-1,3-dihydro-2H-pyrrolo[2,3-b]pyridin-2-one

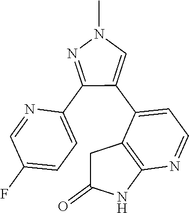 [SMILES: Cn1cc(-c2ccnc3c2CC(=O)N3)c(-c2ccc(F)cn2)n1]

The title compound was prepared in a manner analogous to Example 1, Step A, except using 4-bromo-1H-pyrrolo[2,3-b]pyridin-2(3H)-one instead of 4-bromopyridin-2-amine. MS (ESI): mass calcd. for C₁₆H₁₂FN₅O, 309.1; m/z found, 210.1 [M+H]⁺. ¹H NMR (400 MHz, CDCl₃) δ 8.27 (d, J=2.9 Hz, 1H), 7.99 (d, J=5.5 Hz, 1H), 7.91-7.79 (m, 1H), 7.65 (dd, J=8.7, 4.4 Hz, 1H), 7.44 (d, J=1.9 Hz, 1H), 7.40-7.26 (m, 1H), 6.77 (d, J=5.5 Hz, 1H), 3.95 (s, 3H), 3.15 (s, 2H).

### Example 4: 7-(3-(5-Fluoropyridin-2-yl)-1-methyl-1H-pyrazol-4-yl)-1H-pyrrolo[3,2-b]pyridine

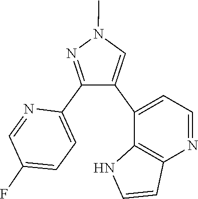 [SMILES: Cn1cc(-c2ccnc3cc[nH]c23)c(-c2ccc(F)cn2)n1]

The title compound was prepared in a manner analogous to Example 1, Step A, except using 7-bromo-1H-pyrrolo[3,2-b]pyridine instead of 4-bromopyridin-2-amine. MS (ESI): mass calcd. for C₁₆H₁₂FN₅, 293.1; m/z found, 294.1 [M+H]⁺.

### Example 5: 7-(3-(5-Fluoropyridin-2-yl)-1-methyl-1H-pyrazol-4-yl)-1H-pyrazolo[4,3-b]pyridine

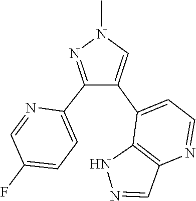 [SMILES: Cn1cc(-c2ccnc3cn[nH]c23)c(-c2ccc(F)cn2)n1]

The title compound was prepared in a manner analogous to Example 1, Step A, except using 7-chloro-1H-pyrazolo[4,3-b]pyridine instead of 4-bromopyridin-2-amine. MS (ESI): mass calcd. for C₁₅H₁₁FN₆, 294.1; m/z found, 295.1 [M+H]⁺. ¹H NMR (400 MHz, Methanol-d₄) δ 8.44 (d, J=4.7 Hz, 1H), 8.23 (s, 1H), 8.18 (d, J=2.9 Hz, 1H), 8.09 (s, 1H), 7.89 (dd, J=8.8, 4.5 Hz, 1H), 7.63 (td, J=8.6, 2.9 Hz, 1H), 7.26 (d, J=4.7 Hz, 1H), 4.09 (s, 3H).

### Example 6: 5-(1-Methyl-4-(1H-pyrrolo[2,3-b]pyridin-4-yl)-1H-pyrazol-3-yl)oxazole

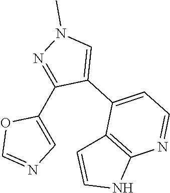 [SMILES: Cn1cc(-c2ccnc3[nH]ccc23)c(-c2cnco2)n1]

Step A. 5-(1-methyl-4-(1-(phenylsulfonyl)-1H-pyrrolo[2,3-b]pyridin-4-yl)-1H-pyrazol-3-yl)oxazole. The title compound was prepared in a manner analogous to Example 1, Step A, except using 4-(3-bromo-1-methyl-1H-pyrazol-4-yl)-1-(phenylsulfonyl)-1H-pyrrolo[2,3-b]pyridine (Intermediate 55) instead of 4-bromopyridin-2-amine and using 5-(4,4,5,5-tetramethyl-1,3,2-dioxaborolan-2-yl)oxazole instead of 2-(3-(5-Fluoropyridin-2-yl)-1-methyl-1H-pyrazol-4-yl)-2-hydroxy-4,4,5,5-tetramethyl-1,3,2-dioxaborolan-2-uide lithium salt (Intermediate 5); using ethanol and toluene instead of dioxane; and heating at 110° C. for 16 h. MS (ESI): mass calcd. for C₂₀H₁₅N₅O₃S, 405.1; m/z found, 406.2 [M+H]⁺.

Step B. 5-(1-Methyl-4-(1H-pyrrolo[2,3-b]pyridin-4-yl)-1H-pyrazol-3-yl)oxazole. To a solution of 5-(1-methyl-4-(1-(phenylsulfonyl)-1H-pyrrolo[2,3-b]pyridin-4-yl)-1H-pyrazol-3-yl)oxazole (120 mg, 0.296 mmol) in tetrahydrofuran (2.5 mL) was added tetrabutylammonium fluoride (592 μL, 0.592 mmol) dropwise under argon. The reaction mixture was stirred at room temperature for 17 h under argon. To the reaction mixture was added tetrabutylammonium fluoride (888 μL, 0.888 mmol) dropwise under argon. The reaction mixture was stirred at room temperature for 24 h, then at 40° C. for 4 h under argon. The reaction mixture was evaporated. The residue was taken up in dichloromethane (60 mL), washed with brine (3×20 mL), dried over magnesium sulfate, filtered, and evaporated. The residue was purified by preparative HPLC Method D. The residue was taken up in ethyl acetate (60 mL), washed with saturated ammonium chloride (4×20 mL) and water (1×15 mL), dried over magnesium sulfate, filtered and evaporated. The residue was triturated with diethyl ether (2×1 mL). The product was purified by silica gel column chromatography eluting with dichloromethane:ethanol (100:4) to give the title compound (18 mg, 0.068 mmol, 23%) as a pale yellow crystalline solid. MS (ESI): mass calcd. for C₁₄H₁₁N₅O, 265.1; m/z found, 266.1 [M+H]⁺. ¹H NMR (500 MHz, DMSO-d₆) δ 11.73-11.63 (m, 1H), 8.32 (s, 1H), 8.23 (s, 1H), 8.18 (d, J=4.9 Hz, 1H), 7.47-7.43 (m, 1H), 7.15 (s, 1H), 6.91 (d, J=4.8 Hz, 1H), 6.27 (dd, J=3.5, 1.9 Hz, 1H), 3.99 (s, 3H).

### Example 7: 2-(1-Methyl-4-(1H-pyrrolo[2,3-b]pyridin-4-yl)-1H-pyrazol-3-yl)oxazole

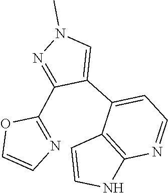 [SMILES: Cn1cc(-c2ccnc3[nH]ccc23)c(-c2ncco2)n1]

Step A: 2-(1-Methyl-4-(1-(phenylsulfonyl)-1H-pyrrolo[2,3-b]pyridin-4-yl)-1H-pyrazol-3-yl)oxazole. In a sealed vessel was placed 4-(3-bromo-1-methyl-1H-pyrazol-4-yl)-1-(phenylsulfonyl)-1H-pyrrolo[2,3-b]pyridine (Intermediate 55, 30 mg, 0.072 mmol), 2-(tributylstannyl)oxazole (46 mg, 2.5 mmol), bis(triphenylphosphine)palladium(II) dichloride (5 mg, 0.007 mmol), cuprous iodide (4.2 mg, 0.022 mmol) and toluene (0.75 mL). The reaction mixture was flushed with N₂ for 2 minutes, sealed then heated to 100° C. for 18 hours. Then it was cooled down and to it 1 mL of EtOAc and KF on Celite® (50 wt %, 84 mg, 0.72 mmol) were added and the resulting mixture was stirred at room temperature for 3 hours. The mixture was filtered through Celite®. Evaporation and purification via silica gel chromatography (0% to 100% EtOAc/DCM) gave the title compound (9.5 mg, 33% yield). MS (ESI): mass calcd. for C₂₀H₁₅N₅O₃S, 405.1; m/z found, 406.1 [M+H]⁺.

Step B: 2-(1-Methyl-4-(1H-pyrrolo[2,3-b]pyridin-4-yl)-1H-pyrazol-3-yl)oxazole. To a solution of 2-(1-methyl-4-(1-(phenylsulfonyl)-1H-pyrrolo[2,3-b]pyridin-4-yl)-1H-pyrazol-3-yl)oxazole (9.5 mg, 0.0234 mmol) in THE (0.3 mL) was added tetrabutylammonium fluoride (1.0 M in THF, 70.3 μL, 0.070 mmol) and the resulting solution was stirred at room temperature for 4 h. The solvent was evaporated, and the residue was diluted with brine (5 mL) and extracted with DCM (3×5 mL). Solvent was evaporated and the residue was purified by reversed phase HPLC Method C: to afford 2.4 mg (39%) of the title compound. MS (ESI): mass calcd. for C₁₄H₁₁N₅O, 265.1; m/z found, 266.1 [M+H]⁺. ¹H NMR (500 MHz, Methanol-d₄) δ 8.06 (d, J=5.1 Hz, 1H), 7.99 (s, 1H), 7.75 (d, J=0.8 Hz, 1H), 7.24 (d, J=3.5 Hz, 1H), 7.12 (s, 1H), 7.01 (d, J=5.0 Hz, 1H), 6.17 (d, J=3.5 Hz, 1H), 3.98 (s, 3H).

### Example 8: 2-(1-Methyl-4-(6-methyl-1H-pyrazolo[3,4-b]pyridin-4-yl)-1H-pyrazol-3-yl)oxazole

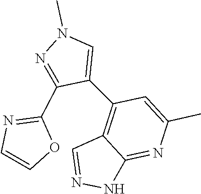 [SMILES: Cc1cc(-c2cn(C)nc2-c2ncco2)c2cn[nH]c2n1]

The title compound was prepared in a manner analogous to Example 19 Step A-B, except using 2-(4-bromo-1-methyl-1H-pyrazol-3-yl)oxazole (Intermediate 37) instead of 4-(4-bromo-1-methyl-1H-pyrazol-3-yl)-3-fluoropyridine (Intermediate 19) and using 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 3) instead of 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 1) in Step C. MS (ESI): mass calcd. for C₁₄H₁₂N₆O, 280.1; m/z found, 281.1 [M+H]⁺. ¹H NMR (500 MHz, CDCl₃) δ 8.40 (s, 1H), 7.83 (s, 1H), 7.50 (d, J=2.3 Hz, 1H), 7.47 (s, 0.5H), 7.41 (s, 1H), 7.05 (s, 0.5H), 6.94 (d, J=2.3 Hz, 1H), 4.06 (s, 3H), 2.73 (s, 3H).

### Example 9: 4-(1-Methyl-3-(1-methyl-1H-imidazol-4-yl)-1H-pyrazol-4-yl)-1H-pyrrolo[2,3-b]pyridine

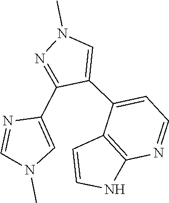 [SMILES: Cn1cnc(-c2nn(C)cc2-c2ccnc3[nH]ccc23)c1]

Step A. 4-(1-Methyl-3-(1-methyl-1H-imidazol-4-yl)-1H-pyrazol-4-yl)-1-(phenylsulfonyl)-1H-pyrrolo[2,3-b]pyridine. A mixture of 4-(3-bromo-1-methyl-1H-pyrazol-4-yl)-1-(phenylsulfonyl)-1H-pyrrolo[2,3-b]pyridine (Intermediate 55, 100 mg, 0.24 mmol), tributyl-(1-methylimidazol-4-yl)stannane (178 mg, 0.48 mmol), [1,1′-bis(diphenylphosphino)ferrocene]dichloropalladium(II) (18 mg, 0.0246 mmol) and copper(I) iodide (14 mg, 0.0735 mmol) in N,N-dimethylformamide (4 mL) was stirred at 120° C. for 16 h under argon. The reaction mixture was evaporated. The residue was purified by gradient silica gel column chromatography eluting with dichloromethane:ethanol (100:1→100:9) to give the title compound (93 mg, 0.222 mmol, 93%) as a brown crystalline solid. MS (ESI): mass calcd. for C₂₁H₁₈N₆O₂S, 418.1; m/z found, 419.0 [M+H]⁺.

Step B. 4-(1-Methyl-3-(1-methyl-1H-imidazol-4-yl)-1H-pyrazol-4-yl)-1H-pyrrolo[2,3-b]pyridine. To a solution of 4-(1-methyl-3-(1-methyl-1H-imidazol-4-yl)-1H-pyrazol-4-yl)-1-(phenylsulfonyl)-1H-pyrrolo[2,3-b]pyridine (90 mg, 0.215 mmol) in methanol (13 mL) was added sodium hydroxide (2 M in water, 3.2 mL, 6.4 mmol). The reaction mixture was stirred at 50° C. for 2 h. The reaction mixture was evaporated. The residue was taken up in water (20 mL) and extracted with chloroform (3×15 mL). The combined organic layers were dried over magnesium sulfate, filtered, and evaporated. The crude product was purified by preparative HPLC Method D; to give the title compound (50 mg, 0.180 mmol, 84%) as a light brown crystalline solid. MS (ESI): mass calcd. for C₁₅H₁₄N₆, 278.1; m/z found, 279.1 [M+H]⁺. ¹H NMR (500 MHz, DMSO-d₆) δ 11.58-11.44 (m, 1H), 8.09 (s, 1H), 8.03 (d, J=5.0 Hz, 1H), 7.52-7.48 (m, 1H), 7.39-7.35 (m, 1H), 7.13-7.09 (m, 2H), 6.37 (dd, J=3.5, 1.7 Hz, 1H), 3.89 (s, 3H), 3.60 (s, 3H).

### Example 10: 4-(1-Methyl-3-(1-methyl-1H-imidazol-5-yl)-1H-pyrazol-4-yl)-1H-pyrrolo[2,3-b]pyridine

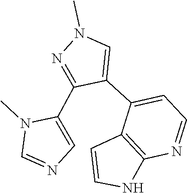 [SMILES: Cn1cc(-c2ccnc3[nH]ccc23)c(-c2cncn2C)n1]

The title compound was prepared in a manner analogous to Example 9, except using 1-methyl-5-(tributylstannyl)-1H-imidazole instead of tributyl-(1-methylimidazol-4-yl)stannane. MS (ESI): mass calcd. for C₁₅H₁₄N₆, 278.1; m/z found, 279.1 [M+H]⁺. ¹H NMR (500 MHz, DMSO-d₆) δ 11.71-11.61 (m, 1H), 8.30 (s, 1H), 8.07 (d, J=5.0 Hz, 1H), 7.65 (s, 1H), 7.45-7.41 (m, 1H), 6.79 (s, 1H), 6.69 (d, J=5.0 Hz, 1H), 6.36 (dd, J=3.5, 1.8 Hz, 1H), 3.98 (s, 3H), 3.38 (s, 3H).

### Example 11: 4-(1-Methyl-3-(1-methyl-1H-imidazol-2-yl)-1H-pyrazol-4-yl)-1H-pyrrolo[2,3-b]pyridine

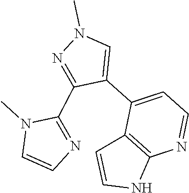 [SMILES: Cn1cc(-c2ccnc3[nH]ccc23)c(-c2nccn2C)n1]

The title compound was prepared in a manner analogous to Example 9, except using 1-methyl-2-(tributylstannyl)-1H-imidazole instead of tributyl-(1-methylimidazol-4-yl)stannane. MS (ESI): mass calcd. for C₁₅H₁₄N₆, 278.1; m/z found, 279.1 [M+H]⁺. ¹H NMR (500 MHz, DMSO-d₆) δ 11.67-11.55 (m, 1H), 8.37 (s, 1H), 8.03 (d, J=5.0 Hz, 1H), 7.44-7.40 (m, 1H), 7.25-7.19 (m, 1H), 6.96-6.90 (m, 1H), 6.79 (d, J=5.0 Hz, 1H), 6.36 (dd, J=3.5, 1.8 Hz, 1H), 4.01 (s, 3H), 3.45 (s, 3H).

### Example 12: 5-(1-Methyl-4-(1H-pyrrolo[2,3-b]pyridin-4-yl)-1H-pyrazol-3-yl)isothiazole

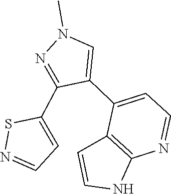 [SMILES: Cn1cc(-c2ccnc3[nH]ccc23)c(-c2ccns2)n1]

Step A. 5-(1-Methyl-4-(1-(phenylsulfonyl)-1H-pyrrolo[2,3-b]pyridin-4-yl)-1H-pyrazol-3-yl)isothiazole. The title compound was prepared in a manner analogous to Example 1, Step A, except using 4-(3-bromo-1-methyl-1H-pyrazol-4-yl)-1-(phenylsulfonyl)-1H-pyrrolo[2,3-b]pyridine (Intermediate 55) instead of 4-bromopyridin-2-amine and using 5-(4,4,5,5-tetramethyl-1,3,2-dioxaborolan-2-yl)isothiazole instead of 2-(3-(5-fluoropyridin-2-yl)-1-methyl-1H-pyrazol-4-yl)-2-hydroxy-4,4,5,5-tetramethyl-1,3,2-dioxaborolan-2-uide lithium salt (Intermediate 5) and a mixture of ethanol/toluene instead of 1,4-dioxane. MS (ESI): mass calcd. for C₂₀H₁₅N₅O₂S₂, 421.1; m/z found, 422.0 [M+H]⁺.

Step B. 5-(1-Methyl-4-(1H-pyrrolo[2,3-b]pyridin-4-yl)-1H-pyrazol-3-yl)isothiazole. To a solution of 5-(1-methyl-4-(1-(phenylsulfonyl)-1H-pyrrolo[2,3-b]pyridin-4-yl)-1H-pyrazol-3-yl)isothiazole (31 mg, 0.074 mmol) in methanol (4.4 mL) was added sodium hydroxide (2 M in water, 1.1 mL, 2.22 mmol). The reaction mixture was stirred at 40° C. for 2 h. The reaction mixture was evaporated. The residue was taken up in water (20 mL) and extracted with chloroform (2×15 mL). The combined organic layers were dried over magnesium sulfate, filtered, and evaporated. The crude product was purified by preparative HPLC Method D; to give the title compound (7 mg, 34% yield). MS (ESI): mass calcd. for C₁₄H₁₁N₅S, 281.1; m/z found, 282.0 [M+H]⁺. ¹H NMR (500 MHz, DMSO-d₆) δ 11.80-11.68 (m, 1H), 8.35 (d, J=1.8 Hz, 1H), 8.21 (d, J=4.9 Hz, 1H), 8.15 (s, 1H), 7.46-7.42 (m, 1H), 7.01 (d, J=1.8 Hz, 1H), 6.99 (d, J=4.8 Hz, 1H), 6.19 (dd, J=3.4, 1.8 Hz, 1H), 3.97 (s, 3H).

### Example 13: 4-(1-Methyl-4-(1H-pyrrolo[2,3-b]pyridin-4-yl)-1H-pyrazol-3-yl)thiazole

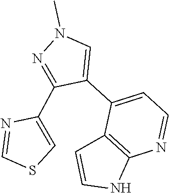 [SMILES: Cn1cc(-c2ccnc3[nH]ccc23)c(-c2cscn2)n1]

Step A. 4-(1-Methyl-4-(1-(phenylsulfonyl)-1H-pyrrolo[2,3-b]pyridin-4-yl)-1H-pyrazol-3-yl)thiazole. A mixture of 4-(3-bromo-1-methyl-1H-pyrazol-4-yl)-1-(phenylsulfonyl)-1H-pyrrolo[2,3-b]pyridine (Intermediate 55, 135 mg, 0.324 mmol), 4-(tributylstannyl)-thiazole (243 mg, 0.649 mmol) and (2-dicyclohexylphosphino-2′,4′,6′-triisopropyl-1,1′-biphenyl)[2-(2′-amino-1,1′-biphenyl)]palladium(II) methanesulfonate (XPhos Pd G3, 27 mg, 0.0319 mmol) in toluene (2 mL) and ethanol (1 mL) was stirred at 110° C. for 20 h under argon. The reaction mixture was evaporated. The residue was purified by gradient silica gel column chromatography eluting with dichloromethane:ethanol (200:1→200:5) to give the title compound (127 mg, 0.301 mmol, 93%) as a light brown gum. MS (ESI): mass calcd. for C₂₀H₁₅N₅O₂S₂, 421.1; m/z found, 422.0 [M+H]⁺.

Step B. 4-(1-Methyl-4-(1H-pyrrolo[2,3-b]pyridin-4-yl)-1H-pyrazol-3-yl)thiazole. To a solution of 4-(1-methyl-4-(1-(phenylsulfonyl)-1H-pyrrolo[2,3-b]pyridin-4-yl)-1H-pyrazol-3-yl)thiazole (125 mg, 0.297 mmol) in methanol (18 mL) was added sodium hydroxide (2 M in water, 4.46 mL, 8.92 mmol). The reaction mixture was stirred at 50° C. for 3 h. The reaction mixture was evaporated. The residue was taken up in water (20 mL) and extracted with ethyl acetate (4×15 mL). The combined organic layers were dried over magnesium sulfate, filtered, and evaporated. The crude product was purified by preparative HPLC Method D. The residue was taken up in saturated sodium bicarbonate (20 mL) and extracted with ethyl acetate (2×15 mL). The combined organic layers were dried over magnesium sulfate, filtered, and evaporated to give the title compound (47 mg, 0.167 mmol, 56%) as a pale yellow crystalline solid. MS (ESI): mass calcd. for C₁₄H₁₁N₅S, 281.1; m/z found, 282.1 [M+H]⁺. ¹H NMR (500 MHz, DMSO-d₆) δ 11.61-11.52 (m, 1H), 9.02 (d, J=2.0 Hz, 1H), 8.19 (s, 1H), 8.08 (d, J=4.9 Hz, 1H), 7.71 (d, J=2.0 Hz, 1H), 7.40-7.35 (m, 1H), 6.85 (d, J=4.9 Hz, 1H), 6.24 (dd, J=3.5, 1.9 Hz, 1H), 3.98 (s, 3H).

### Example 14: 5-(1-Methyl-4-(1H-pyrrolo[2,3-b]pyridin-4-yl)-1H-pyrazol-3-yl)thiazole

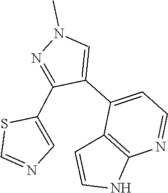 [SMILES: Cn1cc(-c2ccnc3[nH]ccc23)c(-c2cncs2)n1]

The title compound was prepared in a manner analogous to Example 13, except using 5-(tributylstannyl)thiazole instead of 4-(tributylstannyl)-thiazole. MS (ESI): mass calcd. for C₁₄H₁₁N₅S, 281.1; m/z found, 282.0 [M+H]⁺. ¹H NMR (500 MHz, DMSO-d₆) δ 11.77-11.67 (m, 1H), 8.99-8.96 (m, 1H), 8.21 (d, J=4.9 Hz, 1H), 8.13 (s, 1H), 7.59-7.57 (m, 1H), 7.46-7.42 (m, 1H), 6.98 (d, J=4.9 Hz, 1H), 6.23-6.19 (m, 1H), 3.97 (s, 3H).

### Example 15: 3-(1-Methyl-4-(1H-pyrrolo[2,3-b]pyridin-4-yl)-1H-pyrazol-3-yl)isothiazole

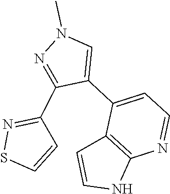 [SMILES: Cn1cc(-c2ccnc3[nH]ccc23)c(-c2ccsn2)n1]

The title compound was prepared in a manner analogous to Example 13, except using 4-(1-methyl-3-(trimethylstannyl)-1H-pyrazol-4-yl)-1-(phenylsulfonyl)-1H-pyrrolo[2,3-b]pyridine (Intermediate 56) instead of 4-(tributylstannyl)thiazole and 3-bromothiazole instead of 4-(3-bromo-1-methyl-1H-pyrazol-4-yl)-1-(phenylsulfonyl)-1H-pyrrolo[2,3-b]pyridine (Intermediate 55). MS (ESI): mass calcd. for C₁₄H₁₁N₅S, 281.1; m/z found, 282.0 [M+H]⁺. ¹H NMR (500 MHz, DMSO-d₆) δ 11.62-11.53 (m, 1H), 9.02 (d, J=4.7 Hz, 1H), 8.16 (s, 1H), 8.10 (d, J=4.9 Hz, 1H), 7.49 (d, J=4.7 Hz, 1H), 7.38-7.34 (m, 1H), 6.97 (d, J=4.9 Hz, 1H), 6.19-6.16 (m, 1H), 3.97 (s, 3H).

### Example 16: 2-(1-Methyl-4-(1H-pyrrolo[2,3-b]pyridin-4-yl)-1H-pyrazol-3-yl)thiazole

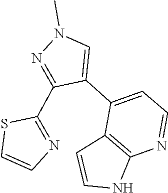 [SMILES: Cn1cc(-c2ccnc3[nH]ccc23)c(-c2nccs2)n1]

The title compound was prepared in a manner analogous to Example 7, except using 2-(tributylstannyl)thiazole instead of 2-(tributylstannyl)oxazole. MS (ESI): mass calcd. for C₁₄H₁₁N₅S, 281.1; m/z found, 282.0 [M+H]⁺. ¹H NMR (400 MHz, Methanol-d₄) δ 8.15 (d, J=5.1 Hz, 1H), 8.07 (s, 1H), 7.80-7.64 (m, 1H), 7.56 (d, J=3.3 Hz, 1H), 7.33 (d, J=3.6 Hz, 1H), 7.16 (d, J=5.1 Hz, 1H), 6.30 (d, J=3.6 Hz, 1H), 4.07 (s, 3H).

### Example 17: 4-[1-Methyl-3-(4-pyridyl)pyrazol-4-yl]-1H-pyrrolo[2,3-b]pyridine

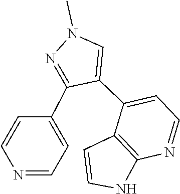 [SMILES: Cn1cc(-c2ccnc3[nH]ccc23)c(-c2ccncc2)n1]

The title compound was prepared in a manner analogous to Example 19, Step A, except using 4-(4-bromo-1-methyl-1H-pyrazol-3-yl)pyridine (Intermediate 59) instead of 4-(4-bromo-1-methyl-1H-pyrazol-3-yl)-3-fluoropyridine (Intermediate 19) and using 4-(4,4,5,5-tetramethyl-1,3,2-dioxaborolan-2-yl)-1 h-pyrrolo[2,3-b]pyridine instead of 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 1). MS (ESI): mass calcd. for C₁₆H₁₃N₅, 275.1; m/z found, 276.1 [M+H]⁺. ¹H NMR (400 MHz, Methanol-d₄) δ 8.46-8.35 (m, 2H), 8.17 (d, J=5.0 Hz, 1H), 8.03 (s, 1H), 7.50-7.41 (m, 2H), 7.33 (d, J=3.6 Hz, 1H), 6.99 (d, J=5.0 Hz, 1H), 6.20 (d, J=3.5 Hz, 1H), 4.09 (s, 3H).

### Example 18: 4-[3-(3-Fluoro-4-pyridyl)-1-methyl-pyrazol-4-yl]-1H-pyrrolo[2,3-b]pyridine

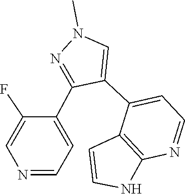 [SMILES: Cn1cc(-c2ccnc3[nH]ccc23)c(-c2ccncc2F)n1]

The title compound was prepared in a manner analogous to Example 19, Step A, except using 4-(4-bromo-1-methyl-1H-pyrazol-3-yl)-3-fluoropyridine (Intermediate 60) instead of 4-(4-bromo-1-methyl-1H-pyrazol-3-yl)-3-fluoropyridine (Intermediate 19) and using 4-(4,4,5,5-tetramethyl-1,3,2-dioxaborolan-2-yl)-1H-pyrrolo[2,3-b]pyridine instead of 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 1). MS (ESI): mass calcd. for C₁₆H₁₂FN₅, 293.1; m/z found, 294.1 [M+H]⁺. ¹H NMR (400 MHz, Methanol-d₄) δ 8.47-8.31 (m, 2H), 8.15 (s, 1H), 8.09 (d, J=5.0 Hz, 1H), 7.57 (dd, J=6.3, 5.0 Hz, 1H), 7.31 (d, J=3.5 Hz, 1H), 6.85 (d, J=5.1 Hz, 1H), 6.24 (d, J=3.5 Hz, 1H), 4.10 (s, 3H).

### Example 19: 4-[3-(3-Fluoro-4-pyridyl)-1-methyl-pyrazol-4-yl]-1H-pyrazolo[3,4-b]pyridine

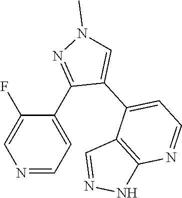 [SMILES: Cn1cc(-c2ccnc3[nH]ncc23)c(-c2ccncc2F)n1]

Step A. 4-(3-(3-Fluoropyridin-4-yl)-1-methyl-1H-pyrazol-4-yl)-1-((2-(trimethylsilyl)ethoxy)methyl)-1H-pyrazolo[3,4-b]pyridine. To a mixture of 4-(4-bromo-1-methyl-1H-pyrazol-3-yl)-3-fluoropyridine (Intermediate 19, 58 mg, 0.125 mmol), 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 1, 70 mg, 0.188 mmol) and cesium carbonate (122 mg, 0.375 mmol) in 2-methyl-2-butanol (1.0 mL) and water (0.25 mL) was added cataCXium® A Pd G3 (9.1 mg, 0.0125 mmol) and the resulting mixture was sparged with N₂ for 2 min. The reaction vial was sealed, and the mixture was stirred at 90° C. for 18 h. The reaction was cooled, diluted with water (5 mL), and extracted with EtOAc (3×5 mL). Solvent was evaporated and the residue was purified by reversed phase HPLC Method C; to afford 32.3 mg (61%) of the title compound. MS (ESI): mass calcd. for C₂₁H₂₅FN₆OSi, 424.2; m/z found, 425.2 [M+H]⁺.

Step B. 4-(3-(3-Fluoropyridin-4-yl)-1-methyl-1H-pyrazol-4-yl)-1H-pyrazolo[3,4-b]pyridine. A solution of 4-(3-(3-fluoropyridin-4-yl)-1-methyl-1H-pyrazol-4-yl)-1-((2-(trimethylsilyl)ethoxy)methyl)-1H-pyrazolo[3,4-b]pyridine (32 mg, 0.075 mmol) in TFA (0.36 mL) and DCM (0.71 mL) was stirred at room temperature for 3 hours. The mixture was evaporated to dryness then was diluted with DCM (0.5 mL) and 2N NH₃ in MeOH (0.5 mL, 1 mmol) and the resulting mixture stirred at room temperature for 18 hours. The mixture was concentrated under reduced pressure and the residue was purified by reversed phase HPLC Method C; to afford 13.4 mg (60%) of the title compound. MS (ESI): mass calcd. for C₁₅H₁₁FN₆, 294.1; m/z found, 295.1 [M+H]⁺. ¹H NMR (400 MHz, Methanol-d₄) δ 8.47 (dd, J=4.9, 1.0 Hz, 1H), 8.43-8.37 (m, 2H), 8.32 (s, 1H), 7.89 (s, 1H), 7.68 (ddd, J=6.3, 5.0, 0.5 Hz, 1H), 6.94 (d, J=4.9 Hz, 1H), 4.13 (s, 3H).

### Example 20: 4-[3-(5-Fluoro-2-pyridyl)-1-methyl-pyrazol-4-yl]-1H-pyrrolo[2,3-b]pyridine

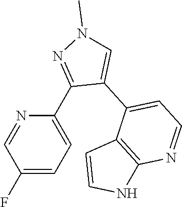 [SMILES: Cn1cc(-c2ccnc3[nH]ccc23)c(-c2ccc(F)cn2)n1]

To a mixture of 2-(4-bromo-1-methyl-pyrazol-3-yl)-5-fluoro-pyridine (Intermediate 18, 1 g, 3.91 mmol), tert-butyl 4-(4,4,5,5-tetramethyl-1,3,2-dioxaborolan-2-yl)pyrrolo[2,3-b]pyridine-1-carboxylate (Intermediate 4, 3.23 g, 9.384 mmol), [1,1′-bis(diphenylphosphino)ferrocene]dichloropalladium(II) (286 mg, 0.391 mmol) and sodium carbonate (2 M in water, 3.9 mL, 7.8 mmol) was added 1,4-dioxane (33 mL). The reaction was split evenly 3 portions and stirred at 130° C. for 4 h under argon under microwave irradiation. To the combined reaction mixtures was added water (100 mL) and ethyl acetate (100 mL). The mixture was filtered through a pad of Celite®. The Celite® was washed with ethyl acetate (1×40 mL). The combined filtrate layers were separated. The aqueous layer was extracted with ethyl acetate (1×60 mL). The combined organic layers were washed with brine (1×80 mL). The organic layer was dried over magnesium sulfate, filtered and evaporated. The crude was purified by gradient silica gel column chromatography eluting with chloroform:methanol (100:0→100:2). The residue was purified by preparative HPLC Method D. The collected fraction was concentrated to remove acetonitrile. The aqueous layer was made basic to pH 8 with saturated sodium bicarbonate (20 mL) and extracted with ethyl acetate (2×50 mL). The combined organic layers were dried over magnesium sulfate, filtered and evaporated. The residue was suspended in ethanol (2 mL) and heated to 78° C. The suspension was cooled to −10° C. The precipitate was collected and the solid was washed with cold ethanol (2×1 mL) and diethyl ether (2×1 mL) to give the title compound (200 mg, 0.682 mmol, 17%) as an off-white powder. MS (ESI): mass calcd. for C₁₆H₁₂FN₅, 293.1; m/z found, 294.1 [M+H]⁺. ¹H NMR (500 MHz, DMSO-d₆) δ 11.54 (br s, 1H), 8.40-8.34 (m, 1H), 8.16 (s, 1H), 8.08 (d, J=4.9 Hz, 1H), 7.75-7.73 (m, 1H), 7.72 (d, J=1.9 Hz, 1H), 7.37-7.33 (m, 1H), 6.83 (d, J=5.0 Hz, 1H), 6.13 (dd, J=3.5, 1.9 Hz, 1H), 3.99 (s, 3H).

### Example 21: 4-[3-(5-Fluoro-3-pyridyl)-1-methyl-pyrazol-4-yl]-1H-pyrrolo[2,3-b]pyridine

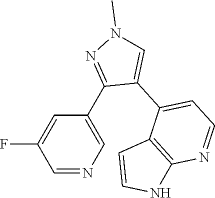 [SMILES: Cn1cc(-c2ccnc3[nH]ccc23)c(-c2cncc(F)c2)n1]

The title compound was prepared in a manner analogous to Example 20, except using 3-(4-bromo-1-methyl-1H-pyrazol-3-yl)-5-fluoropyridine (Intermediate 36) instead of 2-(4-bromo-1-methyl-pyrazol-3-yl)-5-fluoro-pyridine (Intermediate 18). MS (ESI): mass calcd. for C₁₆H₁₂FN₅, 293.1; m/z found, 294.1 [M+H]⁺. ¹H NMR (500 MHz, DMSO-d₆) δ 11.71 (br s, 1H), 8.48 (d, J=2.8 Hz, 1H), 8.34 (t, J=1.8 Hz, 1H), 8.21 (s, 1H), 8.17 (d, J=4.9 Hz, 1H), 7.62-7.57 (m, 1H), 7.43-7.39 (m, 1H), 6.86 (d, J=4.9 Hz, 1H), 6.13 (dd, J=3.5, 1.9 Hz, 1H), 4.01 (s, 3H).

### Example 22: 4-[1-Methyl-3-(6-methyl-3-pyridyl)pyrazol-4-yl]-1H-pyrrolo[2,3-b]pyridine

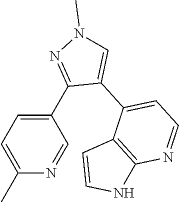 [SMILES: Cc1ccc(-c2nn(C)cc2-c2ccnc3[nH]ccc23)cn1]

The title compound was prepared in a manner analogous to Example 20, except using 5-(4-bromo-1-methyl-1H-pyrazol-3-yl)-2-methylpyridine (Intermediate 31) instead of 2-(4-bromo-1-methyl-1H-pyrazol-3-yl)-5-fluoropyridine (Intermediate 18). MS (ESI): mass calcd. for C₁₇H₁₅N₅, 289.1; m/z found, 290.2 [M+H]⁺. ¹H NMR (500 MHz, DMSO-d₆) δ 11.66 (br s, 1H), 8.39 (d, J=2.2 Hz, 1H), 8.16 (s, 1H), 8.12 (d, J=4.9 Hz, 1H), 7.59 (dd, J=8.0, 2.3 Hz, 1H), 7.42-7.37 (m, 1H), 7.17 (d, J=8.1 Hz, 1H), 6.80 (d, J=4.9 Hz, 1H), 6.18 (dd, J=3.5, 1.9 Hz, 1H), 3.98 (s, 3H), 2.43 (s, 3H).

### Example 23: 4-[3-[6-(Difluoromethoxy)-3-pyridyl]-1-methyl-pyrazol-4-yl]-1H-pyrrolo[2,3-b]pyridine

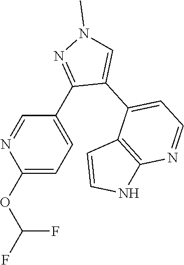 [SMILES: Cn1cc(-c2ccnc3[nH]ccc23)c(-c2ccc(OC(F)F)nc2)n1]

The title compound was prepared in a manner analogous to Example 20, except using 5-(4-bromo-1-methyl-1H-pyrazol-3-yl)-2-(difluoromethoxy)pyridine (Intermediate 32) instead of 2-(4-bromo-1-methyl-1H-pyrazol-3-yl)-5-fluoropyridine (Intermediate 18). MS (ESI): mass calcd. for C₁₇H₁₃F₂N₅₀, 341.1; m/z found, 342.2 [M+H]⁺. ¹H NMR (300 MHz, DMSO-d₆) δ 11.69 (br s, 1H), 8.26-8.08 (m, 3H), 7.87-7.76 (m, 1H), 7.68 (t, J=72.8 Hz, 1H), 7.44-7.34 (m, 1H), 7.03 (d, J=8.5 Hz, 1H), 6.83 (d, J=4.9 Hz, 1H), 6.24-6.11 (m, 1H), 3.99 (s, 3H).

### Example 24: 4-[3-(5-Chloro-2-pyridyl)-1-methyl-pyrazol-4-yl]-1H-pyrrolo[2,3-b]pyridine

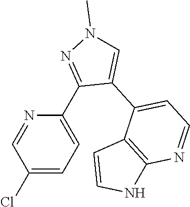 [SMILES: Cn1cc(-c2ccnc3[nH]ccc23)c(-c2ccc(Cl)cn2)n1]

The title compound was prepared in a manner analogous to Example 20, except using 2-(4-bromo-1-methyl-1H-pyrazol-3-yl)-5-chloropyridine (Intermediate 8) instead of 2-(4-bromo-1-methyl-1H-pyrazol-3-yl)-5-fluoropyridine (Intermediate 18). MS (ESI): mass calcd. for C₁₆H₁₂ClN₅, 309.1; m/z found, 310.1 [M+H]⁺. ¹H NMR (300 MHz, DMSO-d₆) δ 11.57 (s, 1H), 8.44-8.37 (m, 1H), 8.17 (s, 1H), 8.09 (d, J=4.9 Hz, 1H), 7.96-7.89 (m, 1H), 7.70 (d, J=8.5 Hz, 1H), 7.41-7.33 (m, 1H), 6.84 (d, J=4.9 Hz, 1H), 6.18-6.08 (m, 1H), 4.00 (s, 3H).

### Example 25: 4-(3-(5-Chloropyridin-2-yl)-1-methyl-1H-pyrazol-4-yl)-1H-pyrazolo[3,4-b]pyridine

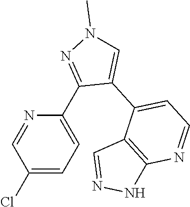 [SMILES: Cn1cc(-c2ccnc3[nH]ncc23)c(-c2ccc(Cl)cn2)n1]

Step A. 4-(3-(5-Chloropyridin-2-yl)-1-methyl-1H-pyrazol-4-yl)-1-((2-(trimethylsilyl)ethoxy)methyl)-1H-pyrazolo[3,4-b]pyridine. In a pressure vessel was dissolved 2-(4-bromo-1-methyl-1H-pyrazol-3-yl)-5-chloropyridine (Intermediate 8, 31 mg, 0.115 mmol), 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 1, 43 mg, 0.115 mmol) and 1,1′-bis(diphenylphosphino)ferrocene-palladium(II)dichloride dichloromethane complex (9 mg, 0.015 mmol) in 1,4-dioxane (0.50 mL) and 2 M Na₂CO₃ (aq) (0.172 mL, 0.344 mmol). The resulting mixture was degassed with N₂ and heated overnight at 100° C. The reaction was cooled to room temperature and partitioned between ethyl acetate and H₂O. The layers were separated and the aqueous was extracted with ethyl acetate (2×25 mL). The organic layers were combined and washed with brine (50 mL), dried (MgSO₄), filtered and concentrated under reduced pressure to afford the title compound (50 mg, 99%). The crude product was used in subsequent step without further purification. MS (ESI): mass calcd. for C₂₁H₂₅ClN₆OSi, 440.2; m/z found, 441.1 [M+H]⁺.

Step B. 4-(3-(5-Chloropyridin-2-yl)-1-methyl-1H-pyrazol-4-yl)-1H-pyrazolo[3,4-b]pyridine. A solution of 4-(3-(5-chloropyridin-2-yl)-1-methyl-1H-pyrazol-4-yl)-1-((2-(trimethylsilyl)ethoxy)methyl)-1H-pyrazolo[3,4-b]pyridine (50 mg, 0.113 mmol) in DCM (2 mL) and TFA (0.50 mL) was stirred at room temperature for 2 h. The mixture was evaporated to dryness then brought up in MeOH/DMSO for purification HPLC Method B; to afford 4-(3-(5-chloropyridin-2-yl)-1-methyl-1H-pyrazol-4-yl)-1H-pyrazolo[3,4-b]pyridine (3.7 mg, 10%). MS (ESI): mass calcd. for C₁₅H₁₁ClN₆, 310.1; m/z found, 311.0 [M+H]⁺. ¹H NMR (400 MHz, DMSO-d₆) δ 13.55 (s, 1H), 8.42-8.37 (m, 2H), 8.35 (s, 1H), 7.99 (dd, J=8.5, 2.5 Hz, 1H), 7.81 (dd, J=8.5, 0.8 Hz, 1H), 7.75 (d, J=1.0 Hz, 1H), 7.01 (d, J=4.8 Hz, 1H), 4.01 (s, 3H).

### Example 26: 4-(3-(5-Chloropyridin-2-yl)-1-methyl-1H-pyrazol-4-yl)-6-methyl-1H-pyrazolo[3,4-b]pyridine

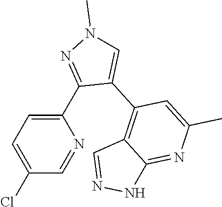 [SMILES: Cc1cc(-c2cn(C)nc2-c2ccc(Cl)cn2)c2cn[nH]c2n1]

Step A. 4-(3-(5-Chloropyridin-2-yl)-1-methyl-1H-pyrazol-4-yl)-6-methyl-1-((2-(trimethylsilyl)ethoxy)methyl)-1H-pyrazolo[3,4-b]pyridine. 2-(4-Bromo-1-methyl-1H-pyrazol-3-yl)-5-chloropyridine (Intermediate 8, 175 mg, 0.642 mmol), 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 3, 250 mg, 0.642 mmol), and Cs₂CO₃ (628 mg, 1.93 mmol) were added to a 20 mL microwave tube and the mixture dissolved in 1,4-dioxane (10 mL) and H₂O (2.5 mL). The mixture was sparged with Ar for 5 minutes and then treated with Pd(dppf)Cl₂ (47 mg, 0.064 mmol). The mixture was sparged with Ar for another 5 minutes and then stirred while heating at 90° C. via microwave irradiation for 1 hour then cooled to room-temperature. The mixture was filtered through a pad of Celite®, and the pad washed with ethyl acetate (20 mL×2). The filtrate was concentrated to dryness under reduced pressure to give the crude product, which was purified by FCC (eluent:petroleum ether:ethyl acetate=0:1 to 3:1) to afford the title compound (270 mg) as a brown solid mass calcd. MS (ESI): mass calcd. for C₂₂H₂₇ClN₆OSi, 454.2; m/z found, 455.2 [M+H]⁺.

Step B. 4-(3-(5-Chloropyridin-2-yl)-1-methyl-1H-pyrazol-4-yl)-6-methyl-1H-pyrazolo[3,4-b]pyridine. 4-(3-(5-Chloropyridin-2-yl)-1-methyl-1H-pyrazol-4-yl)-6-methyl-1-((2-(trimethylsilyl)ethoxy)methyl)-1H-pyrazolo[3,4-b]pyridine (270 mg, 0.593 mol), TFA (2.0 mL), and dichloromethane (2.0 mL) were added to a 10 mL sealed tube. The resultant mixture was stirred at room-temperature for 16 hours. The reaction mixture was poured into sat·NaHCO₃(20 mL) and extracted with dichloromethane (30 mL×2). The combined organic extracts were washed with brine (20 mL), dried over anhydrous Na₂SO₄, filtered, and concentrated to dryness under reduced pressure to give crude product, which was purified by preparative HPLC Method D; to afford pure product. The product was suspended in water (10 mL), the mixture frozen using dry ice/ethanol, and then lyophilized to dryness to afford the title compound afford the title compound (19 mg, 10%) as a white solid. MS (ESI): mass calcd. for C₁₆H₁₃ClN₆, 324.1; m/z found, 325.0 [M+H]⁺. ¹H NMR (400 MHz, DMSO-d₆) δ 13.32 (s, 1H), 8.42-8.36 (m, 1H), 8.29 (s, 1H), 8.00-7.95 (m, 1H), 7.80 (d, J=8.6 Hz, 1H), 7.54 (s, 1H), 6.95 (s, 1H), 4.00 (s, 3H), 2.51 (s, 3H).

### Example 27: 4-[3-(4-Fluoro-2-pyridyl)-1-methyl-pyrazol-4-yl]-1H-pyrrolo[2,3-b]pyridine

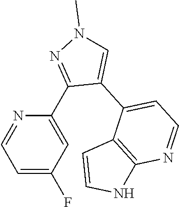 [SMILES: Cn1cc(-c2ccnc3[nH]ccc23)c(-c2cc(F)ccn2)n1]

The title compound was prepared in a manner analogous to Example 20, except using 2-(4-bromo-1-methyl-1H-pyrazol-3-yl)-4-fluoropyridine (Intermediate 29) instead of 2-(4-bromo-1-methyl-pyrazol-3-yl)-5-fluoro-pyridine (Intermediate 18). MS (ESI): mass calcd. for C₁₆H₁₂FN₅, 293.1; m/z found, 294.2 [M+H]⁺. ¹H NMR (500 MHz, DMSO-d₆) δ 11.60-11.50 (m, 1H), 8.38 (dd, J=9.1, 5.6 Hz, 1H), 8.18 (s, 1H), 8.09 (d, J=4.9 Hz, 1H), 7.52 (dd, J=10.4, 2.6 Hz, 1H), 7.36 (dd, J=3.5, 2.5 Hz, 1H), 7.24-7.19 (m, 1H), 6.87 (d, J=5.0 Hz, 1H), 6.15 (dd, J=3.5, 1.9 Hz, 1H), 4.00 (s, 3H).

### Example 28: 4-[3-(6-Fluoro-3-pyridyl)-1-methyl-pyrazol-4-yl]-1H-pyrrolo[2,3-b]pyridine

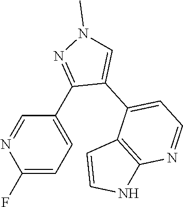 [SMILES: Cn1cc(-c2ccnc3[nH]ccc23)c(-c2ccc(F)nc2)n1]

The title compound was prepared in a manner analogous to Example 20, except using 5-(4-bromo-1-methyl-1H-pyrazol-3-yl)-2-fluoropyridine (Intermediate 30) instead of 2-(4-bromo-1-methyl-1H-pyrazol-3-yl)-5-fluoropyridine (Intermediate 18). MS (ESI): mass calcd. for C₁₆H₁₂FN₅, 293.1; m/z found, 294.1 [M+H]⁺. ¹H NMR (500 MHz, DMSO-d₆) δ 11.73-11.62 (m, 1H), 8.19 (s, 1H), 8.17-8.15 (m, 1H), 8.15 (d, J=4.9 Hz, 1H), 7.88 (td, J=8.2, 2.5 Hz, 1H), 7.42-7.38 (m, 1H), 7.13 (dd, J=8.5, 2.8 Hz, 1H), 6.83 (d, J=4.8 Hz, 1H), 6.15 (dd, J=3.4, 1.9 Hz, 1H), 4.00 (s, 3H).

### Example 29: 4-[3-(6-Fluoro-2-pyridyl)-1-methyl-pyrazol-4-yl]-1H-pyrrolo[2,3-b]pyridine

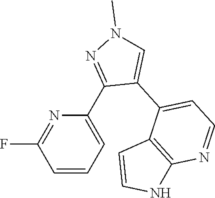 [SMILES: Cn1cc(-c2ccnc3[nH]ccc23)c(-c2cccc(F)n2)n1]

The title compound was prepared in a manner analogous to Example 20, except using 2-(4-bromo-1-methyl-1H-pyrazol-3-yl)-6-fluoropyridine (Intermediate 35) instead of 2-(4-bromo-1-methyl-1H-pyrazol-3-yl)-5-fluoropyridine (Intermediate 18). MS (ESI): mass calcd. for C₁₆H₁₂FN₅, 293.1; m/z found, 294.1 [M+H]⁺. ¹H NMR (500 MHz, DMSO-d₆) δ 11.65-11.51 (m, 1H), 8.17 (s, 1H), 8.11 (d, J=4.9 Hz, 1H), 8.00-7.93 (m, 1H), 7.57 (dd, J=7.5, 2.4 Hz, 1H), 7.39-7.35 (m, 1H), 7.05 (dd, J=8.1, 2.7 Hz, 1H), 6.88 (d, J=4.9 Hz, 1H), 6.15 (dd, J=3.5, 1.9 Hz, 1H), 4.00 (s, 3H).

### Example 30: 6-Methyl-4-(1-methyl-3-(6-methylpyridin-2-yl)-1H-pyrazol-4-yl)-1H-pyrazolo[3,4-b]pyridine

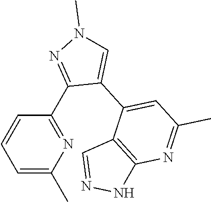 [SMILES: Cc1cccc(-c2nn(C)cc2-c2cc(C)nc3[nH]ncc23)n1]

The title compound was prepared in a manner analogous to Example 26, Steps A-B, using 2-(4-bromo-1-methyl-1H-pyrazol-3-yl)-6-methylpyridine (Intermediate 9) instead of 2-(4-bromo-1-methyl-1H-pyrazol-3-yl)-5-chloropyridine (Intermediate 8) in Step A. MS (ESI): mass calcd. for C₁₇H₁₆N₆, 304.1; m/z, found, 305.0 [M+H]⁺. ¹H NMR (400 MHz, Methanol-d₄) δ 8.19 (s, 1H), 7.75-7.66 (m, 1H), 7.55 (s, 1H), 7.40 (d, J=7.8 Hz, 1H), 7.24 (d, J=7.8 Hz, 1H), 6.99 (s, 1H), 4.07 (s, 3H), 2.56 (s, 3H), 2.36 (s, 3H).

### Example 31: 4-[1-Methyl-3-(5-methyl-2-pyridyl)pyrazol-4-yl]-1H-pyrrolo[2,3-b]pyridine

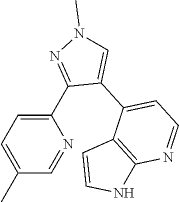 [SMILES: Cc1ccc(-c2nn(C)cc2-c2ccnc3[nH]ccc23)nc1]

The title compound was prepared in a manner analogous to Example 20, except using 2-(4-bromo-1-methyl-1H-pyrazol-3-yl)-5-methylpyridine (Intermediate 21) instead of 2-(4-bromo-1-methyl-1H-pyrazol-3-yl)-5-fluoropyridine (Intermediate 18). MS (ESI): mass calcd. for C₁₇H₁₅N₅, 289.1; m/z found, 290.1 [M+H]⁺. ¹H NMR (500 MHz, DMSO-d₆) δ 11.53 (br s, 1H), 8.24-8.20 (m, 1H), 8.15 (s, 1H), 8.05 (d, J=4.9 Hz, 1H), 7.63-7.58 (m, 1H), 7.56-7.52 (m, 1H), 7.37-7.34 (m, 1H), 6.82 (d, J=4.9 Hz, 1H), 6.20 (dd, J=3.5, 1.9 Hz, 1H), 3.98 (s, 3H), 2.28 (s, 3H).

### Example 32: 4-(1-Methyl-3-(5-methylpyridin-2-yl)-1H-pyrazol-4-yl)-1H-pyrazolo[3,4-b]pyridine

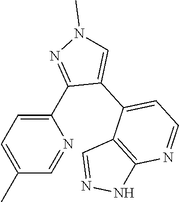 [SMILES: Cc1ccc(-c2nn(C)cc2-c2ccnc3[nH]ncc23)nc1]

The title compound was prepared in a manner analogous to Example 25, Steps A-B except using 2-(4-bromo-1-methyl-1H-pyrazol-3-yl)-5-methylpyridine (Intermediate 21) instead of 2-(4-bromo-1-methyl-1H-pyrazol-3-yl)-5-chloropyridine (Intermediate 8) in Step A. MS (ESI): mass calcd. for C₁₆H₁₄N₆, 290.1; m/z found, 291.1 [M+H]⁺. ¹H NMR (400 MHz, DMSO-d₆) δ 13.42 (br s, 1H), 8.36 (d, J=4.8 Hz, 1H), 8.33 (s, 1H), 8.24-8.19 (m, 1H), 7.76 (s, 1H), 7.70-7.61 (m, 2H), 7.00 (d, J=4.8 Hz, 1H), 4.00 (s, 3H), 2.29 (s, 3H).

### Example 33: 4-(3-(5-(Difluoromethyl)pyridin-2-yl)-1-methyl-1H-pyrazol-4-yl)-1H-pyrazolo[3,4-b]pyridine

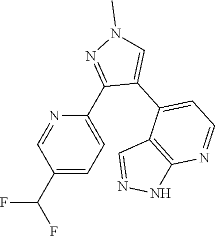 [SMILES: Cn1cc(-c2ccnc3[nH]ncc23)c(-c2ccc(C(F)F)cn2)n1]

The title compound was prepared in a manner analogous to Example 25, Steps A-B except using 2-(4-bromo-1-methyl-1H-pyrazol-3-yl)-5-(difluoromethyl)pyridine (Intermediate 20) instead of 2-(4-bromo-1-methyl-1H-pyrazol-3-yl)-5-chloropyridine (Intermediate 8) in Step A. MS (ESI): mass calcd. for C₁₆H₁₂F₂N₆, 326.1; m/z found, 327.1 [M+H]⁺. ¹H NMR (400 MHz, DMSO-d₆) δ 13.55 (s, 1H), 8.56-8.52 (m, 1H), 8.40 (d, J=4.8 Hz, 1H), 8.36 (s, 1H), 8.09-8.04 (m, 1H), 7.94 (dd, J=8.2, 0.9 Hz, 1H), 7.76 (s, 1H), 7.13 (t, J=55.3 Hz, 1H), 7.03 (d, J=4.8 Hz, 1H), 4.03 (s, 3H).

### Example 34: 4-(3-(6-Methoxypyridin-2-yl)-1-methyl-1H-pyrazol-4-yl)-6-methyl-1H-pyrazolo[3,4-b]pyridine

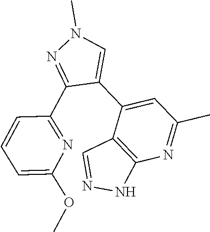 [SMILES: COc1cccc(-c2nn(C)cc2-c2cc(C)nc3[nH]ncc23)n1]

The title compound was prepared in a manner analogous to Example 26, Steps A-B, using 2-(4-bromo-1-methyl-1H-pyrazol-3-yl)-6-methoxypyridine (Intermediate 26) instead of 2-(4-bromo-1-methyl-1H-pyrazol-3-yl)-5-chloropyridine (Intermediate 8) in Step A. MS (ESI): mass calcd. for C₁₇H₁₆N₆O, 320.1; m/z found, 321.1 [M+H]⁺. ¹H NMR (400 MHz, DMSO-d₆) δ 13.30 (s, 1H), 8.19 (s, 1H), 7.76-7.70 (m, 1H), 7.51 (s, 1H), 7.44 (d, J=7.5 Hz, 1H), 6.98 (s, 1H), 6.65 (d, J=8.2 Hz, 1H), 3.98 (s, 3H), 2.97 (s, 3H), 2.51 (s, 3H).

### Example 35: 4-[3-[5-(Difluoromethoxy)-2-pyridyl]-1-methyl-pyrazol-4-yl]-1H-pyrrolo[2,3-b]pyridine

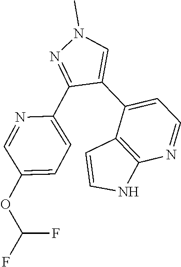 [SMILES: Cn1cc(-c2ccnc3[nH]ccc23)c(-c2ccc(OC(F)F)cn2)n1]

The title compound was prepared in a manner analogous to Example 20, except using 2-(4-bromo-1-methyl-1H-pyrazol-3-yl)-5-(difluoromethoxy)pyridine (Intermediate 33) instead of 2-(4-bromo-1-methyl-1H-pyrazol-3-yl)-5-fluoropyridine (Intermediate 18). MS (ESI): mass calcd. for C₁₇H₁₃F₂N₅₀, 341.1; m/z found, 342.1 [M+H]⁺. ¹H NMR (500 MHz, DMSO-d₆) δ 11.55 (s, 1H), 8.27 (d, J=2.8 Hz, 1H), 8.16 (s, 1H), 8.08 (d, J=4.9 Hz, 1H), 7.74 (d, J=8.6 Hz, 1H), 7.67 (dd, J=8.7, 2.9 Hz, 1H), 7.37-7.34 (m, 1H), 7.31 (t, J=73.4 Hz, 1H), 6.85 (d, J=4.9 Hz, 1H), 6.16 (dd, J=3.4, 1.9 Hz, 1H), 3.99 (s, 3H).

### Example 36: 4-[1-Methyl-3-[5-(trifluoromethoxy)-2-pyridyl]pyrazol-4-yl]-1H-pyrrolo[2,3-b]pyridine

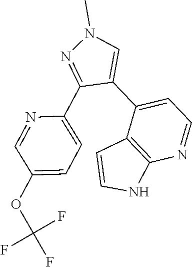 [SMILES: Cn1cc(-c2ccnc3[nH]ccc23)c(-c2ccc(OC(F)(F)F)cn2)n1]

The title compound was prepared in a manner analogous to Example 20, except using 2-(4-bromo-1-methyl-1H-pyrazol-3-yl)-5-(trifluoromethoxy)pyridine (Intermediate 57) instead of 2-(4-bromo-1-methyl-1H-pyrazol-3-yl)-5-fluoropyridine (Intermediate 18). MS (ESI): mass calcd. for C₁₇H₁₂F₃N₅₀, 359.1; m/z found, 360.1 [M+H]⁺. ¹H NMR (300 MHz, DMSO-d₆) δ 11.56 (br s, 1H), 8.51-8.39 (m, 1H), 8.18 (s, 1H), 8.10 (d, J=5.0 Hz, 1H), 7.96-7.86 (m, 1H), 7.83 (d, J=8.6 Hz, 1H), 7.41-7.29 (m, 1H), 6.87 (d, J=5.0 Hz, 1H), 6.17-6.05 (m, 1H), 4.01 (s, 3H).

### Example 37: 4-[3-(3,5-Difluoro-2-pyridyl)-1-methyl-pyrazol-4-yl]-1H-pyrrolo[2,3-b]pyridine

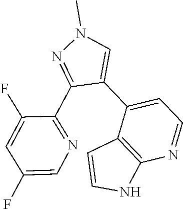 [SMILES: Cn1cc(-c2ccnc3[nH]ccc23)c(-c2ncc(F)cc2F)n1]

The title compound was prepared in a manner analogous to Example 20, except using 2-(4-bromo-1-methyl-1H-pyrazol-3-yl)-3,5-difluoropyridine (Intermediate 22) instead of 2-(4-bromo-1-methyl-1H-pyrazol-3-yl)-5-fluoropyridine (Intermediate 18). MS (ESI): mass calcd. for C₁₆H₁₁F₂N₅, 311.1; m/z found, 312.1 [M+H]⁺. ¹H NMR (300 MHz, DMSO-d₆) δ 11.61 (br s, 1H), 8.57-8.45 (m, 1H), 8.32 (s, 1H), 8.03 (d, J=5.0 Hz, 1H), 8.01-7.89 (m, 1H), 7.44-7.32 (m, 1H), 6.63 (d, J=5.0 Hz, 1H), 6.29-6.14 (m, 1H), 4.02 (s, 3H).

### Example 38: 4-(3-(3,5-Difluoropyridin-2-yl)-1-methyl-1H-pyrazol-4-yl)-1H-pyrazolo[3,4-b]pyridine

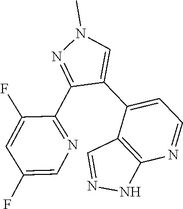 [SMILES: Cn1cc(-c2ccnc3[nH]ncc23)c(-c2ncc(F)cc2F)n1]

The title compound was prepared in a manner analogous to Example 25, Steps A-B, except using 2-(4-bromo-1-methyl-1H-pyrazol-3-yl)-3,5-difluoropyridine (Intermediate 22) instead of 2-(4-bromo-1-methyl-1H-pyrazol-3-yl)-5-chloropyridine (Intermediate 8) in Step A. MS (ESI): mass calcd. for C₁₅H₁₀F₂N₆, 312.1; m/z found, 313.1 [M+H]⁺. ¹H NMR (500 MHz, DMSO-d₆) δ 13.61 (s, 1H), 8.55-8.50 (m, 2H), 8.32 (d, J=4.9 Hz, 1H), 8.02 (td, J=9.4, 2.4 Hz, 1H), 7.90 (s, 1H), 6.73 (d, J=4.8 Hz, 1H), 4.03 (s, 3H).

### Example 39: 4-(3-(5-Chloro-6-methylpyridin-2-yl)-1-methyl-1H-pyrazol-4-yl)-6-methyl-1H-pyrazolo[3,4-b]pyridine

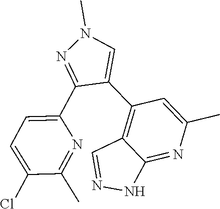 [SMILES: Cc1cc(-c2cn(C)nc2-c2ccc(Cl)c(C)n2)c2cn[nH]c2n1]

The title compound was prepared in a manner analogous to Example 26, Steps A-B, using 6-(4-bromo-1-methyl-1H-pyrazol-3-yl)-3-chloro-2-methylpyridine (Intermediate 27) instead of 2-(4-bromo-1-methyl-1H-pyrazol-3-yl)-5-chloropyridine (Intermediate 8) in Step A. MS (ESI): mass calcd. for C₁₇H₁₅ClN₆, 338.1; m/z found, 339.0 [M+H]⁺. ¹H NMR (400 MHz, DMSO-d₆) δ 13.31 (br.s., 1H), 8.30 (s, 1H), 7.87 (d, J=8.3 Hz, 1H), 7.66 (s, 1H), 7.61 (d, J=8.3 Hz, 1H), 7.13 (s, 1H), 4.00 (s, 3H), 2.48-2.45 (m, 3H), 2.26 (s, 3H).

### Example 40: 4-(3-(5-Fluoro-6-methylpyridin-2-yl)-1-methyl-1H-pyrazol-4-yl)-6-methyl-1H-pyrazolo[3,4-b]pyridine

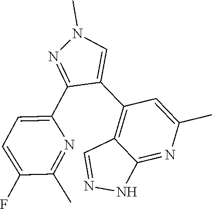 [SMILES: Cc1cc(-c2cn(C)nc2-c2ccc(F)c(C)n2)c2cn[nH]c2n1]

The title compound was prepared in a manner analogous to Example 26, Steps A-B, using 6-(4-bromo-1-methyl-1H-pyrazol-3-yl)-3-fluoro-2-methylpyridine (Intermediate 14) instead of 2-(4-bromo-1-methyl-1H-pyrazol-3-yl)-5-chloropyridine (Intermediate 8) in Step A. MS (ESI): mass calcd. for C₁₇H₁₅FN₆, 322.1; m/z found, 323.0 [M+H]⁺. ¹H NMR (400 MHz, Methanol-d₄) δ 8.15 (s, 1H), 7.56 (s, 1H), 7.52 (s, 1H), 7.51-7.49 (m, 1H), 7.04 (s, 1H), 4.06 (s, 3H), 2.58 (s, 3H), 2.29 (d, J=2.9 Hz, 3H).

### Example 41: 4-(3-(5-Fluoro-6-methoxypyridin-2-yl)-1-methyl-1H-pyrazol-4-yl)-6-methyl-1H-pyrazolo[3,4-b]pyridine

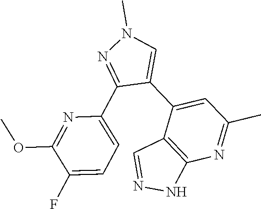

The title compound was prepared in a manner analogous to Example 26, Steps A-B, using 6-(4-bromo-1-methyl-1H-pyrazol-3-yl)-3-fluoro-2-methoxypyridine (Intermediate 17) instead of 2-(4-bromo-1-methyl-1H-pyrazol-3-yl)-5-chloropyridine (Intermediate 8) in Step A. MS (ESI): mass calcd. for C₁₇H₁₅FN₆O, 338.1; m/z found, 339.1 [M+H]⁺. ¹H NMR (400 MHz, DMSO-d₆) δ 13.32 (s, 1H), 8.20 (s, 1H), 7.70 (dd, J=8.2, 10.6 Hz, 1H), 7.53 (s, 1H), 7.46 (dd, J=2.9, 8.2 Hz, 1H), 6.98 (s, 1H), 3.99 (s, 3H), 3.05 (s, 3H), 2.53 (s, 3H)

### Example 42: 4-(3-(5-Chloro-6-methoxypyridin-2-yl)-1-methyl-1H-pyrazol-4-yl)-6-methyl-1H-pyrazolo[3,4-b]pyridine

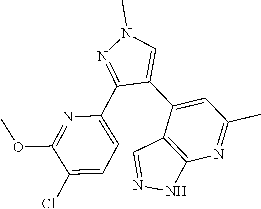

The title compound was prepared in a manner analogous to Example 26, Steps A-B, using 6-(4-bromo-1-methyl-1H-pyrazol-3-yl)-3-chloro-2-methoxypyridine (Intermediate 15) instead of 2-(4-bromo-1-methyl-1H-pyrazol-3-yl)-5-chloropyridine (Intermediate 8), and using cataCXium® A Pd G3 instead of Pd(dppf)Cl₂ in Step A. MS (ESI): mass calcd. for C₁₇H₁₅ClN₆O, 354.1; m/z found, 355.0 [M+H]⁺. ¹H NMR (400 MHz, CDCl₃) δ 12.56 (br s, 1H), 7.70-7.61 (m, 3H), 7.49 (d, J=8.0 Hz, 1H), 7.03 (s, 1H), 4.07 (s, 3H), 3.15 (s, 3H), 2.75 (s, 3H).

### Example 43: 4-[3-(5-Fluoropyrimidin-2-yl)-1-methyl-pyrazol-4-yl]-1H-pyrrolo[2,3-b]pyridine

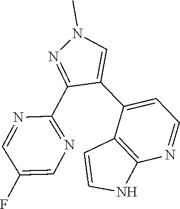 [SMILES: Cn1cc(-c2ccnc3[nH]ccc23)c(-c2ncc(F)cn2)n1]

The title compound was prepared in a manner analogous to Example 20, except using 2-(4-bromo-1-methyl-1H-pyrazol-3-yl)-5-fluoropyrimidine (Intermediate 65) instead of 2-(4-bromo-1-methyl-1H-pyrazol-3-yl)-5-fluoropyridine (Intermediate 18). MS (ESI): mass calcd. for C₁₅H₁₁FN₆, 294.1; m/z found, 295.1 [M+H]⁺. ¹H NMR (500 MHz, DMSO-d₆) δ 11.55 (br s, 1H), 8.85-8.78 (m, 2H), 8.18 (s, 1H), 8.08 (d, J=4.9 Hz, 1H), 7.36-7.33 (m, 1H), 6.80 (d, J=4.9 Hz, 1H), 6.05 (dd, J=3.5, 1.8 Hz, 1H), 4.01 (s, 3H).

### Example 44: 4-(1-Methyl-3-pyrimidin-4-yl-pyrazol-4-yl)-1H-pyrrolo[2,3-b]pyridine

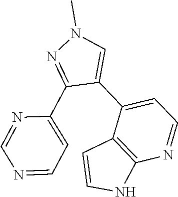 [SMILES: Cn1cc(-c2ccnc3[nH]ccc23)c(-c2ccncn2)n1]

The title compound was prepared in a manner analogous to Example 19, Step A, except using 4-(4-bromo-1-methyl-1H-pyrazol-3-yl)pyrimidine (Intermediate 58) instead of 4-(4-bromo-1-methyl-1H-pyrazol-3-yl)-3-fluoropyridine (Intermediate 19) and using 4-(4,4,5,5-tetramethyl-1,3,2-dioxaborolan-2-yl)-1H-pyrrolo[2,3-b]pyridine instead of 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 1). MS (ESI): mass calcd. for C₁₅H₁₂N₆, 276.1; m/z found, 277.1 [M+H]⁺. ¹H NMR (400 MHz, Methanol-d₄) δ 8.96 (d, J=1.4 Hz, 1H), 8.67 (d, J=5.4 Hz, 1H), 8.17 (d, J=5.1 Hz, 1H), 8.04 (s, 1H), 7.67 (dd, J=5.4, 1.4 Hz, 1H), 7.32 (d, J=3.5 Hz, 1H), 7.07 (d, J=5.1 Hz, 1H), 6.17 (d, J=3.5 Hz, 1H), 4.11 (s, 3H).

### Example 45: 4-(1-Methyl-3-pyrimidin-5-yl-pyrazol-4-yl)-1H-pyrrolo[2,3-b]pyridine

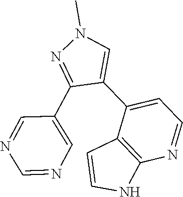 [SMILES: Cn1cc(-c2ccnc3[nH]ccc23)c(-c2cncnc2)n1]

The title compound was prepared in a manner analogous to Example 19, Step A, except using 5-(4-bromo-1-methyl-1H-pyrazol-3-yl)pyrimidine (Intermediate 61) instead of 4-(4-bromo-1-methyl-1H-pyrazol-3-yl)-3-fluoropyridine (Intermediate 19) and using 4-(4,4,5,5-tetramethyl-1,3,2-dioxaborolan-2-yl)-1H-pyrrolo[2,3-b]pyridine instead of 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 1). MS (ESI): mass calcd. for C₁₅H₁₂N₆, 276.1; m/z found, 277.1 [M+H]⁺. ¹H NMR (400 MHz, Methanol-d₄) δ 9.04 (s, 1H), 8.77 (s, 2H), 8.19 (d, J=5.0 Hz, 1H), 8.08 (s, 1H), 7.35 (d, J=3.5 Hz, 1H), 7.01 (d, J=5.0 Hz, 1H), 6.20 (d, J=3.5 Hz, 1H), 4.10 (s, 3H).

### Example 46: 4-(1-Methyl-3-pyrazin-2-yl-pyrazol-4-yl)-1H-pyrrolo[2,3-b]pyridine

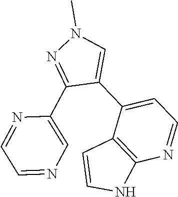 [SMILES: Cn1cc(-c2ccnc3[nH]ccc23)c(-c2cnccn2)n1]

The title compound was prepared in a manner analogous to Example 19, Step A, except using 2-(4-bromo-1-methyl-1H-pyrazol-3-yl)pyrazine (Intermediate 62) instead of 4-(4-bromo-1-methyl-1H-pyrazol-3-yl)-3-fluoropyridine (Intermediate 19) and using 4-(4,4,5,5-tetramethyl-1,3,2-dioxaborolan-2-yl)-1H-pyrrolo[2,3-b]pyridine instead of 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 1). MS (ESI): mass calcd. for C₁₅H₁₂N₆, 276.1; m/z found, 277.1 [M+H]⁺. ¹H NMR (400 MHz, Methanol-d₄) δ 8.77 (d, J=1.3 Hz, 1H), 8.54-8.41 (m, 2H), 8.14 (d, J=5.0 Hz, 1H), 8.07 (s, 1H), 7.30 (d, J=3.6 Hz, 1H), 7.02 (d, J=5.1 Hz, 1H), 6.16 (d, J=3.6 Hz, 1H), 4.11 (s, 3H).

### Example 47: 4-[1-Methyl-3-(5-methylpyrazin-2-yl)pyrazol-4-yl]-1H-pyrrolo[2,3-b]pyridine

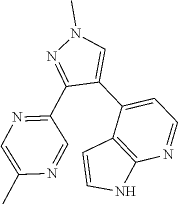 [SMILES: Cc1cnc(-c2nn(C)cc2-c2ccnc3[nH]ccc23)cn1]

The title compound was prepared in a manner analogous to Example 20, except using 2-(4-bromo-1-methyl-1H-pyrazol-3-yl)-5-methylpyrazine (Intermediate 66) instead of 2-(4-bromo-1-methyl-1H-pyrazol-3-yl)-5-fluoropyridine (Intermediate 18). MS (ESI): mass calcd. for C₁₆H₁₄N₆, 290.1; m/z found, 291.2 [M+H]⁺. ¹H NMR (500 MHz, DMSO-d₆) δ 11.58 (br s, 1H), 8.75-8.73 (m, 1H), 8.37-8.32 (m, 1H), 8.21 (s, 1H), 8.09 (d, J=4.9 Hz, 1H), 7.39-7.35 (m, 1H), 6.85 (d, J=4.9 Hz, 1H), 6.16 (dd, J=3.5, 1.9 Hz, 1H), 4.02 (s, 3H), 2.47 (s, 3H).

### Example 48: 4-(1-Methyl-3-pyridazin-3-yl-pyrazol-4-yl)-1H-pyrrolo[2,3-b]pyridine

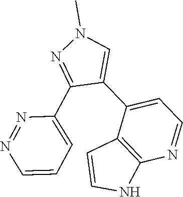 [SMILES: Cn1cc(-c2ccnc3[nH]ccc23)c(-c2cccnn2)n1]

The title compound was prepared in a manner analogous to Example 19, Step A, except using 3-(4-bromo-1-methyl-1H-pyrazol-3-yl)pyridazine (Intermediate 63) instead of 4-(4-bromo-1-methyl-1H-pyrazol-3-yl)-3-fluoropyridine (Intermediate 19) and using 4-(4,4,5,5-tetramethyl-1,3,2-dioxaborolan-2-yl)-1H-pyrrolo[2,3-b]pyridine instead of 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 1). MS (ESI): mass calcd. for C₁₅H₁₂N₆, 276.1; m/z found, 277.1 [M+H]⁺. ¹H NMR (400 MHz, Methanol-d₄) δ 9.07 (dd, J=5.0, 1.7 Hz, 1H), 8.13 (d, J=5.1 Hz, 1H), 8.10 (s, 1H), 7.90 (dd, J=8.6, 1.7 Hz, 1H), 7.69 (dd, J=8.6, 5.0 Hz, 1H), 7.28 (d, J=3.6 Hz, 1H), 7.01 (d, J=5.0 Hz, 1H), 6.15 (d, J=3.5 Hz, 1H), 4.12 (s, 3H).

### Example 49: 4-(1-Methyl-3-pyridazin-4-yl-pyrazol-4-yl)-1H-pyrrolo[2,3-b]pyridine

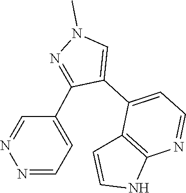 [SMILES: Cn1cc(-c2ccnc3[nH]ccc23)c(-c2ccnnc2)n1]

The title compound was prepared in a manner analogous to Example 19, Step A, except using 4-(4-bromo-1-methyl-1H-pyrazol-3-yl)pyridazine (Intermediate 64) instead of 4-(4-bromo-1-methyl-1H-pyrazol-3-yl)-3-fluoropyridine (Intermediate 19) and using 4-(4,4,5,5-tetramethyl-1,3,2-dioxaborolan-2-yl)-1H-pyrrolo[2,3-b]pyridine instead of 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 1). MS (ESI): mass calcd. for C₁₅H₁₂N₆, 276.1; m/z found, 277.1 [M+H]⁺. ¹H NMR (400 MHz, Methanol-d₄) δ 9.26 (dd, J=2.4, 1.2 Hz, 1H), 9.02 (dd, J=5.4, 1.3 Hz, 1H), 8.23 (d, J=5.0 Hz, 1H), 8.06 (s, 1H), 7.63 (dd, J=5.5, 2.3 Hz, 1H), 7.36 (d, J=3.5 Hz, 1H), 7.06 (d, J=5.0 Hz, 1H), 6.17 (d, J=3.5 Hz, 1H), 4.12 (s, 3H).

### Example 50: 4-[3-(5-Fluoro-2-pyridyl)-1-(oxetan-3-ylmethyl)pyrazol-4-yl]-6-methyl-1H-pyrazolo[3,4-b]pyridine

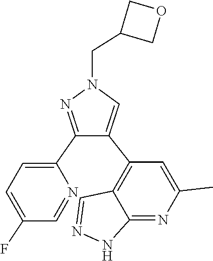 [SMILES: Cc1cc(-c2cn(CC3COC3)nc2-c2ccc(F)cn2)c2cn[nH]c2n1]

The title compound was prepared in a manner analogous to Example 19, except using 2-(4-bromo-1-(oxetan-3-ylmethyl)-1H-pyrazol-3-yl)-5-fluoropyridine (Intermediate 28) instead of 4-(4-bromo-1-methyl-1H-pyrazol-3-yl)-3-fluoropyridine (Intermediate 19) and 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 3) instead of 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 1). MS (ESI): mass calcd. for C₁₉H₁₇FN₆O, 364.1; m/z found, 365.1 [M+H]⁺. ¹H NMR (400 MHz, DMSO-d₆) δ 9.03-8.91 (m, 1H), 8.84 (d, J=2.6 Hz, 1H), 7.87-7.71 (m, 1H), 7.56-7.38 (m, 2H), 6.91-6.77 (m, 1H), 4.96-4.72 (m, 2H), 4.70-4.46 (m, 2H), 4.06-3.77 (m, 1H), 3.69 (d, J=5.0 Hz, 2H), 2.55-2.51 (m, 3H).

### Example 51: 4-(3-(5-Fluoropyridin-2-yl)-1,5-dimethyl-1H-pyrazol-4-yl)-1H-pyrrolo[2,3-b]pyridine

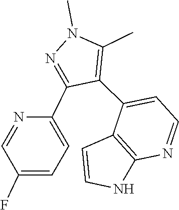 [SMILES: Cc1c(-c2ccnc3[nH]ccc23)c(-c2ccc(F)cn2)nn1C]

The title compound was prepared in a manner analogous to Example 19, Step A, except using 2-(4-bromo-1,5-dimethyl-1H-pyrazol-3-yl)-5-fluoropyridine (Intermediate 38) instead of 4-(4-bromo-1-methyl-1H-pyrazol-3-yl)-3-fluoropyridine (Intermediate 19) and using 4-(4,4,5,5-tetramethyl-1,3,2-dioxaborolan-2-yl)-1H-pyrrolo[2,3-b]pyridine instead of 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 1). MS (ESI): mass calcd. for C₁₇H₁₄FN₅, 307.1; m/z found, 308.1 [M+H]⁺. ¹H NMR (400 MHz, DMSO-d₆) δ 11.52 (br s, 1H) 8.23 (t, J=1.56 Hz, 1H) 8.15 (d, J=4.75 Hz, 1H) 7.68-7.61 (m, 2H) 7.29 (dd, J=3.31, 2.56 Hz, 1H) 6.82 (d, J=4.88 Hz, 1H) 5.85 (dd, J=3.50, 1.88 Hz, 1H) 3.90 (s, 3H) 2.20 (s, 3H).

### Example 52: 4-(5-Cyclobutyl-3-(5-fluoropyridin-2-yl)-1-methyl-1H-pyrazol-4-yl)-1H-pyrrolo[2,3-b]pyridine

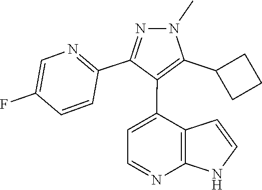 [SMILES: Cn1nc(-c2ccc(F)cn2)c(-c2ccnc3[nH]ccc23)c1C1CCC1]

The title compound was prepared in a manner analogous to Example 19, Step A, except using 2-(4-bromo-5-cyclobutyl-1-methyl-1H-pyrazol-3-yl)-5-fluoropyridine (Intermediate 39) instead of instead of 4-(4-bromo-1-methyl-1H-pyrazol-3-yl)-3-fluoropyridine (Intermediate 19) and using 4-(4,4,5,5-tetramethyl-1,3,2-dioxaborolan-2-yl)-1H-pyrrolo[2,3-b]pyridine instead of 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 1). MS (ESI): mass calcd. for C₂₀H₁₈FN₅, 347.2; m/z found, 348.1 [M+H]⁺. ¹H NMR (500 MHz, DMSO-d₆) δ 11.50 (br s, 1H) 8.19 (d, J=3.00 Hz, 1H) 8.12 (d, J=4.75 Hz, 1H) 7.63-7.56 (m, 1H) 7.56-7.51 (m, 1H) 7.30 (dd, J=3.31, 2.56 Hz, 1H) 6.84 (d, J=4.75 Hz, 1H) 5.93 (dd, J=3.44, 1.94 Hz, 1H) 3.91 (s, 3H) 3.80-3.68 (m, 1H) 2.16-2.04 (m, 1H) 1.96-1.83 (m, 3H) 1.83-1.71 (m, 1H) 1.56-1.45 (m, 1H).

### Example 53: 4-[1-Methyl-3-(3-pyridyl)pyrazol-4-yl]-1H-pyrrolo[2,3-b]pyridine

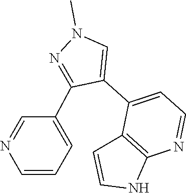 [SMILES: Cn1cc(-c2ccnc3[nH]ccc23)c(-c2cccnc2)n1]

The title compound was prepared in a manner analogous to Example 20, except using 3-(4-bromo-1-methyl-1H-pyrazol-3-yl)pyridine (Intermediate 34) instead of 2-(4-bromo-1-methyl-pyrazol-3-yl)-5-fluoro-pyridine (Intermediate 18). MS (ESI): mass calcd. for C₁₆H₁₃N₅, 275.1; m/z found, 276.2 [M+H]⁺. ¹H NMR (300 MHz, DMSO-d₆) δ 11.68 (br s, 1H), 8.58-8.50 (m, 1H), 8.49-8.39 (m, 1H), 8.19 (s, 1H), 8.13 (d, J=5.0 Hz, 1H), 7.78-7.65 (m, 1H), 7.46-7.36 (m, 1H), 7.36-7.28 (m, 1H), 6.81 (d, J=5.0 Hz, 1H), 6.22-6.09 (m, 1H), 4.00 (s, 3H).

### Example 54: 4-[3-(5-Fluoro-2-pyridyl)-1-(trideuteriomethyl)pyrazol-4-yl]-1H-pyrrolo[2,3-b]pyridine

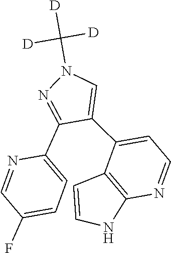 [SMILES: [2H]C([2H])([2H])n1cc(-c2ccnc3[nH]ccc23)c(-c2ccc(F)cn2)n1]

Step A: 4-(3-(5-Fluoropyridin-2-yl)-1-(methyl-d₃)-1H-pyrazol-4-yl)-1-(phenylsulfonyl)-1H-pyrrolo[2,3-b]pyridine. In a pressure vessel was placed 2-(4-bromo-1-(methyl-d₃)-1H-pyrazol-3-yl)-5-fluoropyridine (Intermediate 10, 48 mg, 0.19 mmol), 1-(phenylsulfonyl)-4-(4,4,5,5-tetramethyl-1,3,2-dioxaborolan-2-yl)-1H-pyrrolo[2,3-b]pyridine (Intermediate 42, 73 mg, 0.19 mmol) and Pd(dppf)Cl₂·DCM (15 mg, 0.019 mmol). The vial was sealed and flushed with a stream of N₂ before adding an aqueous solution of Na₂CO₃ (2 M, 0.28 mL) and 1,4-dioxane (1.2 mL). The reaction mixture was degassed with N₂ and heated to 80° C. for 16 hours. It was then filtered through Celite® and rinsed with MeOH. Solvent was evaporated and purification via silica gel chromatography (0% to 100% EtOAc/hexanes) gave the tile compound (48 mg, 59% yield). MS (ESI): mass calcd. for C₂₂H₁₃D₃FN₅₀₂S, 436.1; m/z found, 437.1 [M+H]⁺.

Step B: 4-[3-(5-Fluoro-2-pyridyl)-1-(trideuteriomethyl)pyrazol-4-yl]-1H-pyrrolo[2,3-b]pyridine. To 4-(3-(5-fluoropyridin-2-yl)-1-(methyl-d₃)-1H-pyrazol-4-yl)-1-(phenylsulfonyl)-1H-pyrrolo[2,3-b]pyridine (48 mg, 0.11 mmol) was added TBAF (1 M in THF, 1.1 mL). The reaction mixture was stirred at room temperature for 16 hours. Water and ethyl acetate were added and the aqueous phase was extracted twice with ethyl acetate. The combined organic layers were washed with a saturated aqueous solution of NaCl twice, dried over MgSO₄, filtered and evaporated. Purification via silica gel chromatography (0% to 100% EtOAc/hexanes) gave the title compound (25 mg, 77% yield). MS (ESI): mass calcd. for C₁₆H₉D₃FN₅, 296.1; m/z found, 297.1 [M+H]⁺. ¹H NMR (500 MHz, CDCl₃) δ 10.79 (s, 1H), 8.46 (d, J=2.9 Hz, 1H), 8.26 (d, J=5.0 Hz, 1H), 7.70 (s, 1H), 7.35 (dd, J=8.7, 4.4 Hz, 1H), 7.27 (d, J=3.7 Hz, 1H), 7.23 (td, J=8.4, 2.9 Hz, 1H), 6.98 (d, J=5.0 Hz, 1H), 6.22 (d, J=3.5 Hz, 1H).

### Example 55: 4-[1-Ethyl-3-(5-fluoro-2-pyridyl)pyrazol-4-yl]-1H-pyrrolo[2,3-b]pyridine

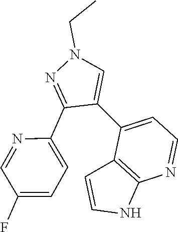 [SMILES: CCn1cc(-c2ccnc3[nH]ccc23)c(-c2ccc(F)cn2)n1]

The title compound was prepared in a manner analogous to Example 54, using 2-(4-bromo-1-ethyl-1H-pyrazol-3-yl)-5-fluoropyridine (Intermediate 23) instead of 2-(4-bromo-1-(methyl-d₃)-1H-pyrazol-3-yl)-5-fluoropyridine (Intermediate 10) in Step A. MS (ESI): mass calcd. for C₁₇H₁₄FN₅, 307.1; m/z found, 308.1 [M+H]⁺. ¹H NMR (500 MHz, CDCl₃) δ 8.46 (d, J=2.9 Hz, 1H), 8.25 (d, J=4.9 Hz, 1H), 7.74 (s, 1H), 7.37 (ddd, J=8.7, 4.4, 0.6 Hz, 1H), 7.26-7.21 (m, 3H), 6.98 (d, J=5.0 Hz, 1H), 6.22 (d, J=3.5 Hz, 1H), 4.35 (q, J=7.3 Hz, 2H), 1.62 (t, J=7.3 Hz, 3H).

### Example 56: 4-(1-Ethyl-3-(5-fluoropyridin-2-yl)-1H-pyrazol-4-yl)-1H-pyrazolo[3,4-b]pyridine

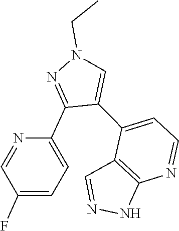 [SMILES: CCn1cc(-c2ccnc3[nH]ncc23)c(-c2ccc(F)cn2)n1]

The title compound was prepared in a manner analogous to Example 25, Steps A-B, except using 2-(4-bromo-1-ethyl-1H-pyrazol-3-yl)-5-fluoropyridine (Intermediate 23) instead of 2-(4-bromo-1-methyl-1H-pyrazol-3-yl)-5-chloropyridine (Intermediate 8) in Step A. MS (ESI): mass calcd. for C₁₆H₁₃FN₆, 308.1; m/z found, 309.1 [M+H]⁺. ¹H NMR (400 MHz, DMSO-d₆) δ 13.53 (s, 1H), 8.41-8.36 (m, 3H), 7.86-7.76 (m, 2H), 7.74 (s, 1H), 7.00 (d, J=4.8 Hz, 1H), 4.30 (q, J=7.3 Hz, 2H), 1.51 (t, J=7.3 Hz, 3H).

### Example 57: 4-(1-Ethyl-3-(5-fluoropyridin-2-yl)-1H-pyrazol-4-yl)-6-methyl-1H-pyrazolo[3,4-b]pyridine

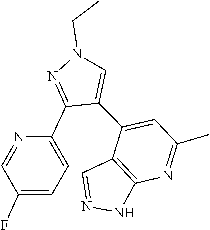 [SMILES: CCn1cc(-c2cc(C)nc3[nH]ncc23)c(-c2ccc(F)cn2)n1]

The title compound was prepared in a manner analogous to Example 25, Steps A-B, except using 2-(4-bromo-1-ethyl-1H-pyrazol-3-yl)-5-fluoropyridine (Intermediate 23) instead of 2-(4-Bromo-1-methyl-1H-pyrazol-3-yl)-5-chloropyridine (Intermediate 8) and 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 3) instead of 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 1) in Step A. MS (ESI): mass calcd. for C₁₇H₁₅FN₆, 322.1; m/z found, 323.1 [M+H]⁺. ¹H NMR (500 MHz, DMSO-d₆) δ 13.29 (s, 1H), 8.37 (d, J=3.0 Hz, 1H), 8.33 (s, 1H), 7.84-7.75 (m, 2H), 7.53 (s, 1H), 6.94 (s, 1H), 4.29 (q, J=7.3 Hz, 2H), 1.51 (t, J=7.3 Hz, 3H). Signal for methyl substituent on pyrazolo pyridine is buried under the DMSO-d₆ peak.

### Example 58: 4-[3-(5-Fluoro-2-pyridyl)-1-isopropyl-pyrazol-4-yl]-1H-pyrrolo[2,3-b]pyridine

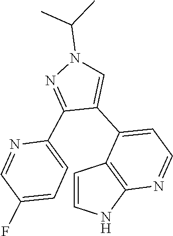 [SMILES: CC(C)n1cc(-c2ccnc3[nH]ccc23)c(-c2ccc(F)cn2)n1]

The title compound was prepared in a manner analogous to Example 54, using 2-(4-bromo-1-isopropyl-1H-pyrazol-3-yl)-5-fluoropyridine (Intermediate 24) instead of 2-(4-bromo-1-(methyl-d₃)-1H-pyrazol-3-yl)-5-fluoropyridine (Intermediate 10) in Step A. MS (ESI): mass calcd. for C₁₈H₁₆FN₅, 321.1; m/z found, 322.1 [M+H]⁺. ¹H NMR (400 MHz, CDCl₃) δ 10.87 (s, 1H), 8.46 (d, J=2.9 Hz, 1H), 8.28 (s, 1H), 7.77 (s, 1H), 7.40 (dd, J=8.7, 4.4 Hz, 1H), 7.25-7.19 (m, 2H), 7.00 (s, 1H), 6.23 (d, J=3.4 Hz, 1H), 4.76-4.63 (m, 1H), 1.63 (d, J=6.7 Hz, 6H).

### Example 59: 4-(3-(5-Fluoropyridin-2-yl)-1-isopropyl-1H-pyrazol-4-yl)-1H-pyrazolo[3,4-b]pyridine

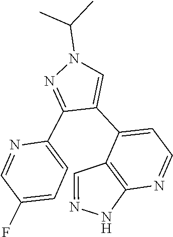 [SMILES: CC(C)n1cc(-c2ccnc3[nH]ncc23)c(-c2ccc(F)cn2)n1]

The title compound was prepared in a manner analogous to Example 25, Steps A-B, except using 2-(4-bromo-1-isopropyl-1H-pyrazol-3-yl)-5-fluoropyridine (Intermediate 24) instead of 2-(4-bromo-1-methyl-1H-pyrazol-3-yl)-5-chloropyridine (Intermediate 8) in Step A. MS (ESI): mass calcd. for C₁₇H₁₅FN₆, 322.1; m/z found, 323.1 [M+H]⁺. ¹H NMR (500 MHz, DMSO-d₆) δ 13.52 (s, 1H), 8.40-8.36 (m, 3H), 7.86-7.76 (m, 2H), 7.73 (s, 1H), 7.00 (d, J=4.8 Hz, 1H), 4.66 (p, J=6.7 Hz, 1H), 1.55 (d, J=6.7 Hz, 6H).

### Example 60: 4-(3-(5-Fluoropyridin-2-yl)-1-isopropyl-1H-pyrazol-4-yl)-6-methyl-1H-pyrazolo[3,4-b]pyridine

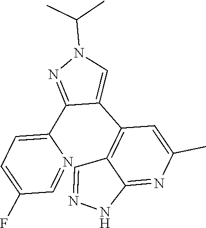 [SMILES: Cc1cc(-c2cn(C(C)C)nc2-c2ccc(F)cn2)c2cn[nH]c2n1]

The title compound was prepared in a manner analogous to Example 25, Steps A-B, except using 2-(4-bromo-1-isopropyl-1H-pyrazol-3-yl)-5-fluoropyridine (Intermediate 24) instead of 2-(4-bromo-1-methyl-1H-pyrazol-3-yl)-5-chloropyridine (Intermediate 8) and 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 3) instead of 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 1) in Step A. MS (ESI): mass calcd. for C₁₈H₁₇FN₆, 336.1; m/z found, 337.1 [M+H]⁺. ¹H NMR (500 MHz, Methanol-d₄) δ 8.32 (d, J=2.9 Hz, 1H), 8.21 (s, 1H), 7.79-7.74 (m, 1H), 7.66 (td, J=8.6, 3.0 Hz, 1H), 7.53 (s, 1H), 7.02 (s, 1H), 4.76-4.67 (m, 1H), 2.61 (s, 3H), 1.65 (d, J=6.7 Hz, 6H).

### Example 61: 4-[3-(5-Fluoro-2-pyridyl)-1-isobutyl-pyrazol-4-yl]-1H-pyrrolo[2,3-b]pyridine

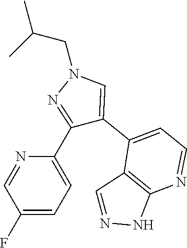 [SMILES: CC(C)Cn1cc(-c2ccnc3[nH]ncc23)c(-c2ccc(F)cn2)n1]

The title compound was prepared in a manner analogous to Example 65, using 2-(4-bromo-1-isobutyl-1H-pyrazol-3-yl)-5-fluoropyridine (Intermediate 25) instead of 2-(4-bromo-1-(difluoromethyl)-1H-pyrazol-3-yl)-5-fluoropyridine (Intermediate 16) in Step A. MS (ESI): mass calcd. for C₁₉H₁₈FN₅, 335.2; m/z found, 336.2 [M+H]⁺. ¹H NMR (400 MHz, CDCl₃) δ 10.89 (s, 1H), 8.45 (d, J=2.9 Hz, 1H), 8.26 (d, J=5.1 Hz, 1H), 7.72 (s, 1H), 7.47-7.34 (m, 1H), 7.31-7.20 (m, 2H), 7.00 (d, J=4.9 Hz, 1H), 6.25 (d, J=3.6 Hz, 1H), 4.06 (d, J=7.3 Hz, 2H), 2.47-2.29 (m, 1H), 1.02 (d, J=6.7 Hz, 6H).

### Example 62: 4-(3-(5-Fluoropyridin-2-yl)-1-isobutyl-1H-pyrazol-4-yl)-1H-pyrazolo[3,4-b]pyridine

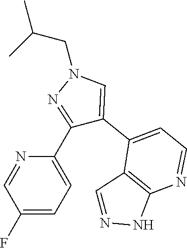 [SMILES: CC(C)Cn1cc(-c2ccnc3[nH]ncc23)c(-c2ccc(F)cn2)n1]

The title compound was prepared in a manner analogous to Example 25, Steps A-B, except using 2-(4-bromo-1-isobutyl-1H-pyrazol-3-yl)-5-fluoropyridine (Intermediate 25) instead of 2-(4-bromo-1-methyl-1H-pyrazol-3-yl)-5-chloropyridine (Intermediate 8) in Step A. MS (ESI): mass calcd. for C₁₈H₁₇FN₆, 336.1; m/z found, 337.1 [M+H]⁺. ¹H NMR (500 MHz, DMSO-d₆) δ 13.50 (s, 1H), 8.41-8.36 (m, 3H), 7.87-7.76 (m, 2H), 7.73 (s, 1H), 7.01 (d, J=4.8 Hz, 1H), 4.08 (d, J=7.2 Hz, 2H), 2.26 (hept, J=6.8 Hz, 1H), 0.95 (d, J=6.7 Hz, 6H).

### Example 63: 4-(3-(5-Fluoropyridin-2-yl)-1-isobutyl-1H-pyrazol-4-yl)-6-methyl-1H-pyrazolo[3,4-b]pyridine

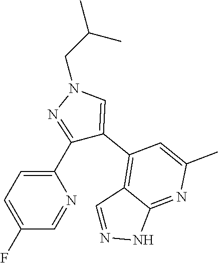 [SMILES: Cc1cc(-c2cn(CC(C)C)nc2-c2ccc(F)cn2)c2cn[nH]c2n1]

The title compound was prepared in a manner analogous to Example 25, Steps A-B, except using 2-(4-bromo-1-isobutyl-1H-pyrazol-3-yl)-5-fluoropyridine (Intermediate 25) instead of 2-(4-bromo-1-methyl-1H-pyrazol-3-yl)-5-chloropyridine (Intermediate 8) in Step A. MS (ESI): mass calcd. for C₁₉H₁₉FN₆, 350.1; m/z found, 351.1 [M+H]⁺. ¹H NMR (400 MHz, CDCl₃) δ 8.37 (d, J=2.9 Hz, 1H), 7.74 (s, 1H), 7.67-7.61 (m, 1H), 7.56 (s, 1H), 7.41-7.34 (m, 1H), 7.01 (s, 1H), 4.07 (d, J=7.3 Hz, 2H), 2.70 (s, 3H), 2.45-2.30 (m, 1H), 1.03 (d, J=6.7 Hz, 6H).

### Example 64: 4-(3-(5-Fluoropyridin-2-yl)-1-(2-methoxyethyl)-1H-pyrazol-4-yl)-6-methyl-1H-pyrazolo[3,4-b]pyridine

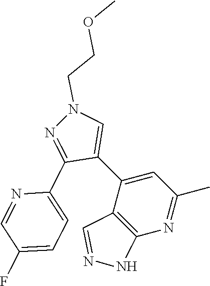 [SMILES: COCCn1cc(-c2cc(C)nc3[nH]ncc23)c(-c2ccc(F)cn2)n1]

Step A. 4-(5-(5-Fluoropyridin-2-yl)-1-(tetrahydro-2H-pyran-2-yl)-1H-pyrazol-4-yl)-6-methyl-1-((2-(trimethylsilyl)ethoxy)methyl)-1H-pyrazolo[3,4-b]pyridine. The title compound was prepared in a manner analogous to Example 19, Step A, except using 2-(4-bromo-1-(tetrahydro-2H-pyran-2-yl)-1H-pyrazol-5-yl)-5-fluoropyridine (Intermediate 45) instead of 4-(4-bromo-1-methyl-1H-pyrazol-3-yl)-3-fluoropyridine (Intermediate 19, and 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 3) instead of 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 1) and heating for 2 hours instead of 18 hours. MS (ESI): mass calcd. for C₂₆H₃₃FN₆O₂Si, 508.2; m/z found, 509.3 [M+H]⁺.

Step B. 4-(3-(5-Fluoropyridin-2-yl)-1H-pyrazol-4-yl)-6-methyl-1-((2-(trimethylsilyl)ethoxy)methyl)-1H-pyrazolo[3,4-b]pyridine. 4-(5-(5-Fluoropyridin-2-yl)-1-(tetrahydro-2H-pyran-2-yl)-1H-pyrazol-4-yl)-6-methyl-1-((2-(trimethylsilyl) ethoxy)methyl)-1H-pyrazolo[3,4-b]pyridine (3.6 g, 7.1 mmol) was added to a solution consisting of AcOH (20 mL) and H₂O (20 mL). The resultant mixture was stirred at 70° C. for 3 hours before then cooled to room-temperature. The mixture was concentrated to dryness under reduced pressure to afford the title compound (3.5 g, crude), which was used in the next step without further purification. MS (ESI): mass calcd. for C₂₁H₂₅FN₆OSi, 424.2; m/z found, 425.2 [M+H]⁺.

Step C. 4-(3-(5-Fluoropyridin-2-yl)-1-(2-methoxyethyl)-1H-pyrazol-4-yl)-6-methyl-1-((2-(trimethylsilyl)ethoxy)methyl)-1H-pyrazolo[3,4-b]pyridine. 1-Bromo-2-methoxyethane (59.0 mg, 0.424 mmol) was added to a solution consisting of 4-(3-(5-fluoropyridin-2-yl)-1H-pyrazol-4-yl)-6-methyl-1-((2-(trimethylsilyl)ethoxy)methyl)-1H-pyrazolo[3,4-b]pyridine (150 mg, 0.353 mmol), Cs₂CO₃ (288 mg, 0.884 mmol), and MeCN (2 mL). The resultant mixture was stirred at room-temperature for 2.5 hours before filtering through a pad of Celite® and the pad washing with ethyl acetate (5 mL). The filtrate was concentrated to dryness under reduced pressure to give the crude product, which was purified by FCC (eluent:petroleum ether:ethyl acetate=1:0 to 1:1) to afford the title compound (109 mg, 61%) as a colorless oil. MS (ESI): mass calcd. for C₂₄H₃₁FN₆O₂Si, 482.2; m/z found, 483.2 [M+H]⁺.

Step D. 4-(3-(5-Fluoropyridin-2-yl)-1-(2-methoxyethyl)-1H-pyrazol-4-yl)-6-methyl-1H-pyrazolo[3,4-b]pyridine. A mixture of 4-(3-(5-fluoropyridin-2-yl)-1-(2-methoxyethyl)-1H-pyrazol-4-yl)-6-methyl-1-((2-(trimethylsilyl)ethoxy)methyl)-1H-pyrazolo[3,4-b]pyridine (94.0 mg, 0.195 mmol) and TFA/DCM (1 mL, 1:3) was stirred at room temperature for 2 hours. The mixture was directly concentrated under reduced pressure and aqueous NaHCO₃ was added to the mixture to adjust it to pH=˜7-8. The reaction mixture was poured into water (3 mL) and extracted with dichloromethane (3 mL×3). The combined organic extracts were concentrated to dryness under reduced pressure to give the crude product, which was purified by preparative HPLC Method E; to afford the title compound. The title compound was suspended in water (5 mL), the mixture frozen using dry ice/ethanol, and then lyophilized to dryness to afford the title compound (13 mg) as a white solid. MS (ESI): mass calcd. for C₁₈H₁₇FN₆O, 352.1; m/z found, 353.0 [M+H]⁺. ¹H NMR (400 MHz, DMSO-d₆) δ 13.32 (s, 1H), 8.38 (d, J=2.6 Hz, 1H), 8.31 (s, 1H), 7.86-7.74 (m, 2H), 7.49 (s, 1H), 6.94 (s, 1H), 4.41 (t, J=5.2 Hz, 2H), 3.82 (t, J=5.3 Hz, 2H), 3.29 (s, 3H), 2.52-2.51 (m, 3H).

### Example 65: 4-[1-(Difluoromethyl)-3-(5-fluoro-2-pyridyl)pyrazol-4-yl]-1H-pyrrolo[2,3-b]pyridine

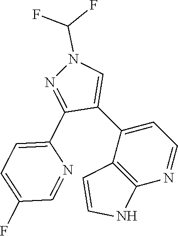 [SMILES: Fc1ccc(-c2nn(C(F)F)cc2-c2ccnc3[nH]ccc23)nc1]

Step A: 4-(1-(Difluoromethyl)-3-(5-fluoropyridin-2-yl)-1H-pyrazol-4-yl)-1-((2-(trimethylsilyl)ethoxy)methyl)-1H-pyrrolo[2,3-b]pyridine. In a pressure vessel was placed 2-(4-bromo-1-(difluoromethyl)-1H-pyrazol-3-yl)-5-fluoropyridine ( [symbol] Intermediate 16 [symbol] , 50 mg, 0.17 mmol), 4-(4,4,5,5-tetramethyl-1,3,2-dioxaborolan-2-yl)-1-((2-(trimethylsilyl)ethoxy)methyl)-1H-pyrrolo[2,3-b]pyridine (Intermediate 6, 64 mg, 0.17 mmol) and Pd(dppf)Cl₂·DCM (14 mg, 0.017 mmol). The vial was sealed and flushed with a stream of N₂ before adding an aqueous solution of Na₂CO₃ (2 M, 0.26 mL) and 1,4-dioxane (1.1 mL). The reaction mixture was degassed with N₂ and heated to 80° C. for 18 hours. It was then filtered through Celite® and rinsed with MeOH. Solvent was evaporated and purification via silica gel chromatography (0% to 100% EtOAc/hexanes) gave the tile compound (37 mg, 47% yield). MS (ESI): mass calcd. for C₂₂H₂₄F₃N₅OSi, 459.2; m/z found, 460.2 [M+H]⁺.

Step B: 4-[1-(Difluoromethyl)-3-(5-fluoro-2-pyridyl)pyrazol-4-yl]-1H-pyrrolo[2,3-b]pyridine. To a solution of 4-(1-(difluoromethyl)-3-(5-fluoropyridin-2-yl)-1H-pyrazol-4-yl)-1-((2-(trimethylsilyl)ethoxy)methyl)-1H-pyrrolo[2,3-b]pyridine (37 mg, 0.08 mmol) in DCM was added TFA (0.12 mL, 1.6 mmol). The reaction mixture was stirred at room temperature for 16 hours then evaporated. The residue was taken up in 2 mL of 2 M NH₃ in MeOH and stirred at room temperature for 16 hours. The solvent was evaporated and purification via basic preparative HPLC Method B; gave the title compound (23 mg, 88% yield). MS (ESI): mass calcd. for C₁₆H₁₀F₃N₅, 329.1; m/z found, 330.1 [M+H]⁺. ¹H NMR (500 MHz, CDCl₃) δ 10.05 (s, 1H), 8.42 (d, J=2.9 Hz, 1H), 8.29 (d, J=5.1 Hz, 1H), 8.15 (s, 1H), 7.57-7.47 (m, 1H), 7.41-7.19 (m, 3H), 7.06 (d, J=5.1 Hz, 1H), 6.25 (d, J=3.6 Hz, 1H).

### Example 66: 4-(1-(Difluoromethyl)-3-(5-fluoropyridin-2-yl)-1H-pyrazol-4-yl)-1H-pyrazolo[3,4-b]pyridine

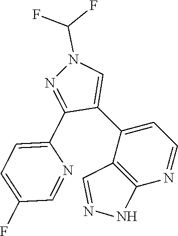 [SMILES: Fc1ccc(-c2nn(C(F)F)cc2-c2ccnc3[nH]ncc23)nc1]

The title compound was prepared in a manner analogous to Example 25, Steps A-B, except using 2-(4-bromo-1-(difluoromethyl)-1H-pyrazol-3-yl)-5-fluoropyridine (Intermediate 16) instead of 2-(4-bromo-1-methyl-1H-pyrazol-3-yl)-5-chloropyridine (Intermediate 8) in Step A. MS (ESI): mass calcd. for C₁₅H₉F₃N₆, 330.1; m/z found, 331.1 [M+H]⁺. ¹H NMR (500 MHz, DMSO-d₆) δ 13.64 (s, 1H), 8.86 (s, 1H), 8.45 (d, J=4.7 Hz, 1H), 8.43-8.38 (m, 1H), 8.10-7.96 (m, 1H), 7.95-7.91 (m, 1H), 7.89-7.84 (m, 1H), 7.72 (s, 1H), 7.06 (d, J=4.8 Hz, 1H).

### Example 67: 4-(1-(Difluoromethyl)-3-(5-fluoropyridin-2-yl)-1H-pyrazol-4-yl)-6-methyl-1H-pyrazolo[3,4-b]pyridine

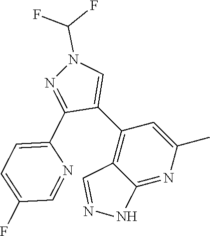 [SMILES: Cc1cc(-c2cn(C(F)F)nc2-c2ccc(F)cn2)c2cn[nH]c2n1]

The title compound was prepared in a manner analogous to Example 25, Steps A-B, except using 2-(4-bromo-1-(difluoromethyl)-1H-pyrazol-3-yl)-5-fluoropyridine (Intermediate 16) instead of 2-(4-bromo-1-methyl-1H-pyrazol-3-yl)-5-chloropyridine (Intermediate 8) and 6-methyl-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 3) instead of 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 1) in Step A. MS (ESI): mass calcd. for C₁₆H₁₁F₃N₆, 344.1; m/z found, 345.0 [M+H]⁺. ¹H NMR (400 MHz, DMSO-d₆) δ 13.41 (s, 1H), 8.81 (s, 1H), 8.40 (d, J=2.8 Hz, 1H), 7.97 (t, J=58.8, 1H), 7.94-7.83 (m, 2H), 7.52-7.45 (m, 1H), 7.00 (s, 1H), 2.54 (s, 3H).

### Example 68: 4-[1-(2-Fluoroethyl)-3-(5-fluoro-2-pyridyl)pyrazol-4-yl]-1H-pyrrolo[2,3-b]pyridine

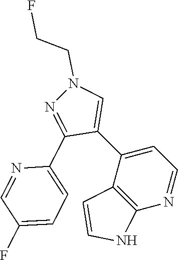 [SMILES: FCCn1cc(-c2ccnc3[nH]ccc23)c(-c2ccc(F)cn2)n1]

The title compound was prepared in a manner analogous to Example 19, Step A, except using 4-[1-ethyl-3-(5-fluoro-2-pyridyl)pyrazol-4-yl]-1H-pyrrolo[2,3-b]pyridine (Intermediate 67) instead of 4-(4-bromo-1-methyl-1H-pyrazol-3-yl)-3-fluoropyridine (Intermediate 19) and using 4-(4,4,5,5-tetramethyl-1,3,2-dioxaborolan-2-yl)-1H-pyrrolo[2,3-b]pyridine instead of 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 1). MS (ESI): mass calcd. for C₁₇H₁₃F₂N₅, 325.1; m/z found, 326.0 [M+H]⁺.

### Example 69: 4-(1-(2,2-Difluoroethyl)-3-(5-fluoropyridin-2-yl)-1H-pyrazol-4-yl)-6-methyl-1H-pyrazolo[3,4-b]pyridine

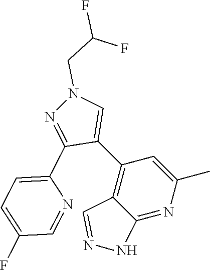 [SMILES: Cc1cc(-c2cn(CC(F)F)nc2-c2ccc(F)cn2)c2cn[nH]c2n1]

The title compound was prepared in a manner analogous to Example 64, Steps A-B, except using 2-bromo-1,1-difluoroethane instead of 1-bromo-2-methoxyethane in Step A. MS (ESI): mass calcd. for C₁₇H₁₃F₃N₆, 358.1; m/z found, 359.0 [M+H]⁺. ¹H NMR (400 MHz, DMSO-d₆) δ 13.36 (s, 1H), 8.39 (s, 2H), 7.90-7.74 (m, 2H), 7.50 (s, 1H), 6.95 (s, 1H), 6.73-6.32 (m, 1H), 4.79 (dt, J=3.6, 15.0 Hz, 2H), 2.52-2.51 (m, 3H).

### Example 70: 4-(3-(5-Fluoropyridin-2-yl)-1-(2,2,2-trifluoroethyl)-1H-pyrazol-4-yl)-6-methyl-1H-pyrazolo[3,4-b]pyridine

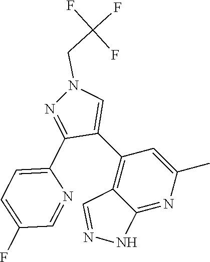 [SMILES: Cc1cc(-c2cn(CC(F)(F)F)nc2-c2ccc(F)cn2)c2cn[nH]c2n1]

Step A. 4-(3-(5-Fluoropyridin-2-yl)-1-(2,2,2-trifluoroethyl)-1H-pyrazol-4-yl)-6-methyl-1-((2-(trimethylsilyl)ethoxy)methyl)-1H-pyrazolo[3,4-b]pyridine. The title compound was prepared in a manner analogous to Example 26, Step A, except using 2-(4-bromo-1-(2,2,2-trifluoroethyl)-1H-pyrazol-3-yl)-5-fluoropyridine (Intermediate 13) instead of 2-(4-bromo-1-methyl-1H-pyrazol-3-yl)-5-chloropyridine (Intermediate 8) and using cataCXium® A Pd G3 instead of Pd(dppf)Cl₂ in Step A. MS (ESI): mass calcd. for C₂₃H₂₆F₄N₆OSi, 506.2; m/z found, 507.5 [M+H]⁺.

Step B. 4-(3-(5-Fluoropyridin-2-yl)-1-(2,2,2-trifluoroethyl)-1H-pyrazol-4-yl)-6-methyl-1H-pyrazolo[3,4-b]pyridine. 4N HCl in 1,4-dioxane (5 mL) and 4-(3-(5-fluoropyridin-2-yl)-1-(2,2,2-trifluoroethyl)-1H-pyrazol-4-yl)-6-methyl-1-((2-(trimethylsilyl)ethoxy)methyl)-1H-pyrazolo[3,4-b]pyridine (130 mg, 0.257 mmol) were added to a 50 mL round-bottomed flask. The resultant mixture was stirred at 75° C. for 2 hours. The reaction mixture was poured into water (10 mL) and extracted with dichloromethane:methanol: 25% NH_{3(aq)} (10:1:0.1, 10 mL×3). The combined organic extracts were dried over anhydrous Na₂SO₄, filtered, and concentrated to dryness under reduced pressure to give the crude product, which was purified by SFC Method B. The pure fractions were collected and the volatiles were removed under reduced pressure. The product was suspended in water (10 mL), the mixture frozen using dry ice/EtOH, and then lyophilized to dryness to afford the title compound (18.3 mg, 18%) as a white solid. MS (ESI): mass calcd. for C₁₇H₁₂F₄N₆, 376.1; m/z found, 377.1 [M+H]⁺. ¹H NMR (400 MHz, DMSO-d₆) δ 13.38 (br s, 1H), 8.45 (s, 1H), 8.39 (d, J=1.9 Hz, 1H), 7.85-7.79 (m, 2H), 7.46 (s, 1H), 6.97 (s, 1H), 5.32 (q, J=9.0 Hz, 2H), 2.52 (s, 3H).

### Example 71: 4-(1-Cyclobutyl-3-(5-fluoropyridin-2-yl)-1H-pyrazol-4-yl)-6-methyl-1H-pyrazolo[3,4-b]pyridine

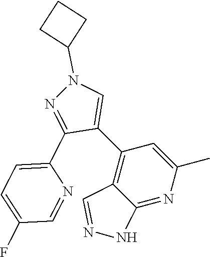 [SMILES: Cc1cc(-c2cn(C3CCC3)nc2-c2ccc(F)cn2)c2cn[nH]c2n1]

The title compound was prepared in a manner analogous to Example 64, Steps A-B, except using bromocyclobutane instead of 1-bromo-2-methoxyethane in Step A. MS (ESI): mass calcd. for C₁₉H₁₇FN₆, 348.2; m/z found, 349.1 [M+H]⁺. ¹H NMR (400 MHz, CDCl₃) δ 11.19 (br s, 1H), 8.36 (d, J=2.7 Hz, 1H), 7.82 (s, 1H), 7.71-7.60 (m, 1H), 7.54 (s, 1H), 7.38 (dt, J=2.8, 8.4 Hz, 1H), 7.00 (s, 1H), 5.00-4.84 (m, 1H), 2.74-2.57 (m, 5H), 2.04-1.89 (m, 2H), 0.95-0.78 (m, 2H).

### Example 72: 4-[3-(5-Fluoro-2-pyridyl)-1-(oxetan-3-yl)pyrazol-4-yl]-1H-pyrrolo[2,3-b]pyridine

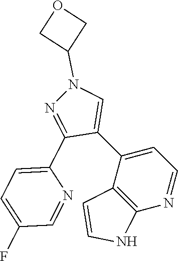 [SMILES: Fc1ccc(-c2nn(C3COC3)cc2-c2ccnc3[nH]ccc23)nc1]

The title compound was prepared in a manner analogous to Example 54, using 2-(4-bromo-1-(oxetan-3-yl)-1H-pyrazol-3-yl)-5-fluoropyridine (Intermediate 11) instead of 2-(4-bromo-1-(methyl-d₃)-1H-pyrazol-3-yl)-5-fluoropyridine (Intermediate 10) in Step A. MS (ESI): mass calcd. for C₁₈H₁₄FN₅O, 335.1; m/z found, 336.1 [M+H]⁺. ¹H NMR (500 MHz, CDCl₃) δ 9.40 (s, 1H), 8.44 (d, J=2.9 Hz, 1H), 8.27 (d, J=5.0 Hz, 1H), 7.98 (s, 1H), 7.51-7.42 (m, 1H), 7.33-7.20 (m, 2H), 7.00 (d, J=5.0 Hz, 1H), 6.21 (dd, J=3.6, 1.8 Hz, 1H), 5.70-5.59 (m, 1H), 5.23-5.06 (m, 4H).

### Example 73: 4-(3-(5-Fluoropyridin-2-yl)-1-(oxetan-3-yl)-1H-pyrazol-4-yl)-6-methyl-1H-pyrazolo[3,4-b]pyridine

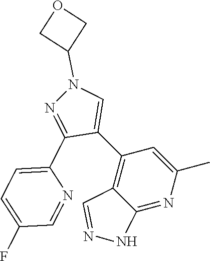 [SMILES: Cc1cc(-c2cn(C3COC3)nc2-c2ccc(F)cn2)c2cn[nH]c2n1]

The title compound was prepared in a manner analogous to Example 26, Steps A-B, using 2-(4-bromo-1-(oxetan-3-yl)-1H-pyrazol-3-yl)-5-fluoropyridine (Intermediate 11) instead of 2-(4-bromo-1-methyl-1H-pyrazol-3-yl)-5-chloropyridine (Intermediate 8), and using cataCXium® A Pd G3 instead of Pd(dppf)Cl₂ in Step A. MS (ESI): mass calcd. for C₁₈H₁₅FN₆O, 350.1; m/z found, 351.2 [M+H]⁺. ¹H NMR (400 MHz, CDCl₃) δ 11.59 (br s, 1H), 8.35 (d, J=2.8 Hz, 1H), 7.97 (s, 1H), 7.75-7.67 (m, 1H), 7.52 (s, 1H), 7.45-7.38 (m, 1H), 7.03 (s, 1H), 5.69-5.58 (m, 1H), 5.26-5.13 (m, 4H), 2.71 (s, 3H).

### Example 74: (R/S)-4-[3-(5-Fluoro-2-pyridyl)-1-tetrahydrofuran-3-yl-pyrazol-4-yl]-1H-pyrrolo[2,3-b]pyridine

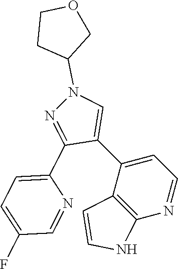 [SMILES: Fc1ccc(-c2nn(C3CCOC3)cc2-c2ccnc3[nH]ccc23)nc1]

The title compound was prepared in a manner analogous to Example 65, using (R/S)-2-(4-bromo-1-(tetrahydrofuran-3-yl)-1H-pyrazol-3-yl)-5-fluoropyridine (Intermediate 12) instead of 2-(4-bromo-1-(difluoromethyl)-1H-pyrazol-3-yl)-5-fluoropyridine (Intermediate 16) in Step A. MS (ESI): mass calcd. for C₁₉H₁₆FN₅O, 349.1; m/z found, 350.1 [M+H]⁺. ¹H NMR (400 MHz, CDCl₃) δ 10.32 (s, 1H), 8.45 (d, J=2.9 Hz, 1H), 8.27 (s, 1H), 7.84 (s, 1H), 7.39 (dd, J=8.8, 4.4 Hz, 1H), 7.30-7.20 (m, 2H), 6.99 (d, J=4.6 Hz, 1H), 6.20 (d, J=3.5 Hz, 1H), 5.24-5.15 (m, 1H), 4.29-4.16 (m, 2H), 4.11 (dd, J=10.0, 5.9 Hz, 1H), 3.98 (td, J=8.7, 5.8 Hz, 1H), 2.65-2.51 (m, 1H), 2.51-2.40 (m, 1H).

### Example 75: 3-Bromo-4-[3-(5-fluoro-2-pyridyl)-1-methyl-pyrazol-4-yl]-1H-pyrrolo[2,3-b]pyridine

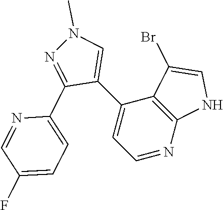 [SMILES: Cn1cc(-c2ccnc3[nH]cc(Br)c23)c(-c2ccc(F)cn2)n1]

To a mixture of 4-(3-(5-fluoropyridin-2-yl)-1-methyl-1H-pyrazol-4-yl)-1H-pyrrolo[2,3-b]pyridine (Example 20, 2.0 g, 6.82 mmol) in DCM (150 mL) at 0° C. was added methanesulfonic acid (0.44 mL, 6.82 mmol) then NBS (1.4 g, 7.84 mmol). After 15 minutes stirring at 0° C., the resulting solution was quenched with sat. sodium bicarbonate solution (100 mL) then extracted with DCM (3×100 mL). Solvent was evaporated and purification via silica gel chromatography (0% to 100% EtOAc/DCM) gave the tile compound (836 mg, 33% yield). MS (ESI): mass calcd. for C₁₆H₁₁BrFN₅, 371.0; m/z found, 372.1 [M+H]⁺. ¹H NMR (600 MHz, CDCl₃) δ 11.04 (s, 1H), 8.32 (d, J=3.0 Hz, 2H), 7.55 (s, 1H), 7.39-7.33 (m, 2H), 7.19 (ddd, J=8.8, 8.1, 2.9 Hz, 1H), 7.03 (d, J=4.8 Hz, 1H), 4.08 (s, 3H).

### Example 76: 3-Chloro-4-[3-(5-fluoro-2-pyridyl)-1-methyl-pyrazol-4-yl]-1H-pyrrolo[2,3-b]pyridine

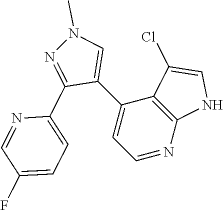 [SMILES: Cn1cc(-c2ccnc3[nH]cc(Cl)c23)c(-c2ccc(F)cn2)n1]

The title compound was prepared in a manner analogous to Example 1, Step A, except using 4-bromo-3-chloro-1H-pyrrolo[2,3-b]pyridine instead of 4-bromopyridin-2-amine. MS (ESI): mass calcd. for C₁₆H₁₁ClFN₅, 327.1; m/z found, 328.1 [M+H]⁺. ¹H NMR (400 MHz, Methanol-d₄) δ 8.27-8.13 (m, 2H), 7.83 (s, 1H), 7.54 (ddd, J=8.8, 4.6, 0.7 Hz, 1H), 7.47 (td, J=8.6, 2.9 Hz, 1H), 7.31 (s, 1H), 7.00 (d, J=5.0 Hz, 1H), 4.07 (s, 3H).

### Example 77: 3-Fluoro-4-[3-(5-fluoro-2-pyridyl)-1-methyl-pyrazol-4-yl]-1H-pyrrolo[2,3-b]pyridine

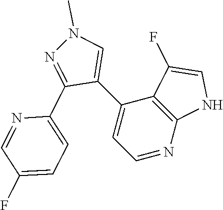 [SMILES: Cn1cc(-c2ccnc3[nH]cc(F)c23)c(-c2ccc(F)cn2)n1]

The title compound was prepared in a manner analogous to Example 1, Step A, except using 4-chloro-3-fluoro-1H-pyrrol[2,3-b]pyridine instead of 4-bromopyridin-2-amine. MS (ESI): mass calcd. for C₁₆H₁₁F₂N₅, 311.1; m/z found, 312.1 [M+H]⁺. ¹H NMR (500 MHz, CDCl₃) δ 9.33 (s, 1H), 8.38 (dt, J=2.9, 0.5 Hz, 1H), 8.23 (d, J=5.0 Hz, 1H), 7.69 (d, J=2.4 Hz, 1H), 7.59-7.49 (m, 1H), 7.38-7.29 (m, 1H), 7.03 (t, J=2.4 Hz, 1H), 6.94 (d, J=5.0 Hz, 1H), 4.06 (s, 3H).

### Example 78: 5-Fluoro-4-[3-(5-fluoro-2-pyridyl)-1-methyl-pyrazol-4-yl]-1H-pyrrolo[2,3-b]pyridine

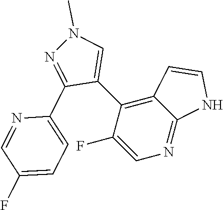 [SMILES: Cn1cc(-c2c(F)cnc3[nH]ccc23)c(-c2ccc(F)cn2)n1]

The title compound was prepared in a manner analogous to Example 1, Step A, except using 4-bromo-5-fluoro-1H-pyrrolo[2,3-b]pyridine instead of 4-bromopyridin-2-amine, dioxane and water instead of dioxane, for 16 hours. MS (ESI): mass calcd. for C₁₆H₁₁F₂N₅, 311.1; m/z found, 312.1 [M+H]⁺. ¹H NMR (500 MHz, CDCl₃) δ 8.97 (s, 1H), 8.35 (d, J=2.9 Hz, 1H), 8.19 (d, J=2.8 Hz, 1H), 7.72 (d, J=0.9 Hz, 1H), 7.48 (dd, J=8.7, 4.4 Hz, 1H), 7.31-7.21 (m, 2H), 6.17-6.10 (m, 1H), 4.09 (s, 3H).

### Example 79: 4-[3-(5-Fluoro-2-pyridyl)-1-methyl-pyrazol-4-yl]-5-methyl-1H-pyrrolo[2,3-b]pyridine

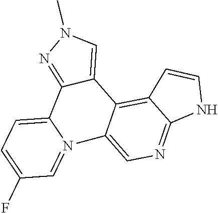

The title compound was prepared in a manner analogous to Example 80, using 4-chloro-5-methyl-1-tosyl-1H-pyrrolo[2,3-b]pyridine (Intermediate 53) instead of 4-chloro-6-methyl-1-tosyl-1H-pyrrolo[2,3-b]pyridine (Intermediate 52) in Step A. MS (ESI): mass calcd. for C₁₇H₁₄FN₅, 307.1; m/z found, 308.1 [M+H]⁺. ¹H NMR (500 MHz, CDCl₃) δ 9.41 (s, 1H), 8.43-8.39 (m, 1H), 8.21 (s, 1H), 7.50 (s, 1H), 7.21 (dd, J=3.5, 2.3 Hz, 1H), 7.15-7.07 (m, 2H), 6.16 (dd, J=3.5, 1.9 Hz, 1H), 4.09 (s, 3H), 2.13 (s, 3H).

### Example 80: 4-[3-(5-Fluoro-2-pyridyl)-1-methyl-pyrazol-4-yl]-6-methyl-1H-pyrrolo[2,3-b]pyridine

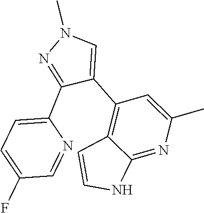 [SMILES: Cc1cc(-c2cn(C)nc2-c2ccc(F)cn2)c2cc[nH]c2n1]

Step A: 4-(3-(5-Fluoropyridin-2-yl)-1-methyl-1H-pyrazol-4-yl)-6-methyl-1-tosyl-1H-pyrrolo[2,3-b]pyridine. The title compound was prepared in a manner analogous to Example 1, Step A, except using 4-chloro-6-methyl-1-tosyl-1H-pyrrolo[2,3-b]pyridine (Intermediate 52) instead of 4-bromopyridin-2-amine. MS (ESI): mass calcd. for C₂₄H₂₀FN₅O₂S, 461.1; m/z found, 462.1 [M+H]⁺.

Step B: 4-[3-(5-Fluoro-2-pyridyl)-1-methyl-pyrazol-4-yl]-6-methyl-1H-pyrrolo[2,3-b]pyridine. To 4-(3-(5-fluoropyridin-2-yl)-1-methyl-1H-pyrazol-4-yl)-6-methyl-1-tosyl-1H-pyrrolo[2,3-b]pyridine (51 mg, 0.11 mmol) was added TBAF (1 M in THF, 1.1 mL). The reaction mixture was stirred at room temperature for 16 hours then heated up to 45° C. for 20 hours followed by the addition of more TBAF (1 M in THF, 1.1 mL). The reaction mixture was heated to 45° C. for another 16 hours. Solvent was evaporated purification via silica gel chromatography (0% to 100% EtOAc/hexanes) gave the title compound (20 mg, 57% yield). MS (ESI): mass calcd. for C₁₇H₁₄FN₅, 307.1; m/z found, 308.1 [M+H]⁺. ¹H NMR (500 MHz, CDCl₃) δ 9.52 (s, 1H), 8.46 (d, J=2.9 Hz, 1H), 7.66 (s, 1H), 7.34 (dd, J=8.7, 4.4 Hz, 1H), 7.23 (td, J=8.5, 3.0 Hz, 1H), 7.13 (dd, J=3.5, 2.1 Hz, 1H), 6.88 (s, 1H), 6.10 (dd, J=3.6, 1.9 Hz, 1H), 4.07 (s, 3H), 2.60 (s, 3H).

### Example 81: 4-(3-(5-Fluoropyridin-2-yl)-1-methyl-1H-pyrazol-4-yl)-2-methyl-1H-pyrrolo[2,3-b]pyridine

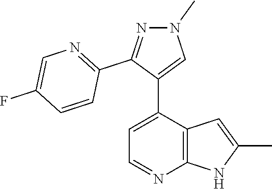 [SMILES: Cc1cc2c(-c3cn(C)nc3-c3ccc(F)cn3)ccnc2[nH]1]

The title compound was prepared in a manner analogous to Example 1, Step A, except using potassium phosphate tribasic instead of aq Na₂CO₃; DMF instead of dioxane; and using 4-chloro-2-methyl-1H-pyrrolo[2,3-b]pyridine instead of 4-bromopyridin-2-amine; and the mixture was heated to 100° C. for 16 h. MS (ESI): mass calcd. for C₁₇H₁₄FN₅, 307.1; m/z found, 308.10 [M+H]⁺. ¹H NMR (600 MHz, DMSO-d₆) δ 12.52-12.27 (m, 1H), 8.48-8.28 (m, 2H), 8.22-8.08 (m, 1H), 7.92-7.76 (m, 2H), 7.14-7.00 (m, 1H), 6.37-6.23 (m, 1H), 4.01 (s, 3H), 2.42 (s, 3H).

### Example 82: 4-[3-(5-Fluoro-2-pyridyl)-1-methyl-pyrazol-4-yl]-3-methyl-1H-pyrrolo[2,3-b]pyridine

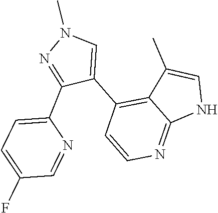 [SMILES: Cc1c[nH]c2nccc(-c3cn(C)nc3-c3ccc(F)cn3)c12]

The title compound was prepared in a manner analogous to Example 1, Step A, except using 4-bromo-3-methyl-1H-pyrrol[2,3-b]pyridine instead of 4-bromopyridin-2-amine. MS (ESI): mass calcd. for C₁₇H₁₄FN₅, 307.3; m/z found, 308.1 [M+H]⁺. ¹H NMR (400 MHz, CDCl₃) δ 8.50 (s, 1H), 8.38-8.28 (m, 1H), 8.17 (d, J=4.9 Hz, 1H), 7.39 (s, 1H), 7.11-6.98 (m, 2H), 6.91 (d, J=2.1 Hz, 1H), 6.84 (d, J=4.9 Hz, 1H), 4.00 (s, 3H), 1.77 (d, J=1.2 Hz, 3H).

### Example 83: 4-(3-(5-Fluoropyridin-2-yl)-1-methyl-1H-pyrazol-4-yl)-2-isopropyl-1H-pyrrolo[2,3-b]pyridine

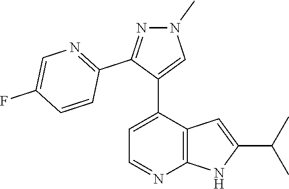 [SMILES: CC(C)c1cc2c(-c3cn(C)nc3-c3ccc(F)cn3)ccnc2[nH]1]

The title compound was prepared in a manner analogous to Example 1, Step A, except using potassium phosphate tribasic instead of aq Na₂CO₃; DMF instead of dioxane; 4-chloro-2-isopropyl-1H-pyrrolo[2,3-b]pyridine (Intermediate 44) instead of 4-bromopyridin-2-amine; and the mixture was heated to 100° C. for 16 h. MS (ESI): mass calcd. for C₁₉H₁₈FN₅, 335.2; m/z found, 336.1 [M+H]⁺. ¹H NMR (400 MHz, DMSO-d₆) δ 12.21 (br s, 1H), 8.38 (br s, 1H), 8.33 (br s, 1H), 8.13 (br s, 1H), 7.81 (br s, 2H), 7.01 (br s, 1H), 6.06 (br s, 1H), 4.02 (br s, 3H), 3.03 (dt, J=13.69, 6.94 Hz, 1H), 1.24 (d, J=6.80 Hz, 6H).

### Example 84: 2-(Difluoromethyl)-4-(3-(5-fluoropyridin-2-yl)-1-methyl-1H-pyrazol-4-yl)-1H-pyrrolo[2,3-b]pyridine

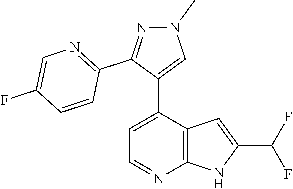 [SMILES: Cn1cc(-c2ccnc3[nH]c(C(F)F)cc23)c(-c2ccc(F)cn2)n1]

Step A. 2-(Difluoromethyl)-4-(3-(5-fluoropyridin-2-yl)-1-methyl-1H-pyrazol-4-yl)-1-(phenylsulfonyl)-1H-pyrrolo[2,3-b]pyridine. The title compound was prepared in a manner analogous to Example 19, Step A, except using 4-bromo-2-(difluoromethyl)-1-(phenylsulfonyl)-1H-pyrrolo[2,3-b]pyridine (Intermediate 43) instead of 4-(4-bromo-1-methyl-1H-pyrazol-3-yl)-3-fluoropyridine (Intermediate 19) and 2-(3-(5-fluoropyridin-2-yl)-1-methyl-1H-pyrazol-4-yl)-2-hydroxy-4,4,5,5-tetramethyl-1,3,2-dioxaborolan-2-uide lithium salt (Intermediate 5) instead of 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 1). MS (ESI): mass calcd. for C₂₃H₁₆F₃N₅O₂S, 483.1; m/z found, 484.1 [M+H]⁺.

Step B. 2-(Difluoromethyl)-4-(3-(5-fluoropyridin-2-yl)-1-methyl-1H-pyrazol-4-yl)-1H-pyrrolo[2,3-b]pyridine. To a solution of 2-(difluoromethyl)-4-(3-(5-fluoropyridin-2-yl)-1-methyl-1H-pyrazol-4-yl)-1-(phenylsulfonyl)-1H-pyrrolo[2,3-b]pyridine (98 mg, 0.2 mmol) in THE (2 mL) was added TBAF (0.61 mL of 1 M in THF, 0.61 mmol) and the mixture stirred at room temperature for 6 h. The reaction mixture was diluted with CH₂Cl₁₂ then washed with H₂O (3×). The organics were concentrated. Purification by chromatography (silica gel, 1% methanol saturated with ammonia/9% methanol/CH₂Cl₂) afforded 45 mg (64%) of the title compound. MS (ESI): mass calcd. for C₁₇H₁₂F₃N₅, 343.1; m/z found, 344.1 [M+H]⁺. ¹H NMR (500 MHz, DMSO-d₆) δ 12.33 (s, 1H) 8.36 (d, J=2.27 Hz, 1H) 8.23 (s, 1H) 8.20 (d, J=4.91 Hz, 1H) 7.74-7.79 (m, 2H) 7.13 (t, J=54.20 Hz, 1H) 6.90 (d, J=4.91 Hz, 1H) 6.51 (d, J=2.08 Hz, 1H) 3.99 (s, 3H).

### Example 85: 4-(3-(5-Fluoropyridin-2-yl)-1-methyl-1H-pyrazol-4-yl)-2-(trifluoromethyl)-1H-pyrrolo[2,3-b]pyridine

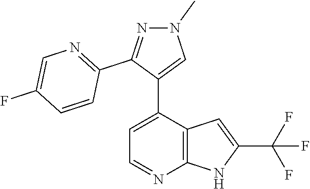 [SMILES: Cn1cc(-c2ccnc3[nH]c(C(F)(F)F)cc23)c(-c2ccc(F)cn2)n1]

Step A. 4-(3-(5-Fluoropyridin-2-yl)-1-methyl-1H-pyrazol-4-yl)-2-(trifluoromethyl)-1-((2-(trimethylsilyl)ethoxy)methyl)-1H-pyrrolo[2,3-b]pyridine. The title compound was prepared in a manner analogous to Example 1, Step A, except using 4-chloro-2-(trifluoromethyl)-1-((2-(trimethylsilyl)ethoxy)methyl)-1H-pyrrolo[2,3-b]pyridine (Intermediate 46) instead of 4-bromopyridin-2-amine; and using cesium carbonate instead of aq Na₂CO₃, dioxane and water instead of dioxane, at 80° C. for 16 hours. MS (ESI): mass calcd. for C₂₃H₂₅F₄N₅OSi, 491.2; m/z found, 492.2 [M+H]⁺.

Step B. 4-(3-(5-Fluoropyridin-2-yl)-1-methyl-1H-pyrazol-4-yl)-2-(trifluoromethyl)-1H-pyrrolo[2,3-b]pyridine. To a solution of 4-(3-(5-fluoropyridin-2-yl)-1-methyl-1H-pyrazol-4-yl)-2-(trifluoromethyl)-1-((2-(trimethylsilyl)ethoxy)methyl)-1H-pyrrolo[2,3-b]pyridine (54 mg, 0.11 mmol) in dichloromethane (0.2 mL) was added trifluoroacetic acid (0.17 mL, 2.2 mmol). The reaction was stirred at room temperature for 2 h then quenched with saturated aqueous Na₂CO₃. The resulting mixture was extracted with 4:1 dichloromethane:isopropanol then the combined organics were concentrated. Purification by chromatography (silica gel, 1% methanol saturated with ammonia/9% methanol/CH₂Cl₂) afforded 23 mg (58%) of the title compound. MS (ESI): mass calcd. for C₁₇H₁₁F₄N₅, 361.1; m/z found, 362.1 [M+H]⁺. ¹H NMR (400 MHz, DMSO-d₆) δ 12.84 (br s, 1H) 8.37 (d, J=2.38 Hz, 1H) 8.29 (d, J=4.37 Hz, 1H) 8.28 (s, 1H) 7.82-7.74 (m, 2H) 6.98 (d, J=5.00 Hz, 1H) 6.72 (d, J=1.13 Hz, 1H) 4.00 (s, 3H).

### Example 86: 4-(3-(5-Fluoropyridin-2-yl)-1-methyl-1H-pyrazol-4-yl)-3-(trifluoromethyl)-1H-pyrrolo[2,3-b]pyridine

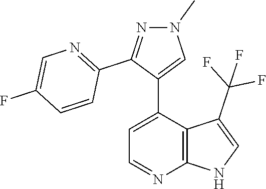 [SMILES: Cn1cc(-c2ccnc3[nH]cc(C(F)(F)F)c23)c(-c2ccc(F)cn2)n1]

The title compound was prepared in a manner analogous to Example 85, Steps A-B, except using 4-chloro-3-(trifluoromethyl)-1-((2-(trimethylsilyl)ethoxy)methyl)-1H-pyrrolo[2,3-b]pyridine (Intermediate 47) instead of 4-chloro-2-(trifluoromethyl)-1-((2-(trimethylsilyl)ethoxy)methyl)-1H-pyrrolo[2,3-b]pyridine (Intermediate 46) in Step A. MS (ESI): mass calcd. for C₁₇H₁₁F₄N₅, 361.1; m/z found, 362.1 [M+H]⁺. ¹H NMR (400 MHz, DMSO-d₆) δ 12.44 (br s, 1H) 8.28 (d, J=4.88 Hz, 1H) 8.13 (d, J=3.00 Hz, 1H) 8.01 (s, 1H) 7.83 (s, 1H) 7.77 (dd, J=8.82, 4.19 Hz, 1H) 7.62 (td, J=8.82, 3.00 Hz, 1H) 6.97 (d, J=4.88 Hz, 1H) 3.96 (s, 3H).

### Example 87: 4-[3-(5-Fluoro-2-pyridyl)-1-methyl-pyrazol-4-yl]-5-(trifluoromethyl)-1H-pyrrolo[2,3-b]pyridine

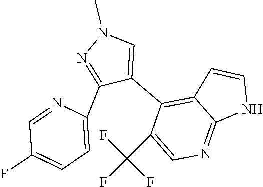 [SMILES: Cn1cc(-c2c(C(F)(F)F)cnc3[nH]ccc23)c(-c2ccc(F)cn2)n1]

The title compound was prepared in a manner analogous to Example 19, Step A, except using 4-chloro-5-(trifluoromethyl)-1H-pyrrolo[2,3-b]pyridine instead of 4-(4-bromo-1-methyl-1H-pyrazol-3-yl)-3-fluoropyridine (Intermediate 19) and 2-(3-(5-fluoropyridin-2-yl)-1-methyl-1H-pyrazol-4-yl)-2-hydroxy-4,4,5,5-tetramethyl-1,3,2-dioxaborolan-2-uide lithium salt (Intermediate 5) instead of 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 1). MS (ESI): mass calcd. for C₁₇H₁₁F₄N₅, 361.1; m/z found, 362.1 [M+H]⁺. ¹H NMR (400 MHz, CDCl₃) δ 10.57 (s, 1H), 8.72 (s, 1H), 8.26 (d, J=2.9 Hz, 1H), 7.53 (s, 1H), 7.29 (dd, J=3.6, 1.7 Hz, 1H), 7.23 (dd, J=8.8, 4.4 Hz, 1H), 7.13 (td, J=8.4, 2.9 Hz, 1H), 6.12 (dd, J=3.6, 1.6 Hz, 1H), 4.09 (s, 3H).

### Example 88: 4-[3-(5-Fluoro-2-pyridyl)-1-methyl-pyrazol-4-yl]-1H-pyrrolo[2,3-b]pyridine-3-carbonitrile

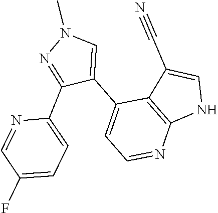 [SMILES: Cn1cc(-c2ccnc3[nH]cc(C#N)c23)c(-c2ccc(F)cn2)n1]

The title compound was prepared in a manner analogous to Example 1, Step A, except using 4-chloro-1H-pyrrolo[2,3-b]pyridine-3-carbonitrile instead of 4-bromopyridin-2-amine. MS (ESI): mass calcd. for C₁₇H₁₁FN₆, 318.1; m/z found, 319.0 [M+H]⁺. ¹H NMR (400 MHz, CDCl₃) δ 8.27 (dd, J=8.9, 5.1 Hz, 1H), 8.13 (d, J=3.0 Hz, 1H), 7.78 (s, 1H), 7.73-7.67 (m, 1H), 7.65 (s, 1H), 7.37-7.18 (m, 2H), 7.07 (d, J=5.0 Hz, 1H), 4.00 (s, 3H).

### Example 89: 4-[3-(5-Fluoro-2-pyridyl)-1-methyl-pyrazol-4-yl]-1H-pyrrolo[2,3-b]pyridine-5-carbonitrile

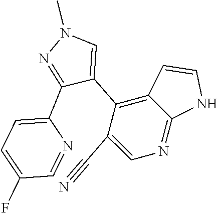 [SMILES: Cn1cc(-c2c(C#N)cnc3[nH]ccc23)c(-c2ccc(F)cn2)n1]

The title compound was prepared in a manner analogous to Example 1, Step A, using 4-chloro-1H-pyrrolo[2,3-b]pyridine-5-carbonitrile instead of 4-bromopyridin-2-amine and using cesium carbonate instead of aq Na₂CO₃, dioxane and water instead of dioxane, at 85° C. for 4 hours. MS (ESI): mass calcd. for C₁₇H₁₁FN₆, 318.1; m/z found, 319.1 [M+H]⁺. ¹H NMR (500 MHz, DMSO-d₆) δ 12.24 (s, 1H), 8.52 (s, 1H), 8.22 (d, J=3.3 Hz, 2H), 7.93 (dd, J=8.8, 4.5 Hz, 1H), 7.76 (td, J=8.8, 2.9 Hz, 1H), 7.59 (dd, J=3.5, 2.5 Hz, 1H), 6.29 (dd, J=3.5, 1.9 Hz, 1H), 4.04 (s, 3H).

### Example 90: 2-[4-[3-(5-Fluoro-2-pyridyl)-1-methyl-pyrazol-4-yl]-1H-pyrrolo[2,3-b]pyridin-5-yl]acetonitrile

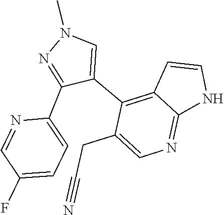 [SMILES: Cn1cc(-c2c(CC#N)cnc3[nH]ccc23)c(-c2ccc(F)cn2)n1]

The title compound was prepared in a manner analogous to Example 1, Step A, using 2-(4-chloro-1H-pyrrolo[2,3-b]pyridin-3-yl)acetonitrile instead of 4-bromopyridin-2-amine; and using cesium carbonate instead of aq Na₂CO₃, dioxane and water instead of dioxane, at 85° C. for 4 hours. MS (ESI): mass calcd. for C₁₈H₁₃FN₆, 332.1; m/z found, 333.1 [M+H]⁺. ¹H NMR (400 MHz, CDCl₃) δ 13.38 (s, 1H), 8.26 (d, J=6.1 Hz, 1H), 8.04 (d, J=2.8 Hz, 1H), 7.94 (dd, J=8.8, 4.4 Hz, 1H), 7.69 (s, 1H), 7.57 (s, 1H), 7.46 (ddd, J=8.8, 7.9, 2.8 Hz, 1H), 7.23 (d, J=6.0 Hz, 1H), 4.11 (s, 3H), 3.42 (s, 2H).

### Example 91: 4-(3-(5-Fluoropyridin-2-yl)-1-methyl-1H-pyrazol-4-yl)-5-(oxetan-3-yl)-1H-pyrrolo[2,3-b]pyridine

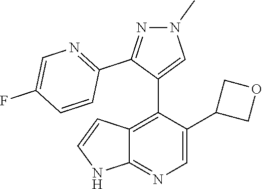 [SMILES: Cn1cc(-c2c(C3COC3)cnc3[nH]ccc23)c(-c2ccc(F)cn2)n1]

The title compound was prepared in a manner analogous to Example 1, Step A, except using 4-chloro-5-(oxetan-3-yl)-1H-pyrrolo[2,3-b]pyridine (Intermediate 48) instead of 4-bromopyridin-2-amine; and using cesium carbonate instead of aq Na₂CO₃, dioxane and water instead of dioxane. MS (ESI): mass calcd. for C₁₉H₁₆FN₅O, 349.1; m/z found, 350.1 [M+H]⁺. ¹H NMR (500 MHz, DMSO-d₆) δ 11.56 (br s, 1H) 8.49 (s, 1H) 8.20 (d, J=2.88 Hz, 1H) 7.91 (s, 1H) 7.80 (dd, J=8.82, 4.57 Hz, 1H) 7.69 (td, J=8.79, 2.94 Hz, 1H) 7.33 (s, 1H) 6.02 (dd, J=3.38, 2.00 Hz, 1H) 4.66 (dd, J=8.32, 5.94 Hz, 1H) 4.58 (t, J=6.44 Hz, 1H) 4.46 (dd, J=6.82, 5.94 Hz, 1H) 4.24-4.10 (m, 2H) 4.00 (s, 3H).

### Example 92: 4-(3-(5-Fluoropyridin-2-yl)-1-methyl-1H-pyrazol-4-yl)-2-(oxetan-3-yl)-1H-pyrrolo[2,3-b]pyridine

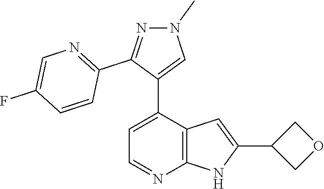 [SMILES: Cn1cc(-c2ccnc3[nH]c(C4COC4)cc23)c(-c2ccc(F)cn2)n1]

Step A. 4-(3-(5-Fluoropyridin-2-yl)-1-methyl-1H-pyrazol-4-yl)-2-(oxetan-3-yl)-1-(phenylsulfonyl)-1H-pyrrolo[2,3-b]pyridine. To a suspension of potassium phosphate tribasic (11 mg, 0.05 mmol) in DMF (0.4 mL) was added XPhos Pd G3 (4 mg, 0.005 mmol), 4-chloro-2-(oxetan-3-yl)-1-(phenylsulfonyl)-1H-pyrrolo[2,3-b]pyridine (Intermediate 54, 25 mg, 0.05 mmol), and lithium 2-(3-(5-fluoropyridin-2-yl)-1-methyl-1H-pyrazol-4-yl)-2-hydroxy-4,4,5,5-tetramethyl-1,3,2-dioxaborolan-2-uide (Intermediate 5, 18 mg, 0.06 mmol). The reaction vial was capped and degassed under vacuum then refilled with N₂. The mixture was heated to 100° C. for 16 h. The reaction mixture was cooled, diluted with H₂O (1 mL), and extracted with EtOAc (3×3 mL). The combined organics were dried (Na₂SO₄) and filtered to yield the title compound which was used in the next step without further purification. MS (ESI): mass calcd. for C₂₅H₂₀FN₅O₃S, 489.1; m/z found, 490.1 [M+H]⁺.

Step B. 4-(3-(5-Fluoropyridin-2-yl)-1-methyl-1H-pyrazol-4-yl)-2-(oxetan-3-yl)-1H-pyrrolo[2,3-b]pyridine. To a solution of 4-(3-(5-fluoropyridin-2-yl)-1-methyl-1H-pyrazol-4-yl)-2-(oxetan-3-yl)-1-(phenylsulfonyl)-1H-pyrrolo[2,3-b]pyridine (24 mg, 0.05 mmol) in THE (0.3 mL) was added sodium tert-butoxide (5 mg, 0.05 mmol) and the mixture heated to 100° C. under microwave irradiation for 4 h. The reaction mixture was filtered and concentrated. Purification by chromatography (silica gel, 1% methanol saturated with ammonia/9% methanol/CH₂Cl₂) afforded 3 mg (17%) of the title compound. MS (ESI): mass calcd. for C₁₉H₁₆FN₅O, 349.1; m/z found, 350.1 [M+H]⁺. ¹H NMR (600 MHz, Methanol-d₄) δ 8.46-8.26 (m, 1H) 8.14-7.99 (m, 2H) 7.70-7.47 (m, 2H) 6.98-6.85 (m, 1H) 6.23-6.11 (m, 1H) 5.06-4.96 (m, 2H) 4.79-4.70 (m, 2H) 4.48-4.35 (m, 1H) 4.16-3.99 (m, 3H).

### Example 93: 4-[3-(5-Fluoro-2-pyridyl)-1-methyl-pyrazol-4-yl]-3-(oxetan-3-yl)-1H-pyrrolo[2,3-b]pyridine

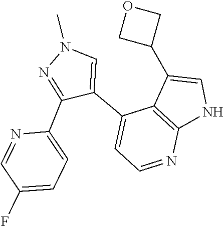 [SMILES: Cn1cc(-c2ccnc3[nH]cc(C4COC4)c23)c(-c2ccc(F)cn2)n1]

Under air, a stock solution of the nickel catalyst was prepared by charging an oven-dried 2-dram vial, equipped with a magnetic stir bar, with NiCl2·glyme (5.4 mg, 25 μmol), 4,4′-di(tert-butyl)-2,2′-dipyridyl (8.0 mg, 30 μmol) and DME (2.0 mL). This nickel solution was stirred under nitrogen for 10 minutes. Under air, a separate 20 mL vial equipped with a magnetic stir bar was charged with 3-bromo-4-(3-(5-fluoropyridin-2-yl)-1-methyl-1H-pyrazol-4-yl)-1H-pyrrolo[2,3-b]pyridine (Example 75, 66.7 mg, 0.179 mmol), Ir(dFCF₃ppy)₂(dtbbpy)(PF₆) (4 mg, 0.0036 mmol), 3-bromooxetane (95.6 μL, 1.08 mmol) and DME (1.5 mL), DMA (1 mL). Tris(trimethylsilyl)silane (0.34 mL, 1.08 mmol) and 2,6-dimethylpyridine (0.21 mL, 1.8 mmol) were added followed by addition of the nickel stock solution (0.23 mL, 2.7 μmol, 0.01 eq) before the reaction vial was capped. The resulting brownish reaction solution was sparged with nitrogen for 15 minutes then the reaction vial was sealed with parafilm and exposed to blue LEDs while stirring for 24 hours. The reaction mixture was filtered and the filtrate was purified by reversed phase HPLC Method C, to afford 18 mg crude product; which was further purified by SFC, Method A, to afford 2 mg (1.3%) of the title compound. MS (ESI): mass calcd. for C₁₉H₁₆FN₅O, 349.1; m/z found, 350.1 [M+H]⁺. ¹H NMR (400 MHz, Methanol-d₄) δ 8.24 (d, J=2.8 Hz, 1H), 8.17 (d, J=5.0 Hz, 1H), 7.76 (s, 1H), 7.54-7.41 (m, 2H), 7.39 (d, J=1.2 Hz, 1H), 6.92 (d, J=4.9 Hz, 1H), 4.51 (dd, J=7.1, 5.7 Hz, 2H), 4.41 (dd, J=8.2, 5.7 Hz, 2H), 4.09 (s, 3H), 4.07-3.95 (m, 1H).

### Example 94: 3-(4-(3-(5-Fluoropyridin-2-yl)-1-methyl-1H-pyrazol-4-yl)-1H-pyrrolo[2,3-b]pyridin-2-yl)oxetan-3-ol

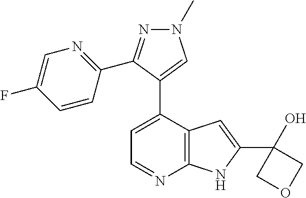 [SMILES: Cn1cc(-c2ccnc3[nH]c(C4(O)COC4)cc23)c(-c2ccc(F)cn2)n1]

The title compound was prepared in a manner analogous to Example 1, Step A, except using 3-(4-chloro-1H-pyrrolo[2,3-b]pyridin-2-yl)oxetan-3-ol (Intermediate 49) instead of 4-bromopyridin-2-amine; and using cesium carbonate instead of aq Na₂CO₃, dioxane and water instead of dioxane. MS (ESI): mass calcd. for C₁₉H₁₆FN₅O₂, 365.1; m/z found, 366.1 [M+H]⁺. ¹H NMR (400 MHz, DMSO-d₆) δ 11.71-11.49 (m, 1H) 8.48-8.32 (m, 1H) 8.26-8.10 (m, 1H) 8.10-7.95 (m, 1H) 7.81-7.66 (m, 2H) 6.89-6.70 (m, 1H) 6.48-6.35 (m, 1H) 6.35-6.16 (m, 1H) 4.76 (d, J=6.50 Hz, 2H) 4.68 (d, J=6.50 Hz, 2H) 4.11-3.88 (m, 3H).

### Example 95: 4-(3-(5-Fluoropyridin-2-yl)-1-methyl-1H-pyrazol-4-yl)-5-(oxetan-3-ylmethyl)-1H-pyrrolo[2,3-b]pyridine

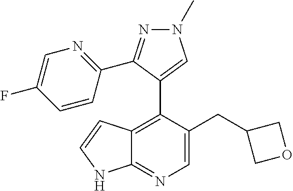 [SMILES: Cn1cc(-c2c(CC3COC3)cnc3[nH]ccc23)c(-c2ccc(F)cn2)n1]

The title compound was prepared in a manner analogous to Example 92, Step A, except using 4-chloro-5-(oxetan-3-ylmethyl)-1H-pyrrolo[2,3-b]pyridine (Intermediate 50) instead of 4-chloro-2-(oxetan-3-yl)-1-(phenylsulfonyl)-1H-pyrrolo[2,3-b]pyridine (Intermediate 54); and using [1,1′-bis(diphenylphosphino)ferrocene]dichloropalladium(II) instead of XPhos Pd G3; potassium phosphate tribasic with water was used; and the reaction was heated to 90° C. for 6 h. MS (ESI): mass calcd. for C₂₀H₁₈FN₅O, 363.2; m/z found, 364.1 [M+H]⁺. ¹H NMR (500 MHz, DMSO-d₆) δ 11.44 (br s, 1H) 8.21 (d, J=2.88 Hz, 1H) 8.03 (s, 1H) 7.92 (s, 1H) 7.76-7.71 (m, 1H) 7.65 (td, J=8.79, 2.94 Hz, 1H) 7.28-7.23 (m, 1H) 5.88 (dd, J=3.44, 1.94 Hz, 1H) 4.48-4.38 (m, 2H) 4.12 (dt, J=16.54, 6.18 Hz, 2H) 4.01 (s, 3H) 3.11-3.00 (m, 1H) 2.91-2.71 (m, 2H).

### Example 96: (R/S)-3-(4-(3-(5-Fluoropyridin-2-yl)-1-methyl-1H-pyrazol-4-yl)-1H-pyrrolo[2,3-b]pyridin-2-yl)tetrahydrofuran-3-ol

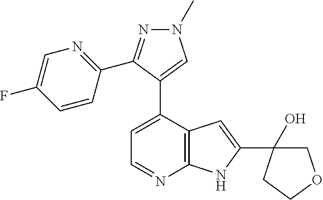 [SMILES: Cn1cc(-c2ccnc3[nH]c(C4(O)CCOC4)cc23)c(-c2ccc(F)cn2)n1]

The title compound was prepared in a manner analogous to Example 1, Step A, except using (R/S)-3-(4-chloro-1H-pyrrolo[2,3-b]pyridin-2-yl)tetrahydrofuran-3-ol (Intermediate 51) instead of 4-bromopyridin-2-amine; and using cesium carbonate instead of aq Na₂CO₃, dioxane and water instead of dioxane. MS (ESI): mass calcd. for C₂₀H₁₈FN₅O₂, 379.1; m/z found, 380.1 [M+H]⁺. ¹H NMR (400 MHz, DMSO-d₆) δ 11.50 (s, 1H) 8.38 (d, J=2.38 Hz, 1H) 8.16 (s, 1H) 8.02 (d, J=5.00 Hz, 1H) 7.75-7.70 (m, 2H) 6.77 (d, J=5.00 Hz, 1H) 6.14 (d, J=2.13 Hz, 1H) 5.53 (s, 1H) 4.02-4.00 (m, 3H) 3.99-3.88 (m, 2H) 3.80 (s, 2H) 2.36-2.25 (m, 2H).

### Example 97: (R/S)-4-[3-(5-Fluoro-2-pyridyl)-1-methyl-pyrazol-4-yl]-3-(tetrahydrofuran-3-ylmethyl)-1H-pyrrolo[2,3-b]pyridine

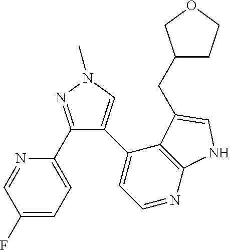 [SMILES: Cn1cc(-c2ccnc3[nH]cc(CC4CCOC4)c23)c(-c2ccc(F)cn2)n1]

The title compound was prepared in a manner analogous to Example 93, except using 3-(bromomethyl)tetrahydrofuran instead of 3-bromooxetane. MS (ESI): mass calcd. for C₂₁H₂₀FN₅O, 377.2; m/z found, 378.2 [M+H]⁺. ¹H NMR (400 MHz, Methanol-d₄) δ 8.31 (d, J=6.1 Hz, 1H), 8.04 (d, J=2.9 Hz, 1H), 8.01-7.95 (m, 2H), 7.67-7.54 (m, 1H), 7.47 (d, J=1.1 Hz, 1H), 7.34 (d, J=6.2 Hz, 1H), 4.11 (s, 3H), 3.76-3.62 (m, 1H), 3.62-3.44 (m, 2H), 3.18 (dd, J=8.4, 6.1 Hz, 1H), 2.45 (dd, J=7.4, 1.0 Hz, 2H), 2.21-2.03 (m, 1H), 1.79-1.64 (m, 1H), 1.46-1.26 (m, 1H).

### Example 98: 4-(3-(5-Fluoropyridin-2-yl)-1-methyl-1H-pyrazol-4-yl)-1H-pyrazolo[3,4-b]pyridine

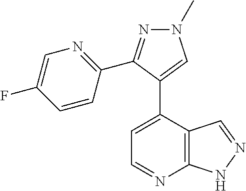 [SMILES: Cn1cc(-c2ccnc3[nH]ncc23)c(-c2ccc(F)cn2)n1]

The title compound was prepared in a manner analogous to Example 1, Step A, except using potassium phosphate tribasic instead of aq Na₂CO₃; DMF instead of dioxane; 4-bromo-1H-pyrazolo[3,4-b]pyridine instead of 4-bromopyridin-2-amine; and the mixture was heated to 100° C. for 16 h. MS (ESI): mass calcd. for C₁₅H₁₁FN₆, 294.1; m/z found, 295.1 [M+H]⁺. ¹H NMR (600 MHz, DMSO-d₆) δ 13.59 (br s, 1H), 8.45-8.31 (m, 3H), 7.98-7.72 (m, 3H), 7.02-6.96 (m, 1H), 4.03-3.98 (m, 3H).

### Example 99: 4-(3-(5-Fluoropyridin-2-yl)-1-methyl-1H-pyrazol-4-yl)-6-methyl-1H-pyrazolo[3,4-b]pyridine

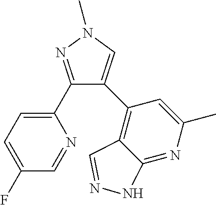 [SMILES: Cc1cc(-c2cn(C)nc2-c2ccc(F)cn2)c2cn[nH]c2n1]

The title compound was prepared in a manner analogous to Example 25, Steps-A-B, except using 4-bromo-6-methyl-1-((2-(trimethylsilyl)ethoxy)methyl)-1H-pyrazolo[3,4-b]pyridine (Intermediate 70) instead of 2-(4-bromo-1-methyl-1H-pyrazol-3-yl)-5-chloropyridine (Intermediate 8) and 2-(3-(5-fluoropyridin-2-yl)-1-methyl-1H-pyrazol-4-yl)-2-hydroxy-4,4,5,5-tetramethyl-1,3,2-dioxaborolan-2-uide lithium salt (Intermediate 5) instead of 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 1) in Step A. MS (ESI): mass calcd. for C₁₆H₁₃FN₆, 308.1; m/z found, 309.1 [M+H]⁺. ¹H NMR (400 MHz, Methanol-d₄) δ 8.31 (dt, J=3.0, 0.7 Hz, 1H), 8.13 (s, 1H), 7.76-7.71 (m, 1H), 7.64 (td, J=8.6, 2.9 Hz, 1H), 7.55 (s, 1H), 6.98 (s, 1H), 4.06 (s, 3H), 2.58 (s, 3H).

### Example 100: 4-[3-(5-Fluoro-2-pyridyl)-1-methyl-pyrazol-4-yl]-3,6-dimethyl-1H-pyrazolo[3,4-b]pyridine

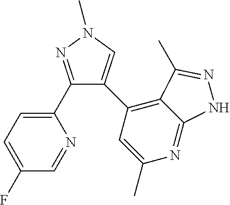 [SMILES: Cc1cc(-c2cn(C)nc2-c2ccc(F)cn2)c2c(C)n[nH]c2n1]

Step A: 1-Benzyl-4-(3-(5-fluoropyridin-2-yl)-1-methyl-1H-pyrazol-4-yl)-3,6-dimethyl-1H-pyrazolo[3,4-b]pyridine. The title compound was prepared in a manner analogous to Example 1, Step A, except using 1-benzyl-4-bromo-3,6-dimethyl-1H-pyrazolo[3,4-b]pyridine (Intermediate 41) instead of 4-bromopyridin-2-amine. MS (ESI): mass calcd. for C₂₅H₂₂FN₅, 411.2; m/z found, 412.3 [M+H]⁺.

Step B: 4-(3-(5-Fluoropyridin-2-yl)-1-methyl-1H-pyrazol-4-yl)-3,6-dimethyl-1H-pyrazolo[3,4-b]pyridine. To a solution of 1-benzyl-4-(3-(5-fluoropyridin-2-yl)-1-methyl-1H-pyrazol-4-yl)-3,6-dimethyl-1H-pyrazolo[3,4-b]pyridine (72 mg, 0.175 mmol) in MeOH (3.6 mL) was added PdCl₂ (31 mg, 0.175 mmol) and 1N HCl (58.3 μL, 0.0583 mmol). The resulting red mixture was stirred under H₂ balloon (vacuumed the reaction vessel and filled with H₂ three times) at 50° C. for 18 hours. The reaction mixture was then filtered through Celite®/diatomaceous earth and the solvent was evaporated. Purification via silica gel chromatography (0% to 100% EtOAc/DCM) gave the title compound (20.8 mg, 37% yield). MS (ESI): mass calcd. for C₁₇H₁₅FN₆, 322.1; m/z found, 323.3 [M+H]⁺. ¹H NMR (400 MHz, Methanol-d₄) δ 8.18 (d, J=2.9 Hz, 1H), 7.86 (s, 1H), 7.70 (dd, J=8.8, 4.4 Hz, 1H), 7.59-7.48 (m, 1H), 6.91 (s, 1H), 4.08 (s, 3H), 2.62 (s, 3H), 2.01 (s, 3H).

### Example 101: 6-Cyclopropyl-4-(3-(5-fluoropyridin-2-yl)-1-methyl-1H-pyrazol-4-yl)-1H-pyrazolo[3,4-b]pyridine

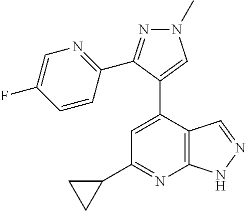 [SMILES: Cn1cc(-c2cc(C3CC3)nc3[nH]ncc23)c(-c2ccc(F)cn2)n1]

Step A. 1-Benzyl-6-cyclopropyl-4-(3-(5-fluoropyridin-2-yl)-1-methyl-1H-pyrazol-4-yl)-1H-pyrazolo[3,4-b]pyridine. The title compound was prepared in a manner analogous to Example 19, Step A, using 2-(3-(5-fluoropyridin-2-yl)-1-methyl-1H-pyrazol-4-yl)-2-hydroxy-4,4,5,5-tetramethyl-1,3,2-dioxaborolan-2-uide lithium salt (Intermediate 5) instead of 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 1) and using 1-benzyl-4-bromo-6-cyclopropyl-1H-pyrazolo[3,4-b]pyridine (Intermediate 40) instead of 4-(4-bromo-1-methyl-1H-pyrazol-3-yl)-3-fluoropyridine (Intermediate 19). MS (ESI): mass calcd. for C₂₅H₂₁FN₆, 424.2; m/z found, 425.3 [M+H]⁺.

Step B. 6-Cyclopropyl-4-(3-(5-fluoropyridin-2-yl)-1-methyl-1H-pyrazol-4-yl)-1H-pyrazolo[3,4-b]pyridine. A solution of 1-benzyl-6-cyclopropyl-4-(3-(5-fluoropyridin-2-yl)-1-methyl-1H-pyrazol-4-yl)-1H-pyrazolo[3,4-b]pyridine in neat H₂SO₄ (0.28 mL, 5.3 mmol) was heated to 70° C. for 1 h. The reaction mixture was cooled then quenched with aqueous NaOH and extracted with 4:1 CH₂Cl₂:IPA. The combined organics were dried (Na₂SO₄) and filtered. Purification by chromatography (silica gel, 1% methanol saturated with ammonia/9% methanol/CH₂Cl₂) afforded 10 mg (29%) of the title compound. MS (ESI): mass calcd. for C₁₈H₁₅FN₆, 334.1; m/z found, 335.2 [M+H]⁺. ¹H NMR (400 MHz, DMSO-d₆) δ 13.25 (s, 1H), 8.39 (d, J=2.38 Hz, 1H), 8.31 (s, 1H), 7.87-7.72 (m, 2H), 7.52 (d, J=1.13 Hz, 1H), 6.94 (s, 1H), 4.00 (s, 3H), 2.17-2.01 (m, 1H), 1.04-0.88 (m, 4H).

### Example 102: 4-(3-(5-Fluoropyridin-2-yl)-1-methyl-1H-pyrazol-4-yl)-7H-pyrrolo[2,3-d]pyrimidine

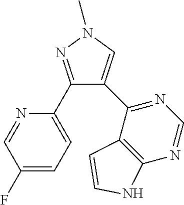 [SMILES: Cn1cc(-c2ncnc3[nH]ccc23)c(-c2ccc(F)cn2)n1]

The title compound was prepared in a manner analogous to Example 25, Step A, using 4-bromo-7H-pyrrolo[2,3-d]pyrimidine instead of 2-(4-bromo-1-methyl-1H-pyrazol-3-yl)-5-chloropyridine (Intermediate 8) and using 2-(3-(5-fluoropyridin-2-yl)-1-methyl-1H-pyrazol-4-yl)-2-hydroxy-4,4,5,5-tetramethyl-1,3,2-dioxaborolan-2-uide lithium salt (Intermediate 5) instead of 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 1). MS (ESI): mass calcd. for C₁₅H₁₁FN₆, 294.1; m/z found, 295.2 [M+H]⁺. ¹H NMR (500 MHz, DMSO-d₆) δ 11.96 (br s, 1H), 8.57 (s, 1H), 8.34-8.33 (m, 2H), 7.83-7.66 (m, 2H), 7.40 (d, J=3.5 Hz, 1H), 6.06 (d, J=3.5 Hz, 1H), 4.00 (s, 3H).

### Example 103: 8-[3-(5-Fluoro-2-pyridyl)-1-methyl-pyrazol-4-yl]-2-methoxy-1,5-naphthyridine

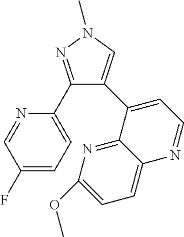 [SMILES: COc1ccc2nccc(-c3cn(C)nc3-c3ccc(F)cn3)c2n1]

The title compound was prepared in a manner analogous to Example 19, Step A, except using 2-(3-(5-fluoropyridin-2-yl)-1-methyl-1H-pyrazol-4-yl)-2-hydroxy-4,4,5,5-tetramethyl-1,3,2-dioxaborolan-2-uide lithium salt (Intermediate 5) instead of 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 1) and using 8-bromo-2-methoxy-1,5-naphthyridine instead of 4-(4-bromo-1-methyl-1H-pyrazol-3-yl)-3-fluoropyridine (Intermediate 19). MS (ESI): mass calcd. for C₁₈H₁₄FN₅O, 335.1; m/z found, [M+H]=236.1 [M+H]⁺. ¹H NMR (400 MHz, Methanol-d₄) δ 8.63 (d, J=4.7 Hz, 1H), 8.29-8.06 (m, 3H), 7.81-7.65 (m, 1H), 7.64-7.43 (m, 2H), 7.13 (d, J=9.0 Hz, 1H), 4.08 (s, 3H), 3.67 (s, 3H).

### Example 104: 4-[3-(5-Fluoro-2-pyridyl)-1-methyl-pyrazol-4-yl]quinoline

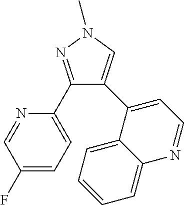 [SMILES: Cn1cc(-c2ccnc3ccccc23)c(-c2ccc(F)cn2)n1]

The title compound was prepared in a manner analogous to Example 25, Step A, using 2-(4-bromo-1-methyl-1H-pyrazol-3-yl)-5-fluoropyridine (Intermediate 18) instead of 2-(4-bromo-1-methyl-1H-pyrazol-3-yl)-5-chloropyridine (Intermediate 8); and using quinoline-4-boronic acid instead of 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 1). MS (ESI): mass calcd. for C₁₈H₁₃FN₄, 304.1; m/z found, 305.1 [M+H]⁺. ¹H NMR (400 MHz, CDCl₃) δ 8.91 (d, J=4.4 Hz, 1H), 8.33-8.23 (m, 1H), 8.21-8.09 (m, 1H), 7.86-7.74 (m, 1H), 7.72-7.63 (m, 1H), 7.59 (s, 1H), 7.43-7.36 (m, 1H), 7.33 (d, J=4.3 Hz, 1H), 7.31-7.29 (m, 1H), 7.23-7.13 (m, 1H), 4.13 (s, 3H).

### Example 105: 7-Chloro-4-[3-(5-fluoro-2-pyridyl)-1-methyl-pyrazol-4-yl]quinoline

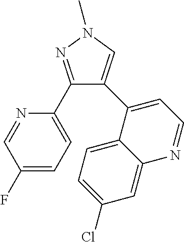 [SMILES: Cn1cc(-c2ccnc3cc(Cl)ccc23)c(-c2ccc(F)cn2)n1]

The title compound was prepared in a manner analogous to Example 19, Step A, except using 2-(4-bromo-1-methyl-1H-pyrazol-3-yl)-5-fluoropyridine (Intermediate 18) instead of 4-(4-bromo-1-methyl-1H-pyrazol-3-yl)-3-fluoropyridine (Intermediate 19); using 7-chloro-4-(4,4,5,5-tetramethyl-1,3,2-dioxaborolan-2-yl)quinoline instead of 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 1); using sodium carbonate instead of cesium carbonate and dioxane/water instead of 2-methyl-2-butanol. MS (ESI): mass calcd. for C₁₈H₁₂ClFN₄, 338.1; m/z found, 339.0 [M+H]⁺. ¹H NMR (400 MHz, DMSO-d₆+CCl₄) δ 8.80 (d, J=4.4 Hz, 1H), 8.00 (d, J=2.2 Hz, 1H), 7.98-7.93 (m, 1H), 7.93 (d, J=2.5 Hz, 1H), 7.90 (s, 1H), 7.70 (d, J=9.0 Hz, 1H), 7.54 (td, J=8.7, 8.6, 3.0 Hz, 1H), 7.31-7.25 (m, 2H), 4.05 (s, 3H).

### Example 106: 7-Fluoro-4-[3-(5-fluoro-2-pyridyl)-1-methyl-pyrazol-4-yl]quinoline

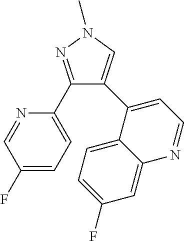 [SMILES: Cn1cc(-c2ccnc3cc(F)ccc23)c(-c2ccc(F)cn2)n1]

The title compound was prepared in a manner analogous to Example 1, Step A, except using 4-chloro-7-fluoroquinoline instead of 4-bromopyridin-2-amine, dioxane and water instead of dioxane, for 16 hours. MS (ESI): mass calcd. for C₁₈H₁₂F₂N₄, 322.1; m/z found, 323.0 [M+H]⁺. ¹H NMR (400 MHz, DMSO-d₆+CCl₄) δ 8.78 (d, J=4.5 Hz, 1H), 7.97-7.91 (m, 2H), 7.90 (s, 1H), 7.76 (dd, J=9.3, 6.2 Hz, 1H), 7.64 (dd, J=10.2, 2.7 Hz, 1H), 7.53 (td, J=8.7, 8.7, 2.9 Hz, 1H), 7.25 (d, J=4.5 Hz, 1H), 7.17 (td, J=8.7, 8.5, 2.7 Hz, 1H), 4.05 (s, 3H).

### Example 107: 4-[3-(5-Fluoro-2-pyridyl)-1-methyl-pyrazol-4-yl]-7-(trifluoromethyl)quinoline

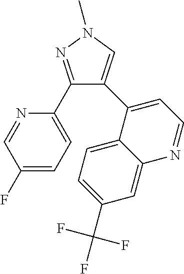 [SMILES: Cn1cc(-c2ccnc3cc(C(F)(F)F)ccc23)c(-c2ccc(F)cn2)n1]

The title compound was prepared in a manner analogous to Example 1, Step A, except using 4-chloro-7-(trifluoromethyl)quinoline instead of 4-bromopyridin-2-amine, dioxane and water instead of dioxane, for 16 hours. MS (ESI): mass calcd. for C₁₉H₁₂F₄N₄, 372.1; m/z found, 373.0 [M+H]⁺. ¹H NMR (400 MHz, CDCl₃) δ 8.95 (d, J=4.3 Hz, 1H), 8.41 (s, 1H), 8.11 (d, J=2.9 Hz, 1H), 7.85 (d, J=8.8 Hz, 1H), 7.55 (s, 1H), 7.51-7.46 (m, 2H), 7.39 (d, J=4.4 Hz, 1H), 7.24-7.17 (m, 1H), 4.08 (s, 3H).

### Example 108: 4-[3-(5-Fluoro-2-pyridyl)-1-methyl-pyrazol-4-yl]-7-methoxy-quinoline

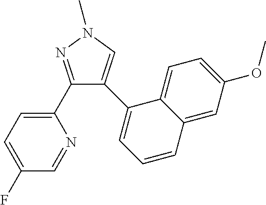 [SMILES: COc1ccc2c(-c3cn(C)nc3-c3ccc(F)cn3)cccc2c1]

The title compound was prepared in a manner analogous to Example 1, Step A, except using 4-chloro-7-methoxyquinoline instead of 4-bromopyridin-2-amine, dioxane and water instead of dioxane, for 16 hours. MS (ESI): mass calcd. for C₁₉H₁₅FN₄O, 334.1; m/z found, 335.0 [M+H]⁺. ¹H NMR (400 MHz, DMSO-d₆+CCl₄) δ 8.67 (d, J=4.5 Hz, 1H), 7.98 (d, J=2.9 Hz, 1H), 7.92-7.86 (m, 1H), 7.86 (s, 1H), 7.59 (d, J=9.2 Hz, 1H), 7.52 (td, J=8.6, 8.6, 2.9 Hz, 1H), 7.33 (d, J=2.6 Hz, 1H), 7.10 (d, J=4.4 Hz, 1H), 6.94 (dd, J=9.2, 2.7 Hz, 1H), 4.04 (s, 3H), 3.94 (s, 3H).

### Example 109: 4-[3-(5-Fluoro-2-pyridyl)-1-methyl-pyrazol-4-yl]-7-(trifluoromethoxy)quinoline

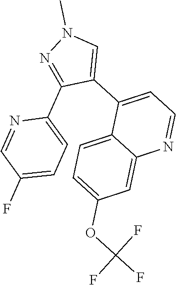 [SMILES: Cn1cc(-c2ccnc3cc(OC(F)(F)F)ccc23)c(-c2ccc(F)cn2)n1]

The title compound was prepared in a manner analogous to Example 1, Step A, except using 4-chloro-7-(trifluoromethoxy)quinoline instead of 4-bromopyridin-2-amine, dioxane and water instead of dioxane, for 16 hours. MS (ESI): mass calcd. for C₁₉H₁₂F₄N₄O, 388.1; m/z found, 389.1 [M+H]⁺. ¹H NMR (500 MHz, Methanol-d₄) δ 8.86 (d, J=4.6 Hz, 1H), 8.03 (d, J=2.9 Hz, 1H), 7.95 (s, 1H), 7.91-7.86 (m, 2H), 7.80 (dd, J=8.8, 4.5 Hz, 1H), 7.54 (td, J=8.6, 8.6, 2.9 Hz, 1H), 7.45 (d, J=4.6 Hz, 1H), 7.36-7.30 (m, 1H), 4.09 (s, 3H).

### Example 110: 4-[3-(5-Fluoro-2-pyridyl)-1-methyl-pyrazol-4-yl]-7-methoxy-2-methyl-quinoline

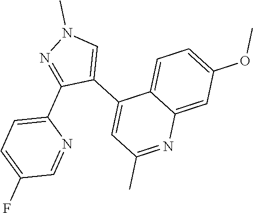 [SMILES: COc1ccc2c(-c3cn(C)nc3-c3ccc(F)cn3)cc(C)nc2c1]

The title compound was prepared in a manner analogous to Example 1, Step A, except using 4-chloro-7-methoxy-2-methylquinoline instead of 4-bromopyridin-2-amine, dioxane and water instead of dioxane, for 16 hours. MS (ESI): mass calcd. for C₂₀H₁₇FN₄O, 348.1; m/z found, 349.2 [M+H]⁺. ¹H NMR (400 MHz, CDCl₃) δ 8.27 (d, J=2.8 Hz, 1H), 7.54-7.47 (m, 2H), 7.35 (d, J=2.6 Hz, 1H), 7.20 (dd, J=4.5, 8.8 Hz, 1H), 7.11 (td, J=2.8, 8.4 Hz, 1H), 7.05 (s, 1H), 6.89 (dd, J=2.4, 9.1 Hz, 1H), 4.05 (s, 3H), 3.89 (s, 3H), 2.67 (s, 3H).

### Example 111: 7-Fluoro-4-[3-(5-fluoro-2-pyridyl)-1-methyl-pyrazol-4-yl]-6-methoxy-quinoline

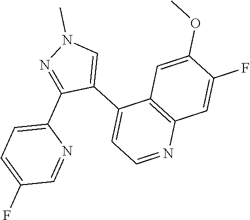 [SMILES: COc1cc2c(-c3cn(C)nc3-c3ccc(F)cn3)ccnc2cc1F]

The title compound was prepared in a manner analogous to Example 1, Step A, except using 4-chloro-7-fluoro-6-methoxyquinoline instead of 4-bromopyridin-2-amine, dioxane and water instead of dioxane, for 16 hours. MS (ESI): mass calcd. for C₁₉H₁₄F₂N₄O, 352.1; m/z found, 353.2 [M+H]⁺. ¹H NMR (400 MHz, CDCl₃) δ 8.75 (d, J=4.4 Hz, 1H), 8.27 (d, J=2.8 Hz, 1H), 7.71 (d, J=11.9 Hz, 1H), 7.56 (s, 1H), 7.27 (d, J=4.4 Hz, 1H), 7.20 (dd, J=4.5, 8.8 Hz, 1H), 7.14 (td, J=2.7, 8.4 Hz, 1H), 6.94 (d, J=9.1 Hz, 1H), 4.08 (s, 3H), 3.57 (s, 3H).

### Example 112: 4-[3-(5-Fluoro-2-pyridyl)-1-methyl-pyrazol-4-yl]-1,7-naphthyridine

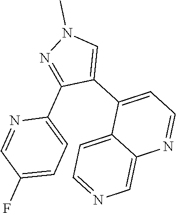 [SMILES: Cn1cc(-c2ccnc3cnccc23)c(-c2ccc(F)cn2)n1]

The title compound was prepared in a manner analogous to Example 1, Step A, except using 4-bromo-1,7-naphthyridine instead of 4-bromopyridin-2-amine, dioxane and water instead of dioxane, for 16 hours. MS (ESI): mass calcd. for C₁₇H₁₂FN₅, 305.1; m/z found, 306.0 [M+H]⁺. ¹H NMR (500 MHz, Methanol-d₄) δ 9.37 (s, 1H), 8.99 (d, J=4.5 Hz, 1H), 8.37 (d, J=5.8 Hz, 1H), 8.00-7.96 (m, 2H), 7.90 (dd, J=4.4, 8.8 Hz, 1H), 7.66 (d, J=4.4 Hz, 1H), 7.64 (d, J=5.9 Hz, 1H), 7.57 (td, J=3.0, 8.7 Hz, 1H), 4.10 (s, 3H).

### Example 113: 4-[3-(5-Fluoro-2-pyridyl)-1-methyl-pyrazol-4-yl]-5,6,7,8-tetrahydro-1,7-naphthyridine

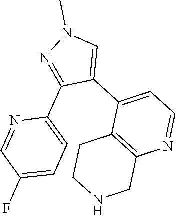 [SMILES: Cn1cc(-c2ccnc3c2CCNC3)c(-c2ccc(F)cn2)n1]

The title compound was prepared in a manner analogous to Example 19, Step A, except using 4-bromo-5,6,7,8-tetrahydro-1,7-naphthyridine dihydrobromide instead of 4-(4-bromo-1-methyl-1H-pyrazol-3-yl)-3-fluoropyridine (Intermediate 19); using 2-(3-(5-fluoropyridin-2-yl)-1-methyl-1H-pyrazol-4-yl)-2-hydroxy-4,4,5,5-tetramethyl-1,3,2-dioxaborolan-2-uide lithium salt (Intermediate 5) instead of 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 1); using sodium carbonate instead of cesium carbonate and dioxane/water instead of 2-methyl-2-butanol. MS (ESI): mass calcd. for C₁₇H₁₆FN₅, 309.1; m/z found, 310.2 [M+H]⁺. ¹H NMR (500 MHz, Methanol-d₄) δ 8.27 (d, J=2.9 Hz, 1H), 8.24 (d, J=5.0 Hz, 1H), 7.79-7.73 (m, 2H), 7.58 (td, J=8.7, 8.7, 3.0 Hz, 1H), 7.06 (d, J=5.0 Hz, 1H), 4.07 (s, 2H), 4.02 (s, 3H), 2.99 (t, J=5.9, 5.9 Hz, 2H), 2.57 (t, J=5.9, 5.9 Hz, 2H).

### Example 114: 5-[3-(5-Fluoro-2-pyridyl)-1-methyl-pyrazol-4-yl]-1,2,3,4-tetrahydro-1,8-naphthyridine

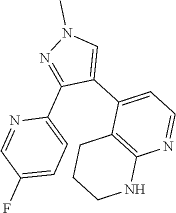 [SMILES: Cn1cc(-c2ccnc3c2CCCN3)c(-c2ccc(F)cn2)n1]

The title compound was prepared in a manner analogous to Example 25, Step A, except using 5-chloro-1,2,3,4-tetrahydro-1,8-naphthyridine instead of 2-(4-bromo-1-methyl-1H-pyrazol-3-yl)-5-chloropyridine (Intermediate 8); 2-(3-(5-fluoropyridin-2-yl)-1-methyl-1H-pyrazol-4-yl)-2-hydroxy-4,4,5,5-tetramethyl-1,3,2-dioxaborolan-2-uide lithium salt (Intermediate 5) instead of 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 1) and using potassium carbonate instead of sodium carbonate. MS (ESI): mass calcd. for C₁₇H₁₆FN₅, 309.1; m/z found, 310.2 [M+H]⁺. ¹H NMR (400 MHz, Methanol-d₄) δ 8.36 (t, J=1.8 Hz, 1H), 7.68 (s, 1H), 7.63 (d, J=5.3 Hz, 1H), 7.56 (dd, J=6.5, 1.8 Hz, 2H), 6.32 (d, J=5.3 Hz, 1H), 3.99 (s, 3H), 3.31 (s, 1H), 3.28 (s, 1H), 2.37 (t, J=6.3 Hz, 2H), 1.79-1.57 (m, 2H).

### Example 115: N-(4-(3-(5-Fluoropyridin-2-yl)-1-methyl-1H-pyrazol-4-yl)pyridin-2-yl)acetamide

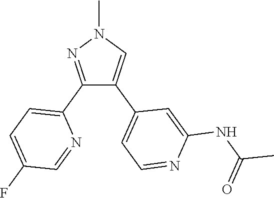 [SMILES: CC(=O)Nc1cc(-c2cn(C)nc2-c2ccc(F)cn2)ccn1]

The title compound was prepared in a manner analogous to Example 1, Step A, except using N-(4-bromopyridin-2-yl)acetamide instead of 4-bromopyridin-2-amine, dioxane and water instead of dioxane, for 16 hours. MS (ESI): mass calcd. for C₁₆H₁₄FN₅O, 311.1; m/z found, 312.2 [M+H]⁺. ¹H NMR (500 MHz, DMSO-d₆) δ 10.22 (s, 1H), 8.35 (s, 1H), 8.13 (s, 1H), 8.06 (d, J=4.9 Hz, 1H), 7.97 (s, 1H), 7.82-7.77 (m, 1H), 7.59 (t, J=8.5 Hz, 1H), 6.93 (d, J=4.7 Hz, 1H), 3.97 (s, 3H), 2.07 (s, 3H).

### Example 116: N-(4-(3-(5-Fluoropyridin-2-yl)-1-methyl-1H-pyrazol-4-yl)pyridin-2-yl)cyclopropanecarboxamide

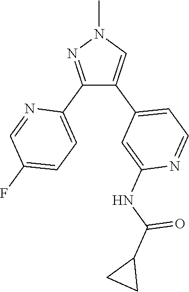 [SMILES: Cn1cc(-c2ccnc(NC(=O)C3CC3)c2)c(-c2ccc(F)cn2)n1]

The title compound was prepared in a manner analogous to Example 1, Step A, except using N-(4-bromopyridin-2-yl)cyclopropanecarboxamide instead of 4-bromopyridin-2-amine, dioxane and water instead of dioxane, for 16 hours. MS (ESI): mass calcd. for C₁₈H₁₆FN₅O, 337.1; m/z found, 338.1 [M+H]⁺. ¹H NMR (400 MHz, CDCl₃) δ 8.91 (s, 1H), 8.43 (d, J=2.9 Hz, 1H), 8.26 (s, 1H), 8.10 (d, J=5.3 Hz, 1H), 7.65 (s, 1H), 7.59 (dd, J=8.7, 4.4 Hz, 1H), 7.37 (td, J=8.5, 8.4, 2.9 Hz, 1H), 6.92 (dd, J=5.3, 1.6 Hz, 1H), 3.95 (s, 3H), 1.61-1.50 (m, 1H), 1.10-1.00 (m, 2H), 0.91-0.80 (m, 2H).

### Example 117: 4-(3-(5-Fluoropyridin-2-yl)-1-methyl-1H-pyrazol-4-yl)-6,7-dihydro-5H-pyrrolo[3,4-b]pyridine

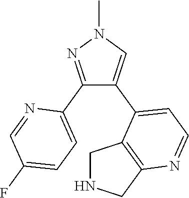 [SMILES: Cn1cc(-c2ccnc3c2CNC3)c(-c2ccc(F)cn2)n1]

The title compound was prepared in a manner analogous to Example 19, Step A, except using 4-chloro-5H,6H,7H-pyrrolo[3,4-b]pyridine dihydrochloride instead of 4-(4-bromo-1-methyl-1H-pyrazol-3-yl)-3-fluoropyridine (Intermediate 19); using 2-(3-(5-fluoropyridin-2-yl)-1-methyl-1H-pyrazol-4-yl)-2-hydroxy-4,4,5,5-tetramethyl-1,3,2-dioxaborolan-2-uide lithium salt (Intermediate 5) instead of 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 1); using sodium carbonate instead of cesium carbonate and dioxane/water instead of 2-methyl-2-butanol. MS (ESI): mass calcd. for C₁₆H₁₄FN₅, 295.1.1; m/z found, 296.2 [M+H]⁺. ¹H NMR (500 MHz, Methanol-d₄) δ 8.31 (d, J=3.0 Hz, 1H), 8.23 (d, J=5.3 Hz, 1H), 7.87 (s, 1H), 7.77 (dd, J=8.8, 4.5 Hz, 1H), 7.63 (td, J=8.6, 8.6, 3.0 Hz, 1H), 7.04 (d, J=5.3 Hz, 1H), 4.16 (s, 2H), 3.99 (s, 3H), 3.93 (s, 2H).

### Example 118: 7-(3-(5-Fluoropyridin-2-yl)-1-methyl-1H-pyrazol-4-yl)thieno[3,2-b]pyridine

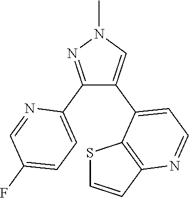 [SMILES: Cn1cc(-c2ccnc3ccsc23)c(-c2ccc(F)cn2)n1]

The title compound was prepared in a manner analogous to Example 1, Step A, except using 7-chlorothieno[3,2-b]pyridine instead of 4-bromopyridin-2-amine, dioxane and water instead of dioxane, for 16 hours. MS (ESI): mass calcd. for C₁₆H₁₁FN₄S, 310.1; m/z found, 311.1 [M+H]⁺. ¹H NMR (400 MHz, CDCl₃) δ 8.59 (d, J=4.9 Hz, 1H), 8.34 (d, J=2.9 Hz, 1H), 7.78 (s, 1H), 7.66 (d, J=5.6 Hz, 1H), 7.55 (d, J=5.6 Hz, 1H), 7.47 (dd, J=4.4, 8.8 Hz, 1H), 7.29 (td, J=2.9, 8.4 Hz, 1H), 7.10 (d, J=4.9 Hz, 1H), 4.05 (s, 3H).

### Example 119: 4-[3-(5-Fluoro-2-pyridyl)-1-methyl-pyrazol-4-yl]-1H-pyrrolo[2,3-b]pyridin-6-amine

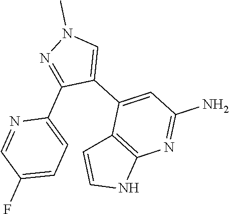 [SMILES: Cn1cc(-c2cc(N)nc3[nH]ccc23)c(-c2ccc(F)cn2)n1]

The title compound was prepared in a manner analogous to Example 1, Step A, except using 4-bromo-1H-pyrrolo[2,3-b]pyridin-6-amine instead of 4-bromopyridin-2-amine, dioxane and water instead of dioxane, for 16 hours. MS (ESI): mass calcd. for C₁₆H₁₃FN₆, 308.1; m/z found, 309.0 [M+H]⁺. ¹H NMR (500 MHz, Methanol-d₄) δ 8.39 (s, 1H), 7.95 (s, 1H), 7.56-7.49 (m, 2H), 6.86 (d, J=3.6 Hz, 1H), 6.27 (s, 1H), 5.93 (d, J=3.7 Hz, 1H), 4.05 (s, 3H).

### Example 120: 2-[4-[3-(5-Fluoro-2-pyridyl)-1-methyl-pyrazol-4-yl]-1H-pyrrolo[2,3-b]pyridin-3-yl]acetonitrile

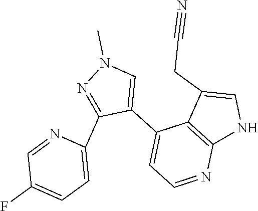 [SMILES: Cn1cc(-c2ccnc3[nH]cc(CC#N)c23)c(-c2ccc(F)cn2)n1]

The title compound was prepared in a manner analogous to Example 1, Step A, except using 2-(4-chloro-1H-pyrrolo[2,3-b]pyridin-3-yl)acetonitrile instead of 4-bromopyridin-2-amine, dioxane and water instead of dioxane, for 16 hours. MS (ESI): mass calcd. for C₁₈H₁₃FN₆, 332.1; m/z found, 333.0 [M+H]⁺. ¹H NMR (500 MHz, Methanol-d₄) δ 8.21 (d, J=2.9 Hz, 1H), 8.19 (d, J=4.9 Hz, 1H), 7.82 (s, 1H), 7.55 (dd, J=8.8, 4.5 Hz, 1H), 7.46 (td, J=8.6, 8.6, 2.9 Hz, 1H), 7.37 (s, 1H), 6.97 (dd, J=4.9, 1.2 Hz, 1H), 4.07 (s, 3H), 3.47 (s, 2H).

### Example 121: 1-Ethyl-5-[3-(5-fluoro-2-pyridyl)-1-methyl-pyrazol-4-yl]pyrazolo[3,4-b]pyridine

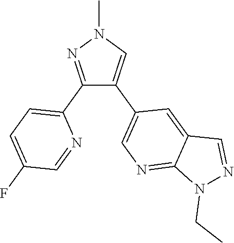 [SMILES: CCn1ncc2cc(-c3cn(C)nc3-c3ccc(F)cn3)cnc21]

The title compound was prepared in a manner analogous to Example 1, Step A, except using 5-bromo-1-ethyl-1H-pyrazolo[3,4-b]pyridine instead of 4-bromopyridin-2-amine, dioxane and water instead of dioxane, for 16 hours. MS (ESI): mass calcd. for C₁₇H₁₅FN₆, 322.1; m/z found, 323.1 [M+H]⁺. ¹H NMR (500 MHz, DMSO-d₆) δ 8.44 (s, 1H), 8.25 (s, 1H), 8.08 (s, 1H), 7.92 (s, 1H), 7.91-7.87 (m, 1H), 7.85 (s, 1H), 7.57 (t, J=8.6 Hz, 1H), 4.51 (q, J=13.4, 6.3 Hz, 2H), 3.99 (s, 3H), 1.52 (t, J=6.7 Hz, 3H).

### Example 122: 4-[3-(5-Fluoro-2-pyridyl)-1-methyl-pyrazol-4-yl]-3-isopropyl-1H-pyrazolo[3,4-b]pyridine

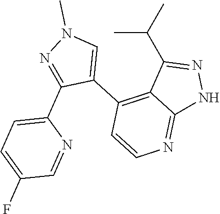 [SMILES: CC(C)c1n[nH]c2nccc(-c3cn(C)nc3-c3ccc(F)cn3)c12]

The title compound was prepared in a manner analogous to Example 1, Step A, except using 4-chloro-3-(propan-2-yl)-1H-pyrazolo[3,4-b]pyridine instead of 4-bromopyridin-2-amine, dioxane and water instead of dioxane, for 16 hours. MS (ESI): mass calcd. for C₁₈H₁₇FN₆, 336.2; m/z found, 337.2 [M+H]⁺. ¹H NMR (500 MHz, Methanol-d₄) δ 8.41 (d, J=4.9 Hz, 1H), 8.14 (d, J=3.0 Hz, 1H), 7.87 (s, 1H), 7.67 (dd, J=8.8, 4.4 Hz, 1H), 7.50 (td, J=8.6, 8.6, 3.0 Hz, 1H), 6.99 (d, J=4.6 Hz, 1H), 4.07 (s, 3H), 2.93 (m, 1H), 0.98 (d, J=6.6 Hz, 6H).

### Example 123: 4-[3-(5-Fluoro-2-pyridyl)-1-methyl-pyrazol-4-yl]-2-methyl-7H-pyrrolo[2,3-d]pyrimidine Trifluoroacetate Salt

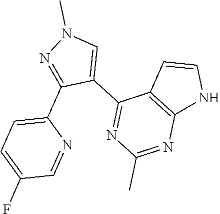 [SMILES: Cc1nc(-c2cn(C)nc2-c2ccc(F)cn2)c2cc[nH]c2n1]

The title compound was prepared in a manner analogous to Example 1, Step A, except using 4-chloro-2-methyl-7H-pyrrolo[2,3-d]pyrimidine instead of 4-bromopyridin-2-amine, dioxane and water instead of dioxane, for 16 hours. MS (ESI): mass calcd. for C₁₆H₁₃FN₆, 308.1; m/z found, 309.0 [M+H]⁺. ¹H NMR (500 MHz, DMSO-d₆) δ 11.51 (s, 1H), 8.21 (s, 1H), 8.18-8.09 (m, 1H), 7.86-7.67 (m, 1H), 7.56 (t, J=11.5 Hz, 1H), 7.05 (s, 1H), 5.86 (s, 1H), 4.03 (s, 3H), 2.55 (s, 3H).

### Example 124: 4-[3-(5-Fluoro-2-pyridyl)-1-methyl-pyrazol-4-yl]-6-methyl-1H-pyrazolo[3,4-d]pyrimidine

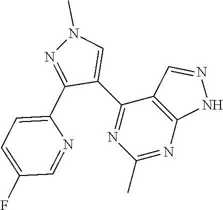 [SMILES: Cc1nc(-c2cn(C)nc2-c2ccc(F)cn2)c2cn[nH]c2n1]

The title compound was prepared in a manner analogous to Example 19, Step A, except using 4-chloro-6-methyl-1H-pyrazolo[3,4-d]pyrimidine instead of 4-(4-bromo-1-methyl-1H-pyrazol-3-yl)-3-fluoropyridine (Intermediate 19); using 2-(3-(5-fluoropyridin-2-yl)-1-methyl-1H-pyrazol-4-yl)-2-hydroxy-4,4,5,5-tetramethyl-1,3,2-dioxaborolan-2-uide lithium salt (Intermediate 5) instead of 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 1); using sodium carbonate instead of cesium carbonate and dioxane/water instead of 2-methyl-2-butanol. MS (ESI): mass calcd. for C₁₅H₁₂FN₇, 309.1; m/z found, 310.2 [M+H]⁺. ¹H NMR (500 MHz, Methanol-d₄) δ 8.38 (s, 1H), 8.28 (s, 1H), 7.89 (dd, J=8.8, 4.5 Hz, 1H), 7.74-7.65 (m, 2H), 4.09 (s, 3H), 2.64 (s, 3H).

### Example 125: 2-Cyclopropyl-4-[3-(5-fluoro-2-pyridyl)-1-methyl-pyrazol-4-yl]-7H-pyrrolo[2,3-d]pyrimidine

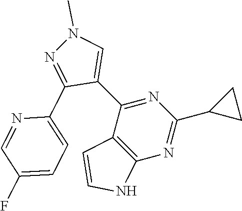 [SMILES: Cn1cc(-c2nc(C3CC3)nc3[nH]ccc23)c(-c2ccc(F)cn2)n1]

The title compound was prepared in a manner analogous to Example 19, Step A, except using 4-chloro-2-cyclopropyl-7H-pyrrolo[2,3-d]pyrimidine instead of 4-(4-bromo-1-methyl-1H-pyrazol-3-yl)-3-fluoropyridine (Intermediate 19); using 2-(3-(5-fluoropyridin-2-yl)-1-methyl-1H-pyrazol-4-yl)-2-hydroxy-4,4,5,5-tetramethyl-1,3,2-dioxaborolan-2-uide lithium salt (Intermediate 5) instead of 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 1); using sodium carbonate instead of cesium carbonate and dioxane/water instead of 2-methyl-2-butanol. MS (ESI): mass calcd. for C₁₈H₁₅FN₆, 334.1; m/z found, 335.2 [M+H]⁺. ¹H NMR (500 MHz, DMSO-d₆) δ 11.51 (s, 1H), 8.26 (s, 1H), 8.22-8.11 (m, 1H), 7.70 (s, 1H), 7.62-7.48 (m, 1H), 7.07 (s, 1H), 6.02 (s, 1H), 4.03 (s, 3H) 2.10-1.92 (m, 1H), 0.84-0.63 (m, 4H).

### Example 126: 4-[3-(5-Fluoro-2-pyridyl)-1-methyl-pyrazol-4-yl]-1,5-naphthyridine

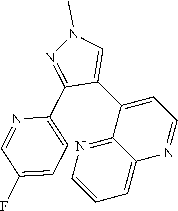 [SMILES: Cn1cc(-c2ccnc3cccnc23)c(-c2ccc(F)cn2)n1]

The title compound was prepared in a manner analogous to Example 19, Step A, except using 4-chloro-1,5-naphthyridine instead of 4-(4-bromo-1-methyl-1H-pyrazol-3-yl)-3-fluoropyridine (Intermediate 19); using 2-(3-(5-fluoropyridin-2-yl)-1-methyl-1H-pyrazol-4-yl)-2-hydroxy-4,4,5,5-tetramethyl-1,3,2-dioxaborolan-2-uide lithium salt (Intermediate 5) instead of 4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 1); using sodium carbonate instead of cesium carbonate and dioxane/water instead of 2-methyl-2-butanol. MS (ESI): mass calcd. for C₁₇H₁₂FN₅, 305.1; m/z found, 306.0 [M+H]⁺. ¹H NMR (500 MHz, Methanol-d₄) δ 8.85 (d, J=4.6 Hz, 1H), 8.72 (dd, J=4.2, 1.7 Hz, 1H), 8.40 (dd, J=8.6, 1.7 Hz, 1H), 8.18 (s, 1H), 8.07 (d, J=2.9 Hz, 1H), 7.78 (dd, J=8.8, 4.5 Hz, 1H), 7.71 (dd, J=8.5, 4.1 Hz, 1H), 7.63 (d, J=4.5 Hz, 1H), 7.59 (td, J=8.6, 8.6, 2.9 Hz, 1H), 4.09 (s, 3H).

### Example 127: 2-Fluoro-8-[3-(5-fluoro-2-pyridyl)-1-methyl-pyrazol-4-yl]-1,5-naphthyridine

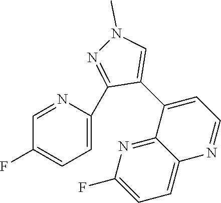 [SMILES: Cn1cc(-c2ccnc3ccc(F)nc23)c(-c2ccc(F)cn2)n1]

The title compound was prepared in a manner analogous to Example 1, Step A, except using 8-bromo-2-fluoro-1,5-naphthyridine instead of 4-bromopyridin-2-amine, dioxane and water instead of dioxane, for 16 hours. MS (ESI): mass calcd. for C₁₇H₁₁F₂N₅, 323.1; m/z found, 324.1 [M+H]⁺. ¹H NMR (400 MHz, DMSO-d₆) δ 8.78 (d, J=4.5 Hz, 1H), 7.94 (q, J=5.0 Hz, 2H), 7.90 (s, 1H), 7.76 (dd, J=9.3, 6.2 Hz, 1H), 7.64 (dd, J=10.2, 2.7 Hz, 1H), 7.53 (td, J=8.7, 2.9 Hz, 1H), 7.25 (d, J=4.5 Hz, 1H), 7.17 (td, J=8.7, 2.7 Hz, 1H), 4.05 (s, 3H).

### Example 128: 2-Ethoxy-8-[3-(5-fluoro-2-pyridyl)-1-methyl-pyrazol-4-yl]-1,5-naphthyridine

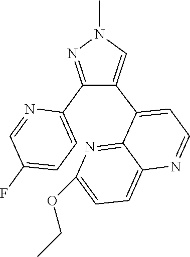 [SMILES: CCOc1ccc2nccc(-c3cn(C)nc3-c3ccc(F)cn3)c2n1]

The title compound was prepared in a manner analogous to Example 1, Step A, except using 8-chloro-2-ethoxy-1,5-naphthyridine instead of 4-bromopyridin-2-amine. MS (ESI): mass calcd. for C₁₉H₁₆FN₅O, 349.1; m/z found, 350.2 [M+H]⁺. ¹H NMR (400 MHz, Methanol-d₄) δ 8.62 (d, J=4.7 Hz, 1H), 8.26-8.08 (m, 3H), 7.69 (ddd, J=8.8, 4.5, 0.7 Hz, 1H), 7.63-7.48 (m, 2H), 7.11 (d, J=9.1 Hz, 1H), 4.08 (s, 3H), 4.05 (q, J=7.1 Hz, 2H), 1.26 (t, J=7.1 Hz, 3H).

### Example 129: 4-[3-(5-Fluoro-2-pyridyl)-1-methyl-pyrazol-4-yl]-6-methoxy-quinoline

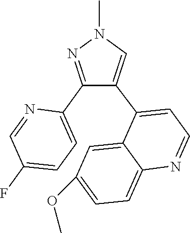 [SMILES: COc1ccc2nccc(-c3cn(C)nc3-c3ccc(F)cn3)c2c1]

The title compound was prepared in a manner analogous to Example 1, Step A, except using 4-bromo-6-methoxyquinoline instead of 4-bromopyridin-2-amine. MS (ESI): mass calcd. for C₁₉H₁₅FN₄O, 334.1; m/z found, 335.0 [M+H]⁺. ¹H NMR (400 MHz, CDCl₃) δ 8.77 (d, J=4.5 Hz, 1H), 8.35 (d, J=2.5 Hz, 1H), 8.02 (d, J=9.3 Hz, 1H), 7.59 (s, 1H), 7.32 (s, 1H), 7.31-7.29 (m, 1H), 7.20-7.11 (m, 2H), 6.92 (d, J=2.8 Hz, 1H), 4.12 (s, 3H), 3.57 (s, 3H)

### Example 130: 4-[3-(5-Fluoro-2-pyridyl)-1-methyl-pyrazol-4-yl]-1,6-naphthyridine

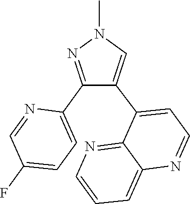 [SMILES: Cn1cc(-c2ccnc3cccnc23)c(-c2ccc(F)cn2)n1]

The title compound was prepared in a manner analogous to Example 1, Step A, except using 4-chloro-1,6-naphthyridine instead of 4-bromopyridin-2-amine. MS (ESI): mass calcd. for C₁₇H₁₂FN₅, 305.1; m/z found, 306.2 [M+H]⁺. ¹H NMR (400 MHz, Methanol-d₄) δ 9.09 (d, J=0.9 Hz, 1H), 9.05 (d, J=4.6 Hz, 1H), 8.63 (d, J=6.0 Hz, 1H), 8.04 (s, 1H), 8.02-7.89 (m, 3H), 7.70-7.44 (m, 2H), 4.12 (s, 3H).

### Example 131: 8-[3-(5-Fluoro-2-pyridyl)-1-methyl-pyrazol-4-yl]-2-methyl-1,5-naphthyridine

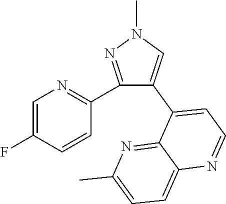 [SMILES: Cc1ccc2nccc(-c3cn(C)nc3-c3ccc(F)cn3)c2n1]

The title compound was prepared in a manner analogous to Example 1, Step A, except using 8-chloro-2-methyl-1,5-naphthyridine instead of 4-bromopyridin-2-amine. MS (ESI): mass calcd. for C₁₈H₁₄FN₅, 319.1; m/z found, 320.2 [M+H]⁺. ¹H NMR (400 MHz, Methanol-d₄) δ 8.78 (d, J=4.6 Hz, 1H), 8.23 (d, J=8.7 Hz, 1H), 8.19 (s, 1H), 8.09 (d, J=2.8 Hz, 1H), 7.73 (dd, J=8.9, 4.5 Hz, 1H), 7.64 (d, J=4.6 Hz, 1H), 7.60 (td, J=8.6, 2.9 Hz, 1H), 7.55 (d, J=8.7 Hz, 1H), 4.10 (s, 3H), 2.44 (s, 3H).

### Example 132: 7-[3-(5-Fluoro-2-pyridyl)-1-methyl-pyrazol-4-yl]-3-methyl-thieno[3,2-b]pyridine

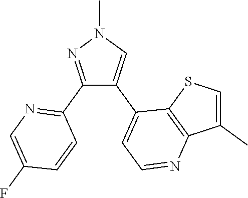 [SMILES: Cc1csc2c(-c3cn(C)nc3-c3ccc(F)cn3)ccnc12]

To a mixture of 2-(3-(5-Fluoropyridin-2-yl)-1-methyl-1H-pyrazol-4-yl)-2-hydroxy-4,4,5,5-tetramethyl-1,3,2-dioxaborolan-2-uide lithium salt (Intermediate 5, 30 mg, 0.1 mmol), 7-chloro-3-methylthieno[3,2-b]pyridine (18 mg, 0.1 mmol) and potassium carbonate (25 mg, 0.18 mmol) in dimethylformamide (0.9 mL) and water (0.15 mL) was added Pd(amphos)Cl₂ (3.5 mg, 0.005 mmol) under nitrogen. The reaction mixture was stirred at 110° C. for 2 hours under an atmosphere of N₂ and then filtered. Purification by preparative HPLC Method D; gave the title compound (10 mg, 12%). MS (ESI): mass calcd. for C₁₇H₁₃FN₄S, 324.1; m/z found, 325.0 [M+H]⁺. ¹H NMR (400 MHz, CDCl₃) δ 8.65 (d, J=4.8 Hz, 1H), 8.39 (d, J=2.9 Hz, 1H), 7.82 (s, 1H), 7.45 (dd, J=4.4, 8.7 Hz, 1H), 7.36 (d, J=1.1 Hz, 1H), 7.31 (dt, J=2.9, 8.4 Hz, 1H), 7.13 (d, J=4.8 Hz, 1H), 4.09 (s, 3H), 2.56 (d, J=1.1 Hz, 3H).

### Example 133: 4-[3-(5-Fluoro-2-pyridyl)-1-methyl-pyrazol-4-yl]-8-methyl-1,5-naphthyridine

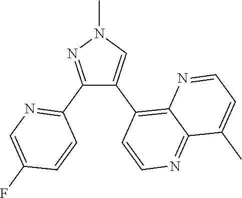 [SMILES: Cc1ccnc2c(-c3cn(C)nc3-c3ccc(F)cn3)ccnc12]

The title compound was prepared in a manner analogous to Example 132, except using 4-bromo-8-methyl-1,5-naphthyridine instead of 7-chloro-3-methylthieno[3,2-b]pyridine. MS (ESI): mass calcd. for C₁₈H₁₄FN₅, 319.1; m/z found, 320.2 [M+H]⁺. ¹H NMR (400 MHz, CDCl₃) δ 8.85 (d, J=4.5 Hz, 1H), 8.72 (d, J=4.3 Hz, 1H), 8.32 (d, J=2.8 Hz, 1H), 8.05 (s, 1H), 7.57 (dd, J=4.5, 8.8 Hz, 1H), 7.49 (d, J=4.6 Hz, 1H), 7.46 (d, J=4.3 Hz, 1H), 7.36-7.29 (m, 1H), 4.09 (s, 3H), 2.87 (s, 3H)

### Example 134: 4-[1-Cyclopropyl-3-(5-fluoro-2-pyridyl)pyrazol-4-yl]-6-methyl-1H-pyrazolo[3,4-b]pyridine

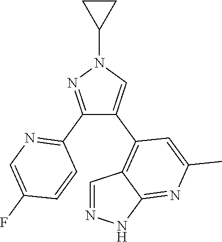 [SMILES: Cc1cc(-c2cn(C3CC3)nc2-c2ccc(F)cn2)c2cn[nH]c2n1]

The title compound was prepared in a manner analogous to Example 26, except using in Step A 2-(4-bromo-1-cyclopropyl-1H-pyrazol-3-yl)-5-fluoropyridine (Intermediate 68) instead of 2-(4-bromo-1-methyl-1H-pyrazol-3-yl)-5-chloropyridine (Intermediate 8); 2-methyl-2-butanol instead of 1,4-dioxane; and the mixture was heated at 90° C. instead of 90° C. via microwave irradiation. MS (ESI): mass calcd. for C₁₈H₁₅FN₆, 334.1; m/z found, 335.1 [M+H]⁺. ¹H NMR (400 MHz, Methanol-d₄) δ 8.30 (d, J=2.6 Hz, 1H), 8.21 (s, 1H), 7.79-7.71 (m, 1H), 7.68-7.60 (m, 1H), 7.50 (s, 1H), 7.00 (s, 1H), 3.91-3.81 (m, 1H), 2.59 (s, 3H), 1.34-1.26 (m, 2H), 1.19-1.10 (m, 2H).

### Example 135: 6-Methyl-4-[1-methyl-3-(2-methyl-4-pyridyl)pyrazol-4-yl]-1H-pyrazolo[3,4-b]pyridine

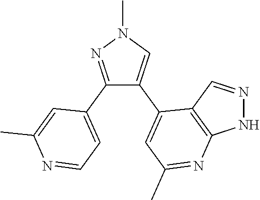 [SMILES: Cc1cc(-c2nn(C)cc2-c2cc(C)nc3[nH]ncc23)ccn1]

Step A: 1-(4-Methoxybenzyl)-6-methyl-4-(1-methyl-3-(2-methylpyridin-4-yl)-1H-pyrazol-4-yl)-1H-pyrazolo[3,4-b]pyridine. A mixture consisting of 4-(3-bromo-1-methyl-1H-pyrazol-4-yl)-1-(4-methoxybenzyl)-6-methyl-1H-pyrazolo[3,4-b]pyridine (Intermediate 71, 50 mg, 0.12 mmol), (2-methylpyridin-4-yl)boronic acid (25 mg, 0.18 mmol), Pd(dtbpf)Cl₂ (7.9 mg, 0.012 mmol), K₂CO₃ (67.0 mg, 0.485 mmol), and 1,4-dioxane/H₂O (4:1) (2 mL) was stirred at 60° C. for 2 h under N₂. The reaction mixture was cooled then added to H₂O (5 mL). The resulting mixture was extracted with EtOAc (10 mL×3). The combined organic phases were dried over anhydrous Na₂SO₄, filtered, and concentrated to dryness under reduced pressure to afford the title compound (65 mg) as a brown oil which was used without further purification.

Step B: 6-Methyl-4-[1-methyl-3-(2-methyl-4-pyridyl)pyrazol-4-yl]-1H-pyrazolo[3,4-b]pyridine. A solution of 1-(4-methoxybenzyl)-6-methyl-4-(1-methyl-3-(2-methylpyridin-4-yl)-1H-pyrazol-4-yl)-1H-pyrazolo[3,4-b]pyridine (65 mg) in TFA (1 mL) was stirred overnight at 50° C. The reaction mixture was concentrated under reduced pressure. MeOH (2 mL) was added and the aqueous phase was adjusted to pH-8 with saturated aqueous NaHCO₃. Purification (preparative HPLC DB using a Boston Prime C18 150×30 mm×5 μm column (eluent: 20% to 50% (v/v) water (0.05% NH₃H₂O+10 mM NH₄HCO₃)−ACN) afforded the title compound. The title compound was suspended in water (10 mL), and the resulting mixture was frozen using dry ice/ethanol, and then lyophilized to dryness to afford the title compound (17.9 mg, 38.4%) as a white solid. MS (ESI): mass calcd. for C₁₇H₁₆N₆, 304.3; m/z found, 305.1 [M+H]⁺. ¹H NMR (400 MHz, CD₃CN): δ 11.50 (br s, 1H), 8.31 (d, J=5.2 Hz, 1H), 7.95 (s, 1H), 7.64 (s, 1H), 7.29 (s, 1H), 7.11 (dd, J=0.8, 4.8 Hz, 1H), 6.91 (s, 1H), 3.99 (s, 3H), 2.53 (s, 3H), 2.41 (s, 3H).

### Example 136: 6-Methyl-4-[1-methyl-3-(3-methyl-4-pyridyl)pyrazol-4-yl]-1H-pyrazolo[3,4-b]pyridine

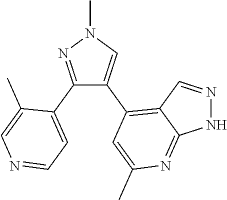 [SMILES: Cc1cc(-c2cn(C)nc2-c2ccncc2C)c2cn[nH]c2n1]

The title compound was prepared in a manner analogous to Example 135 using (3-methylpyridin-4-yl)boronic acid instead of (2-methylpyridin-4-yl)boronic acid in Step A. MS (ESI): mass calcd. for C₁₇H₁₆N₆, 304.3; m/z found, 305.3 [M+H]⁺. ¹H NMR (400 MHz, CDCl₃) δ 11.65 (br s, 1H), 8.42 (s, 1H), 8.37 (d, J=4.9 Hz, 1H), 7.82 (s, 1H), 7.72 (s, 1H), 7.16 (d, J=5.0 Hz, 1H), 6.54 (s, 1H), 4.03 (s, 3H), 2.49 (s, 3H), 2.03 (s, 3H).

### Example 137: 6-Methyl-4-[1-methyl-3-(5-methyl-3-pyridyl)pyrazol-4-yl]-1H-pyrazolo[3,4-b]pyridine

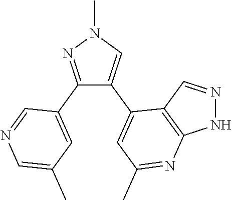 [SMILES: Cc1cncc(-c2nn(C)cc2-c2cc(C)nc3[nH]ncc23)c1]

The title compound was prepared in a manner analogous to Example 135 using (5-methylpyridin-3-yl)boronic acid instead of (2-methylpyridin-4-yl)boronic acid in Step A. MS (ESI): mass calcd. for C₁₇H₁₆N₆, 304.3; m/z found, 305.1 [M+H]⁺. ¹H NMR (400 MHz, CDCl₃) δ 11.74 (s, 1H), 8.42 (s, 1H), 8.37 (s, 1H), 7.75 (s, 1H), 7.67 (d, J=2.2 Hz, 2H), 6.85 (s, 1H), 4.07 (s, 3H), 2.63 (s, 3H), 2.29 (s, 3H).

### Example 138: 4-[3-(3-Chloro-4-pyridyl)-1-methyl-pyrazol-4-yl]-6-methyl-1H-pyrazolo[3,4-b]pyridine

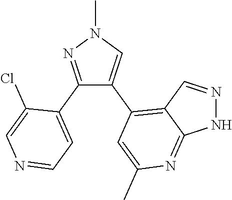 [SMILES: Cc1cc(-c2cn(C)nc2-c2ccncc2Cl)c2cn[nH]c2n1]

The title compound was prepared in a manner analogous to Example 135 using (3-chloropyridin-4-yl)boronic acid instead of (2-methylpyridin-4-yl)boronic acid in Step A. MS (ESI): mass calcd. for C₁₆H₁₃ClN₆, 324.8; m/z found, 325.2 [M+H]⁺. ¹H NMR (400 MHz, CDCl₃) δ 11.31 (br s, 1H), 7.95 (s, 1H), 7.89 (s, 1H), 7.39-7.30 (m, 1H), 7.27 (s, 1H), 7.10-6.95 (m, 1H), 6.67 (s, 1H), 4.14 (s, 3H), 2.57 (s, 3H).

### Example 139: 4-[3-(5-Chloro-3-pyridyl)-1-methyl-pyrazol-4-yl]-6-methyl-1H-pyrazolo[3,4-b]pyridine

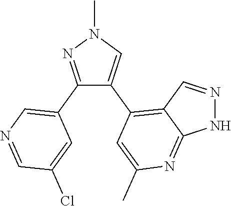 [SMILES: Cc1cc(-c2cn(C)nc2-c2cncc(Cl)c2)c2cn[nH]c2n1]

The title compound was prepared in a manner analogous to Example 135 using (5-chloropyridin-3-yl)boronic acid instead of (2-methylpyridin-4-yl)boronic acid in Step A. MS (ESI): mass calcd. for C₁₆H₁₃ClN₆, 324.8; m/z found, 325.2 [M+H]⁺. ¹H NMR (400 MHz, CDCl₃) δ 11.24 (br s, 1H), 8.61-8.48 (m, 2H), 7.91 (t, J=2.0 Hz, 1H), 7.78 (s, 1H), 7.72 (s, 1H), 6.89 (s, 1H), 4.11 (s, 3H), 2.67 (s, 3H).

### Example 140: 4-[3-(3-Fluoro-5-methyl-4-pyridyl)-1-methyl-pyrazol-4-yl]-6-methyl-1H-pyrazolo[3,4-b]pyridine

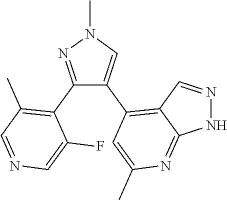 [SMILES: Cc1cc(-c2cn(C)nc2-c2c(C)cncc2F)c2cn[nH]c2n1]

The title compound was prepared in a manner analogous to Example 135 using (3-fluoro-5-methylpyridin-4-yl)boronic acid instead of (2-methylpyridin-4-yl)boronic acid in Step A. MS (ESI): mass calcd. for C₁₇H₁₅FN₆, 322.3; m/z found, 323.2 [M+H]⁺. ¹H NMR (400 MHz, CDCl₃) δ 11.00 (br s, 1H), 8.28 (s, 1H), 8.26 (s, 1H), 7.87 (s, 1H), 7.77 (s, 1H), 6.51 (s, 1H), 4.05 (s, 3H), 2.46 (s, 3H), 2.11 (s, 3H).

### Example 141: 4-[3-(6-Methoxy-5-methyl-3-pyridyl)-1-methyl-pyrazol-4-yl]-6-methyl-1H-pyrazolo[3,4-b]pyridine

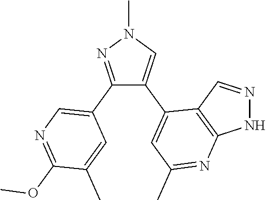 [SMILES: COc1ncc(-c2nn(C)cc2-c2cc(C)nc3[nH]ncc23)cc1C]

The title compound was prepared in a manner analogous to Example 135 using (6-methoxy-5-methylpyridin-3-yl)boronic acid instead of (2-methylpyridin-4-yl)boronic acid in Step A. MS (ESI): mass calcd. for C₁₈H₁₈N₆O, 334.4; m/z found, 335.1 [M+H]⁺. ¹H NMR (400 MHz, DMSO-d₆): 13.41 (s, 1H), 8.29 (s, 1H), 7.89-7.88 (m, 1H), 7.68 (d, J=0.8 Hz, 1H), 7.60 (dd, J=1.2, 2.4 Hz, 1H), 6.86 (s, 1H), 3.97 (s, 3H), 3.86 (s, 3H), 2.49 (s, 3H), 2.10 (s, 3H).

### Example 142: 4-[3-(5-Chloro-6-methoxy-3-pyridyl)-1-methyl-pyrazol-4-yl]-6-methyl-1H-pyrazolo[3,4-b]pyridine

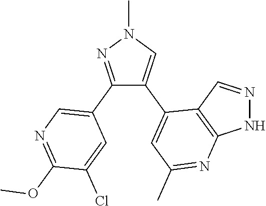 [SMILES: COc1ncc(-c2nn(C)cc2-c2cc(C)nc3[nH]ncc23)cc1Cl]

The title compound was prepared in a manner analogous to Example 135 using (5-chloro-6-methoxypyridin-3-yl)boronic acid instead of (2-methylpyridin-4-yl)boronic acid in Step A. MS (ESI): mass calcd. for C₁₇H₁₅ClN₆O, 354.8; m/z found, 355.0 [M+H]⁺. ¹H NMR (400 MHz, CDCl₃) δ 11.34 (s, 1H), 8.01 (d, J=2.0 Hz, 1H), 7.87 (d, J=2.2 Hz, 1H), 7.70 (d, J=8.1 Hz, 2H), 6.88 (s, 1H), 4.05 (s, 3H), 3.98 (s, 3H), 2.65 (s, 3H).

### Example 143: 6-Methyl-4-[1-methyl-3-(6-methylpyridazin-4-yl)pyrazol-4-yl]-1H-pyrazolo[3,4-b]pyridine

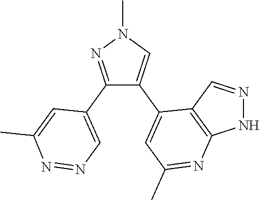 [SMILES: Cc1cc(-c2nn(C)cc2-c2cc(C)nc3[nH]ncc23)cnn1]

The title compound was prepared in a manner analogous to Example 135 using (6-methylpyridazin-4-yl)boronic acid instead of (2-methylpyridin-4-yl)boronic acid in Step A. MS (ESI): mass calcd. for C₁₆H₁₅N₇, 305.3; m/z found, 306.1 [M+H]⁺. ¹H NMR (400 MHz, DMSO-d₆): δ 13.51 (br s, 1H), 8.94 (d, J=2.0 Hz, 1H), 8.39 (s, 1H), 7.73 (s, 1H), 7.55 (d, J=2.0 Hz, 1H), 6.91 (s, 1H), 4.04 (s, 3H), 2.56 (s, 3H), 2.53 (s, 3H).

### Example 144: 4-[3-(5-Fluoro-2-pyridyl)-1-methyl-pyrazol-4-yl]-1-methyl-pyrrolo[2,3-b]pyridine

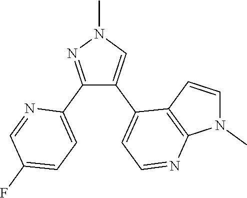 [SMILES: Cn1cc(-c2ccnc3c2ccn3C)c(-c2ccc(F)cn2)n1]

A mixture of 2-(3-(5-fluoropyridin-2-yl)-1-methyl-1H-pyrazol-4-yl)-2-hydroxy-4,4,5,5-tetramethyl-1,3,2-dioxaborolan-2-uide lithium salt (Intermediate 5, 50 mg, 0.157 mmol), 4-bromo-1-methyl-1H-pyrrolo[2,3-b]pyridine (38.3 mg, 0.181 mmol), Pd(amphos)Cl₂ (5.8 mg, 0.008 mmol), and K₂CO₃ (45.6 mg, 0.33 mol) in DMF (1.5 mL) and water (0.25 mL), was heated under N₂, at 110° C. for 2 h. The reaction mixture was cooled to room temperature and filtered. The resulting filtrate was concentrated under reduced pressure and purified (HPLC: Column: Boston Prime C18 150*30 mm*5 μm; Condition: A: water (0.05% NH₃H₂O)/B: CH₃C; at the beginning: A (74%) and B (26%) gradient to at the end: A: (44%) and B (56%); Gradient Time (min) 7; 100% B Hold Time (min) 0; Flow Rate(ml/min) 30) to afford the title compound. The title compound was lyophilized to give an off-white solid (21 mg, 43%). MS (ESI): mass calcd. for C₁₇H₁₄FN₅, 307.1; m/z found, 308.1 [M+H]⁺. ¹H NMR (400 MHz, CDCl₃) δ 8.47 (d, J=3.0 Hz, 1H), 8.28 (d, J=4.8 Hz, 1H), 7.68 (s, 1H), 7.33-7.28 (m, 1H), 7.26-7.19 (m, 1H), 7.10 (d, J=3.5 Hz, 1H), 6.95 (d, J=5.0 Hz, 1H), 6.16 (d, J=3.5 Hz, 1H), 4.08 (s, 3H), 3.90 (s, 3H).

### Example 145: 4-[3-(5-Fluoro-2-pyridyl)-1-methyl-pyrazol-4-yl]thieno[2,3-b]pyridine

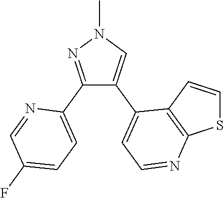 [SMILES: Cn1cc(-c2ccnc3sccc23)c(-c2ccc(F)cn2)n1]

The title compound was prepared in a manner analogous to Example 144, using 4-chlorothieno[2,3-b]pyridine instead of 4-bromo-1-methyl-1H-pyrrolo[2,3-b]pyridine. MS (ESI): mass calcd. for C₁₆H₁₁FN₄S, 310.1; m/z found, 311.0 [M+H]⁺. ¹H NMR (400 MHz, CDCl₃) δ 8.53 (d, J=4.8 Hz, 1H), 8.36 (d, J=2.9 Hz, 1H), 7.64 (s, 1H), 7.42 (dd, J=4.4, 8.8 Hz, 1H), 7.39 (d, J=6.1 Hz, 1H), 7.31-7.25 (m, 1H), 7.19 (d, J=4.9 Hz, 1H), 7.03 (d, J=6.1 Hz, 1H), 4.09 (s, 3H).

### Example 146: 6-(Difluoromethyl)-4-[3-(5-fluoro-3-pyridyl)-1-methyl-pyrazol-4-yl]-1H-pyrazolo[3,4-b]pyridine

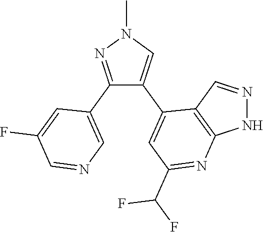 [SMILES: Cn1cc(-c2cc(C(F)F)nc3[nH]ncc23)c(-c2cncc(F)c2)n1]

Step A: 6-(Difluoromethyl)-4-(3-(5-fluoropyridin-3-yl)-1-methyl-1H-pyrazol-4-yl)-1-((2-(trimethylsilyl)ethoxy)methyl)-1H-pyrazolo[3,4-b]pyridine. 3-(4-Bromo-1-methyl-1H-pyrazol-3-yl)-5-fluoropyridine (Intermediate 72, 180 mg, 0.703 mmol), 6-(difluoromethyl)-4-(4,4,5,5-tetramethyl-1,3,2-dioxaborolan-2-yl)-1-((2-(trimethylsilyl)ethoxy)methyl)-1H-pyrazolo[3,4-b]pyridine (Intermediate 73, 299 mg, 0.703 mmol), Cs₂CO₃ (687 mg, 2.11 mmol) were combined in 2-methyl-2-butanol (10 mL) and water (2 mL). The resultant mixture was sparged with N₂ for 5 minutes and then CataCXium® A-Pd-G3 (51 mg, 0.070 mmol) was added. The resulting reaction mixture was sparged with N₂ for another 5 minutes and then stirred at 90° C. for 16 hours. The reaction mixture was cooled to room-temperature and combined with an earlier batch of the same reaction mixture. The combined reaction mixtures were filtered, and the filter cake was washed with ethyl acetate (5 mL×3). The resulting filtrate was concentrated to dryness under reduced pressure. The resulting residue was purified (FCC, SiO₂, eluent:petroleum ether:ethyl acetate=1:0 to 2:1) to afford the title compound as a brown solid. MS (ESI): mass calcd. for C₂₂H₂₅F₃N₆OSi, 474.2; m/z found, 475.1 [M+H]⁺. ¹H NMR (400 MHz, DMSO-d₆): δ 8.61-8.53 (m, 2H), 8.43-8.38 (m, 1H), 8.10 (s, 1H), 7.76-7.65 (m, 1H), 7.26 (s, 1H), 7.02 (t, J=56.0 Hz, 1H), 5.79 (s, 2H), 4.04 (s, 3H), 3.60 (t, J=8.0 Hz, 2H), 0.82 (t, J=8.0 Hz, 2H), −0.12 (s, 9H).

Step B: 6-Methyl-4-(1-methyl-3-(6-methylpyridazin-4-yl)-1H-pyrazol-4-yl)-1H-pyrazolo[3,4-b]pyridine. A solution of TFA (8 mL) and 6-(difluoromethyl)-4-(3-(5-fluoropyridin-3-yl)-1-methyl-1H-pyrazol-4-yl)-1-((2-(trimethylsilyl)ethoxy)methyl)-1H-pyrazolo[3,4-b]pyridine (129 mg, 0.272 mmol) was stirred at room temperature for one hour. The resulting reaction mixture was combined with an earlier batch and concentrated to dryness under reduced pressure. 2 M NH₃ in MeOH (6 mL) was added to the reaction mixture and the resulting mixture was stirred for 10 mins before being concentrated under reduced pressure. Purification (preparative HPLC using a Boston Prime C18 150*30 mm*5 um (eluent: 28% to 58% (v/v) CH₃CN and H₂O with 0.05% NH₃H₂O)) afforded the title compound (42.5 mg, 94%). MS (ESI): mass calcd. for C₁₆H₁₁F₃N₆, 344.1; m/z found, 345.0 [M+H]⁺. ¹H NMR (400 MHz, DMSO-d₆): δ 13.99 (br s, 1H), 8.57 (d, J=2.9 Hz, 1H), 8.53 (s, 1H), 8.41-8.37 (m, 1H), 7.99 (s, 1H), 7.75-7.69 (m, 1H), 7.17 (s, 1H), 6.98 (t, J=52.0 Hz, 1H), 4.03 (s, 3H).

### Example 147: 8-Fluoro-4-[3-(5-fluoro-2-pyridyl)-1-methyl-pyrazol-4-yl]quinoline

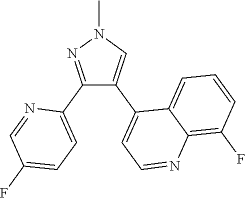 [SMILES: Cn1cc(-c2ccnc3c(F)cccc23)c(-c2ccc(F)cn2)n1]

The title compound was prepared in a manner analogous to Example 144, using 4-chloro-8-fluoroquinoline instead of 4-bromo-1-methyl-1H-pyrrolo[2,3-b]pyridine. MS (ESI): mass calcd. for C₁₈H₁₂F₂N₄, 322.1; m/z found, 323.0 [M+H]⁺. ¹H NMR (400 MHz, CDCl₃) δ 8.94 (d, J=4.4 Hz, 1H), 8.22 (d, J=2.7 Hz, 1H), 7.59 (s, 1H), 7.55 (d, J=8.3 Hz, 1H), 7.41-7.33 (m, 3H), 7.33-7.28 (m, 1H), 7.22 (dt, J=2.9, 8.4 Hz, 1H), 4.11 (s, 3H).

### Example 148: 4-[3-(5-Fluoro-2-pyridyl)-1-methyl-pyrazol-4-yl]-8-methoxy-quinoline

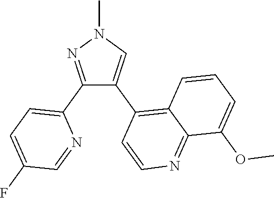 [SMILES: COc1cccc2c(-c3cn(C)nc3-c3ccc(F)cn3)ccnc12]

The title compound was prepared in a manner analogous to Example 150, using 4-bromo-8-methoxyquinoline instead of 4-bromoquinoline-6-carbonitrile. MS (ESI): mass calcd. for C₁₉H₁₅FN₄O, 334.1; m/z found, 335.1 [M+H]⁺. ¹H NMR (400 MHz, CDCl₃) δ 8.91 (d, J=4.3 Hz, 1H), 8.29 (d, J=2.8 Hz, 1H), 7.57 (s, 1H), 7.34 (d, J=4.3 Hz, 1H), 7.32 (d, J=1.8 Hz, 1H), 7.30 (d, J=7.3 Hz, 1H), 7.23-7.17 (m, 1H), 7.17-7.10 (m, 1H), 7.03 (dd, J=1.5, 7.3 Hz, 1H), 4.11 (s, 3H), 4.10 (s, 3H).

### Example 149: 4-[3-(5-Fluoro-2-pyridyl)-1-methyl-pyrazol-4-yl]quinoline-7-carbonitrile

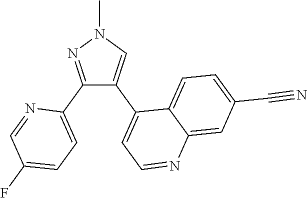 [SMILES: Cn1cc(-c2ccnc3cc(C#N)ccc23)c(-c2ccc(F)cn2)n1]

To a mixture of 2-(3-(5-fluoropyridin-2-yl)-1-methyl-1H-pyrazol-4-yl)-2-hydroxy-4,4,5,5-tetramethyl-1,3,2-dioxaborolan-2-uide lithium salt_(Intermediate 5, 30 mg, 0.1 mmol), 4-bromoquinoline-7-carbonitrile (23 mg, 0.1 mmol) and potassium carbonate (34 mg, 0.25 mmol) in dioxane (1.5 mL) and H₂O (0.2 mL) was added 1,1′-bis(di-tert-butylphosphino)ferrocene palladium dichloride (3 mg, 0.005 mmol) under N₂ flow. The reaction mixture was stirred under N₂ at 60° C. for 2 h. The resulting reaction mixture was concentrated under reduced pressure. The resulting residue was purified (FCC, SiO₂, gradient: petroleum ether/ethyl acetate from 100/0 to 70/30) to afford the title compound (7 mg, 21%) as gray solid. MS (ESI): mass calcd. for C₁₉H₁₂FN₅, 329.1; m/z found, 330.0 [M+H]⁺. ¹H NMR (400 MHz, CDCl₃) δ 8.98 (d, J=4.3 Hz, 1H), 8.40 (dd, J=1.1, 8.4 Hz, 1H), 7.93-7.86 (m, 2H), 7.82 (d, J=2.8 Hz, 1H), 7.73 (dd, J=7.4, 8.4 Hz, 1H), 7.54 (s, 1H), 7.46 (d, J=4.3 Hz, 1H), 7.34-7.28 (m, 1H), 4.11 (s, 3H).

### Example 150: 4-[3-(5-Fluoro-2-pyridyl)-1-methyl-pyrazol-4-yl]quinoline-6-carbonitrile

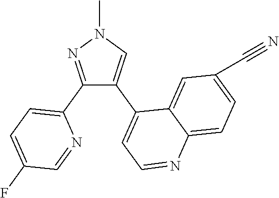 [SMILES: Cn1cc(-c2ccnc3ccc(C#N)cc23)c(-c2ccc(F)cn2)n1]

To a mixture of 2-(3-(5-fluoropyridin-2-yl)-1-methyl-1H-pyrazol-4-yl)-2-hydroxy-4,4,5,5-tetramethyl-1,3,2-dioxaborolan-2-uide lithium salt (Intermediate 5, 50 mg, 0.16 mmol) and 4-bromoquinoline-6-carbonitrile (40 mg, 0.17 mmol) in sat. aq. sodium carbonate (0.35 mL) and dioxane (1.25 mL) was added to XPhos Pd G3 (7 mg, 0.008 mmol, 0.05 eq.) under N₂ flow. The reaction mixture was stirred under N₂ at 90° C. for 2 h. The resulting reaction mixture was concentrated under reduced pressure. Purification (preparative HPLC using a Boston Prime C18 150*30 mm*5 um (eluent: 71% to 41% (v/v) CH₃CN and H₂O with 0.05% NH₃H₂O). The resulting title compound was lyophilized to afford the title compound (14.2 mg, 27%) as off-white solid. MS (ESI): mass calcd. for C₁₉H₁₂FN₅, 329.1; m/z found, 330.1 [M+H]⁺. ¹H NMR (400 MHz, CDCl₃) δ 9.00 (dd, J=3.3, 4.3 Hz, 1H), 8.25-8.15 (m, 2H), 8.12-7.97 (m, 1H), 7.85-7.77 (m, 1H), 7.77-7.67 (m, 1H), 7.60 (d, J=3.0 Hz, 1H), 7.42 (dd, J=3.1, 4.1 Hz, 1H), 7.35 (tdd, J=3.0, 5.6, 8.3 Hz, 1H), 7.30-7.23 (m, 1H), 4.13 (d, J=2.8 Hz, 3H).

### Example 151: 4-[3-(5-Fluoro-2-pyridyl)-1-methyl-pyrazol-4-yl]-5,7-dimethoxy-quinoline

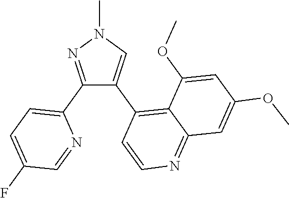 [SMILES: COc1cc(OC)c2c(-c3cn(C)nc3-c3ccc(F)cn3)ccnc2c1]

The title compound was prepared in a manner analogous to Example 144, using 4-chloro-5,7-dimethoxyquinoline instead of 4-bromo-1-methyl-1H-pyrrolo[2,3-b]pyridine. MS (ESI): mass calcd. for C₂₀H₁₇FN₄O₂, 364.1; m/z found, 365.1 [M+H]⁺. ¹H NMR (400 MHz, CDCl₃) δ 8.75 (d, J=4.6 Hz, 1H), 8.24 (d, J=2.9 Hz, 1H), 7.42 (s, 1H), 7.22 (dd, J=4.4, 8.8 Hz, 1H), 7.13 (dd, J=2.9, 8.3 Hz, 1H), 7.11-7.06 (m, 2H), 6.30 (d, J=2.2 Hz, 1H), 4.05 (s, 3H), 3.94 (s, 3H), 3.31 (s, 3H).

### Example 152: 3-Fluoro-8-[3-(5-fluoro-2-pyridyl)-1-methyl-pyrazol-4-yl]-1,5-naphthyridine

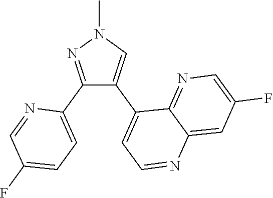 [SMILES: Cn1cc(-c2ccnc3cc(F)cnc23)c(-c2ccc(F)cn2)n1]

The title compound was prepared in a manner analogous to Example 144, using 8-chloro-3-fluoro-1,5-naphthyridine instead of 4-bromo-1-methyl-1H-pyrrolo[2,3-b]pyridine. MS (ESI): mass calcd. for C₁₇H₁₁F₂N₅, 323.1; m/z found, 324.0 [M+H]⁺. ¹H NMR (400 MHz, CDCl₃) δ 8.86 (d, J=4.5 Hz, 1H), 8.75 (d, J=2.7 Hz, 1H), 8.26 (d, J=2.9 Hz, 1H), 8.07-8.00 (m, 2H), 7.67 (dd, J=4.4, 8.7 Hz, 1H), 7.50 (d, J=4.5 Hz, 1H), 7.38 (dt, J=2.9, 8.4 Hz, 1H), 4.09 (s, 3H).

### Example 153: 3-Bromo-8-[3-(5-fluoro-2-pyridyl)-1-methyl-pyrazol-4-yl]-1,5-naphthyridine

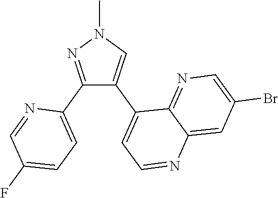 [SMILES: Cn1cc(-c2ccnc3cc(Br)cnc23)c(-c2ccc(F)cn2)n1]

A solution of 2-(3-(5-fluoropyridin-2-yl)-1-methyl-1H-pyrazol-4-yl)-2-hydroxy-4,4,5,5-tetramethyl-1,3,2-dioxaborolan-2-uide lithium salt (Intermediate 5, 50 mg, 0.16 mmol), Pd(PPh₃)₄ (9.5 mg, 0.008 mmol), 3,8-dibromo-1,5-naphthyridine (50 mg, 0.17 mmol), sat. aq. sodium carbonate (0.3 mL), in dioxane (0.9 mL), was heated under N₂, at 110° C. for 2 h. The reaction mixture was cooled to room temperature and filtered. The resulting filtrate was concentrated under reduced pressure and purified (HPLC, A: water (0.05% HCl)/B: CH₃CN at the beginning: A (55%) and B (45%) at the end: A: (45%) and B (55%); Gradient Time (min) 9; 100% B Hold Time (min) 4; Flow Rate(mL/min) 30). The resulting title compound was lyophilized to afford (29 mg, 41%) as a yellow solid, HCl salt. MS (ESI): mass calcd. for C₁₇H₁₁BrFN₅, 383.0; m/z found, 384.8 [M+H]⁺. ¹H NMR (400 MHz, CDCl₃) δ 8.94 (d, J=4.6 Hz, 1H), 8.81 (d, J=2.1 Hz, 1H), 8.73 (d, J=2.1 Hz, 1H), 8.24 (s, 1H), 8.12 (d, J=2.9 Hz, 1H), 7.87 (dd, J=4.5, 8.8 Hz, 1H), 7.74 (dt, J=3.0, 8.8 Hz, 1H), 7.65 (d, J=4.5 Hz, 1H), 4.02 (s, 3H).

### Example 154: 4-[3-(5-Fluoro-2-pyridyl)-1-methyl-pyrazol-4-yl]-7-methoxy-1,6-naphthyridine

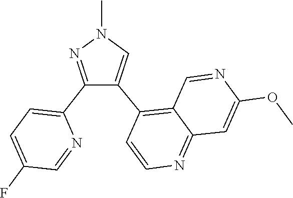 [SMILES: COc1cc2nccc(-c3cn(C)nc3-c3ccc(F)cn3)c2cn1]

The title compound was prepared in a manner analogous to Example 144, using 4-chloro-7-methoxy-1,6-naphthyridine instead of 4-bromo-1-methyl-1H-pyrrolo[2,3-b]pyridine. MS (ESI): mass calcd. for C₁₈H₁₄FN₅O, 335.1; m/z found, 336.0 [M+H]⁺. ¹H NMR (400 MHz, CDCl₃) δ 8.92 (d, J=4.7 Hz, 1H), 8.89 (s, 1H), 8.15 (d, J=2.9 Hz, 1H), 7.65-7.58 (m, 2H), 7.35-7.28 (m, 1H), 7.28 (s, 1H), 7.17 (d, J=4.4 Hz, 1H), 4.10 (s, 3H), 4.03 (s, 3H).

### Example 155: 8-[3-(5-Fluoro-2-pyridyl)-1-methyl-pyrazol-4-yl]-3-methoxy-1,5-naphthyridine

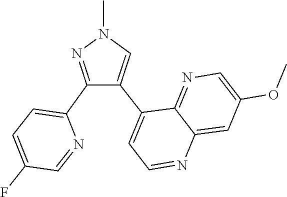 [SMILES: COc1cnc2c(-c3cn(C)nc3-c3ccc(F)cn3)ccnc2c1]

The title compound was prepared in a manner analogous to Example 144, using 8-chloro-3-methoxy-1,5-naphthyridine instead of 4-bromo-1-methyl-1H-pyrrolo[2,3-b]pyridine. MS (ESI): mass calcd. for C₁₈H₁₄FN₅O, 335.1; m/z found, 336.1 [M+H]⁺. ¹H NMR (400 MHz, CDCl₃) δ 8.76 (d, J=4.4 Hz, 1H), 8.61 (d, J=2.9 Hz, 1H), 8.31 (d, J=2.9 Hz, 1H), 8.02 (s, 1H), 7.66 (d, J=2.7 Hz, 1H), 7.59 (dd, J=4.5, 8.7 Hz, 1H), 7.40-7.30 (m, 2H), 4.08 (s, 3H), 4.00 (s, 3H).

### Example 156: 7-Fluoro-8-[3-(5-fluoro-2-pyridyl)-1-methyl-pyrazol-4-yl]-2-methoxy-1,5-naphthyridine

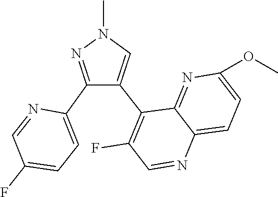 [SMILES: COc1ccc2ncc(F)c(-c3cn(C)nc3-c3ccc(F)cn3)c2n1]

The title compound was prepared in a manner analogous to Example 144, using 4-chloro-7-methoxy-1,6-naphthyridine instead of 4-bromo-1-methyl-1H-pyrrolo[2,3-b]pyridine. MS (ESI): mass calcd. for C₁₈H₁₃F₂N₅O, 353.1; m/z found, 354.1 [M+H]⁺. ¹H NMR (400 MHz, CDCl₃) δ 8.68 (d, J=1.0 Hz, 1H), 8.21 (s, 1H), 8.19 (s, 1H), 7.87 (s, 1H), 7.61 (dd, J=4.3, 8.8 Hz, 1H), 7.30 (t, J=8.3 Hz, 1H), 7.01 (d, J=9.0 Hz, 1H), 4.10 (s, 3H), 3.68 (s, 3H).

### BIOLOGICAL DATA

### Purified Enzyme Assay

In this assay, CSNK1D phosphorylates a substrate peptide PLSRTL-pS-VASLPGL in the presence of ATP. This substrate peptide has been modeled after the sequences surrounding three main cyclic AMP-dependent protein kinase sites of glycogen synthase. This assay monitors CSNK1D kinase activity by measuring the amount of ADP produced in the assay. A substrate mix is prepared by diluting peptide substrate (final concentration 150 μM) with ATP (final concentration 20 μM) in assay buffer (50 mM Tris/HCl pH 7.4+10 mM MgCl₂+1 mM DTT+0.1% BSA). The substrate mix is added to each well of a low volume, 384-well, white opaque plate. Test compounds were diluted in HBSS and added in a dose-response to the plate. To start the reaction, 2 nM of constitutively active human recombinant GST cleaved CSNK1D (University of Dundee, clone DU 19064, stored at 0.28 mg/mL in 50 mM Tris/HCl pH 7.5, 150 mM NaCl, 270 mM Sucrose, 0.1 mM EGTA, 0.1% 2-mercaptoethanol, 0.02% Brij-35.1 mM benzamidine, 0.2 mM PMSF) was added to each well and the plate centrifuged for 5 minutes at 1500 rpm. The total volume of each reaction is 5 ul (2 μL of substrate mix, 1 μL of diluted compounds, and 2 μL of human recombinant CSNK1D). The plates are incubated for 45 minutes at room temperature.

ADP was quantified using the ADP-Glo™ Kinase Assay. ADP-Glo Reagent (5 μL) was added to each well. After a 1 hour incubation at room temperature, Kinase Detection Reagent (10 μL) was added to each well and incubated for 30 minutes. Luminescence was measured on the Perkin Elmer Wallac EnVision 2104 Multi-label Reader. The raw data from the Envision is used to calculate percent activity. Percent activity is then graphed against the log of compound concentrations and these graphs are used to determine IC₅₀ of each compound.

Whole Cell nBRET CSNK1D Binding Assay

This cellular binding assay uses a bioluminescence resonance energy transfer to measure human CSNK1D binding activity in living Chinese Hamster Ovary (CHO) cells that are stably expressing human CSNK1D tagged with nanoluciferase. The cells were grown to confluency in growth media (DMEM:F12, 50 u/mLPen/Strep, 40 mM glutamine, and 0.6 mg/mL G418) in 10 cm² dishes. Cells were seeded onto white, opaque 384-well plates (Corning, cat #3704) at a density of 8,000 cells/well in serum-free OptiMEM and left to incubated overnight at 37° C., 500 CO₂. The next day, test compounds were added in a dose response to the plate followed by a NanoBret tracer (130 nM). The plate is mixed on an orbital shaker for 30 seconds, then placed in a 37° C. incubator for 2 hours. NanoBret Nano-Glo Substrate solution (20 μL) is added to all wells and incubated for 3 minutes at room temperature. Donor (450 nm) and acceptor (630 nm) emissions are then measured within 10 minutes using a ClarioStar plate reader. The data is used to calculate millibret units, defined as (E630/E450)*1000. Bret emissions are then graphed against the log of compound concentrations and these graphs are used to determine the IC₅₀ of each compound.

TABLE 4

| | CSNK1D | ADP-Glo ™ |
|---|---|---|
| | BRET | CSNK1D |
| Example | IC₅₀ | AC₅₀ |
| # | (μM) | (μM) |
| 1 | 0.107 | 0.005 |
| 2 | 0.955 | 0.037 |
| 3 | 1.498 | 0.058 |
| 4 | 2.361 | 0.118 |
| 5 | 0.460 | 0.015 |
| 6 | 1.423 | 0.041 |
| 7 | 0.128 | 0.038 |
| 8 | >10 | 0.783 |
| 9 | 0.568 | 0.016 |
| 10 | >10 | 2.280 |
| 11 | 4.364 | 0.111 |
| 12 | 0.678 | 0.050 |
| 13 | 0.626 | 0.028 |
| 14 | 3.128 | 0.083 |
| 15 | 0.181 | 0.007 |
| 16 | 0.319 | 0.015 |
| 17 | 1.642 | 0.101 |
| 18 | 1.770 | 0.026 |
| 19 | 0.797 | 0.092 |
| 20 | 0.070 | 0.003 |
| 21 | 0.356 | 0.014 |
| 22 | 1.168 | 0.044 |
| 23 | >10 | 0.551 |
| 24 | 0.251 | 0.034 |
| 25 | 0.306 | 0.693 |
| 26 | 2.094 | 0.058 |
| 27 | 0.178 | 0.004 |
| 28 | 0.165 | 0.008 |
| 29 | 0.186 | 0.007 |
| 30 | 0.388 | 0.024 |
| 31 | 3.118 | 0.050 |
| 32 | 8.939 | 0.128 |
| 33 | 17.848 | 0.317 |
| 34 | 1.977 | 0.100 |
| 35 | >10 | 9.181 |
| 36 | >10 | 6.476 |
| 37 | 0.078 | 0.015 |
| 38 | 0.390 | 0.046 |
| 39 | 3.252 | 0.128 |
| 40 | 0.288 | 0.019 |
| 41 | 4.995 | 0.107 |
| 42 | 2.770 | 0.134 |
| 43 | 0.268 | 0.031 |
| 44 | 0.469 | 0.054 |
| 45 | >10 | 0.746 |
| 46 | 2.191 | 0.084 |
| 47 | >10 | 0.346 |
| 48 | 3.474 | 0.039 |
| 49 | >10 | 0.451 |
| 50 | 0.122 | 0.005 |
| 51 | 0.028 | 0.005 |
| 52 | 0.227 | 0.013 |
| 53 | 0.359 | 0.068 |
| 54 | 0.054 | 0.005 |
| 55 | 0.093 | 0.007 |
| 56 | 0.205 | 0.005 |
| 57 | 0.219 | 0.014 |
| 58 | 0.097 | 0.017 |
| 59 | 0.326 | 0.044 |
| 60 | 0.372 | 0.011 |
| 61 | 0.115 | 0.014 |
| 62 | 0.477 | 0.008 |
| 63 | 0.253 | 0.006 |
| 64 | 0.393 | 0.019 |
| 65 | 0.171 | 0.021 |
| 66 | 1.066 | 0.526 |
| 67 | 1.308 | 0.018 |
| 68 | 0.080 | 0.004 |
| 69 | 0.311 | 0.006 |
| 70 | 0.236 | 0.012 |
| 71 | 0.113 | 0.003 |
| 72 | 0.136 | 0.008 |
| 73 | 0.576 | 0.020 |
| 74 | 0.218 | 0.036 |
| 75 | 0.024 | 0.001 |
| 76 | 0.017 | 0.001 |
| 77 | 0.053 | 0.007 |
| 78 | 0.050 | 0.004 |
| 79 | 0.052 | 0.007 |
| 80 | 0.041 | 0.001 |
| 81 | 1.462 | 0.084 |
| 82 | 0.059 | 0.006 |
| 83 | 1.569 | 0.054 |
| 84 | 0.224 | 0.023 |
| 85 | 0.255 | 0.022 |
| 86 | 1.059 | 0.048 |
| 87 | 0.094 | 0.004 |
| 88 | 0.144 | 0.016 |
| 89 | 0.071 | 0.003 |
| 90 | 7.163 | 0.075 |
| 91 | 0.653 | 0.019 |
| 92 | 0.184 | NT |
| 93 | 3.664 | 0.123 |
| 94 | 0.187 | 0.010 |
| 95 | 0.376 | 0.002 |
| 96 | 0.148 | 0.004 |
| 97 | >10 | 0.384 |
| 98 | 2.351 | 0.076 |
| 99 | 0.238 | 0.011 |
| 100 | 0.197 | 0.012 |
| 101 | 0.829 | 0.034 |
| 102 | 1.284 | 0.048 |
| 103 | 0.147 | 0.010 |
| 104 | 1.325 | 0.170 |
| 105 | 0.817 | 0.025 |
| 106 | 0.564 | 0.039 |
| 107 | 0.264 | 0.031 |
| 108 | 0.373 | 0.013 |
| 109 | 0.468 | 0.041 |
| 110 | >10 | 5.365 |
| 111 | 0.491 | 0.028 |
| 112 | 0.953 | 0.051 |
| 113 | >10 | 0.144 |
| 114 | 4.234 | 0.166 |
| 115 | 0.315 | 0.009 |
| 116 | 0.076 | 0.003 |
| 117 | >10 | 0.101 |
| 118 | 0.376 | 0.024 |
| 119 | 0.032 | 0.002 |
| 120 | 8.714 | 0.031 |
| 121 | 1.598 | 0.043 |
| 122 | >10 | 0.015 |
| 123 | 0.234 | 0.004 |
| 124 | 1.941 | 0.052 |
| 125 | 1.914 | 0.066 |
| 126 | 4.621 | 0.026 |
| 127 | 0.962 | 0.046 |
| 128 | 0.103 | 0.011 |
| 129 | 0.785 | 0.109 |
| 130 | 4.390 | 0.128 |
| 131 | 4.477 | 0.310 |
| 132 | 5.167 | 0.361 |
| 133 | >10 | 7.031 |
| 134 | 0.329 | 0.030 |
| 135 | 7.119 | 0.168 |
| 136 | >10 | 1.042 |
| 137 | 0.890 | 0.038 |
| 138 | 8.624 | 0.217 |
| 139 | 0.506 | 0.018 |
| 140 | >10 | 1.167 |
| 141 | 2.015 | 0.044 |
| 142 | 0.382 | 0.028 |
| 143 | >10 | 1.540 |
| 144 | 1.72 | 0.077 |
| 145 | 0.778 | 0.040 |
| 146 | 6.568 | 0.171 |
| 147 | >10 | 1.103 |
| 148 | >10 | 7.093 |
| 149 | >10 | 0.124 |
| 150 | 1.821 | 0.113 |
| 151 | 0.453 | 0.038 |
| 152 | 0.578 | 0.073 |
| 153 | 0.579 | 0.039 |
| 154 | 0.522 | 0.025 |
| 155 | 0.318 | 0.022 |
| 156 | 0.803 | 0.052 |

NT means Not Tested.

1. A compound of Formula (I), 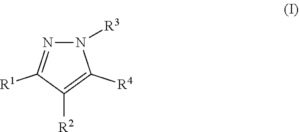
wherein
R¹ is selected from the group consisting of:
(a) 5-membered heteroaryl selected from the group consisting of: 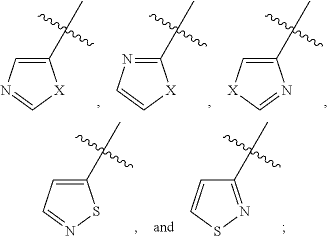
(b) pyridinyl substituted with one or two halo members;
(c) pyrimidinyl; pyrimidinyl substituted with halo; pyrazinyl; pyrazinyl substituted with C₁₋₆ alkyl; pyridazinyl; and pyridazinyl substituted with C₁₋₆ alkyl;
R² is selected from the group consisting of:
(d) 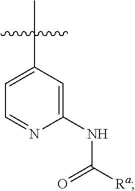
(e) 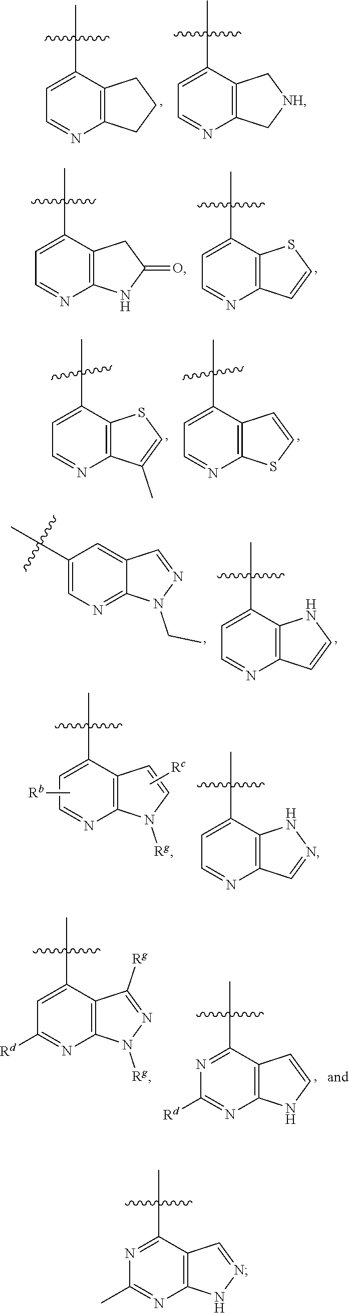
wherein
R^{a} is C₁₋₃ alkyl or C₃₋₆ cycloalkyl;
R^{b} is selected from the group consisting of: H, halo, C₁₋₃ alkyl, C₁₋₃ haloalkyl, CN, CH₂CN, NH₂, oxetanyl, and CH₂-oxetanyl;
R^{c} is selected from the group consisting of: H, halo, C₁₋₃ alkyl, C₁₋₃ haloalkyl, CN, CH₂CN, oxetanyl, oxetanyl substituted with OH, tetrahydrofuranyl substituted with OH, and CH₂-tetrahydrofuranyl;
R^{d} is selected from the group consisting of: H, C₁₋₃ alkyl, C₁₋₃ haloalkyl, and C₃₋₆ cycloalkyl;
R^{g} is H or C₁₋₃ alkyl;
X is O, S, or N—CH₃;
R³ is selected from the group consisting of: C₁₋₆ alkyl, CH₂CH₂OCH₃, C₁₋₆ haloalkyl, C₃₋₆ cycloalkyl, oxetanyl, CH₂-oxetanyl, and tetrahydrofuranyl; and
R⁴ is selected from the group consisting of: H, C₁₋₃ alkyl, and C₃₋₆ cycloalkyl;
and pharmaceutically acceptable salts, N-oxides or solvates of compounds of Formula (I).

2. The compound of claim 1, wherein R¹ is

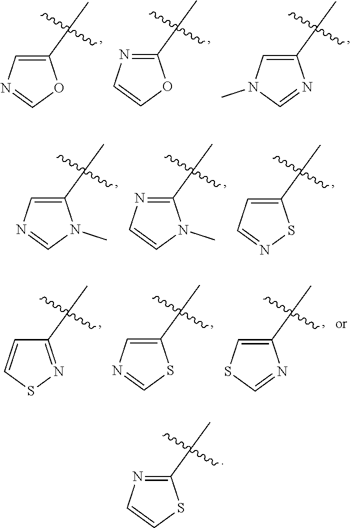

3. The compound of claim 1, wherein R¹ is

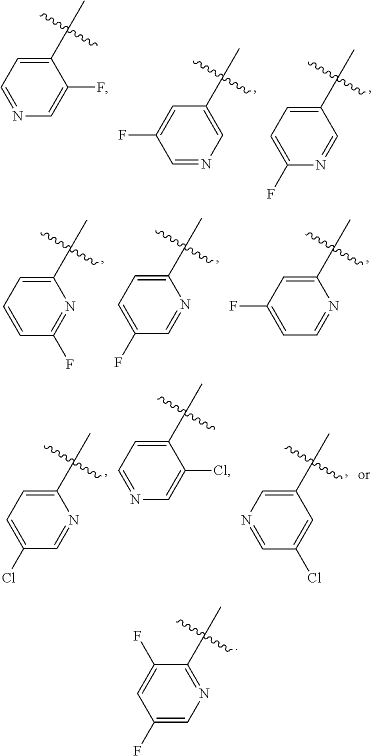

4. The compound of claim 1, wherein R¹ is

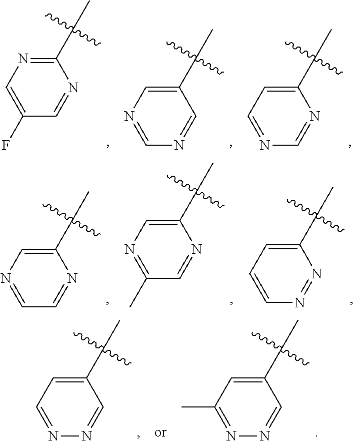

5. The compound of claim 1, wherein R¹ is

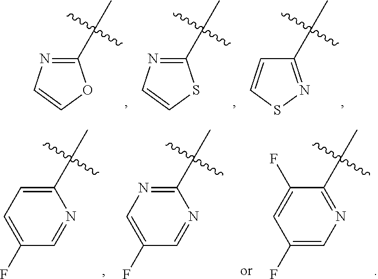 [SMILES: *c1ncco1 ; *c1nccs1 ; *c1ccsn1 ; *c1ccc(F)cn1 ; *c1ncc(F)cn1 ; *c1ncc(F)cc1F]

6. The compound of claim 1, wherein R² is

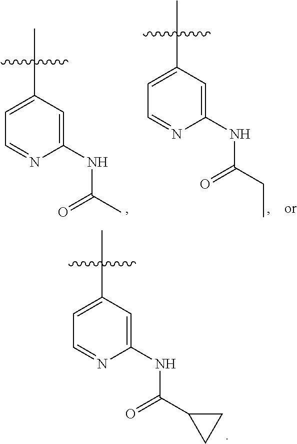 [SMILES: *c1ccnc(NC(C)=O)c1 ; *c1ccnc(NC(=O)CC)c1 ; *c1ccnc(NC(=O)C2CC2)c1]

7. The compound of claim 1, wherein R² is

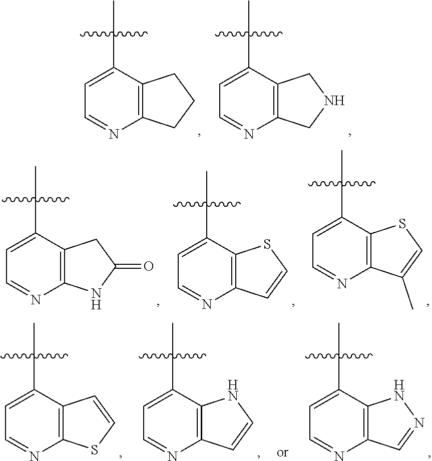

8. The compound of claim 1, wherein R² is

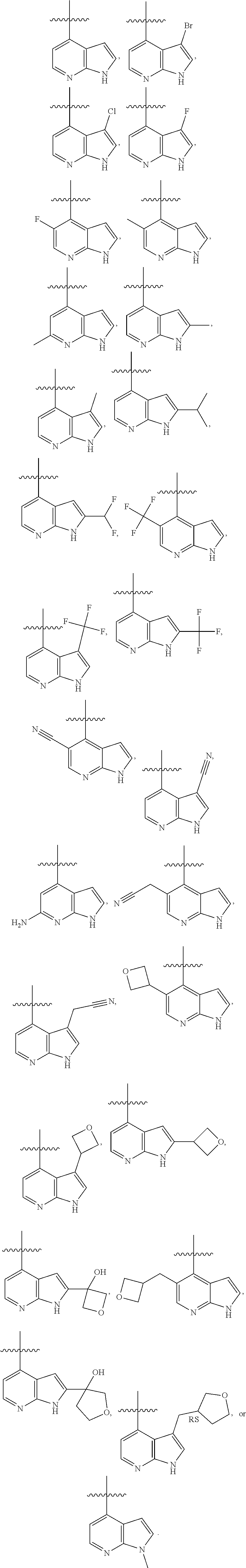

9. The compound of claim 1, wherein R² is

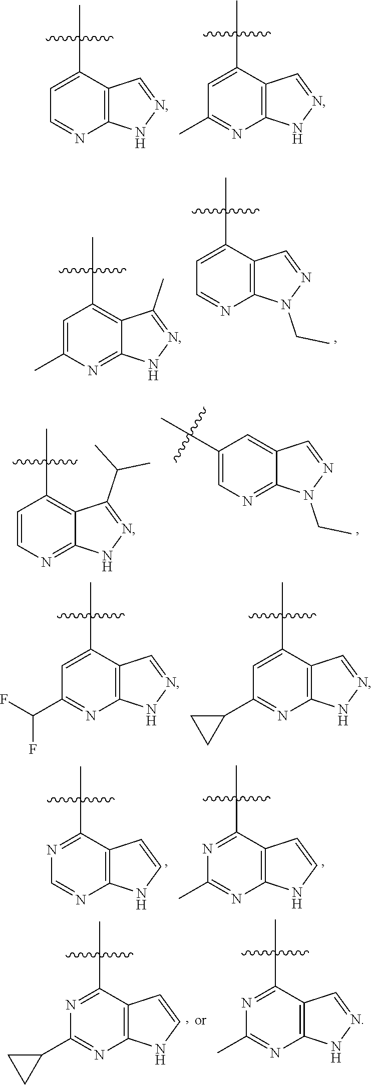

10. The compound of claim 1, wherein R² is

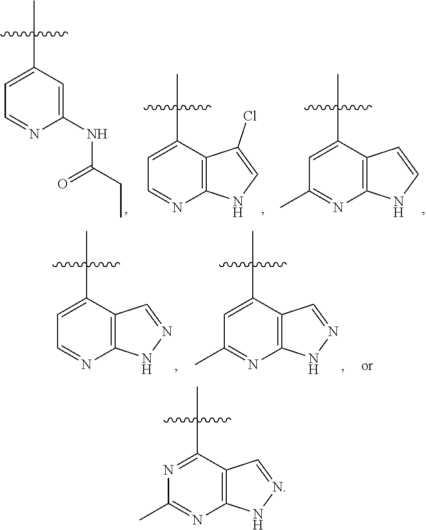

11. The compound of claim 1, wherein R³ is CH₃, CH₂CH₃, CH(CH₃)₂, CH₂CH(CH₃)₂, CH₂CH₂OCH₃, CHF₂, CH₂CH₂F, CH₂CHF₂, CH₂CF₃, cyclopropyl, cyclobutyl,

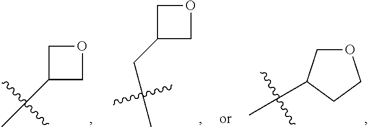 [SMILES: *C1COC1 ; *CC1COC1 ; *C1CCOC1]

12. (canceled)

13. (canceled)

14. (canceled)

15. (canceled)

16. (canceled)

17. A The compound of claim 1, wherein R⁴ is CH₃ or cyclobutyl.

18. (canceled)

19. The compound of claim 1, wherein X is O.

20. (canceled)

21. (canceled)

22. The compound of claim 1, wherein the compound is
N-(4-(3-(5-Fluoropyridin-2-yl)-1-methyl-1H-pyrazol-4-yl)pyridin-2-yl)propionamide;
4-(3-(5-Fluoropyridin-2-yl)-1-methyl-1H-pyrazol-4-yl)-6,7-dihydro-5H-cyclopenta[b]pyridine;
4-(3-(5-Fluoropyridin-2-yl)-1-methyl-1H-pyrazol-4-yl)-1,3-dihydro-2H-pyrrolo[2,3-b]pyridin-2-one;
7-(3-(5-Fluoropyridin-2-yl)-1-methyl-1H-pyrazol-4-yl)-1H-pyrrolo[3,2-b]pyridine;
7-(3-(5-Fluoropyridin-2-yl)-1-methyl-1H-pyrazol-4-yl)-1H-pyrazolo[4,3-b]pyridine;
5-(1-Methyl-4-(1H-pyrrolo[2,3-b]pyridin-4-yl)-1H-pyrazol-3-yl)oxazole;
2-(1-Methyl-4-(1H-pyrrolo[2,3-b]pyridin-4-yl)-1H-pyrazol-3-yl)oxazole;
2-(1-Methyl-4-(6-methyl-1H-pyrazolo[3,4-b]pyridin-4-yl)-1H-pyrazol-3-yl)oxazole;
4-(1-Methyl-3-(1-methyl-1H-imidazol-4-yl)-1H-pyrazol-4-yl)-1H-pyrrolo[2,3-b]pyridine;
4-(1-Methyl-3-(1-methyl-1H-imidazol-5-yl)-1H-pyrazol-4-yl)-1H-pyrrolo[2,3-b]pyridine;
4-(1-Methyl-3-(1-methyl-1H-imidazol-2-yl)-1H-pyrazol-4-yl)-1H-pyrrolo[2,3-b]pyridine;
5-(1-Methyl-4-(1H-pyrrolo[2,3-b]pyridin-4-yl)-1H-pyrazol-3-yl)isothiazole;
4-(1-Methyl-4-(1H-pyrrolo[2,3-b]pyridin-4-yl)-1H-pyrazol-3-yl)thiazole;
5-(1-Methyl-4-(1H-pyrrolo[2,3-b]pyridin-4-yl)-1H-pyrazol-3-yl)thiazole;
3-(1-Methyl-4-(1H-pyrrolo[2,3-b]pyridin-4-yl)-1H-pyrazol-3-yl)isothiazole;
2-(1-Methyl-4-(1H-pyrrolo[2,3-b]pyridin-4-yl)-1H-pyrazol-3-yl)thiazole;
4-[3-(3-Fluoro-4-pyridyl)-1-methyl-pyrazol-4-yl]-1H-pyrrolo[2,3-b]pyridine;
4-[3-(3-Fluoro-4-pyridyl)-1-methyl-pyrazol-4-yl]-1H-pyrazolo[3,4-b]pyridine;
4-[3-(5-Fluoro-2-pyridyl)-1-methyl-pyrazol-4-yl]-1H-pyrrolo[2,3-b]pyridine;
4-[3-(5-Fluoro-3-pyridyl)-1-methyl-pyrazol-4-yl]-1H-pyrrolo[2,3-b]pyridine;
4-[3-(5-Chloro-2-pyridyl)-1-methyl-pyrazol-4-yl]-1H-pyrrolo[2,3-b]pyridine;
4-(3-(5-Chloropyridin-2-yl)-1-methyl-1H-pyrazol-4-yl)-1H-pyrazolo[3,4-b]pyridine;
4-(3-(5-Chloropyridin-2-yl)-1-methyl-1H-pyrazol-4-yl)-6-methyl-1H-pyrazolo[3,4-b]pyridine;
4-[3-(4-Fluoro-2-pyridyl)-1-methyl-pyrazol-4-yl]-1H-pyrrolo[2,3-b]pyridine;
4-[3-(6-Fluoro-3-pyridyl)-1-methyl-pyrazol-4-yl]-1H-pyrrolo[2,3-b]pyridine;
4-[3-(6-Fluoro-2-pyridyl)-1-methyl-pyrazol-4-yl]-1H-pyrrolo[2,3-b]pyridine;
4-[3-(3,5-Difluoro-2-pyridyl)-1-methyl-pyrazol-4-yl]-1H-pyrrolo[2,3-b]pyridine;
4-(3-(3,5-Difluoropyridin-2-yl)-1-methyl-1H-pyrazol-4-yl)-1H-pyrazolo[3,4-b]pyridine;
4-[3-(5-Fluoropyrimidin-2-yl)-1-methyl-pyrazol-4-yl]-1H-pyrrolo[2,3-b]pyridine;
4-(1-Methyl-3-pyrimidin-4-yl-pyrazol-4-yl)-1H-pyrrolo[2,3-b]pyridine;
4-(1-Methyl-3-pyrimidin-5-yl-pyrazol-4-yl)-1H-pyrrolo[2,3-b]pyridine;
4-(1-Methyl-3-pyrazin-2-yl-pyrazol-4-yl)-1H-pyrrolo[2,3-b]pyridine;
4-[1-Methyl-3-(5-methylpyrazin-2-yl)pyrazol-4-yl]-1H-pyrrolo[2,3-b]pyridine;
4-(1-Methyl-3-pyridazin-3-yl-pyrazol-4-yl)-1H-pyrrolo[2,3-b]pyridine;
4-(1-Methyl-3-pyridazin-4-yl-pyrazol-4-yl)-1H-pyrrolo[2,3-b]pyridine;
4-[3-(5-Fluoro-2-pyridyl)-1-(oxetan-3-ylmethyl)pyrazol-4-yl]-6-methyl-1H-pyrazolo[3,4-b]pyridine;
4-(3-(5-Fluoropyridin-2-yl)-1,5-dimethyl-1H-pyrazol-4-yl)-1H-pyrrolo[2,3-b]pyridine;
4-(5-Cyclobutyl-3-(5-fluoropyridin-2-yl)-1-methyl-1H-pyrazol-4-yl)-1H-pyrrolo[2,3-b]pyridine;
4-[3-(5-Fluoro-2-pyridyl)-1-(trideuteriomethyl)pyrazol-4-yl]-1H-pyrrolo[2,3-b]pyridine;
4-[1-Ethyl-3-(5-fluoro-2-pyridyl)pyrazol-4-yl]-1H-pyrrolo[2,3-b]pyridine;
4-(1-Ethyl-3-(5-fluoropyridin-2-yl)-1H-pyrazol-4-yl)-1H-pyrazolo[3,4-b]pyridine;
4-(1-Ethyl-3-(5-fluoropyridin-2-yl)-1H-pyrazol-4-yl)-6-methyl-1H-pyrazolo[3,4-b]pyridine;
4-[3-(5-Fluoro-2-pyridyl)-1-isopropyl-pyrazol-4-yl]-1H-pyrrolo[2,3-b]pyridine;
4-(3-(5-Fluoropyridin-2-yl)-1-isopropyl-1H-pyrazol-4-yl)-1H-pyrazolo[3,4-b]pyridine;
4-(3-(5-Fluoropyridin-2-yl)-1-isopropyl-1H-pyrazol-4-yl)-6-methyl-1H-pyrazolo[3,4-b]pyridine;
4-[3-(5-Fluoro-2-pyridyl)-1-isobutyl-pyrazol-4-yl]-1H-pyrrolo[2,3-b]pyridine;
4-(3-(5-Fluoropyridin-2-yl)-1-isobutyl-1H-pyrazol-4-yl)-1H-pyrazolo[3,4-b]pyridine;
4-(3-(5-Fluoropyridin-2-yl)-1-isobutyl-1H-pyrazol-4-yl)-6-methyl-1H-pyrazolo[3,4-b]pyridine;
4-(3-(5-Fluoropyridin-2-yl)-1-(2-methoxyethyl)-1H-pyrazol-4-yl)-6-methyl-1H-pyrazolo[3,4-b]pyridine;
4-[1-(Difluoromethyl)-3-(5-fluoro-2-pyridyl)pyrazol-4-yl]-1H-pyrrolo[2,3-b]pyridine;
4-(1-(Difluoromethyl)-3-(5-fluoropyridin-2-yl)-1H-pyrazol-4-yl)-1H-pyrazolo[3,4-b]pyridine;
4-(1-(Difluoromethyl)-3-(5-fluoropyridin-2-yl)-1H-pyrazol-4-yl)-6-methyl-1H-pyrazolo[3,4-b]pyridine;
4-[1-(2-Fluoroethyl)-3-(5-fluoro-2-pyridyl)pyrazol-4-yl]-1H-pyrrolo[2,3-b]pyridine;
4-(1-(2,2-Difluoroethyl)-3-(5-fluoropyridin-2-yl)-1H-pyrazol-4-yl)-6-methyl-1H-pyrazolo[3,4-b]pyridine;
4-(3-(5-Fluoropyridin-2-yl)-1-(2,2,2-trifluoroethyl)-1H-pyrazol-4-yl)-6-methyl-1H-pyrazolo[3,4-b]pyridine;
4-(1-Cyclobutyl-3-(5-fluoropyridin-2-yl)-1H-pyrazol-4-yl)-6-methyl-1H-pyrazolo[3,4-b]pyridine;
4-[3-(5-Fluoro-2-pyridyl)-1-(oxetan-3-yl)pyrazol-4-yl]-1H-pyrrolo[2,3-b]pyridine;
4-(3-(5-Fluoropyridin-2-yl)-1-(oxetan-3-yl)-1H-pyrazol-4-yl)-6-methyl-1H-pyrazolo[3,4-b]pyridine;
(R/S)-4-[3-(5-Fluoro-2-pyridyl)-1-tetrahydrofuran-3-yl-pyrazol-4-yl]-1H-pyrrolo[2,3-b]pyridine;
3-Bromo-4-[3-(5-fluoro-2-pyridyl)-1-methyl-pyrazol-4-yl]-1H-pyrrolo[2,3-b]pyridine;
3-Chloro-4-[3-(5-fluoro-2-pyridyl)-1-methyl-pyrazol-4-yl]-1H-pyrrolo[2,3-b]pyridine;
3-Fluoro-4-[3-(5-fluoro-2-pyridyl)-1-methyl-pyrazol-4-yl]-1H-pyrrolo[2,3-b]pyridine;
5-Fluoro-4-[3-(5-fluoro-2-pyridyl)-1-methyl-pyrazol-4-yl]-1H-pyrrolo[2,3-b]pyridine;
4-[3-(5-Fluoro-2-pyridyl)-1-methyl-pyrazol-4-yl]-5-methyl-1H-pyrrolo[2,3-b]pyridine;
4-[3-(5-Fluoro-2-pyridyl)-1-methyl-pyrazol-4-yl]-6-methyl-1H-pyrrolo[2,3-b]pyridine;
4-(3-(5-Fluoropyridin-2-yl)-1-methyl-1H-pyrazol-4-yl)-2-methyl-1H-pyrrolo[2,3-b]pyridine;
4-[3-(5-Fluoro-2-pyridyl)-1-methyl-pyrazol-4-yl]-3-methyl-1H-pyrrolo[2,3-b]pyridine;
4-(3-(5-Fluoropyridin-2-yl)-1-methyl-1H-pyrazol-4-yl)-2-isopropyl-1H-pyrrolo[2,3-b]pyridine;
2-(Difluoromethyl)-4-(3-(5-fluoropyridin-2-yl)-1-methyl-1H-pyrazol-4-yl)-1H-pyrrolo[2,3-b]pyridine;
4-(3-(5-Fluoropyridin-2-yl)-1-methyl-1H-pyrazol-4-yl)-2-(trifluoromethyl)-1H-pyrrolo[2,3-b]pyridine;
4-(3-(5-Fluoropyridin-2-yl)-1-methyl-1H-pyrazol-4-yl)-3-(trifluoromethyl)-1H-pyrrolo[2,3-b]pyridine;
4-[3-(5-Fluoro-2-pyridyl)-1-methyl-pyrazol-4-yl]-5-(trifluoromethyl)-1H-pyrrolo[2,3-b]pyridine;
4-[3-(5-Fluoro-2-pyridyl)-1-methyl-pyrazol-4-yl]-1H-pyrrolo[2,3-b]pyridine-3-carbonitrile;
4-[3-(5-Fluoro-2-pyridyl)-1-methyl-pyrazol-4-yl]-1H-pyrrolo[2,3-b]pyridine-5-carbonitrile;
2-[4-[3-(5-Fluoro-2-pyridyl)-1-methyl-pyrazol-4-yl]-1H-pyrrolo[2,3-b]pyridin-5-yl]acetonitrile;
4-(3-(5-Fluoropyridin-2-yl)-1-methyl-1H-pyrazol-4-yl)-5-(oxetan-3-yl)-1H-pyrrolo[2,3-b]pyridine;
4-(3-(5-Fluoropyridin-2-yl)-1-methyl-1H-pyrazol-4-yl)-2-(oxetan-3-yl)-1H-pyrrolo[2,3-b]pyridine;
4-[3-(5-Fluoro-2-pyridyl)-1-methyl-pyrazol-4-yl]-3-(oxetan-3-yl)-1H-pyrrolo[2,3-b]pyridine;
3-(4-(3-(5-Fluoropyridin-2-yl)-1-methyl-1H-pyrazol-4-yl)-1H-pyrrolo[2,3-b]pyridin-2-yl)oxetan-3-ol;
4-(3-(5-Fluoropyridin-2-yl)-1-methyl-1H-pyrazol-4-yl)-5-(oxetan-3-ylmethyl)-1H-pyrrolo[2,3-b]pyridine;
3-(4-(3-(5-Fluoropyridin-2-yl)-1-methyl-1H-pyrazol-4-yl)-1H-pyrrolo[2,3-b]pyridin-2-yl)tetrahydrofuran-3-ol;
(R/S)-4-[3-(5-Fluoro-2-pyridyl)-1-methyl-pyrazol-4-yl]-3-(tetrahydrofuran-3-ylmethyl)-1H-pyrrolo[2,3-b]pyridine;
4-(3-(5-Fluoropyridin-2-yl)-1-methyl-1H-pyrazol-4-yl)-1H-pyrazolo[3,4-b]pyridine;
4-(3-(5-Fluoropyridin-2-yl)-1-methyl-1H-pyrazol-4-yl)-6-methyl-1H-pyrazolo[3,4-b]pyridine;
4-[3-(5-Fluoro-2-pyridyl)-1-methyl-pyrazol-4-yl]-3,6-dimethyl-1H-pyrazolo[3,4-b]pyridine;
6-Cyclopropyl-4-(3-(5-fluoropyridin-2-yl)-1-methyl-1H-pyrazol-4-yl)-1H-pyrazolo[3,4-b]pyridine;
4-(3-(5-Fluoropyridin-2-yl)-1-methyl-1H-pyrazol-4-yl)-7H-pyrrolo[2,3-d]pyrimidine;
N-(4-(3-(5-Fluoropyridin-2-yl)-1-methyl-1H-pyrazol-4-yl)pyridin-2-yl)acetamide;
N-(4-(3-(5-Fluoropyridin-2-yl)-1-methyl-1H-pyrazol-4-yl)pyridin-2-yl)cyclopropanecarboxamide;
4-(3-(5-Fluoropyridin-2-yl)-1-methyl-1H-pyrazol-4-yl)-6,7-dihydro-5H-pyrrolo[3,4-b]pyridine;
7-(3-(5-Fluoropyridin-2-yl)-1-methyl-1H-pyrazol-4-yl)thieno[3,2-b]pyridine;
4-[3-(5-Fluoro-2-pyridyl)-1-methyl-pyrazol-4-yl]-1H-pyrrolo[2,3-b]pyridin-6-amine;
2-[4-[3-(5-Fluoro-2-pyridyl)-1-methyl-pyrazol-4-yl]-1H-pyrrolo[2,3-b]pyridin-3-yl]acetonitrile;
1-Ethyl-5-[3-(5-fluoro-2-pyridyl)-1-methyl-pyrazol-4-yl]pyrazolo[3,4-b]pyridine;
4-[3-(5-Fluoro-2-pyridyl)-1-methyl-pyrazol-4-yl]-3-isopropyl-1H-pyrazolo[3,4-b]pyridine;
4-[3-(5-Fluoro-2-pyridyl)-1-methyl-pyrazol-4-yl]-2-methyl-7H-pyrrolo[2,3-d]pyrimidine;
4-[3-(5-Fluoro-2-pyridyl)-1-methyl-pyrazol-4-yl]-6-methyl-1H-pyrazolo[3,4-d]pyrimidine;
2-Cyclopropyl-4-[3-(5-fluoro-2-pyridyl)-1-methyl-pyrazol-4-yl]-7H-pyrrolo[2,3-d]pyrimidine;
7-[3-(5-Fluoro-2-pyridyl)-1-methyl-pyrazol-4-yl]-3-methyl-thieno[3,2-b]pyridine;
4-[1-Cyclopropyl-3-(5-fluoro-2-pyridyl)pyrazol-4-yl]-6-methyl-1H-pyrazolo[3,4-b]pyridine;
4-[3-(3-Chloro-4-pyridyl)-1-methyl-pyrazol-4-yl]-6-methyl-1H-pyrazolo[3,4-b]pyridine;
4-[3-(5-Chloro-3-pyridyl)-1-methyl-pyrazol-4-yl]-6-methyl-1H-pyrazolo[3,4-b]pyridine;
6-Methyl-4-[1-methyl-3-(6-methylpyridazin-4-yl)pyrazol-4-yl]-1H-pyrazolo[3,4-b]pyridine;
4-[3-(5-Fluoro-2-pyridyl)-1-methyl-pyrazol-4-yl]-1-methyl-pyrrolo[2,3-b]pyridine;
4-[3-(5-Fluoro-2-pyridyl)-1-methyl-pyrazol-4-yl]thieno[2,3-b]pyridine; or
6-(Difluoromethyl)-4-[3-(5-fluoro-3-pyridyl)-1-methyl-pyrazol-4-yl]-1H-pyrazolo[3,4-b]pyridine;
and pharmaceutically acceptable salts, isotopes, N-oxides, solvates, and stereoisomers thereof.

23. The compound of claim 1, wherein the compound is
4-[1-Methyl-3-(4-pyridyl)pyrazol-4-yl]-1H-pyrrolo[2,3-b]pyridine;
4-[1-Methyl-3-(6-methyl-3-pyridyl)pyrazol-4-yl]-1H-pyrrolo[2,3-b]pyridine;
4-[3-[6-(Difluoromethoxy)-3-pyridyl]-1-methyl-pyrazol-4-yl]-1H-pyrrolo[2,3-b]pyridine;
6-Methyl-4-(1-methyl-3-(6-methylpyridin-2-yl)-1H-pyrazol-4-yl)-1H-pyrazolo[3,4-b]pyridine;
4-[1-Methyl-3-(5-methyl-2-pyridyl)pyrazol-4-yl]-1H-pyrrolo[2,3-b]pyridine;
4-(1-Methyl-3-(5-methylpyridin-2-yl)-1H-pyrazol-4-yl)-1H-pyrazolo[3,4-b]pyridine;
4-(3-(5-(Difluoromethyl)pyridin-2-yl)-1-methyl-1H-pyrazol-4-yl)-1H-pyrazolo[3,4-b]pyridine;
4-(3-(6-Methoxypyridin-2-yl)-1-methyl-1H-pyrazol-4-yl)-6-methyl-1H-pyrazolo[3,4-b]pyridine;
4-[3-[5-(Difluoromethoxy)-2-pyridyl]-1-methyl-pyrazol-4-yl]-1H-pyrrolo[2,3-b]pyridine;
4-[1-Methyl-3-[5-(trifluoromethoxy)-2-pyridyl]pyrazol-4-yl]-1H-pyrrolo[2,3-b]pyridine;
4-(3-(5-Chloro-6-methylpyridin-2-yl)-1-methyl-1H-pyrazol-4-yl)-6-methyl-1H-pyrazolo[3,4-b]pyridine;
4-(3-(5-Fluoro-6-methylpyridin-2-yl)-1-methyl-1H-pyrazol-4-yl)-6-methyl-1H-pyrazolo[3,4-b]pyridine;
4-(3-(5-Fluoro-6-methoxypyridin-2-yl)-1-methyl-1H-pyrazol-4-yl)-6-methyl-1H-pyrazolo[3,4-b]pyridine;
4-(3-(5-Chloro-6-methoxypyridin-2-yl)-1-methyl-1H-pyrazol-4-yl)-6-methyl-1H-pyrazolo[3,4-b]pyridine;
4-[1-Methyl-3-(3-pyridyl)pyrazol-4-yl]-1H-pyrrolo[2,3-b]pyridine;
8-[3-(5-Fluoro-2-pyridyl)-1-methyl-pyrazol-4-yl]-2-methoxy-1,5-naphthyridine;
4-[3-(5-Fluoro-2-pyridyl)-1-methyl-pyrazol-4-yl]quinoline;
7-Chloro-4-[3-(5-fluoro-2-pyridyl)-1-methyl-pyrazol-4-yl]quinoline;
7-Fluoro-4-[3-(5-fluoro-2-pyridyl)-1-methyl-pyrazol-4-yl]quinoline;
4-[3-(5-Fluoro-2-pyridyl)-1-methyl-pyrazol-4-yl]-7-(trifluoromethyl)quinoline;
4-[3-(5-Fluoro-2-pyridyl)-1-methyl-pyrazol-4-yl]-7-methoxy-quinoline;
4-[3-(5-Fluoro-2-pyridyl)-1-methyl-pyrazol-4-yl]-7-(trifluoromethoxy)quinoline;
4-[3-(5-Fluoro-2-pyridyl)-1-methyl-pyrazol-4-yl]-7-methoxy-2-methyl-quinoline;
7-Fluoro-4-[3-(5-fluoro-2-pyridyl)-1-methyl-pyrazol-4-yl]-6-methoxy-quinoline;
4-[3-(5-Fluoro-2-pyridyl)-1-methyl-pyrazol-4-yl]-1,7-naphthyridine;
4-[3-(5-Fluoro-2-pyridyl)-1-methyl-pyrazol-4-yl]-5,6,7,8-tetrahydro-1,7-naphthyridine;
5-[3-(5-Fluoro-2-pyridyl)-1-methyl-pyrazol-4-yl]-1,2,3,4-tetrahydro-1,8-naphthyridine;
4-[3-(5-Fluoro-2-pyridyl)-1-methyl-pyrazol-4-yl]-1,5-naphthyridine;
2-Fluoro-8-[3-(5-fluoro-2-pyridyl)-1-methyl-pyrazol-4-yl]-1,5-naphthyridine;
2-Ethoxy-8-[3-(5-fluoro-2-pyridyl)-1-methyl-pyrazol-4-yl]-1,5-naphthyridine;
4-[3-(5-Fluoro-2-pyridyl)-1-methyl-pyrazol-4-yl]-6-methoxy-quinoline;
4-[3-(5-Fluoro-2-pyridyl)-1-methyl-pyrazol-4-yl]-1,6-naphthyridine;
8-[3-(5-Fluoro-2-pyridyl)-1-methyl-pyrazol-4-yl]-2-methyl-1,5-naphthyridine;
4-[3-(5-Fluoro-2-pyridyl)-1-methyl-pyrazol-4-yl]-8-methyl-1,5-naphthyridine;
6-Methyl-4-[1-methyl-3-(2-methyl-4-pyridyl)pyrazol-4-yl]-1H-pyrazolo[3,4-b]pyridine;
6-Methyl-4-[1-methyl-3-(3-methyl-4-pyridyl)pyrazol-4-yl]-1H-pyrazolo[3,4-b]pyridine;
6-Methyl-4-[1-methyl-3-(5-methyl-3-pyridyl)pyrazol-4-yl]-1H-pyrazolo[3,4-b]pyridine;
4-[3-(3-Fluoro-5-methyl-4-pyridyl)-1-methyl-pyrazol-4-yl]-6-methyl-1H-pyrazolo[3,4-b]pyridine;
4-[3-(6-Methoxy-5-methyl-3-pyridyl)-1-methyl-pyrazol-4-yl]-6-methyl-1H-pyrazolo[3,4-b]pyridine;
4-[3-(5-Chloro-6-methoxy-3-pyridyl)-1-methyl-pyrazol-4-yl]-6-methyl-1H-pyrazolo[3,4-b]pyridine;
8-Fluoro-4-[3-(5-fluoro-2-pyridyl)-1-methyl-pyrazol-4-yl]quinoline;
4-[3-(5-Fluoro-2-pyridyl)-1-methyl-pyrazol-4-yl]-8-methoxy-quinoline;
4-[3-(5-Fluoro-2-pyridyl)-1-methyl-pyrazol-4-yl]quinoline-7-carbonitrile;
4-[3-(5-Fluoro-2-pyridyl)-1-methyl-pyrazol-4-yl]quinoline-6-carbonitrile;
4-[3-(5-Fluoro-2-pyridyl)-1-methyl-pyrazol-4-yl]-5,7-dimethoxy-quinoline;
3-Fluoro-8-[3-(5-fluoro-2-pyridyl)-1-methyl-pyrazol-4-yl]-1,5-naphthyridine;
3-Bromo-8-[3-(5-fluoro-2-pyridyl)-1-methyl-pyrazol-4-yl]-1,5-naphthyridine;
4-[3-(5-Fluoro-2-pyridyl)-1-methyl-pyrazol-4-yl]-7-methoxy-1,6-naphthyridine;
8-[3-(5-Fluoro-2-pyridyl)-1-methyl-pyrazol-4-yl]-3-methoxy-1,5-naphthyridine; or
7-Fluoro-8-[3-(5-fluoro-2-pyridyl)-1-methyl-pyrazol-4-yl]-2-methoxy-1,5-naphthyridine;
and pharmaceutically acceptable salts, isotopes, N-oxides, solvates, and stereoisomers thereof.

24. The compound of claim 1, wherein the compound is
4-[3-(5-Fluoro-2-pyridyl)-1-methyl-pyrazol-4-yl]-2-methyl-7H-pyrrolo[2,3-d]pyrimidine;
3-Chloro-4-[3-(5-fluoro-2-pyridyl)-1-methyl-pyrazol-4-yl]-1H-pyrrolo[2,3-b]pyridine;
N-(4-(3-(5-Fluoropyridin-2-yl)-1-methyl-1H-pyrazol-4-yl)pyridin-2-yl)propionamide;
4-(3-(5-Fluoropyridin-2-yl)-1-methyl-1H-pyrazol-4-yl)-6-methyl-1H-pyrazolo[3,4-b]pyridine;
4-(1-(2,2-Difluoroethyl)-3-(5-fluoropyridin-2-yl)-1H-pyrazol-4-yl)-6-methyl-1H-pyrazolo[3,4-b]pyridine; or
4-[3-(5-Fluoro-2-pyridyl)-1-methyl-pyrazol-4-yl]-6-methyl-1H-pyrrolo[2,3-b]pyridine;
and pharmaceutically acceptable salts, isotopes, N-oxides, solvates, and stereoisomers thereof.

25. The compound of claim 1, and pharmaceutically acceptable salts, isotopes, N-oxides, solvates, and stereoisomers thereof, having the structure of Formula (IA): 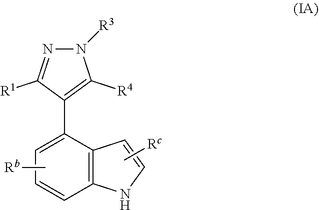
wherein
R¹ is selected from the group consisting of:
(a) 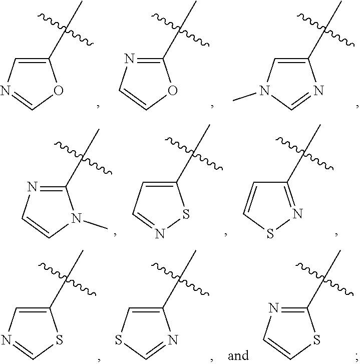
(b) 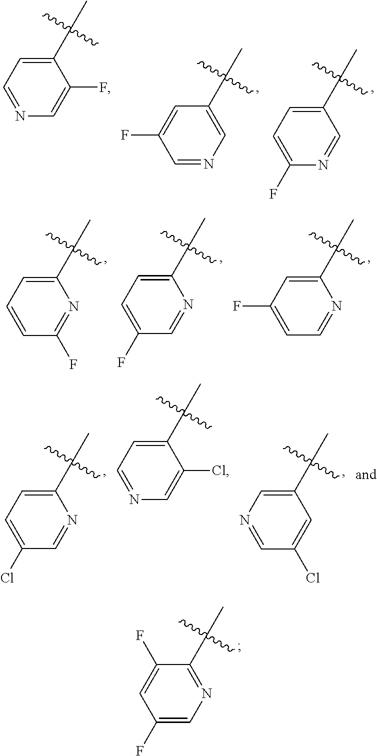
and
(c) 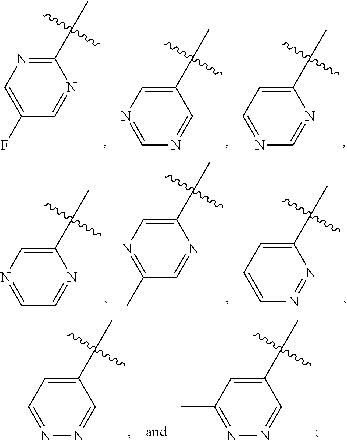
R^{b} is selected from the group consisting of: H, F, CH₃, CF₃, CN, NH₂, 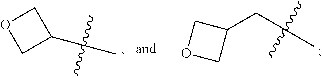 [SMILES: *C1COC1 ; *CC1COC1]
R^{c} is selected from the group consisting of: H, Br, Cl, F, CH₃, CH(CH₃)₂, CHF₂, CF₃, CN, 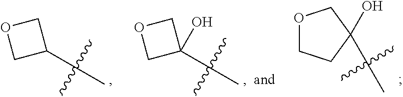 [SMILES: *C1COC1 ; *C1(O)COC1 ; *C1(O)CCOC1]
R³ is selected from the group consisting of: CH₃, CH₂CH₃, CH(CH₃)₂, CH₂CH(CH₃)₂, CHF₂, CH₂CH₂F, 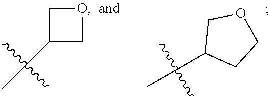
and
R⁴ is selected from the group consisting of: H, CH₃, and cyclobutyl.

26. The compound of claim 1, and pharmaceutically acceptable salts, isotopes, N-oxides, solvates, and stereoisomers thereof, having the structure of Formula (IB):
wherein 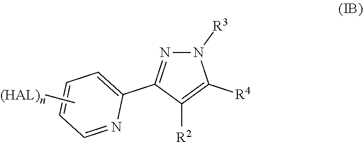
R² is selected from the group consisting of:
(a) 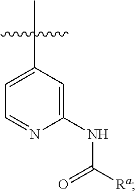
and
(b) 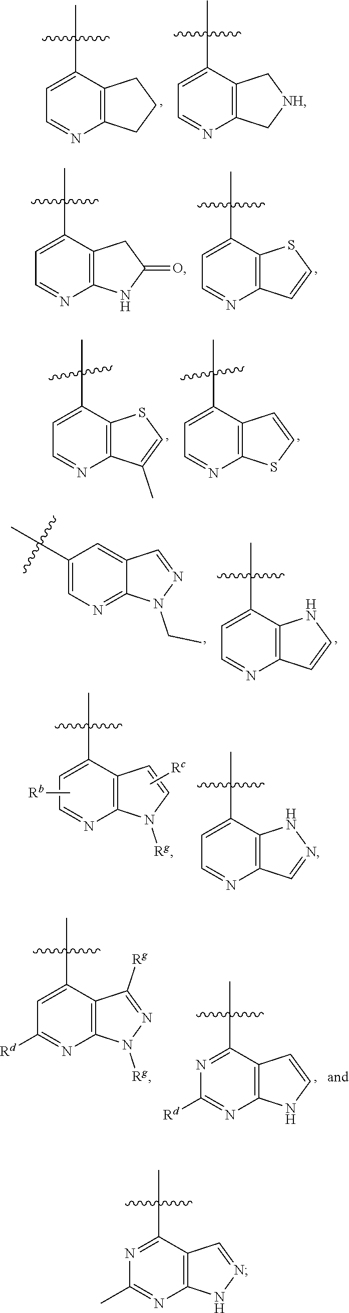
wherein
R^{a} is selected from the group consisting of: CH₃, CH₂CH₃, and cyclopropyl;
R^{b} is selected from the group consisting of: H, F, CH₃, CF₃, CN, NH₂, 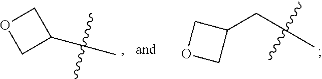 [SMILES: *C1COC1 ; *CC1COC1]
R^{c} is selected from the group consisting of: H, Br, Cl, F, CH₃, CH(CH₃)₂, CHF₂, CF₃, CN, 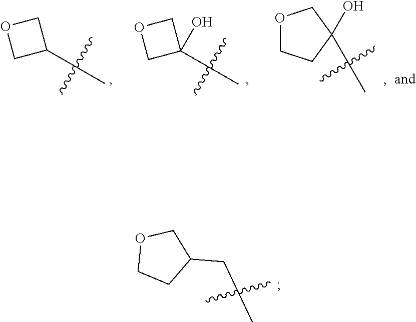 [SMILES: *C1COC1 ; *C1(O)COC1 ; *C1(O)CCOC1 ; *CC1CCOC1]
R^{d} is selected from the group consisting of: H, CH₃, CF₂H, and cyclopropyl;
R^{g} is selected from the group consisting of: H, CH₃, and CH₂CH₃;
HAL is independently selected from: Cl and F;
n is 1 or 2;
R³ is selected from the group consisting of: CH₃, CH₂CH₃, CH(CH₃)₂, CH₂CH(CH₃)₂, CHF₂, CH₂CH₂F, CH₂CHF₂, CH₂CH₂OCH₃, cyclopropyl, cyclobutyl, 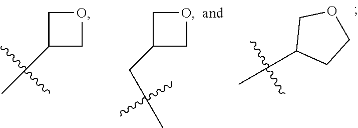
and
R⁴ is selected from the group consisting of: H, CH₃, and cyclobutyl.

27. A pharmaceutical composition comprising:
(A) therapeutically effective amount of at east one compound of Formula (I): 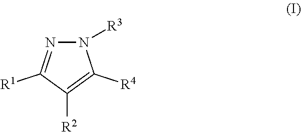
wherein
R¹ is selected from the group consisting of:
(a) 5-membered heteroaryl selected from the group consisting of: 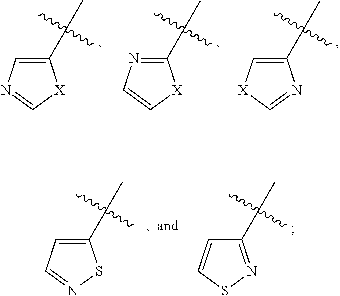
(b) pyridinyl substituted with one or two halo members;
(c) pyrimidinyl; pyrimidinyl substituted with halo; pyrazinyl; pyrazinyl substituted with C₁₋₆ alkyl; pyridazinyl; and pyridazinyl substituted with C₁₋₆ alkyl;
R² is selected from the group consisting of:
(d) 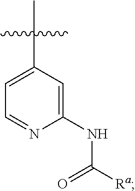
(e) 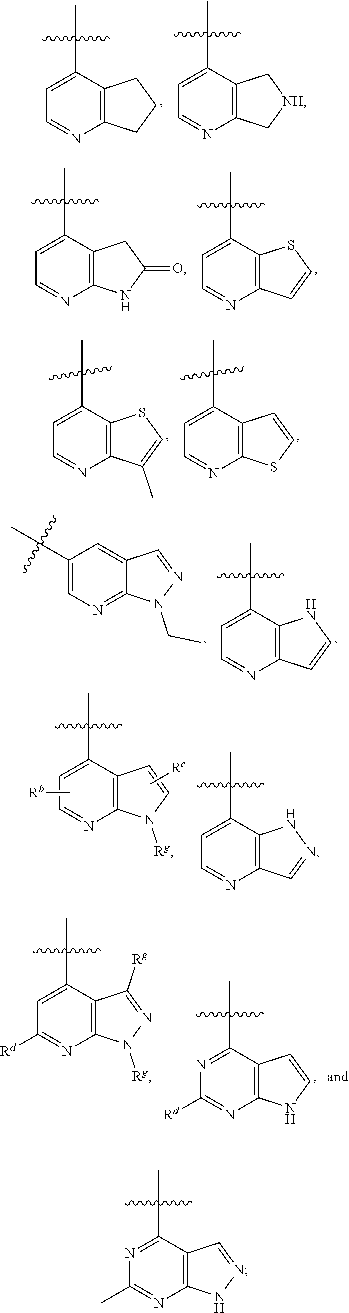
wherein
R^{a} is C₁₋₃ alkyl or C₃₋₆ cycloalkyl;
R^{b} is selected from the group consisting of: H, halo, C₁₋₃ alkyl, C₁₋₃ haloalkyl, CN, CH₂CN, NH₂, oxetanyl, and CH₂-oxetanyl;
R^{c} is selected from the group consisting of: H, halo, C₁₋₃ alkyl, C₁₋₃ haloalkyl, CN, CH₂CN, oxetanyl, oxetanyl substituted with OH, tetrahydrofuranyl substituted with OH, and CH₂-tetrahydrofuranyl;
R^{d} is selected from the group consisting of: H, C₁₋₃ alkyl, C₁₋₃ haloalkyl, and C₃₋₆ cycloalkyl;
R^{g} is H or C₁₋₃ alkyl;
X is O, S, or N—CH₃;
R³ is selected from the group consisting of: C₁₋₆ alkyl, CH₂CH₂OCH₃, C₁₋₆ haloalkyl, C₃₋₆ cycloalkyl, oxetanyl, CH₂-oxetanyl, and tetrahydrofuranyl; and
R⁴ is selected from the group consisting of: H, C₁₋₃ alkyl, and C₃₋₆ cycloalkyl;
and pharmaceutically acceptable salts, isotopes, N-oxides, solvates, and stereoisomers of compounds of Formula (I); and
(B) at least one pharmaceutically acceptable excipient.

28. (canceled)

29. A method of treating a subject suffering from or diagnosed with a disease, disorder, or condition mediated by CSNK1D, comprising administering to a subject in need of such treatment a therapeutically effective amount of at least one compound of Formula (II): 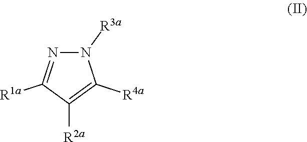
wherein
R^{1a} is selected from the group consisting of:
(a) 5-membered heteroaryl selected from the group consisting of: 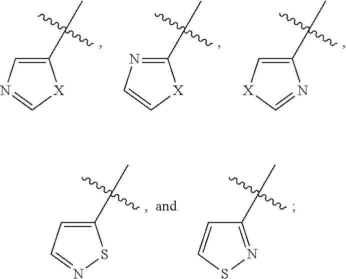
(b) pyridinyl, pyridinyl substituted with one or two members each independently selected from the group consisting of: halo, C₁₋₆ alkyl, C₁₋₆ haloalkyl, OC₁₋₆ alkyl, and OC₁₋₆ haloalkyl;
(c) pyrimidinyl; pyrimidinyl substituted with halo; pyrazinyl; pyrazinyl substituted with C₁₋₆ alkyl; pyridazinyl; and pyridazinyl substituted with C₁₋₆ alkyl;
R^{2a} is selected from the group consisting of:
(d) 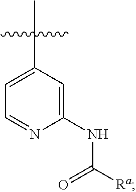
(e) 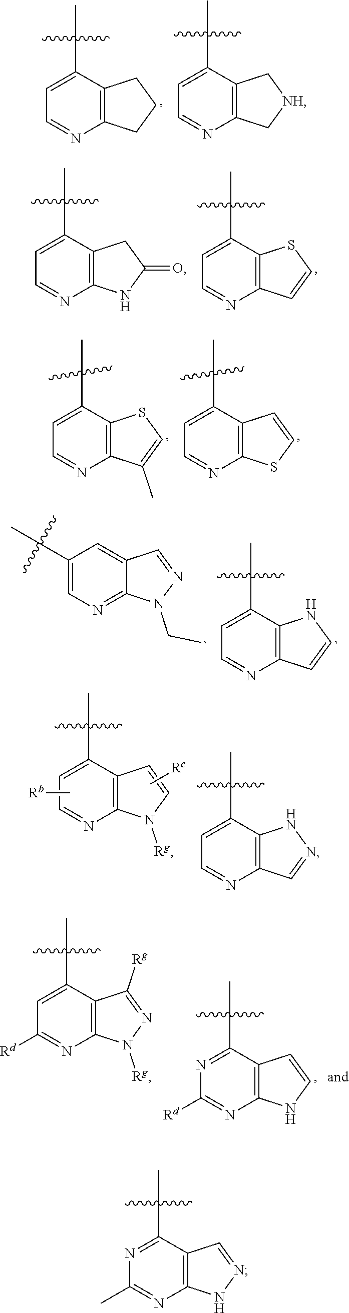
and
(f) 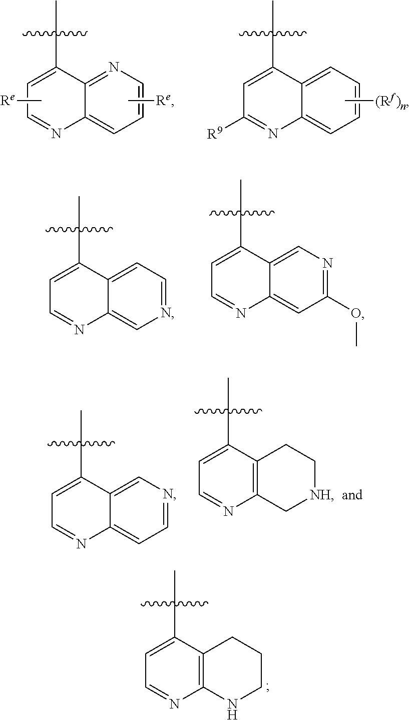
wherein
R^{a} is C₁₋₃ alkyl or C₃₋₆ cycloalkyl;
R^{b} is selected from the group consisting of: H, halo, C₁₋₃ alkyl, C₁₋₃ haloalkyl, CN, CH₂CN, NH₂, oxetanyl, and CH₂-oxetanyl;
R^{c} is selected from the group consisting of: H, halo, C₁₋₃ alkyl, C₁₋₃ haloalkyl, CN, CH₂CN, oxetanyl, oxetanyl substituted with OH, tetrahydrofuranyl substituted with OH, and CH₂-tetrahydrofuranyl;
R^{d} is selected from the group consisting of: H, C₁₋₃ alkyl, C₁₋₃ haloalkyl, and C₃₋₆ cycloalkyl;
R^{e} is selected from the group consisting of: H, halo, C₁₋₃ alkyl, and OC₁₋₃ alkyl;
R^{f} is independently selected from the group consisting of: H, halo, C₁₋₃ alkyl, C₁₋₃ haloalkyl, OC₁₋₃ alkyl, OC₁₋₃ haloalkyl, and CN;
R^{g} is H or C₁₋₃ alkyl;
X is O, S, or N—CH₃;
n is 1 or 2;
R^{3a} is selected from the group consisting of: H, C₁₋₆ alkyl, CH₂CH₂OCH₃, C₁₋₆ haloalkyl, C₃₋₆ cycloalkyl, oxetanyl, CH₂-oxetanyl, and tetrahydrofuranyl; and
R^{4a} is selected from the group consisting of: H, C₁₋₃ alkyl, and C₃₋₆ cycloalkyl;
and pharmaceutically acceptable salts, isotopes, N-oxides, solvates, and stereoisomers of compounds of Formula (II).

30-32. (canceled)
